(19) United States
(12) Patent Application Publication
(10) Pub. No.: US 2024/0092919 A1
(43) Pub. Date: Mar. 21, 2024

(54) T CELL RECRUITING POLYPEPTIDES BASED ON TCR ALPHA/BETA REACTIVITY

(71) Applicants: Ablynx N.V., Zwijnaarde (BE); Sanofi, Paris (FR)

(72) Inventors: Katrijn Neyt, Zwijnaarde (BE); Annelies Roobrouck, Zwijnaarde (BE); Stephanie Staelens, Zwijnaarde (BE); Tom VAN BELLE, Zwijnaarde (BE); Judith Verhelst, Zwijnaarde (BE); Diane Van Hoorick, Zwijnaarde (BE)

(73) Assignees: AblynX N. V., Zwijnaarde (BE); Sanofi, Paris (FR)

(21) Appl. No.: 18/267,257

(22) PCT Filed: Dec. 20, 2021
(86) PCT No.: PCT/EP2021/086843

§ 371 Date: Jun. 14, 2023

(30) Foreign Application Priority Data
Dec. 18, 2020 (EP) 20306608.9
Dec. 17, 2021 (EP) EP21306822.4
Dec. 17, 2021 (WO) PCT/EP2021/086556

(51) Int. Cl. C07K 16/28; A61P 35/02

(52) U.S. Cl. CPC C07K 16/2866; A61P 35/02; C07K 16/2809; A61K 2039/505; C07K 2317/21; C07K 2317/31; C07K 2317/565; C07K 2317/94

(57) ABSTRACT

The present technology provides T cell recruiting polypeptides that specifically bind to the constant domain of a human and of a non-human primate TCR. The present technology also provides nucleic acids, vectors, and compositions. The polypeptides can be used in methods for treatment of cancer.

### 1 FIELD OF THE PRESENT TECHNOLOGY

The present technology provides multispecific T cell recruiting polypeptides binding both to the constant domain of a human T cell receptor (TCR) on a T cell and to the constant domain of a non-human primate TCR on a T cell, and at least one antigen on a target cell. It also relates to the monovalent TCR binding polypeptides for use in these multispecific polypeptides. The present technology further provides nucleic acids encoding said polypeptides as well as vectors, hosts and methods for the production of these polypeptides. Moreover, the present technology relates to methods for treatment making use of the polypeptides of the present technology and kits providing the same.

### 2 TECHNOLOGICAL BACKGROUND

Cancer takes an enormous human toll around the world. It is nowadays the second leading cause of death globally, only preceded by heart disease and stroke. Cancers figure among the leading causes of morbidity and mortality worldwide, with approximately 18.1 million new cases and 9.6 million cancer related deaths in 2018. The number of new cases is expected to rise further over the next decades. Population growth, ageing and lifestyle changes have been described as contributing factors to the increasing cancer burden. (source: WHO Cancer) The total economic impact of premature death and disability from cancer worldwide was already about $900 billion in 2008, which represented 1.5% of the world's gross domestic product at that time.

Available treatment regimens for solid tumours typically include a combination of surgical resection, chemotherapy and radiotherapy. In 40 years of clinical experience little progress has been achieved, especially in advanced stages of cancer. New therapies combatting cancer are eagerly awaited.

Antibody therapy is now an important part of the physician's armamentarium to battle diseases and especially cancer. Monoclonal antibodies have been established as a key therapeutic approach for a range of diseases already for several years. Most of the contemporaneously approved antibody therapies rely on monospecific monoclonal antibodies (mAbs). Until today, most of the targets of the mAbs require either an agonistic or an antagonistic approach. Whereas targeting of cell-surface antigens themselves can mediate antitumour activity through the induction of apoptosis, most mAb-based activity against hematologic malignancies is reliant on either Fc-mediated effector functions such as complement dependent cytotoxicity (CDC) and antibody-dependent cell-mediated cytotoxicity (ADCC).

More recently, immunotherapy has emerged as a rapidly growing area of cancer research. Immunotherapy is directing the body's immune surveillance system, and in particular T cells, to cancer cells.

Cytotoxic T cells (CTL) are T lymphocytes that kill cancer cells, cells that are infected (particularly with viruses), or cells that are damaged in other ways. T lymphocytes (or T cells) express the T cell receptor or TCR molecule and the CD3 receptor on the cell surface. The αβ TCR-CD3 complex (or “TCR complex”) is composed of six different type I single-spanning transmembrane proteins: the TCRα and TCRβ chains that form the TCR heterodimer responsible for ligand recognition, and the non-covalently associated CD3γ, CD3δ, CD3ε and ζ chains, which bear cytoplasmic sequence motifs that are phosphorylated upon receptor activation and recruit a large number of signalling components (Call et al. 2004, Molecular Immunology 40: 1295-1305).

Both α and β chains of the T cell receptor consist of a constant domain and a variable domain. Physiologically, the at chains of the T cell receptor recognize the peptide loaded MHC complex and couple upon engagement to the CD3 chains. These CD3 chains subsequently transduce the engagement signal to the intracellular environment.

Considering the potential of naturally occurring cytotoxic T lymphocytes (CTLs) to mediate cell lysis, various strategies have been explored to recruit immune cells to mediate tumour cell killing. Since T lymphocytes lack the expression of Fc receptors, they are not recruited to a tumour site through the Fc tail of an anti-tumour monoclonal antibody. As an alternative, the patient's T cells were modified with a second TCR of known specificity for a defined tumour antigen. This adoptive cell transfer is by nature highly personalized and labour intensive. However, the main problem of T cell therapies remains the large number of immune escape mechanisms known to occur in cancer patients (Nagorsen et al. 2012, Pharmacology & Therapeutics 136: 334-342).

Rather than eliciting specific T cell responses, which rely on expression by cancer cells of MHC molecules and the presence, generation, transport and display of specific peptide antigens, more recent developments have attempted to combine the advantages of immunotherapy with antibody therapy by engaging all T cells of a patient in a polyclonal fashion via recombinant antibody based technologies: “bispecifics”.

Bispecific antibodies have been engineered that have a tumour recognition part on the one arm (target-binding arm) whereas the other arm of the molecule has specificity for a T cell antigen (effector-binding arm), often CD3. Through the simultaneous binding of the two arms to their respective antigens, T lymphocytes are directed towards and activated at the tumour cell where they can exert their cytolytic function.

The concept of using bispecific antibodies to activate T cells against tumour cells was described more than 30 years ago, but manufacturing problems and clinical failures sent the field into stagnation. Further progress was made when smaller format bispecifics, resulting from the reduction of antibodies to their variable fragments, were developed.

Currently, only one bispecific antibody, Blinatumomab (a BiTE molecule recognizing CD19 and CD3), is on the market for use in the clinic for treatment of cancer. Although this T cell engaging format was approved in December 2014 for second line treatment by the FDA, many hurdles had to be overcome. The first clinical trials of Blinatumomab were prematurely stopped due to neurologic adverse events, cytokine release syndrome (CRS) and infections on the one hand and the absence of objective clinical responses or robust signs of biological activity on the other hand. CRS is the most important adverse event reported in the first T cell-engaging therapies.

In order to minimise the risk for adverse events and systemic side effects, such as cytokine storms, utmost care must be taken upon selection of the T cell antigen arm. The latter must bind to the TCR complex in a monovalent fashion and may not trigger T cell signaling in the absence of the targeted cancer cells. Only the specific binding of both arms of the bispecific antibody to their targets (the tumour and the T cell antigen) may trigger the formation of the cytolytic synapses and subsequent killing of the tumour cells.

Non-human primates, such as cynomolgus or rhesus monkeys, are generally considered to be the most suitable animal species for preclinical studies, including efficacy and toxicity studies. To enable assessment of toxicity of a bispecific T cell engaging antibody in non-human primates, good species cross-reactivity for human and non-human primate TCR of an antibody is advisable.

While the majority of T cell activating bispecific antibodies target the CD3 receptor on the T cell, some bispecific antibodies that thereto target the constant domain of the T cell receptor have been described in WO2016180969A1.

However, there remains a need for additional multispecific T cell engaging formats, in particular multispecific T cell engaging formats targeting T cell receptors different from CD3.

### 3 SUMMARY OF THE PRESENT TECHNOLOGY

An objective of the present technology is to provide
Immunoglobulin single variable domains (ISVDs), and polypeptides comprising the same, with improved binding to a constant domain of a human TCR;
ISVDs, and polypeptides comprising the same, with improved binding to a constant domain of non-human primate TCR;
ISVDs, and polypeptides comprising the same, with improved binding to a constant domain of a human and of a non-human primate TCR; and/or
ISVDs, and polypeptides comprising the same, with improved cross-reactivity for binding to human and non-human primate TCR;
ISVDs with improved formatting properties;
polypeptides comprising an ISVD with improved binding to a constant domain of a human and/or of a non-human primate TCR and an ISVD that binds to at least one antigen on a target cell; and
polypeptides comprising an ISVD with improved cross-reactivity for binding to human and non-human primate TCR and an ISVD that binds to at least one antigen on a target cell.

The present inventors conducted dedicated studies to improve the binding characteristics of ISVD T01700056G05, previously described in WO2016180969. ISVD T01700056G05 is capable of lysing the target cells when formatted into a multispecific format with one or more tumour binding ISVDs that bind to one or more antigens on the target cells. Tumour cell killing was demonstrated upon combination of ISVD T01700056G05 with ISVDs binding to different tumour associated antigens, reflecting the broad applicability of these bispecific antibodies. Moreover, these multispecific formats remained active when bound to albumin, contributing to a favourable PK profile and patient compliance. While ISVD T0170056G05 was shown to enable tumour cell killing when formatted into a multispecific polypeptide in combination with one or more ISVD which binds to an antigen on a target cell, the affinity of said ISVD for binding to cynomolgus TCR was found to be considerably lower compared to the affinity of said ISVD for binding to human TCR. To enable assessment of toxicity of a multispecific T cell engaging polypeptide in preclinical studies conducted on non-human primates, good species cross-reactivity for human and non-human primate of an antibody is highly advisable.

The present inventors identified certain amino acid mutations—and combinations thereof—which upon introduction in the CDRs of ISVD T0170056G05 result in improved binding of said ISVD to the constant domains of a human TCR and/or a non-human primate TCR. Additionally, amino acid mutations and combinations thereof were identified that result in improved cross-reactivity for binding to human and non-humane primate TCR, which is beneficial for assessment of toxicity in non-humane primate species, such as cynomolgus or rhesus monkeys. Polypeptides of the present technology, comprising such an ISVD with improved binding properties and further comprising one or more ISVDs specifically binding to an antigen on a target cell, were capable of redirecting a T cell to a target cell and subsequently inducing T cell activation resulting in lysis of the target cell.

The present inventors identified an ISVD, which in the form of a multispecific T cell engaging polypeptide, surprisingly showed improved potency for T cell activation and improved cross-reactivity for binding to human and non-human primate TCR both when positioned in a C-terminal or N-terminal position in said polypeptide. These polypeptides with improved human/cyno cross-reactivity and potency only showed effects when bound both to the T cell and the target cell. Additionally, no safety related issues could be observed for these polypeptides with improved human/cyno cross-reactivity in an efficacy study in cynomolgues monkeys. Prolonged efficacy could be observed for such a polypeptide in cynomolgus monkeys, as compared to the same polypeptide comprising ISVD T0170056G05 instead.

In some embodiments, the present technology thus provides a polypeptide comprising a first and at least one further ISVD, wherein said first ISVD specifically binds to a constant domain of a human and/or of a non-human primate TCR present on a T cell, and the at least one further ISVD specifically binds to an antigen on a target cell.

In some embodiments, the polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units. For example, the binding unit can be an ISVD that binds to a (human) serum protein, such as human serum albumin.

Also provided is a nucleic acid molecule encoding the polypeptide of the present technology or a vector comprising the nucleic acid.

The present technology also relates to a host cell transformed or transfected with the nucleic acid or vector that encodes the polypeptide according to the present technology.

Further provided is a process for the production of the polypeptide of the present technology, said method comprising the steps of culturing a host cell transformed or transfected with a nucleic acid or vector that encodes the polypeptide according to the present technology under conditions allowing the expression of said polypeptide, and recovering the produced polypeptide from the culture.

In some embodiments, the polypeptide of the present technology is comprised in a composition, such as a pharmaceutical composition.

Moreover, the present technology relates to the polypeptides for use as a medicament. In one embodiment, the polypeptide for use in the treatment of a proliferative disease, an inflammatory disease, an infectious disease or an autoimmune disease. In one embodiment, said proliferative disease is cancer. The present technology also provides methods for treating those diseases comprising the step of administering the polypeptides to a subject in need thereof, as well as the polypeptides for use in the preparation of a medicament for treating those disease.

In particular, the present technology provides the following embodiments:
Embodiment 1. Polypeptide comprising a first and a second immunoglobulin single variable domain (ISVD), wherein
   said first ISVD specifically binds to the constant domain of a human and of a non-human primate T cell receptor (TCR) present on a T cell;
   said second ISVD specifically binds to a first antigen on a target cell;
   wherein said first antigen is different from said TCR;
   wherein said target cell is different from said T cell; and
   wherein said first ISVD essentially consists of 4 framework regions (FR1 to FR4, respectively) and 3 complementarity determining regions (CDR1 to CDR3, respectively), wherein
   (i) the amino acid sequence of CDR1 is Xaa Xaa Val His Xaa Xaa Xaa Xaa Xaa Xaa (SEQ ID NO: 295), wherein
      Xaa at position 1 is Gly, Trp, Gln, or Tyr,
      Xaa at position 2 is Asp, Glu, Tyr, Ser, or Ala,
      Xaa at position 5 is Lys, or Gln,
      Xaa at position 6 is lie, or Leu,
      Xaa at position 7 is Asn, or Phe,
      Xaa at position 8 is Phe, Val, or Tyr,
      Xaa at position 9 is Leu, Tyr, or Phe,
      Xaa at position 10 is Gly, Thr, or Arg,
   (ii) the amino acid sequence of CDR2 is Xaa lie Ser lie Xaa Asp Xaa Xaa Asp (SEQ ID NO: 296), wherein
      Xaa at position 1 is His, or Val,
      Xaa at position 5 is Gly, or Ala,
      Xaa at position 7 is Gln, Val, or Glu,
      Xaa at position 8 is Thr, Ser, or Val,
   (iii) the amino acid sequence of CDR3 is Xaa Ser Xaa lie Xaa Pro Tyr Xaa Xaa (SEQ ID NO: 297), wherein
      Xaa at position 1 is Phe, Leu or Met,
      Xaa at position 3 is Arg, Lys, or Ser,
      Xaa at position 5 is Tyr, or Trp,
      Xaa at position 8 is Asp, or Gln,
      Xaa at position 9 is Tyr, or Trp, and
   wherein CDR1, CDR2, CDR3 of said first ISVD are not simultaneously the amino acid sequences of SEQ ID NO's: 153, 209 and 223, respectively.

Preferably, the amino acid sequence of CDR1 as defined in Embodiment 1 has 1, or 2, or 3, or 4, or 5, or 6, or 7, or 8 amino acid difference(s) with SEQ ID NO: 153. More preferably, the amino acid sequence of CDR1 as defined in Embodiment 1 has 1, or 2, or 3, or 4, or 5, or 6 amino acid difference(s) with SEQ ID NO: 153. Even more preferably, the amino acid sequence of CDR1 as defined in Embodiment 1 has 1, or 2 amino acid difference(s) with SEQ ID NO: 153.

Preferably, the amino acid sequence of CDR2 as defined in Embodiment 1 has 1, or 2, or 3, or 4 amino acid difference(s) with SEQ ID NO: 209. More preferably, the amino acid sequence of CDR2 as defined in Embodiment 1 has 1 amino acid difference(s) with SEQ ID NO: 209. Even more preferably, the amino acid sequence of CDR2 as defined in Embodiment 1 is SEQ ID NO: 209.

Preferably, the amino acid sequence of CDR3 as defined in Embodiment 1 has 1, or 2, or 3, or 4, or 5 amino acid difference(s) with SEQ ID NO: 223. More preferably, the amino acid sequence of CDR3 as defined in Embodiment 1 has 1, or 2, or 3, or 4 amino acid difference(s) with SEQ ID NO: 223. Even more preferably, the amino acid sequence of CDR3 as defined in Embodiment 1 has 1, or 2 amino acid difference(s) with SEQ ID NO: 223.

Preferably, said first ISVD as defined in Embodiment 1 essentially consists of 4 framework regions (FR1 to FR4, respectively) and 3 complementarity determining regions (CDR1 to CDR3, respectively), wherein
(i) the amino acid sequence of CDR1 is as defined in Embodiment 1 and has 1, or 2, or 3, or 4, or 5, or 6, or 7, or 8 amino acid difference(s) with SEQ ID NO: 153, preferably has 1, or 2, or 3, or 4, or 5, or 6 amino acid difference(s) with SEQ ID NO: 153 and even more preferably has 1, or 2 amino acid difference(s) with SEQ ID NO: 153;
(ii) the amino acid sequence of CDR2 is is as defined in Embodiment 1 and has 1, or 2, or 3, or 4 amino acid difference(s) with SEQ ID NO: 209, more preferably has 1 amino acid difference(s) with SEQ ID NO: 209, and even more preferably, is SEQ ID NO: 209;
(iii) the amino acid sequence of CDR3 is as defined in Embodiment 1 and has 1, or 2, or 3, or 4, or 5 amino acid difference(s) with SEQ ID NO: 223, more preferably has 1, or 2, or 3, or 4 amino acid difference(s) with SEQ ID NO: 223, and even more preferably, has 1, or 2 amino acid difference(s) with SEQ ID NO: 223.
Embodiment 2. The polypeptide according to embodiment 1, wherein said first ISVD specifically binds to the constant domain of a human T cell receptor α (TCR-α) (SEQ ID NO: 291) and/or the constant domain of the human T cell receptor β (TCR-β) (SEQ ID NO: 292), or polymorphic variants or isoforms thereof.
Embodiment 3. Polypeptide according to embodiment 1 or 2, wherein the non-human primate TCR is a macaque or rhesus TCR.
Embodiment 4. Polypeptide according to embodiment 3, wherein the macaque or rhesus TCR comprises the constant domain of a TCR-α of SEQ ID NO: 293 and/or of a TCR-β of SEQ ID NO: 294, or polymorphic variants or isoforms thereof.
Embodiment 5 Polypeptide according to any of embodiments 1 to 4, wherein the first ISVD binds to human TCR with the same or lower off rate constant (k_{off}) compared to an ISVD of SEQ ID NO: 2, preferably as measured by surface plasmon resonance (SPR) preferably performed on a ProteOn XPR36 instrument, preferably at 25° C.

Preferably, the first ISVD of the polypeptide as defined in Embodiment 5 binds to human TCR with the same or lower K_{D} compared to an ISVD of SEQ ID NO: 2, preferably as measured by surface plasmon resonance (SPR) preferably performed on a ProteOn XPR36 instrument, preferably at 25° C.
Embodiment 6. Polypeptide according to any of embodiments 1 to 5, wherein the first ISVD binds to non-human primate TCR with the same or lower k_{off} compared to an ISVD of SEQ ID NO: 2, preferably as measured by SPR preferably performed on a ProteOn XPR36 instrument, preferably at 25° C.

Preferably, the first ISVD of the polypeptide as defined in Embodiment 6 binds to non-human primate TCR with the same or lower K_{D} compared to an ISVD of SEQ ID NO: 2, preferably as measured by surface plasmon resonance (SPR) preferably performed on a ProteOn XPR36 instrument, preferably at 25° C.
Embodiment 7. Polypeptide according to embodiment 5 or 6, wherein the first ISVD has a k_{off} for binding to human TCR of at most about 10⁻³ s⁻¹, preferably as measured by surface plasmon resonance (SPR), preferably performed on a ProteOn XPR36 instrument, preferably at 25° C.
Embodiment 8. Polypeptide according to any of embodiments 5 to 7, wherein the first ISVD has an off rate constant (k_{off}) for binding to non-human primate TCR selected from the group consisting of at most about 10⁻² s⁻¹, preferably as measured by SPR), preferably performed on a ProteOn XPR36 instrument, preferably at 25° C.
Embodiment 9. Polypeptide according to embodiment 7 or 8, wherein the first ISVD has a k_{off} for binding to non-human primate TCR which is within 5-fold range of the k_{off} for binding to human TCR, preferably as measured by SPR), preferably performed on a ProteOn XPR36 instrument, preferably at 25° C.
Embodiment 10. The polypeptide according to any of embodiments 1 to 9, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 154 to 169.
Embodiment 11. The polypeptide according to any of embodiments 1 to 10, wherein said first ISVD essentially consists of 4 framework regions (FR1 to FR4, respectively) and 3 complementarity determining regions (CDR1 to CDR3, respectively), in which CDR2 is chosen from the group of amino acid sequences of SEQ ID NO's: 210 to 215.
Embodiment 12. The polypeptide according to any of embodiments 1 to 11, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 224 to 230.
Embodiment 13. The polypeptide according to any of embodiments 1 to 10, wherein said first ISVD essentially consists of 4 framework regions (FR1 to FR4, respectively) and 3 complementarity determining regions (CDR1 to CDR3, respectively), in which:
   (i) CDR1 is chosen from the group of amino acid sequences of SEQ ID NO's: 154 to 169;
   (ii) CDR2 consists of the amino acid sequence HISIGDQTD (SEQ ID NO: 209); and
   (iii) CDR3 consists of the amino acid sequence FSRIYPYDY (SEQ ID NO: 223).
Embodiment 14. The polypeptide according to any of embodiments 1 to 9 or 11, wherein said first ISVD essentially consists of 4 framework regions (FR1 to FR4, respectively) and 3 complementarity determining regions (CDR1 to CDR3, respectively), in which:
   (i) CDR1 consists of the amino acid sequence GDVHKINFLG (SEQ ID NO: 153);
   (ii) CDR2 is chosen from the group of amino acid sequences of SEQ ID NO's: 210 to 215; and
   (iii) CDR3 consists of the amino acid sequence FSRIYPYDY (SEQ ID NO: 223).
Embodiment 15. The polypeptide according to any of embodiments 1 to 9 or 12, wherein said first ISVD essentially consists of 4 framework regions (FR1 to FR4, respectively) and 3 complementarity determining regions (CDR1 to CDR3, respectively), in which:
   (i) CDR1 consists of the amino acid sequence GDVHKINFLG (SEQ ID NO: 153);
   (ii) CDR2 consists of the amino acid sequence HISIGDQTD (SEQ ID NO: 209); and
   (iii) CDR3 is chosen from the group of amino acid sequences of SEQ ID NO's: 224 to 230.
Embodiment 16. The polypeptide according to any of embodiments 1 to 9, wherein said first ISVD essentially consists of 4 framework regions (FR1 to FR4, respectively) and 3 complementarity determining regions (CDR1 to CDR3, respectively), in which:
   (i) the amino acid sequence of CDR1 is Xaa Xaa Val His Xaa Xaa Asn Phe Xaa Xaa (SEQ ID NO: 298), wherein
      Xaa at position 1 is Gly, Trp, or Tyr,
      Xaa at position 2 is Asp, Glu, Tyr, Ser, or Ala,
      Xaa at position 5 is Lys, or Gln,
      Xaa at position 6 is lie, or Leu,
      Xaa at position 9 is Leu, or Tyr,
      Xaa at position 10 is Gly, or Thr,
   (ii) the amino acid sequence of CDR2 is His lie Ser lie Gly Asp Gln Thr Asp (SEQ ID NO: 209), and
   (iii) the amino acid sequence of CDR3 is Xaa Ser Xaa lie Xaa Pro Tyr Asp Xaa (SEQ ID NO: 299), wherein
      Xaa at position 1 is Phe, Leu or Met,
      Xaa at position 3 is Arg, or Lys,
      Xaa at position 5 is Tyr, or Trp, and
      Xaa at position 9 is Tyr, or Trp.

Preferably, the amino acid sequence of CDR1 as defined in Embodiment 16 has 1, or 2, or 3, or 4, or 5, or 6 amino acid difference(s) with SEQ ID NO: 153. More preferably, the amino acid sequence of CDR1 as defined in Embodiment 16 has 1, or 2, or 3, or 4 amino acid difference(s) with SEQ ID NO: 153. Even more preferably, the amino acid sequence of CDR1 as defined in Embodiment 16 has 1, or 2 amino acid difference(s) with SEQ ID NO: 153.

Preferably, the amino acid sequence of CDR3 as defined in Embodiment 16 has 1, or 2, or 3, or 4 amino acid difference(s) with SEQ ID NO: 223. More preferably, the amino acid sequence of CDR3 as defined in Embodiment 16 has 1, or 2, or 3 amino acid difference(s) with SEQ ID NO: 223. Even more preferably, the amino acid sequence of CDR3 as defined in Embodiment 16 has 1, or 2 amino acid difference(s) with SEQ ID NO: 223.

Preferably, said first ISVD as defined in Embodiment 16 essentially consists of 4 framework regions (FR1 to FR4, respectively) and 3 complementarity determining regions (CDR1 to CDR3, respectively), wherein
(i) the amino acid sequence of CDR1 is as defined in Embodiment 16 and has 1, or 2, or 3, or 4, or 5, or 6 amino acid difference(s) with SEQ ID NO: 153, preferably has 1, or 2, or 3, or 4 amino acid difference(s) with SEQ ID NO: 153 and even more preferably has 1, or 2 amino acid difference(s) with SEQ ID NO: 153;
(ii) the amino acid sequence of CDR2 is His lie Ser lie Gly Asp Gln Thr Asp (SEQ ID NO: 209), and
(iii) the amino acid sequence of CDR3 is as defined in Embodiment 16 and has 1, or 2, or 3, or 4 amino acid difference(s) with SEQ ID NO: 223, more preferably has 1, or 2, or 3 amino acid difference(s) with SEQ ID NO: 223, and even more preferably, has 1, or 2 amino acid difference(s) with SEQ ID NO: 223.
Embodiment 17. The polypeptide according to embodiment 16, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 154 to 163.
Embodiment 18. The polypeptide according to embodiment 16 or 17, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 154 to 158.
Embodiment 19. The polypeptide according to any of embodiments 16 to 18, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 154 and 155.
Embodiment 20. The polypeptide according to any of embodiments 16 to 19, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 224 to 228.
Embodiment 21. The polypeptide according to any of embodiments 16 to 20, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 224 to 226.
Embodiment 22. The polypeptide according to any of embodiments 16 to 21, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 224 and 225.
Embodiment 23. The polypeptide according to embodiment 16, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 171 to 207.
Embodiment 24. The polypeptide according to embodiments 16 or 23, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 154, 161, 171 to 175, and 177 to 191.
Embodiment 25. The polypeptide according to any of embodiments 16, 23 or 24 wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 171 to 175.
Embodiment 26. The polypeptide according to any of embodiments 16, or 23 to 25, wherein said first ISVD essentially consists of 4 framework regions (FR1 to FR4, respectively) and 3 complementarity determining regions (CDR1 to CDR3, respectively), in wherein CDR1 consists of the amino acid sequence of GYVHKINFYG (SEQ ID NO: 171).
Embodiment 27. The polypeptide according to any of embodiments 16, or 23 to 26, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 235 to 247.
Embodiment 28. The polypeptide according to any of embodiments 16, or 23 to 27, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 226, 227, and 235 to 243.
Embodiment 29. The polypeptide according to any of embodiments 16, or 23 to 28, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 235 and 236.
Embodiment 30. The polypeptide according to any of embodiments 16, or 23 to 28, wherein said first ISVD essentially consists of 4 framework regions (FR1 to FR4, respectively) and 3 complementarity determining regions (CDR1 to CDR3, respectively), in wherein CDR3 consists of the amino acid sequence of LSRIWPYDY (SEQ ID NO: 235).
Embodiment 31. The polypeptide according to any of embodiments 1 to 9, 16, or 23 to 30, wherein said first ISVD essentially consists of 4 framework regions (FR1 to FR4, respectively) and 3 complementarity determining regions (CDR1 to CDR3, respectively), in which:

a)
CDR1 consists of the amino acid sequence of
(SEQ ID NO: 171)
GYVHKINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
b)
CDR1 consists of the amino acid sequence of
(SEQ ID NO: 172)
WDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
c)
CDR1 consists of the amino acid sequence of
(SEQ ID NO: 173)
YDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
d)
CDR1 consists of the amino acid sequence of
(SEQ ID NO: 174)
WDVHQINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
e)
CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
f)
CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 236)
LSKIWPYDY.

Embodiment 32. The polypeptide according to any of embodiments 1 to 9, 16, or 23 to 31, wherein said first ISVD essentially consists of 4 framework regions (FR1 to FR4, respectively) and 3 complementarity determining regions (CDR1 to CDR3, respectively), in which CDR1 consists of the amino acid sequence of GYVHKINFYG (SEQ ID NO: 171), CDR2 consists of the amino acid sequence of HISIGDQTD (SEQ ID NO: 209), and CDR3 consists of the amino acid sequence of LSRIWPYDY (SEQ ID NO: 235).
Embodiment 33. The polypeptide according to any of embodiments 1 to 32, wherein the amino acid sequence of the first ISVD has at least 80% sequence identity with at least one of the amino acid sequences of SEQ ID NO's: 1 to 152, 261 or 262 in which for the purposes of determining the degree of sequence identity, the amino acid residues that form the CDR sequences are disregarded.
Embodiment 34. The polypeptide according to any of embodiments 1 to 33, wherein the amino acid sequence of the first ISVD has at least 90%, preferably at least 95%, more preferably at least 99% sequence identity with at least one of the amino acid sequences of SEQ ID NO's: 1 to 152, 261 or 262, in which for the purposes of determining the degree of sequence identity, the amino acid residues that form the CDR sequences are disregarded.
Embodiment 35. The polypeptide according to any of embodiments 1 to 34, comprising a first ISVD in which:
   (i) FR1 is chosen from the group of amino acid sequences of SEQ ID NO's: 248 to 250,
   (ii) FR2 is the amino acid sequence of SEQ ID NO: 251,
   (iii) FR3 is chosen from the group of amino acid sequences of SEQ ID NO's: 252 to 259, and
   (iv) FR4 is the amino acid sequence of SEQ ID NO: 260.

Preferably, the polypeptide as defined in Embodiment 35 comprises a a first ISVD in which:
(i) FR1 is the amino acid sequence of SEQ ID NO: 249,
(ii) FR2 is the amino acid sequence of SEQ ID NO: 251,
(iii) FR3 is chosen from the group of amino acid sequences of SEQ ID NO's: 253 and 256, and
(iv) FR4 is the amino acid sequence of SEQ ID NO: 260. Embodiment 36. The polypeptide according to any of embodiments 1 to 35 wherein said first ISVD is chosen from the group of amino acid sequences of SEQ ID NO's: 1 to 20, 22 to 27, 35 to 41, 46 to 147, 150 to 152 and 261 to 262.
Embodiment 37. The polypeptide according to any of embodiments 1 to 36, wherein said first ISVD is chosen from the group of amino acid sequences of SEQ ID NO's: 1 to 20, 22 to 27, and 35 to 41.
Embodiment 38. The polypeptide according to any of embodiments 1 to 36, wherein said first ISVD is chosen from the group of amino acid sequences of SEQ ID NO's: 46 to 147, 150 to 152, 261 and 262.
Embodiment 39. The polypeptide according to any of embodiments 1 to 36, or 38, wherein said first ISVD is chosen from the group of amino acid sequences of SEQ ID NO's: 46 to 50, 147 and 150 to 152, 261 and 262.
Embodiment 40. The polypeptide according to any of embodiments 1 to 36, 38 or 39, wherein said first ISVD is chosen from the group of amino acid sequences of SEQ ID NO's: 46, 150 to 152, 261 and 262.
Embodiment 41. The polypeptide according to any one of the preceding embodiments, wherein said polypeptide directs the T cell to the target cell.
Embodiment 42. The polypeptide according to any one of the preceding embodiments, wherein said polypeptide induces T cell activation.
Embodiment 43. The polypeptide according to any one of the preceding embodiments, wherein said T cell activation is independent from MHC recognition.
Embodiment 44. The polypeptide according to any one of the preceding embodiments, wherein said T cell activation depends on presenting said polypeptide bound to said first antigen on a target cell to a T cell.
Embodiment 45. The polypeptide according to embodiments 42 to 44, wherein said T cell activation causes one or more cellular response of said T cell, wherein said cellular response is selected from the group consisting of proliferation, differentiation, cytokine secretion, cytotoxic effector molecule release, cytotoxic activity, expression of activation markers and redirected target cell lysis.
Embodiment 46. The polypeptide according to embodiments 42 to 45, wherein said T cell activation causes lysis of the target cell, by more than about 10%, such as 20%, 30%, or 40%, or even more than 50%, such as more than 60%.
Embodiment 47. The polypeptide according to any one of the preceding embodiments, wherein said polypeptide has an on rate constant (kₒₙ) for binding to the human TCR selected from the group consisting of at least about 10³ M⁻¹ s⁻¹, at least about 10⁴ M⁻¹s⁻¹, and at least about 10⁵ M⁻¹ s⁻¹, preferably as measured by SPR, preferably performed on a ProteOn XPR36 instrument, preferably at 25°.
Embodiment 48. The polypeptide according to any one of the preceding embodiments, wherein said polypeptide has a kₒₙ for binding to the non-human primate TCR selected from the group consisting of at least about 10³ M⁻¹ s⁻¹, at least about 10⁴ M⁻¹ s⁻¹, and at least about 10⁵ M⁻¹ s⁻¹, preferably as measured by SPR, preferably performed on a ProteOn XPR36 instrument, preferably at 25° C.
Embodiment 49. The polypeptide according to any one of the preceding embodiments, wherein said polypeptide has a k_{off} for binding to the human TCR selected from the group consisting of at most about 10⁻² s⁻¹, at most about 10⁻³ s⁻¹, and at most about 10⁻⁴ s⁻¹, preferably as measured by SPR, preferably performed on a ProteOn XPR36 instrument, preferably at 25° C.
Embodiment 50. The polypeptide according to any one of the preceding embodiments, wherein said polypeptide has a k_{off} for binding to the non-human primate TCR selected from the group consisting of at most about 10⁻¹ s⁻¹, at most about 10⁻² s⁻¹, at most about 10⁻³ s⁻¹, and at most about 10⁻⁴ s⁻¹, preferably as measured by SPR, preferably performed on a ProteOn XPR36 instrument, preferably at 25° C.
Embodiment 51. The polypeptide according to any one of the preceding embodiments, wherein said polypeptide has an affinity (KD) for binding to the human TCR selected from the group consisting of at most about 10⁻⁶ M, at most about 10⁻⁷ M, at most about 10⁻⁸ M, at most about 10⁻⁸ M, and at most about 10⁻⁹ M, preferably as measured by SPR, preferably performed on a ProteOn XPR36 instrument, preferably at 25° C.
Embodiment 52. The polypeptide according to any one of the preceding embodiments, wherein said polypeptide has a KD for binding to the non-human primate TCR selected from the group consisting of at most about 10⁻⁶ M, at most about 10⁻⁷ M, and at most about 10⁻⁸ M, preferably as measured by SPR, preferably performed on a ProteOn XPR36 instrument, preferably at 25° C.

Preferably, the polypeptide according to any one of the preceding embodiments has (i) an affinity (KD) for binding to the human TCR selected from the group consisting of at most about 10⁻⁶ M, at most about 10⁻⁷ M, at most about 10⁻⁸ M, at most about 10⁻⁸ M, and at most about 10⁻⁹ M and (ii) has a KD for binding to the non-human primate TCR selected from the group consisting of at most about 10⁻⁶ M, at most about 10⁻⁷ M, and at most about 10⁻⁸ M, preferably as measured by SPR, preferably performed on a ProteOn XPR36 instrument, preferably at 25° C.
Embodiment 53. The polypeptide according to any one of the preceding embodiments, wherein said polypeptide binds to the human TCR with a lower KD than that of the same polypeptide wherein the first ISVD is replaced by an ISVD of SEQ ID NO: 1, said KD preferably determined by SPR, preferably performed on a ProteOn XPR36 instrument, preferably at 25° C.
Embodiment 54. The polypeptide according to any one of the preceding embodiments, wherein said polypeptide binds to the non-human primate TCR with a lower K_{D} than that of the same polypeptide wherein the first ISVD is replaced by an ISVD of SEQ ID NO: 1, said K_{D} preferably determined by SPR, preferably performed on a ProteOn XPR36 instrument, preferably at 25° C.

Preferably, the polypeptide according to any one of the preceding embodiments shows improved cross-reactivity, i.e., lower K_{D} for binding to human and non-human primate TCR as compared to the same polypeptide wherein the first ISVD is replaced by an ISVD of SEQ ID NO: 1, said K_{D} preferably determined by SPR, preferably performed on a ProteOn XPR36 instrument, preferably at 25° C.

For instance, the polypeptide according to any one of the preceding embodiments shows a lower difference in human-cynomolgus cross-reactivity based on K_{D} compared to the difference in human-cynomolgus cross-reactivity for the same polypeptide wherein the first ISVD is replaced by an ISVD of SEQ ID NO: 1. Preferably, the KD is determined by SPR, preferably performed on a ProteOn XPR36 instrument, preferably at 25° C.
Embodiment 55. The polypeptide according to any one of the preceding embodiments, wherein said polypeptide causes a human T cell to lyse the target cell with an EC50 value selected from the group consisting of at most about 10⁻⁹ M, and at most about 10⁻¹⁰M, said EC50 value as determined in a T cell mediated killing assay.
Embodiment 56. The polypeptide according to any one of the preceding embodiments, wherein said polypeptide causes a non-human primate T cell to lyse the target cell with an EC50 value selected from the group consisting of at most about 10⁻⁹ M, and at most about 10⁻¹⁰ M, said EC50 value as determined in a T cell mediated killing assay.
Embodiment 57. The polypeptide according to any one of the preceding embodiments, wherein said polypeptide causes a human T cell to lyse the target cell with a lower EC50 value than that of the same polypeptide wherein the first ISVD is replaced by an ISVD of SEQ ID NO: 1, said EC50 value as determined in a T cell mediated killing assay.
Embodiment 58. The polypeptide according to any one of the preceding embodiments, wherein said polypeptide causes a non-human primate T cell to lyse the target cell with a lower EC50 value than that of the same polypeptide wherein the first ISVD is replaced by an ISVD of SEQ ID NO: 1, said EC50 value as determined in a T cell mediated killing assay.
Embodiment 59. The polypeptide according to any one of the previous embodiments, wherein the first ISVD is positioned N-terminally from the second ISVD, wherein the first ISVDs is optionally positioned at the N-terminus of said polypeptide.
Embodiment 60. The polypeptide according to any one of embodiments 1 to 58, wherein the first ISVD is positioned C-terminally from the second ISVD.
Embodiment 61. The polypeptide according to any one of the preceding embodiments, wherein said first antigen on a target cell is a tumour antigen, preferably a tumour associated antigen (TAA).
Embodiment 62. The polypeptide according to any one of the preceding embodiments, further comprising a third ISVD, which specifically binds to a second antigen on a target cell.
Embodiment 63. The polypeptide according to embodiment 62, wherein said second antigen is different from said first antigen.
Embodiment 64. The polypeptide according to embodiment 62, wherein said second antigen is the same as the first antigen.
Embodiment 65. The polypeptide according to embodiment 64, wherein said second and third ISVD bind to the same epitope of the antigen.
Embodiment 66. The polypeptide according to embodiment 64, wherein said second and third ISVD bind to a different epitope of the antigen.
Embodiment 67. The polypeptide according to embodiment 66, wherein the third and second ISVD are able to simultaneously bind to two different epitopes on the same antigen molecule.
Embodiment 68. The polypeptide according to any of embodiments 61 to 67, wherein said second antigen on a target cell is a tumour antigen, preferably a tumour associated antigen (TAA).
Embodiment 69. The polypeptide according to any of embodiments 62 to 68, wherein said first antigen and said second antigen are present on the same target cell.
Embodiment 70. The polypeptide according to any of embodiments 62 to 69, wherein said first antigen and said second antigen are present on different target cells.
Embodiment 71. The polypeptide according to any of embodiments 61 to 70, wherein said TAA (is) are independently chosen from the group consisting of Melanoma-associated Chondroitin Sulfate Proteoglycan (MCSP), Epidermal Growth Factor Receptor (EGFR), Fibroblast Activation Protein (FAP), MART-1, carcinoembryonic antigen (CEA), gp100, MAGE-1, HER-2, Lewis antigens, CD123, CD44, CLL-1, CD96, CD47, CD32, CXCR4, Tim-3, CD25, TAG-72, Ep-CAM, PSMA, PSA, GD2, GD3, CD4, CD5, CD19, CD20, CD22, CD36, CD45, CD52, CD147, growth factor receptors including ErbB3 and ErbB4, Cytokine receptors including Interleukin-2 receptor gamma chain (CD132 antigen), Interleukin-10 receptor alpha chain (IL-10R-A), Interleukin-10 receptor beta chain (IL-10R-B), Interleukin-12 receptor beta-1 chain (IL-12R-betal), Interleukin-12 receptor beta-2 chain (IL-12 receptor beta-2), Interleukin-13 receptor alpha-1 chain (IL-13-alpha-I) (CD213al antigen), Interleukin-13 receptor alpha-2 chain (Interleukin-13 binding protein), Interleukin-17 receptor (IL-17 receptor), Interleukin-17B receptor (IL-17B receptor), Interleukin 21 receptor precursor (IL-21R), Interleukin-1 receptor type I (IL-IR-1) (CD121a), Interleukin-1 receptor type II (IL-1R-beta) (CDwl21b), Interleukin-1 receptor antagonist protein (IL-Ira), Interleukin-2 receptor alpha chain (CD25 antigen), Interleukin-2 receptor beta chain (CD122 antigen), Interleukin-3 receptor alpha chain (IL-3R-alpha) (CD123 antigen), CD30, IL23R, IGF-1R, IL5R, IgE, CD248 (endosialin), CD44v6, gpA33, Ron, Trop2, PSCA, claudin 6, claudin 18.2, CLEC12A, CD38, ephA2, c-Met, CD56, MUC16, EGFRvIII, AGS-16, CD27L, Nectin-4, SLITRK6, mesothelin, folate receptor, tissue factor, axl, Glypican-3, CA9, Cripto, CD138, CD37, MUCI, CD70, gastrin releasing peptide receptor, PAP, CEACAM5, CEACAM6, CXCR7, N-cadherin, FXYD2 gamma a, CD21, CD133, Na/K-ATPase, mIgM (membrane-bound IgM), mlgA (membrane-bound IgA), Mer, Tyro2, CD120, CD95, CA 195, DR5, DR6, DcR3 and CAIX, and related polymorphic variants and isoforms.
Embodiment 72. The polypeptide according to embodiment 61, wherein said TAA is CD123 or Glypican-3.
Embodiment 73. The polypeptide according to any one of embodiments 64 to 70, wherein said first and second antigen are CD123 or Glypican-3.
Embodiment 74. The polypeptide according to any of embodiments 62 to 71, or 73, wherein the first ISVD is positioned N-terminally from the second and the third ISVD, wherein the first ISVDs is optionally positioned at the N-terminus of said polypeptide.
Embodiment 75. The polypeptide according to any of embodiments 62 to 71, or 73, wherein the first ISVD is positioned C-terminally from the second and the third ISVD.
Embodiment 76. The polypeptide according to any of embodiments 62 to 71, or 73, wherein the first ISVD is positioned in between the second and the third ISVD.
Embodiment 77. The polypeptide according to any one of the preceding embodiments, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units.
Embodiment 78. The polypeptide according to embodiment 77, in which said one or more other groups, residues, moieties or binding units that provide the polypeptide with increased half-life is chosen from the group consisting of a polyethylene glycol molecule, serum proteins or fragments thereof, binding units that can bind to serum proteins, an Fc portion, and small proteins or peptides that can bind to serum proteins.
Embodiment 79. The polypeptide according to embodiment 77 or 78, in which said one or more other groups, residues, moieties or binding units that provide the polypeptide with increased half-life is chosen from the group consisting of binding units that can bind to serum albumin (such as human serum albumin) or a serum immunoglobulin (such as IgG).
Embodiment 80. The polypeptide according to any of embodiments 77 to 79, in which said one or more other groups, residues, moieties or binding units that provide the polypeptide with increased half-life is a binding unit that can bind to human serum albumin.
Embodiment 81. The polypeptide according to any of embodiments 77 to 80, in which said one or more other groups, residues, moieties or binding units that provide the polypeptide with increased half-life is an ISVD that can bind to human serum albumin.
Embodiment 82. The polypeptide according to embodiment 81, wherein said ISVD that can bind to human serum albumin essentially consists of 4 framework regions (FR1 to FR4, respectively) and 3 complementarity determining regions (CDR1 to CDR3 respectively), wherein the amino acid sequence of CDR1 is GFTFRSFGMS (SEQ ID NO: 357) or GFTFSSFGMS (SEQ ID NO: 358), the amino acid sequence of CDR2 is SISGSGSDTL (SEQ ID NO: 359), and the amino acid sequence of CDR3 is GGSLSR (SEQ ID NO: 360).
Embodiment 83. The polypeptide according to embodiment 82 wherein said ISVD binding serum albumin is selected from SEQ ID NOs: 361 to 378.
Embodiment 84. The polypeptide according to any one of preceding embodiments, wherein said ISVDs are directly linked to each other or are linked via a linker.
Embodiment 85. The polypeptide according to any one of embodiments 1 to 84, wherein said first ISVD and/or said second ISVD and/or possibly said third ISVD and/or possibly said ISVD binding to (human) serum albumin are linked via a linker.
Embodiment 86. The polypeptide according to embodiment 85, wherein said linker is chosen from the group consisting of linkers of 3A, 5GS, 7GS, 8GS, 9GS, 10GS, 15GS, 18GS, 20GS, 25GS, 30GS, 35GS, 40GS, G1 hinge, 9GS-G1 hinge, a llama upper long hinge region, and a G3 hinge (SEQ ID NOs: 322 to 338).
Embodiment 87. The polypeptide according to any one of the preceding embodiments, wherein said ISVD is a V_{HH}, a humanized V_{HH}, or a camelized V_{H}.
Embodiment 88. The polypeptide according to any one of the preceding embodiments, wherein said first ISVD is chosen from the group consisting of SEQ ID NOs: 46 to 50, 147, 150 to 152, 261 and 262, and wherein said second ISVD is the amino acid sequence of SEQ ID NOs: 263.
Embodiment 89. The polypeptide according to embodiment 88, wherein said first ISVD is chosen from the group consisting of SEQ ID NOs: 46, 150 to 152, 261 and 262, and wherein said second ISVD consists of the amino acid sequence of SEQ ID NOs: 263.
Embodiment 90. The polypeptide according to embodiment 89, chosen from the group of amino acid sequences of SEQ ID NO: 275 and 276.
Embodiment 91. The polypeptide according to embodiment 88 to 90, further comprising a third ISVD which consists of the amino acid sequence of SEQ ID NO: 265.
Embodiment 92. A polypeptide according to embodiment 91, chosen from the group of amino acid sequences of SEQ ID NO: 283, 284, 288, 289 and 290.
Embodiment 93. The polypeptide according to any one of the preceding embodiments further comprising a C-terminal extension (X)n, in which n is 1 to 5, such as 1, 2, 3, 4 or 5, and in which X is a naturally occurring amino acid, preferably no cysteine.
Embodiment 94. A nucleic acid encoding the polypeptide as defined in any one of embodiments 1 to 93.
Embodiment 95. A vector comprising the nucleic acid as defined in embodiment 94.
Embodiment 96. A host cell transformed or transfected with the nucleic acid as defined in embodiment 94 or with the vector as defined in embodiment 95.
Embodiment 97. A process for the production of the polypeptide according to any one of embodiments 1 to 93, said process comprising culturing a host cell as defined in embodiment 96 under conditions allowing the expression of the polypeptide as defined in any one of embodiments 1 to 93 and recovering the produced polypeptide from the culture.
Embodiment 98. A composition comprising the polypeptide according to any of embodiments 1 to 93, or the polypeptide produced according to the process of embodiment 97.
Embodiment 99. A composition according to embodiment 98, which is a pharmaceutical composition.
Embodiment 100. The pharmaceutical composition according to embodiment 99, further comprising at least one pharmaceutically acceptable carrier, diluent or excipient and/or adjuvant, and optionally one or more further pharmaceutically active polypeptides and/or compounds.
Embodiment 101. The polypeptide according to any one of embodiments 1 to 93, or the composition according to any one of embodiments 98 to 100, for use as a medicament.
Embodiment 102. The polypeptide according to any one of embodiments 1 to 93, or the composition according to any one of embodiments 98 to 100, for use in treating a subject in need thereof.
Embodiment 103. The polypeptide according to any one of embodiments 1 to 93, or the composition according to any one of embodiments 98 to 100, for use in the prevention, treatment or amelioration of a disease selected from the group consisting of a proliferative disease, an inflammatory disease, an infectious disease and an autoimmune disease.
Embodiment 104. A method for delivering a prophylactic or therapeutic polypeptide to a specific location, tissue or cell type in the body, the method comprising the step of administering to a subject a polypeptide according to any one of embodiments 1 to 93, or the composition according to any one of embodiments 98 to 100.
Embodiment 105. A method for treating a subject in need thereof, the method comprising the step of administering to a subject a polypeptide according to any one of embodiments 1 to 93, or the composition according to any one of embodiments 98 to 100.
Embodiment 106. A method for the treatment or amelioration of a disease selected from the group consisting of a proliferative disease, an inflammatory disease, an infectious disease and an autoimmune disease, comprising the step of administering to a subject in need thereof the polypeptide according to any one of embodiments 1 to 93, or the composition according to any one of embodiments 98 to 100.
Embodiment 107. The polypeptide or the composition for use according to embodiment 103, or the method according to embodiment 106, wherein said proliferative disease is cancer.
Embodiment 108. The polypeptide, or the composition for use according to embodiment 107, or the method according to embodiment 107, wherein said cancer is chosen from the group consisting of carcinomas, gliomas, mesotheliomas, melanomas, lymphomas, leukemias (including acute myeloid leukemia, chronic myeloid leukemia, acute B lymphoblastic leukemia, chronic lymphocytic leukemia and hairy cell leukemia), myelodysplastic syndrome, blastic plasmacytoid dendritic cell neoplasm, systemic mastocytosis, adenocarcinomas: breast cancer, ovarian cancer, cervical cancer, glioblastoma, multiple myeloma (including monoclonal gammopathy of undetermined significance, asymptomatic and symptomatic myeloma), prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease or AIDS-associated primary effusion lymphoma, neuroectodermal tumors, rhabdomyosarcoma; as well as any metastasis of any of the above cancers.
Embodiment 109. The polypeptide or the composition for use according to embodiment 103, or the method according to embodiment 106, wherein said inflammatory disease is an inflammatory disease chosen from the group consisting of Autoimmune Lupus (SLE), allergy, asthma and rheumatoid arthritis.
Embodiment 110. The polypeptide, or the composition for use according to any one of embodiments 107 to 109, or the method according to any one of embodiments 107 to 109, wherein the treatment is a combination treatment.
Embodiment 111. A kit comprising a polypeptide as defined in any one of embodiments 1 to 93, a nucleic acid as defined in embodiment 94, a vector as defined in embodiment 95, or a host cell as defined in embodiment 96.

### 4 BRIEF DESCRIPTION OF THE DRAWINGS

FIG. 1: Sequence of ISVD T0170056G05, with indication of the frameworks (FR) and complementarity determining regions (CDR). Numbering of the sequence is according to Kabat, CDRs are determined based on the AbM definition (see Antibody Engineering, Vol2 by Kontermann & DObel (Eds), Springer Verlag Heidelberg Berlin, 2010).

FIG. 2: Dose dependent binding of the second wave ISVD variants to cynomolgus primary T cells. The MFI (Mean fluorescence intensity) value is plotted against the concentration of the ISVD.

FIG. 3: Dose dependent binding of T017000624 (diamonds), T0170056G05 (dots) and cAbLys3 (squares) to human and cynomolgus TCR/CD3 transfected cells. The MFI (Mean fluorescence intensity) value is plotted against the concentration of the ISVD.

FIG. 4: Dose dependent binding of T017000624 (diamonds), T0170056G05 (dots) and cAbLys3 (squares) to human and cynomolgus primary T cells. The MFI (Mean fluorescence intensity) value is plotted against the concentration of the ISVD.

FIG. 5: Dose response curves for human and cynomolgus T cell mediated killing by CD123/TCR ISVD formats in a flow cytometry based MOLM-13 and KG-1a killing assay using an E:T ratio of 10:1. The % cell death (% of TOPRO positive cells) is plotted against the concentration of the ISVD formats.

FIG. 6: Dose response curves for human and cynomolgus T cell mediated killing by CD123/TCR ISVD formats in an impedance-based (xCELLigence) T cell mediated CHO Flp-In huCD123 cell killing assay using an E:T ratio of 15:1. The cell index (CI) is plotted against the concentration of ISVD formats.

FIG. 7: Dose-dependent T cell mediated killing of target cell lines by trivalent CD123/TCR bispecific ISVD formats via flow cytometry. E:T ratio of 10:1 on MOLM-13 and KG-1a. The % cell death (% of TOPRO positive cells) is plotted against the concentration of the ISVD formats.

FIG. 8: Dose dependent human and cynomolgus T cell mediated killing of CHO Flp-In huCD123 target cells or the parental CHO Flp-In cell line using an effector to target ratio of 15 to 1 in an impedance-based assay (xCELLigence). The cell index (CI) is plotted against the concentration of the ISVD formats.

FIG. 9: Dose dependent competition of T017000679 (open circle, dotted line), T017000681 (open triangle, dotted line), T017000680 (full circle, full line) to human primary T cells and HSC-F cynomolgus T cell line. The MFI (Median or Mean fluorescence intensity) value is plotted against the concentration of the ISVD.

FIG. 10: CD123hi cell counts in the PBMC fraction of peripheral blood of treated cynomolgus monkey over time. The absolute number of CD123^{hi} cells per μL PBMCs on a given test day is expressed as a percentage of the average absolute number of CD123^{hi} cells per μL PBMC during the pretreatment days (TD-7, -6, -4 and TD1 predosing)±SEM for the different treatment groups: vehicle (open diamonds, n=4), reference DART molecule (closed diamonds, n=2), TCR/CD123 ISVD format T017000139 (black triangles, n=4) irrelevant/TCR ISVD format T017000674 (open squares, n=4), TCR/irrelevant ISVD format T017000672 (open circles, n=4), CD123/TCR ISVD format T017000675 (black squares, n=4) and TCR/CD123 ISVD format T017000670 (black circles, n=6). Vertical lines reflect the start of the 4-days continuous infusion treatment periods. Horizontal line reflects the baseline level of CD123^{hi} cells (average number of CD123^{hi} cells TD-7, -6, -4 and TD1 predosing).

FIG. 11: CD123′^{int} cell counts in the PBMC fraction of peripheral blood of treated cynomolgus monkey over time. The absolute number of CD123′^{int} cells per μL PBMCs on a given test day is expressed as a percentage of the average absolute number of CD123^{int} cells per μL PBMC during the pretreatment days (TD-7, -6, -4 and TD1 predosing)±SEM for the different treatment groups: vehicle (open diamonds, n=4), reference DART molecule (closed diamonds, n=2), irrelevant/TCR ISVD format T017000674 (open squares, n=4), TCR/irrelevant ISVD format T017000672 (open circles, n=4), CD123/TCR ISVD format T017000675 (black squares, n=4) and TCR/CD123 ISVD format T017000139 (black triangles, n=4) TCR/CD123 ISVD format T017000670 (black circles, n=6). Vertical lines reflect the start of the 4-days continuous infusion treatment periods. Horizontal line reflects the baseline level of CD123^{hi} cells (average number of CD123^{hi} cells TD-7, -6, -4 and TD1 predosing).

FIG. 12: Helper T cell (Th; CD4+CD3+ cells) counts in the PBMC fraction of peripheral blood of treated cynomolgus monkey over time. The absolute number of Th cells per μL PBMCs on a given test day is expressed relative to the average absolute number of Th cells per μL PBMC during the pretreatment days (TD-7, -6, -4 and TD1 predosing)±SEM for the different treatment groups: vehicle (open diamonds, n=4), reference DART molecule (closed diamonds, n=2), TCR/CD123 ISVD format T017000139 (black triangles, n=4), irrelevant/TCR ISVD format T017000674 (open squares, n=4), TCR/irrelevant ISVD format T017000672 (open circles, n=4), CD123/TCR ISVD format T017000675 (black squares, n=4) and TCR/CD123ISVD format T017000670 (black circles, n=6). Vertical lines reflect the start of the 4-days continuous infusion treatment periods. Horizontal line at 100 reflects the baseline level of Th cells (average number of Th cells TD-7, -6, -4 and TD1 predosing).

FIG. 13: Cytotoxic T cell (Tc; CD8⁺CD3⁺ cells) counts in the PBMC fraction of peripheral blood of treated cynomolgus monkey over time. The absolute number of Tc cells per μL PBMCs on a given test day is expressed relative to the average absolute number of Tc cells per μL PBMC during the pretreatment days (TD-7, -6, -4 and TD1 predosing)±SEM for the different treatment groups: vehicle (open diamonds, n=4), reference DART molecule (closed diamonds, n=2), TCR/CD123 ISVD format T017000139 (black triangles, n=4), irrelevant/TCR ISVD format T017000674 (open squares, n=4), TCR/irrelevant ISVD format T017000672 (open circles, n=4), CD123/TCR ISVD format T017000675 (black squares, n=4) and TCR/CD123 ISVD format T017000670 (black circles, n=6). Vertical lines reflect the start of the 4-days continuous infusion treatment periods. Horizontal line at 100 reflects the baseline level of Tc cells (average number of Th ells TD-7, -6, -4 and TD1 predosing).

FIG. 14: Percentage of PD-1+Th (CD4+CD3+) cells in the PBMC fraction of peripheral blood of treated cynomolgus monkey over time. The average percentage of PD-1+ cells on a given test day is shown±SEM for the different treatment groups: vehicle (open diamonds, n=4), reference DART molecule (closed diamonds, n=2), TCR/CD123 ISVD format T017000139 (black triangles, n=4), irrelevant/TCR ISVD format T017000674 (open squares, n=4), TCR/irrelevant ISVD format T017000672 (open circles, n=4), CD123/TCR ISVD format T017000675 (black squares, n=4) and TCR/CD123 ISVD format T017000670 (black circles, n=6). Vertical lines reflect the start of the 4-days continuous infusion treatment periods.

FIG. 15: Percentage of PD-1+Tc (CD8+CD3+) cells in the PBMC fraction of peripheral blood of treated cynomolgus monkey over time. The average percentage of PD-1+ cells on a given test day is shown ±SEM for the different treatment groups: vehicle (open diamonds, n=4), reference DART molecule (closed diamonds, n=2), TCR/CD123 ISVD format T017000139 (black triangles, n=4), irrelevant/TCR ISVD format T017000674 (open squares, n=4), TCR/irrelevant ISVD format T017000672 (open circles, n=4), CD123/TCR ISVD format T017000675 (black squares, n=4) and TCR/CD123 ISVD format T017000670 (black circles, n=6). Vertical lines reflect the start of the 4-days continuous infusion treatment periods.

FIG. 16: Dose-dependent killing effect of the trispecific GPC3 ISVD T-cell engagers in the Incucyte based human TDC HepG2-Nuclight green assay using an effector to target ratio of 15:1, analyzed at 72 h after seeding. Controls (left) are: No compound (open square) and reference for 100% killing (open circle).

FIG. 17: Dose-dependent killing effect of the trispecific GPC3 ISVD T-cell engagers in the Incucyte based human TDC HepG2-Nuclight green assay using an effector to target ratio of 15:1, analyzed at 60 h after seeding. Controls (left) are: No compound (open square) and reference for 100% killing (open circle).

FIG. 18: Dose-dependent killing effect of the trispecific GPC3 ISVD T-cell engagers in the Incucyte based human TDC HepG2-Nuclight green assay (FIG. 18A) and in the xCELLigence based human TDC Huh7 assay (FIG. 18B), using an effector to target ratio of 15:1, analyzed at 60 h after seeding. Controls (left) are: No compound (open square) and reference for 100% killing (open circle).

FIG. 19: Dose-dependent killing effect of the trispecific GPC3 ISVD T-cell engagers in the Incucyte based human TDC HepG2-Nuclight green assay (FIG. 19A) and in the xCELLigence based human TDC Huh7 assay (FIG. 19B), using an effector to target ratio of 15:1, analyzed at 60 h after seeding. Controls (left) are: (open square) No compound and (open circle) reference for 100% killing.

FIG. 20: Dose-dependent killing effect of the trispecific GPC3 ISVD T-cell engagers in the Incucyte based human TDC HepG2-Nuclight green assay (FIG. 20A) and in the xCELLigence based human TDC Huh7 assay (FIG. 20B), using an effector to target ratio of 15:1, analyzed at 60 h after seeding. Controls (left) are: (open square) No compound and (open circle) reference for 100% killing.

FIG. 21: Dose-dependent killing effect of the trispecific GPC3 T-cell engagers in the xCELLigence based human TDC assay on different tumor cell lines expressing decreasing expression levels of GPC3 using an effector to target ratio of 15:1: HepG2 analysed at 60 h (FIG. 21A), NCI-H661 analysed at 75 h (FIG. 21B), Huh-7 analysed at 60 h (FIG. 21C), MKN-45 analysed at 65 h (FIG. 21D), BxPC-3 analysed at 65 h (FIG. 21E), NCI-H292 analysed at 60 h (FIG. 21F). Controls (left) are: (open square) No compound (effector and T cells only).

FIG. 22: Dose-dependent killing effect of the five selected trispecific GPC3 ISVD based T-cell engagers in the xCELLigence based human TDC assay on two tumor cell lines using an effector to target ratio of 15:1 and analyzed at 60 h, NCI-H661 (FIG. 22A) and BxPC-3 (FIG. 22B). Controls (left) are: No compound (open square, effector and T cells only) and T017000698 at 30 nM (open diamond).

FIG. 23: Study design for efficacy model. Huh-7 tumor cells were subcutaneously injected in NOG mice. The tumors grew until the mean tumor volume of approximately 150 mm³ was reached. At this point, in vitro expanded T cells were injected into each mouse intraperitoneally (DO). The treatment with A022600424 injected intravenously started on DO, 3 h after T cell injection and continued on D3, D6, D9 and D12 (q3d). Four dose levels of A022600424 were tested (0.1 mg/kg, 0.2 mg/kg, 0.7 mg/kg and 2 mg/kg). The control T017000698 was injected in a control group at 2 mg/kg on DO, D3, D6, D9 and D12 (q3d). Survival blood sampling was done on D6 and D12 prior to administration of test compounds. All mice were sacrificed on D15, blood and tumor samples were collected.

FIG. 24: Results of the efficacy model. Four dose levels of A022600424 were tested (0.1 mg/kg, 0.2 mg/kg, 0.7 mg/kg and 2 mg/kg). The control T017000698 was injected in a control group at 2 mg/kg.

FIG. 25: Schematic presentation of the multispecific ISVD construct according to the invention showing from the N-terminus to the C-terminus the monovalent building blocks/ISVDs TCRαβ, CD33, CD123, and Alb connected via linkers.

FIG. 26: Binding of the monovalent CD33 (left) and CD123 binding BB (right) to human (top) or cynomolgus (bottom) transfected CD33, respectively CD123 cells.

FIG. 27: Binding of A025001562 to human CD33 and/or human CD123 expressing cells.

FIG. 28: Dose dependent inhibition of A025001562 (blue dots) and (grey dots) in the competition assays on primary T cells in the absence (dotted curves) or presence (full curves) of clinical grade HSA.

FIG. 29: Dose dependent human (top) or cynomolgus (bottom) T cell mediated killing of corresponding species CD33 (left) or CD123 (right) transfected cells, using an effector to target ratio of 15 to 1 in an impedance-based assay (xCELLigence) in the presence of 50 μM HSA.

FIG. 30: Dose dependent human (left) or cynomolgus (right) T cell mediated MOLM-13 cell killing in a flow cytometry-based assay using an effector to target ratio of 10:1. The % TO-PRO®-3 positive target cells is plotted against the concentration of the ISVD.

FIG. 31: Dose dependent human T cell mediated cell killing using an effector to target ratio of 15 to 1 in an impedance-based assay (xCELLigence). The cell index (CI) after 32-35 h of incubation is plotted against the concentration of the ISVDs.

FIG. 32: A025001562 inhibition of Molm13-luc AML tumor growth by in vivo bioluminescence imaging.

FIG. 33: A025001562 inhibition of Molm13-luc AML tumor growth by ex vivo bioluminescence imaging.

FIG. 34: Dose dependent human T cell mediated killing of U937 or KG1a cells in a flow cytometry-based assay using an effector to target ratio of 10:1. The % TO-PRO®-3+target cells is plotted against the concentration of the ISVD.

### 5 DETAILED DESCRIPTION OF THE PRESENT TECHNOLOGY

### 5.1 Certain Definitions

Unless indicated or defined otherwise, all terms used have their usual meaning in the art, which will be clear to the skilled person. Reference is for example made to the standard handbooks such as Sambrook et al. (1989, Molecular Cloning: A Laboratory Manual (2nd Ed.) Vols. 1-3, Cold Spring Harbor Laboratory Press), F. Ausubel et al. (1987, Current protocols in molecular biology, Green Publishing and Wiley Interscience, New York), Lewin (1985, Genes II, John Wiley & Sons, New York, N.Y.), Old et al. (1981, Principles of Gene Manipulation: An Introduction to Genetic Engineering (2nd Ed.) University of California Press, Berkeley, CA), Roitt et al. (2001, Immunology (6th Ed.) Mosby/Elsevier, Edinburgh), Roitt et al. (2001, Roitt's Essential Immunology (10th Ed.) Blackwell Publishing, UK), and Janeway et al. (2005, Immunobiology (6th Ed.) Garland Science Publishing/Churchill Livingstone, New York), as well as to the general background art cited herein.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews; Presta (2006, Adv. Drug Deliv. Rev. 58 (5-6): 640-56), Levin and Weiss (2006, Mol. Biosyst. 2(1): 49-57), Irving et al. (2001, J. Immunol. Methods 248(1-2): 31-45), Schmitz et al. (2000, Placenta 21 Suppl. A: S106-12), Gonzales et al. (2005, Tumour Biol. 26(1): 31-43), which describe techniques for protein engineering, such as affinity maturation and other techniques for improving the specificity and other desired properties of proteins such as immunoglobulins.

The term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acids or nucleotide sequences encoding the same, unless the context requires a more limited interpretation.

“Amino acid sequences” are interpreted to mean a single amino acid or an unbranched sequence of two or more amino acids, depending of the context. Nucleotide sequences are interpreted to mean an unbranched sequence of 3 or more nucleotides.

Amino acids are those L-amino acids commonly found in naturally occurring proteins and are listed in Table B-1 below. Those amino acid sequences containing D-amino acids are not intended to be embraced by this definition. Any amino acid sequence that contains post-translationally modified amino acids may be described as the amino acid sequence that is initially translated using the symbols shown in the Table below with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition.

The terms “protein”, “peptide”, “protein/peptide”, and “polypeptide” are used interchangeably throughout the disclosure and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptides and a protein; that polypeptides may be made by chemical synthesis or recombinant methods; and that proteins are generally made in vitro or in vivo by recombinant methods as known in the art.

Amino acid residues will be indicated interchangeably herein according to the standard three-letter or one-letter amino acid code, as mentioned in Table B-1 below.

TABLE B-1
Common amino acids

| 1-Letter | 3-Letter | |
|---|---|---|
| Code | Code | Name |
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Unspecified |
| Y | Tyr | Tyrosine |

When an amino acid residue is indicated as “X” or “Xaa”, it means that the amino acid residue is unspecified, unless the context requires a more limited interpretation. For example, if the description provides a amino acid sequence of a CDR wherein one (or more) of the amino acid residue(s) is (are) indicated with Xaa, the description may further specify which amino acid residue(s) is (can be) present at that specific position of the CDR. As an example, when a amino acid sequence such as a CDR is said to consist of Gly Xaa Val His Xaa (SEQ ID NO: 429), wherein Xaa at position 2 is Asp or Glu, and Xaa at position 5 is Lys or Gln, said CDR will have at its second position starting from the left as amino acid residue either an Asp or Glu and at its fifth position starting from the left either a Lys or Gln. In this example, said CDR would thus be one of the 4 following sequences (SEQ ID NOs: 430-433): Gly Asp Val His Lys; Gly Glu Val His Lys; Gly Asp Val His Gln; or Gly Glu Val His Gln. Further examples will be clear for the skilled person based on the disclosure herein.

For the purposes of comparing two or more ISVDs or other amino acid sequences such e.g. the polypeptides of the present technology etc., the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position).

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

An “amino acid difference” as used herein refers to a deletion, insertion or substitution of a single amino acid residue vis-à-vis a reference sequence, and preferably is a substitution. Fewer amino acid differences with a given reference sequence are generally preferred. For example, where a CDR has 2 or 1 amino acid difference with a given SEQ ID NO, 1 amino acid difference is preferred.

In one embodiment, amino acid substitutions are conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

In one embodiment, conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into lie or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

For instance, when a nucleotide sequence, amino acid sequence or polypeptide is said to “comprise” another nucleotide sequence, amino acid sequence or polypeptide, respectively, or to “essentially consist of” another nucleotide sequence, amino acid sequence or polypeptide, this may mean that the latter nucleotide sequence, amino acid sequence or polypeptide has been incorporated into the first mentioned nucleotide sequence, amino acid sequence or polypeptide, respectively, but more usually this generally means that the first mentioned nucleotide sequence, amino acid sequence or polypeptide comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when a polypeptide of the present technology is said to comprise an immunoglobulin single variable domain, this may mean that said immunoglobulin single variable domain sequence has been incorporated into the sequence of the polypeptide of the present technology, but more usually this generally means that the polypeptide of the present technology contains within its sequence the sequence of the immunoglobulin single variable domains irrespective of how said polypeptide of the present technology has been generated or obtained. Also, when a nucleic acid or nucleotide sequence is said to comprise another nucleotide sequence, the first mentioned nucleic acid or nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the first mentioned, larger nucleic acid or nucleotide sequence).

When a amino acid sequence or polypeptide is said to “essentially consist of” an immunoglobulin single variable domain, it is meant that said amino acid sequence or polypeptide either is exactly the same as the immunoglobulin single variable domain or corresponds to polypeptide or amino acid sequence which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the immunoglobulin single variable domain. When “consist of” is used, it is meant that the amino acid sequence or polypeptide is exactly the same as the immunoglobulin single variable domain.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the present technology described herein. Such equivalents are intended to be encompassed by the present technology.

The term “and/or” wherever used herein includes the meaning of “and”, “or” and “all or any other combination of the elements connected by said term”.

The term “about” or “approximately” as used herein means within 20%, preferably within 15%, more preferably within 10%, and most preferably within 5% of a given value or range.

### 5.2 the Polypeptides of the Present Technology

### Multispecific-Multivalent Polypeptides

The present technology relates to multispecific-multivalent polypeptides, which comprise at least one ISVD that specifically binds to the constant domain of a human and of a non-human primate TCR as described in detail in below (section 5.3: “Immunoglobulin single variable domains”), and one or more ISVDs that specifically bind to an antigen on a target cell.

In one aspect, the multispecific-multivalent polypeptide of the present technology is at least bispecific, but it can also be e.g., trispecific, tetraspecific, pentaspecific, etc. Moreover, the multispecific-multivalent polypeptide is at least bivalent, but can also be e.g., trivalent, tetravalent, pentavalent, hexavalent, etc.

The terms “bispecific”, “trispecific”, “tetraspecific”, “pentaspecific”, etc., all fall under the term “multispecific” and refer to binding to two, three, four, five, etc., different target molecules, respectively. The term “multispecific” refers to binding to multiple different target molecules.

The terms “bivalent”, “trivalent”, “tetravalent”, “pentavalent”, “hexavalent”, etc. all fall under the term “multivalent” and indicate the presence of two, three, four, five, six, etc., binding units/building blocks, respectively, such as ISVDs. The term “multivalent” indicates the presence of multiple binding units/building blocks.

For example, the polypeptide may be bispecific-bivalent, such as a polypeptide comprising or consisting of at least two ISVDs, wherein one ISVD specifically binds to the constant domain of a human and of a non-human primate TCR and one ISVD specifically binds to a first antigen on a target cell. In another example, the polypeptide may be bispecific-trivalent, such a polypeptide comprising or consisting of three ISVDs, wherein one ISVD specifically binds to the constant domain of a human and of a non-human primate TCR, one ISVD specifically binds to a first antigen on a target cell, and one ISVD specifically binds to a second antigen on a target cell, wherein said second antigen is the same as the first antigen. In yet another example, the trispecific-trivalent polypeptide, next to one ISVD that specifically binds to the constant domain of a human and of a non-human primate TCR and one ISVD that specifically binds to a first antigen on a target cell, comprises one ISVD that specifically binds to human serum albumin. Further examples of multispecific-multivalent polypeptides will be clear for the skilled person based on the disclosure herein.

As will be clear from the further description above and herein, the ISVDs of the present technology can be used as “building blocks” to form polypeptides of the present technology, e.g., by suitably combining them with other groups, residues, moieties or binding units, in order to form compounds or constructs as described herein (such as, without limitations, the bi-/tri-/tetra-/multivalent and bi-/tri-/tetra-/multispecific polypeptides of the present technology described herein) which combine within one molecule one or more desired properties or biological functions. A polypeptide with multiple ISVDs is also referred to herein as a “construct” or “ISVD format”.

The inventors found that the introduction of certain amino acid mutations—and combinations thereof—in the CDRs of ISVD T0170056G05 (disclosed as SEQ ID NO: 50 in WO2016180969) resulted in improved binding to the constant domains of a human TCR and/or of a non-human primate TCR.

In one embodiment, the multispecific-multivalent polypeptide of the present technology comprises a first and a second immunoglobulin single variable domain (ISVD), wherein
said first ISVD specifically binds to a constant domain of a human and of a non-human primate TCR present on a T cell;
said second ISVD specifically binds to a first antigen on a target cell;
wherein said first antigen is different from said TCR;
wherein said target cell is different from said T cell; and
wherein said first ISVD essentially consists of 4 framework regions (FR1 to FR4, respectively) and 3 complementarity determining regions (CDR1 to CDR3, respectively), wherein
(i) the amino acid sequence of CDR1 is Xaa Xaa Val His Xaa Xaa Xaa Xaa Xaa Xaa (SEQ ID NO: 295), wherein
   Xaa at position 1 is Gly, Trp, Gln, or Tyr,
   Xaa at position 2 is Asp, Glu, Tyr, Ser, or Ala,
   Xaa at position 5 is Lys, or Gln,
   Xaa at position 6 is lie, or Leu,
   Xaa at position 7 is Asn, or Phe,
   Xaa at position 8 is Phe, Val, or Tyr,
   Xaa at position 9 is Leu, Tyr, or Phe,
   Xaa at position 10 is Gly, Thr, or Arg;
(ii) the amino acid sequence of CDR2 is Xaa lie Ser lie Xaa Asp Xaa Xaa Asp (SEQ ID NO: 296), wherein
   Xaa at position 1 is His, or Val,
   Xaa at position 5 is Gly, or Ala,
   Xaa at position 7 is Gln, Val, or Glu,
   Xaa at position 8 is Thr, Ser, or Val; and
(iii) the amino acid sequence of CDR3 is Xaa Ser Xaa lie Xaa Pro Tyr Xaa Xaa (SEQ ID NO: 297), wherein
   Xaa at position 1 is Phe, Leu or Met,
   Xaa at position 3 is Arg, Lys, or Ser,
   Xaa at position 5 is Tyr, or Trp,
   Xaa at position 8 is Asp, or Gln,
   Xaa at position 9 is Tyr, or Trp, wherein Xaa at position 1, 2, 5, 6, 7, 8, 9 and 10 of CDR1 are not simultaneously Gly, Asp, Lys, lie, Asn, Phe, Leu, and Gly, respectively;
wherein Xaa at position 1, 5, 7, and 8 of CDR2 are not simultaneously His, Gly, Gln, and Thr, respectively; and wherein Xaa at position 1, 3, 5, 8, and 9 of CDR3 are not simultaneously Phe, Arg, Tyr, Asp, and Tyr, respectively.

As shown in Example 4 and Tables 2, 3 and 4 further provided herein, the introduction of these particular amino acid residues at these specific positions in a CDR results in improved binding of the ISVD to the constant domain of a human and/or a non-human primate TCR as compared to the binding of ISVD T0170000141. T0170000141 comprises the CDR sequences of T01700056G05, namely a CDR1 of SEQ ID NO: 153, a CDR2 of SEQ ID NO: 209, and a CDR3 of SEQ ID NO: 223.

When the first ISVD comprised in the multispecific-multivalent polypeptides of the present technology is said to comprise a CDR1 of SEQ ID NO: 295, wherein Xaa at position 1, 2, 5, 6, 7, 8, 9 and 10 are not simultaneously Gly, Asp, Lys, lie, Asn, Phe, Leu, and Gly, respectively; a CDR2 of SEQ ID NO: 296, wherein Xaa at position 1, 5, 7, and 8 are not simultaneously His, Gly, Gln, and Thr, respectively; and a CDR3 of SEQ ID NO: 297, wherein Xaa at position 1, 3, 5, 8, and 9 of CDR3 are not simultaneously Phe, Arg, Tyr, Asp, and Tyr, respectively; it means that in said first ISVD the CDR1, CDR2, CDR3 are not simultaneously the amino acid sequences of SEQ ID NO's: 153, 209, 223, respectively. The first ISVD comprised in the multispecific-multivalent polypeptides of the present technology, thus comprises different CDRs than those comprised in ISVD T0170056G05, or T017000141. For the sake of clarity, the first ISVD in the multispecific-multivalent polypeptides of the present technology does neither comprise the same CDRs as T0170056G05 (which is disclosed as SEQ ID NO: 50 in WO2016180969), nor the same CDRs as T017000141 (disclosed herein as SEQ ID NO: 2).

Accordingly, in in one embodiment, the multispecific-multivalent polypeptide of the present technology comprises a first and a second immunoglobulin single variable domain (ISVD), wherein
said first ISVD specifically binds both to a constant domain of a human TCR on a T cell and to the constant domain of a non-human primate TCR present on a T cell;
said second ISVD specifically binds to a first antigen on a target cell;
wherein said first antigen is different from said TCR;
wherein said target cell is different from said T cell; and
wherein said first ISVD essentially consists of 4 framework regions (FR1 to FR4, respectively) and 3 complementarity determining regions (CDR1 to CDR3, respectively), wherein
(i) the amino acid sequence of CDR1 is Xaa Xaa Val His Xaa Xaa Xaa Xaa Xaa Xaa (SEQ ID NO: 295), wherein
   Xaa at position 1 is Gly, Trp, Gin, or Tyr,
   Xaa at position 2 is Asp, Glu, Tyr, Ser, or Ala,
   Xaa at position 5 is Lys, or Gln,
   Xaa at position 6 is lie, or Leu,
   Xaa at position 7 is Asn, or Phe,
   Xaa at position 8 is Phe, Val, or Tyr,
   Xaa at position 9 is Leu, Tyr, or Phe,
   Xaa at position 10 is Gly, Thr, or Arg;
(ii) the amino acid sequence of CDR2 is Xaa lie Ser lie Xaa Asp Xaa Xaa Asp (SEQ ID NO: 296), wherein
   Xaa at position 1 is His, or Val,
   Xaa at position 5 is Gly, or Ala,
   Xaa at position 7 is Gln, Val, or Glu,
   Xaa at position 8 is Thr, Ser, or Val; and
(iii) the amino acid sequence of CDR3 is Xaa Ser Xaa lie Xaa Pro Tyr Xaa Xaa (SEQ ID NO: 297), wherein
   Xaa at position 1 is Phe, Leu or Met,
   Xaa at position 3 is Arg, Lys, or Ser,
   Xaa at position 5 is Tyr, or Trp,
   Xaa at position 8 is Asp, or Gln,
   Xaa at position 9 is Tyr, or Trp, and
wherein CDR1, CDR2, CDR3 of said first ISVD are not simultaneously the amino acid sequences of SEQ ID NO's: 153, 209 and 223, respectively.

The first ISVD comprised in the multispecific-multivalent polypeptide of the present technology specifically binds to the constant domain of a human and of a non-human primate TCR.

In one embodiment, the first ISVD specifically binds to the constant domain of a human T cell receptor α (TCR-α) of SEQ ID NO: 291; and/or the constant domain of the human T cell receptor β (TCR-β) of SEQ ID NO: 292, or polymorphic variants or isoforms thereof.

The non-human primate TCR bound by the first ISVD comprised in the multispecific-multivalent polypeptides of the present technology, may be for example a TCR from macaque origin or a TCR from rhesus origin. In one embodiment, the non-human primate TCR is thus a macaque or rhesus TCR. In one embodiment, the macaque or rhesus TCR comprises the constant domain of a TCR-α of SEQ ID NO: 293 and/or of a TCR-β of SEQ ID NO: 294, or polymorphic variants or isoforms thereof.

In one embodiment, the polypeptides of the current technology comprise a first ISVD with improved binding characteristics compared to a corresponding ISVD which comprises the CDR sequences of T0170056G05, i.e. a CDR1 of SEQ ID NO: 153, a CDR2 of SEQ ID NO: 209, and a CDR3 of SEQ ID NO: 223, such as the ISVD T017000141 with SEQ ID NO:2. The binding characteristics of the ISVDs part of the polypeptides of the present technology is discussed in more detail below and in section 5.4: “specificity”.

The inventors identified amino acid mutations in the CDRs that resulted in improved off-rate on human and/or non-human primate TCR, compared to ISVD T017000141 with SEQ ID NO:2. Accordingly, in some embodiments, the first ISVD thus specifically binds to human TCR with the same or lower off rate constant (k_{off}) compared to an ISVD of SEQ ID NO: 2. In some embodiments, the first ISVD specifically binds to non-human primate TCR with the same or lower k_{off} compared to an ISVD of SEQ ID NO: 2.

In some embodiments, the first ISVD has a k_{off} for binding to the constant domain of a human TCR selected from the group consisting of at most about 10⁻³ s⁻¹, at most about 10⁻⁴ s⁻¹, and at most about 10⁻⁵ s⁻¹, preferably as measured by surface plasmon resonance (SPR). In some embodiments, the first ISVD has a k_{off} for binding to the constant domain of a non-human primate TCR selected from the group consisting of at most about 10⁻² s⁻¹, at most about 10⁻³ s⁻¹, and at most about 10⁻⁴ s⁻¹, preferably as measured by SPR.

The inventors also identified specific amino acid residues at specified position in the CDRs, contributing to improved cross-reactivity for binding to human and non-human primate TCR. Accordingly, in one embodiment, the first ISVD has a k_{off} for binding to non-human primate TCR which is within 5-fold range of the k_{off} for binding to human TCR, preferably as measured by SPR.

In particular, the inventors identified specific CDR sequences that resulted in improved binding characteristics compared to T017000141 and which comprise only one amino acid difference compared to the CDR sequences of T017000141. Replacing the CDR1 of SEQ ID NO: 153, the CDR2 of SEQ ID NO: 209, or the CDR3 of SEQ ID NO: 223 comprised in T017000141, by a CDR1 chosen from SEQ ID NO's: 154 to 169, a CDR2 chosen from SEQ ID NO's: 210 to 215, or a CDR3 chosen from SEQ ID NO's: 224 to 230, improved the binding to human and/or non-human primate TCR.

Accordingly, in the multispecific-multivalent polypeptides of the current technology, in one embodiment, in the first ISVD the CDR1 is chosen from the group of amino acid sequences of SEQ ID NO's: 154 to 169. In one embodiment, in the first ISVD the CDR2 is chosen from the group of amino acid sequences of SEQ ID NO's: 210 to 215. In one embodiment, in the first ISVD the CDR3 is chosen from the group of amino acid sequences of SEQ ID NO's: 224 to 230.

In one particular embodiment, the multispecific-multivalent polypeptides of the current technology comprise a first ISVD in which:
(i) CDR1 is chosen from the group of amino acid sequences of SEQ ID NO's: 153 to 169;
(ii) CDR2 is chosen from the group of amino acid sequences of SEQ ID NO's: 209 to 215; and
(iii) CDR3 is chosen from the group of amino acid sequences of SEQ ID NO's: 223 to 230, and
wherein CDR1, CDR2, CDR3 of said first ISVD are not simultaneously the amino acid sequences of SEQ ID NO's: 153, 209 and 223, respectively.

In one particular embodiment, the multispecific-multivalent polypeptides of the current technology comprise a first ISVD in which:
(i) CDR1 is chosen from the group of amino acid sequences of SEQ ID NO's: 154 to 169;
(ii) CDR2 consists of the amino acid sequence HISIGDQTD (SEQ ID NO: 209); and
(iii) CDR3 consists of the amino acid sequence FSRIYPYDY (SEQ ID NO: 223).

In another particular embodiment, the multispecific-multivalent polypeptides of the current technology comprise a first ISVD in which:
(i) CDR1 consists of the amino acid sequence GDVHKINFLG (SEQ ID NO: 153);
(ii) CDR2 is chosen from the group of amino acid sequences of SEQ ID NO's: 210 to 215; and
(iii) CDR3 consists of the amino acid sequence FSRIYPYDY (SEQ ID NO: 223).

In yet another particular embodiment, the multispecific-multivalent polypeptides of the current technology comprise a first ISVD in which:
(i) CDR1 consists of the amino acid sequence GDVHKINFLG (SEQ ID NO: 153);
(ii) CDR2 consists of the amino acid sequence HISIGDQTD (SEQ ID NO: 209); and
(iii) CDR3 is chosen from the group of amino acid sequences of SEQ ID NO's: 224 to 230.

As described in the second part of Example 4 and based on the screening data depicted in Tables 2, 3 and 4, a set of 12 single mutations divided over 9 positions in CDR1 and CDR3—as indicated in Table 5—were selected by the inventors for combination. ISVD variants with combinations of these mutations in their CDRs were subsequently screened to assess the impact of these mutations on the binding of the ISVD to human and/or non-human primate TCR. The mutations that were selected for combination were those present in the CDR1 with the amino acid sequences of SEQ ID NO's: 154 to 163 as compared to the CDR1 of TO17000141, and the mutations present in the CDR3 with the amino acid sequences of SEQ ID NO's: 224 to 228 as compared to the CDR3 of TO17000141. The present technology also relates to polypeptides which comprise these specific amino acid mutations in their CDRs or which comprise the particular CDR1 and/or CDR3 sequences mentioned above.

Accordingly, in a further embodiment, the multispecific-multivalent polypeptide of the current technology comprises a first ISVD in which:
(i) the amino acid sequence of CDR1 is Xaa Xaa Val His Xaa Xaa Asn Phe Xaa Xaa (SEQ ID NO: 298), wherein
   Xaa at position 1 is Gly, Trp, or Tyr,
   Xaa at position 2 is Asp, Glu, Tyr, Ser, or Ala,
   Xaa at position 5 is Lys, or Gln,
   Xaa at position 6 is lie, or Leu,
   Xaa at position 9 is Leu, or Tyr,
   Xaa at position 10 is Gly, or Thr,
(ii) the amino acid sequence of CDR2 is His lie Ser lie Gly Asp Gln Thr Asp (SEQ ID NO: 209), and
(iii) the amino acid sequence of CDR3 is Xaa Ser Xaa lie Xaa Pro Tyr Asp Xaa (SEQ ID NO: 299), wherein
   Xaa at position 1 is Phe, Leu or Met,
   Xaa at position 3 is Arg, or Lys,
   Xaa at position 5 is Tyr, or Trp, and
   Xaa at position 9 is Tyr, or Trp, wherein CDR1, CDR2, CDR3 of said first ISVD are not simultaneously the amino acid sequences of SEQ ID NO's: 153, 209 and 223, respectively.

In one embodiment, the polypeptide of the current technology comprises a first ISVD in which CDR1 is chosen from the group of amino acid sequences of SEQ ID NO's: 154 to 163.

In one embodiment, the polypeptide of the current technology comprises a first ISVD in which CDR3 is chosen from the group of amino acid sequences of SEQ ID NO's: 224 to 228.

In one particular embodiment, the multispecific-multivalent polypeptides of the current technology comprise a first ISVD in which:
(i) CDR1 is chosen from the group of amino acid sequences of SEQ ID NO's: 153 to 163;
(ii) CDR2 is the amino acid sequences of SEQ ID NO's: 209; and
(iii) CDR3 is chosen from the group of amino acid sequences of SEQ ID NO's: 223 to 228,
wherein CDR1, CDR2, CDR3 of said first ISVD are not simultaneously the amino acid sequences of SEQ ID NO's: 153, 209 and 223, respectively.

Contemplated positions for mutations in the CDRs, include those CDR positions which are mutated in ISVD T017000624, i.e. positions 2 and 9 of CDR1, and positions 1 or 5 of CDR3. The present technology also relates to polypeptides which comprise an amino acid mutation in their CDRs at those specific positions.

Accordingly, in a specific embodiment, the multispecific-multivalent polypeptide of the present technology comprises a first ISVD in which CDR1 is chosen from the group of amino acid sequences of SEQ ID NO's: 154 to 158. In another specific embodiment, the polypeptide comprises a first ISVD in which CDR3 is chosen from the group of amino acid sequences of SEQ ID NO's: 224 to 226.

In one particular embodiment, the multispecific-multivalent polypeptides of the current technology comprise a first ISVD in which:
(i) CDR1 is chosen from the group of amino acid sequences of SEQ ID NO's: 153 to 158;
(ii) CDR2 is the amino acid sequences of SEQ ID NO's: 209; and
(iii) CDR3 is chosen from the group of amino acid sequences of SEQ ID NO's: 223 to 226, wherein CDR1, CDR2, CDR3 of said first ISVD are not simultaneously the amino acid sequences of SEQ ID NO's: 153, 209 and 223, respectively.

Contemplated amino acid mutations in the CDRs include those comprised in the CDR sequences of T017000624, i.e. a Tyr at position 2 of CDR1, a Tyr at position 9 of CDR1, a Leu at position 1 of CDR3, or a Trp at position 5 of CDR3.

Accordingly, in a specific embodiment, the multispecific-multivalent polypeptide of the present technology comprises a first ISVD in which CDR1 is chosen from the group of amino acid sequences of SEQ ID NO's: 154 and 155. In another specific embodiment, the polypeptide comprises a first ISVD in which CDR3 is chosen from the group of amino acid sequences of SEQ ID NO's: 224 and 225.

In one particular embodiment, the multispecific-multivalent polypeptides of the current technology comprise a first ISVD in which:
(i) CDR1 is chosen from the group of amino acid sequences of SEQ ID NO's: 153 to 155;
(ii) CDR2 is the amino acid sequences of SEQ ID NO's: 209; and
(iii) CDR3 is chosen from the group of amino acid sequences of SEQ ID NO's: 223 to 225,
wherein CDR1, CDR2, CDR3 of said first ISVD are not simultaneously the amino acid sequences of SEQ ID NO's: 153, 209 and 223, respectively.

As is described in Examples 4 and 5, and as depicted in Tables 6 and 7, the random combination of the set of 12 single mutations divided over 9 positions in CDR1 and CDR3, resulted in the identification of CDR1 and CDR3 sequences with more than one mutation, which in combination conferred the resulting ISVD with improved binding properties as compared to the reference ISVD T017000141 with non-mutated CDRs. The CDR1 and CDR3 sequences comprised in these improved ISVDs, were the CDR1 sequences with any of SEQ ID NO's: 171 to 207 and the CDR3 sequences with any of SEQ ID NO's: 235 to 247.

Accordingly, in a specific embodiment, the multispecific-multivalent polypeptide of the present technology comprises a first ISVD in which CDR1 is chosen from the group of amino acid sequences of SEQ ID NO's: 171 to 207. In another specific embodiment, the polypeptide comprises a first ISVD in which CDR3 is chosen from the group of amino acid sequences of SEQ ID NO's: 235 to 247.

In one particular embodiment, the multispecific-multivalent polypeptides of the current technology comprise a first ISVD in which:
(i) CDR1 is chosen from the group of amino acid sequences of SEQ ID NO's: 153, and 171 to 207;
(ii) CDR2 is the amino acid sequences of SEQ ID NO's: 209; and
(iii) CDR3 is chosen from the group of amino acid sequences of SEQ ID NO's: 223, and 235 to 247,
wherein CDR1, CDR2, CDR3 of said first ISVD are not simultaneously the amino acid sequences of SEQ ID NO's: 153, 209 and 223, respectively.

Advantageous CDR sequences include those that provide the ISVD with improved cross-reactivity for binding to human and non-human primate TCR as compared to T017000141. Particularly advantageous CDR sequences include those that provide the ISVD with a k_{off} for binding to non-human primate TCR which is within 5-fold range of the k_{off} for binding to human TCR, i.e. the CDR1 of SEQ ID NO's: 154, 161, 171 to 175, and 177 to 191, and the CDR3 sequences of SEQ ID NO's: 226, 227, and 235 to 243.

Accordingly, in a specific embodiment, the multispecific-multivalent polypeptide of the present technology comprises a first ISVD in which CDR1 is chosen from the group of amino acid sequences of SEQ ID NO's: 154, 161, 171 to 175, and 177 to 191. In yet another specific embodiment, the polypeptide comprises a first ISVD in which CDR3 is chosen from the group of amino acid sequences of SEQ ID NO's: 226, 227, and 235 to 243.

In one particular embodiment, the multispecific-multivalent polypeptides of the current technology comprise a first ISVD in which:
(i) CDR1 is chosen from the group of amino acid sequences of SEQ ID NO's: 153, 154, 161, 171 to 175, and 177 to 191;
(ii) CDR2 is the amino acid sequences of SEQ ID NO's: 209; and
(iii) CDR3 is chosen from the group of amino acid sequences of SEQ ID NO's: 223, 226, 227, and 235 to 243,
wherein CDR1, CDR2, CDR3 of said first ISVD are not simultaneously the amino acid sequences of SEQ ID NO's: 153, 209 and 223, respectively.

Based on their binding characteristics, a panel of 6 TCR binding ISVDs, i.e. T017000623, T017000624, T017000625, T017000635, T017000638, and T017000641, was selected for further characterisation in the form of a multispecific-multivalent polypeptide of the current technology. As described in Example 6, bispecific CD123/TCR binding polypeptides were generated, some with the TCR binding ISVDs at the N-terminus and others with the TCR binding ISVDs at the C-terminus. The inventors found that all formats with one of the selected TCR binding ISVD variants at the N-terminal position were at least as potent in the human T cell mediated cell killing assay as the same format wherein the selected TCR binding ISVD variant was replaced by T0170056G05. Improved potency could be observed for all the formats with a selected TCR binding ISVD variant at the N-terminal position in cynomolgus T cell mediated cell killing assay compared to the same format wherein the selected TCR binding ISVD variant was replaced by T0170056G05. The CDR1 and CDR3 sequences comprised in these improved ISVDs, i.e. the CDR1 sequences with SEQ ID NO's: 171 to 175 and the CDR3 sequences with SEQ ID NO's: 235 and 236, are contemplated CDRs for the first ISVD in the multispecific-multivalent polypeptide of the present technology.

Accordingly, in a specific embodiment, the multispecific-multivalent polypeptide of the present technology comprises a first ISVD in which CDR1 is chosen from the group of amino acid sequences of SEQ ID NO's: 171 to 175. In another specific embodiment, the polypeptide of the present technology comprises a first ISVD in which CDR3 is chosen from the group of amino acid sequences of SEQ ID NO's: 235 and 236.

In one particular embodiment, the multispecific-multivalent polypeptides of the current technology comprise a first ISVD in which:
(i) CDR1 is chosen from the group of amino acid sequences of SEQ ID NO's: 153, and 171 to 175;
(ii) CDR2 is the amino acid sequences of SEQ ID NO's: 209; and
(iii) CDR3 is chosen from the group of amino acid sequences of SEQ ID NO's: 223, 235 and 236,
wherein CDR1, CDR2, CDR3 of said first ISVD are not simultaneously the amino acid sequences of SEQ ID NO's: 153, 209 and 223, respectively.

The inventors surprisingly discovered that ISVD T017000624 not only exhibited improved binding to human and non-human primate TCR and improved cross-reactivity for binding to TCR from human and non-human primate origin (Example 5). Upon formatting into a bispecific CD123/TCR binding polypeptide, these multispecific-multivalent polypeptides showed improved potency in both the cynomolgus and human T cell mediated cell killing assay, as compared to the same multispecific-multivalent polypeptides which comprise the parental ISVD T0170056G05 instead. Opposed to the other characterized TCR binding ISVD variants, however, this effect could also be observed when T017000624 was located at the C-terminal position. The TCR binding ISVD T017000624, which comprises a CDR1 sequence of SEQ ID NO: 171 and a CDR3 sequence of SEQ ID NO: 235, thus has better formatting properties compared to the other characterized TCR binding ISVDs. The CDR1 and CDR3 sequence comprised in T017000624, i.e. the CDR1 sequence of SEQ ID NO: 171 and the CDR3 sequence of SEQ ID NO: 235, are advantageous CDRs for the first ISVD in the multispecific-multivalent polypeptide of the present technology.

Accordingly, in a specific embodiment, the multispecific-multivalent polypeptides of the present technology comprise a first ISVD wherein CDR1 consists of the amino acid sequence of GYVHKINFYG (SEQ ID NO: 171). In another specific embodiment, the polypeptide of the present technology comprises a first ISVD wherein CDR3 consists of the amino acid sequence of LSRIWPYDY (SEQ ID NO: 235).

TCR binding ISVDs with improved binding to human and/or non-human primate TCR include T017000623, T017000624, T017000625, T017000635, T017000638, and T017000641. The sequences of these ISVDs and their CDRs are listed in Table A-2. In one embodiment of the present technology, the multispecific-multivalent polypeptide thus comprises a first ISVD, in which:

a)
CDR1 consists of the amino acid sequence of
(SEQ ID NO: 171)
GYVHKINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
b)
CDR1 consists of the amino acid sequence of
(SEQ ID NO: 172)
WDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
c)
CDR1 consists of the amino acid sequence of
(SEQ ID NO: 173)
YDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
d)
CDR1 consists of the amino acid sequence of
(SEQ ID NO: 174)
WDVHQINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
e)
CDR1 consists of the amino acid sequence of
(SEQ ID NO: 175)
WDVHKINFYT,
f)
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 236)
LSKIWPYDY.

In a specific embodiment of the present technology, the multispecific-multivalent polypeptides comprise a first ISVD which comprises the CDR sequences of T017000624. Accordingly, in a specific embodiment, the multispecific-multivalent polypeptide of the present technology comprises a first ISVD in which CDR1 consists of the amino acid sequence of GYVHKINFYG (SEQ ID NO: 171), CDR2 consists of the amino acid sequence of HISIGDQTD (SEQ ID NO: 209), and CDR3 consists of the amino acid sequence of LSRIWPYDY (SEQ ID NO: 235).

As described in more detail below (section 5.3: “Immunoglobulin single variable domains”), the first ISVD comprised in the multispecific-multivalent polypeptides of the present technology may have framework sequences that are a suitable combination of immunoglobulin framework sequences or framework sequences that have been derived from immunoglobulin framework sequences, for example, by humanization or camelization. For example, the framework sequences may be framework sequences derived from a light chain variable domain (e.g. a V_{L}-sequence) and/or from a heavy chain variable domain (e.g. a V_{H}-sequence or V_{HH} sequence). In one aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the present technology may contain one or more of hallmark residues (as defined herein), such that the ISVD sequence is a Nanobody®, such as a V_{HH}, including a humanized V_{HH} or camelized V_{H}. Some non-limiting examples of (suitable combinations of) such framework sequences will become clear from the further disclosure herein.

More in particular, the present technology provides multispecific-multivalent polypeptides comprising a first ISVD that specifically binds to the constant domain of a human and of a non-human primate TCR, that is an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and which:
i) have at least 80%, more preferably 90% amino acid identity with at least one of the amino acid sequences of SEQ ID NOs: 1 to 152, 261 or 262 (see Table A-1), in which for the purposes of determining the degree of amino acid identity, the amino acid residues that form the CDR sequences are disregarded. In this respect, reference is also made to Table A-2, which lists the framework 1 sequences (SEQ ID NOs: 248-250), framework 2 sequences (SEQ ID NO: 251), framework 3 sequences (SEQ ID NOs: 252-259) and framework 4 sequences (SEQ ID NOs: 260) of the ISVDs of SEQ ID NOs: 1 to 152, 261 or 262 (see Table A-1);
and in which:
ii) preferably one or more of the amino acid residues at positions 11, 37, 44, 45, 47, 83, 84, 103, 104 and 108 according to the Kabat numbering are chosen from the Hallmark residues mentioned in Table A-3 to Table A-8 of WO 08/020079.

In one embodiment, the multispecific-multivalent polypeptides of the present technology thus comprise a first ISVD which has at least 80%, more preferably 90% sequence identity with at least one of the amino acid sequences of SEQ ID NO's: 1 to 152, 261 or 262 in which for the purposes of determining the degree of sequence identity, the amino acid residues that form the CDR sequences are disregarded.

In one specific embodiment of the present technology, the polypeptide comprises a first ISVD wherein the amino acid residue at position 73 according to Kabat numbering is not asparagine. In one specific embodiment, the amino acid residue at position 73 according to Kabat numbering is glutamic acid.

In one embodiment, the multispecific-multivalent polypeptides of the present technology as described herein, comprise a first ISVD in which
(i) FR1 is chosen from the group of amino acid sequences of SEQ ID NO's: 248 to 250,
(ii) FR2 is the amino acid sequence of SEQ ID NO: 251,
(iii) FR3 is chosen from the group of amino acid sequences of SEQ ID NO's: 252 to 259, and
(iv) FR4 is the amino acid sequence of SEQ ID NO: 260.

In one embodiment, the present technology provides polypeptides as described herein, wherein the first ISVD exhibits improved binding to human and/or non-human primate TCR as compared to T0170056G05. In one embodiment, the first ISVD comprised in the polypeptides of the present technology is chosen from the group of amino acid sequences of SEQ ID NO's: 1 to 20, 22 to 27, 35 to 41, 46 to 147, 150 to 152 and 261 to 262.

In one embodiment, in the multispecific-multivalent polypeptides as described herein, the first ISVD with improved binding properties may comprise only one amino acid difference in one of its CDRs as compared to the CDR sequences of T017000141 or T0170056G05. In one embodiment, the multispecific-multivalent polypeptides of the present technology thus comprise a first ISVD chosen from the group of amino acid sequences of SEQ ID NO's: 1 to 20, 22 to 27, and 35 to 41.

In another embodiment, the first ISVD with improved binding properties comprises more than one amino acid difference in its CDRs when compared to the CDR sequences of T017000141 or T0170056G05. Accordingly, in one embodiment, the multispecific-multivalent polypeptides as described herein comprise a first ISVD chosen from the group of amino acid sequences of SEQ ID NO's: 46 to 147, 150 to 152, 261 and 262.

The inventors found that ISVDs with the amino acid sequences of SEQ ID NO's: 46 to 50, 147 and 150 to 152, 261 and 262, when positioned at the N-terminal position of the polypeptides of the present technology, resulted in increased potency in a cyno T cell mediated cell killing assay as compared to the potency of the same format wherein the first ISVD is replaced by T0170056G05. In one embodiment, the polypeptides as described herein thus comprise a first ISVD chosen from the group of amino acid sequences of SEQ ID NO's: 46 to 50, 147 and 150 to 152, 261 and 262.

ISVDs for use in the polypeptides of the present technology, include those which comprise the CDR sequences of T017000624. As described above, ISVD T017000624, which comprises a CDR1 sequence of SEQ ID NO: 171, a CDR2 sequence of SEQ ID NO: 209 and a CDR3 sequence of SEQ ID NO: 235, has better formatting properties compared to the other characterized TCR binding ISVDs. Accordingly, in a specific embodiment, the polypeptides as described herein thus comprise a first ISVD chosen from the group of amino acid sequences of SEQ ID NO's: 46, 150 to 152, 261 and 262.

Typically, the multispecific-multivalent polypeptides of the current technology combine high affinity antigen recognition on the target cell with T cell activation, resulting in an activation that is independent of the T cells' natural specificity. The first ISVD of the polypeptide of the present technology has high affinity for/specifically binds to an effector cell, preferably the TCR of said effector cell, and even more preferably the constant domain of the TCR.

An effector cell is a cell comprising a TCR complex, preferably an immune cell, such as a T cell, preferably a CD4+T-helper cell (also known as CD4 cell, T-helper cell or T4 cell), more preferably a Cytotoxic T cell (also known as TC cell, CTL or CD8+ T cells) or Natural Killer T cells (NKT cells). In some embodiments, the cell is present in vivo. In some embodiments, the cell is present in vitro. The effector cell of the present technology relates in particular to mammalian cells, preferably to primate cells, and even more preferably to human cells.

The mode of action of the binding molecules that bind both to a cell surface molecule on a target cell such as a tumour antigen and to the T cell TCR is commonly known. Bringing a T cell in close vicinity to a target cell, i.e., engaging said T cell and clustering of the TCR complex results in killing of the target cell by the T cell. In the present technology, this process is exploited in fighting against proliferative disease, inflammatory disease, infectious disease and autoimmune disease. Generally, T cells are equipped with granules containing a deadly combination of pore-forming proteins, called perforins, and cell death-inducing proteases, called granzymes. Preferably, these proteins are delivered into target cells via a cytolytic synapse that forms if T cells are in close vicinity with a target cell that is aimed to be killed. Normally, close vicinity between a T cell and a target cell is achieved by the T cell binding to an MHC/peptide complex using its matching T cell receptor. The multispecific-multivalent polypeptides of the current technology bring a T cell into such close vicinity to a target cell in the absence of T cell receptor/MHC interaction.

Accordingly, the present technology provides a multispecific-multivalent polypeptide as described herein, wherein said polypeptide directs the T cell to the target cell.

With one arm (first ISVD), the multispecific polypeptide has high affinity for/specifically binds to the constant domain of the TCR subunit, a protein component of the signal-transducing complex of the T cell receptor on T cells. With another arm (second ISVD and/or third ISVD, etc.), the multispecific polypeptide recognizes, has high affinity for/specifically binds an antigen(s) on target cells. Preferably, T cell activation is only seen when the multispecific polypeptides are presented to T cells on the surface of target cells. Antigen dependence on target cells for activation results in a favourable safety profile. In an embodiment, the multispecific-multivalent polypeptides transiently tether T cells and target cells. Preferably, the multispecific-multivalent polypeptide can induce resting polyclonal T cells, such as CD4⁺ and/or CD8⁺ T cells into activation, for highly potent redirected lysis of target cells. Preferably, the T cell is directed to a next target cell after lysis of the first target cell.

“T cell activation” as used herein refers to one or more cellular response(s) of a T cell, e.g. a cytotoxic T cell, such as selected from: proliferation, differentiation, cytokine secretion, cytotoxic effector molecule release, cytotoxic activity, expression of activation markers, and redirected target cell lysis. The multispecific-multivalent polypeptides of the current technology are capable of inducing T cell activation. Suitable assays to measure T cell activation are known in the art described herein, for instance as described in WO 99/54440 or by Schlereth et al. 2005 (Cancer Immunol. Immunother. 20: 1-12), or as exemplified in the examples or below.

In an embodiment, the present technology relates to a multispecific-multivalent polypeptide as described herein, wherein said polypeptide induces T cell activation. Preferably, the polypeptide of the present technology induces T cell activation only when said second and/or further ISVD is bound to an antigen on a target cell.

In an embodiment, the present technology relates to a multispecific-multivalent polypeptide as described herein, wherein said T cell activation depends on presenting said polypeptide bound to said first antigen on a target cell to a T cell.

T cell activation by the polypeptides of the present technology can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase. Preferably, redirected lysis of target cells by multispecific-multivalent polypeptides is independent of T cell receptor specificity, presence of MHC class I and/or β2 microglobulin, and/or of any co-stimulatory stimuli.

In an embodiment, the present technology relates to a multispecific-multivalent polypeptide as described herein, wherein said T cell activation is independent from MHC recognition.

The multispecific-multivalent polypeptides of the present technology show redirected lysis in vitro with previously unstimulated peripheral polyclonal CD8⁺- and CD4⁺-positive T cells. The redirected lysis of target cells via the recruitment of T cells by the polypeptides of the present technology involves cytolytic synapse formation and delivery of perforin and granzymes. Cell lysis by T cells has been described, e.g. by Atkinson and Bleackley 1995 (Crit. Rev. Immunol 15(3-4):359-384). Preferably, the engaged T cells are capable of serial target cell lysis, and are not affected by immune escape mechanisms interfering with peptide antigen processing and presentation, or clonal T cell differentiation (see, for example, WO 2007/042261). In vitro, redirected lysis is seen at low picomolar concentrations, suggesting that very low numbers of the polypeptides of the present technology need to be bound to target cells for triggering T cells. As demonstrated in the examples, the low effector to target ratio might be indicative for serial target cell lysis. Accordingly, the present technology relates to potent polypeptides. Preferably, the multispecific-multivalent polypeptide of the current technology mediates killing of target cells, e.g. cancer cells, such as stimulating T cells in pore formation and delivering pro-apoptotic components of cytotoxic T cell granules.

In an embodiment, the present technology relates to a multispecific-multivalent polypeptide as described herein, wherein said T cell activation causes one or more cellular response of said T cell, wherein said cellular response is selected from the group consisting of proliferation, differentiation, cytokine secretion, cytotoxic effector molecule release, cytotoxic activity, expression of activation markers and redirected target cell lysis.

As used herein, the term “potency” is a measure of the biological activity of an agent, such as a polypeptide or an ISVD. Potency of an agent can be determined by any suitable method known in the art, such as for instance as described in the experimental section. Cell culture based potency assays are often the preferred format for determining biological activity since they measure the physiological response elicited by the agent and can generate results within a relatively short period of time. Various types of cell based assays, based on the mechanism of action of the product, can be used, including but not limited to proliferation assays, cytotoxicity assays, cell killing assays, reporter gene assays, cell surface receptor binding assays, and assays to measure induction/inhibition of functionally essential proteins or other signal molecules (such as phosphorylated proteins, enzymes, cytokines, cAMP and the like), Ramos B cell depletion model, T cell mediated tumour cell killing assay (for instance as set out in the Examples section), all well known in the art. Results from cell based potency assays can be expressed as “relative potency” as determined by comparison of the multispecific-multivalent polypeptide of the present technology to the response obtained for the corresponding reference monovalent ISVD, e.g. a polypeptide comprising only one ISVD, optionally further comprising an irrelevant ISVD (cf. experimental section).

In one embodiment, the multispecific-multivalent polypeptides of the present technology showed improved potency and efficacy in both human and cyno T cell mediated killing assays, compared to the same format wherein the first ISVD is replaced by T0170056G05 (Example 11).

The “efficacy” (of the polypeptide of the present technology) measures the maximum strength of the effect itself, at saturating polypeptide concentrations. Efficacy indicates the maximum response achievable by the polypeptide of the present technology. It refers to the ability of a polypeptide to produce the desired (therapeutic) effect.

Accordingly, in an embodiment, the present technology relates to a multispecific-multivalent polypeptide as described herein, wherein said T cell activation causes inhibition of an activity of said target cell, such as to delay or minimize the spread of the target cell, to inhibit or delay growth and/or proliferation of the target cell, and/or to kill the target cell (e.g., cause regression of the disorder and/or symptoms), by more than about 10%, such as 20%, 30%, or 40%, or even more than 50%, such as more than 60%, such as 70%, 80% or even more than 90%, such as 100%. In a particular embodiment, the T cell activation causes lysis of the target cell, by more than about 10%, such as 20%, 30%, or 40%, or even more than 50%, such as more than 60%.

In one embodiment, the multispecific-multivalent polypeptide as described herein causes a human T cell to lyse the target cell with an EC50 value selected from the group consisting of at most about 10⁻⁹ M, at most about 10⁻¹⁰ M, and at most about 10⁻¹¹ M, said EC50 value as determined in a T cell mediated killing assay. For instance, the EC50 is determined in a flow cytometry-based assay as set out in the example 11 or 13.

In one embodiment, the multispecific-multivalent polypeptides as described herein causes a non-human primate T cell to lyse the target cell with an EC50 value selected from the group consisting of at most about 10⁻⁹ M, and at most about 10⁻¹⁰ M, and at most about 10⁻¹¹ M, said EC50 value as determined in a T cell mediated killing assay. For instance, the EC50 is determined in a flow cytometry-based assay as set out in the example 11.

The half maximal inhibitory concentration (IC50) is a measure of the effectiveness of a compound in inhibiting a biological or biochemical function, e.g. a pharmacological effect.

This quantitative measure indicates how much of the ISVD is needed to inhibit a given biological process (or component of a process, i.e. an enzyme, cell, cell receptor, chemotaxis, anaplasia, metastasis, invasiveness, etc) by half. In other words, it is the half maximal (50%) inhibitory concentration (IC) of a substance (50% IC, or IC50). The IC50 of a drug can be determined by constructing a dose-response curve and examining the effect of different concentrations of antagonist such as the ISVD of the present technology on reversing agonist activity. IC50 values can be calculated for a given antagonist such as the ISVD of the present technology by determining the concentration needed to inhibit half of the maximum biological response of the agonist.

The term half maximal effective concentration (EC50) refers to the concentration of a compound which induces a response halfway between the baseline and maximum after a specified exposure time. In the present context it is used as a measure of a polypeptide's, or ISVD's potency. The EC50 of a graded dose response curve represents the concentration of a compound where 50% of its maximal effect is observed. Concentration is preferably expressed in molar units.

In biological systems, small changes in ligand concentration typically result in rapid changes in response, following a sigmoidal function. The inflection point at which the increase in response with increasing ligand concentration begins to slow is the EC50. This can be determined mathematically by derivation of the best-fit line. Relying on a graph for estimation is convenient in most cases. In case the EC50 is provided in the examples section, the experiments were designed to reflect the KD as accurate as possible. In other words, the EC50 values may then be considered as KD values. The term “average KD” relates to the average KD value obtained in at least 1, but preferably more than 1, such as at least 2 experiments. The term “average” refers to the mathematical term “average” (sums of data divided by the number of items in the data).

It is also related to IC50 which is a measure of a compound's inhibition (50% inhibition). For competition binding assays and functional antagonist assays, IC50 is the most common summary measure of the dose-response curve. For agonist/stimulator assays the most common summary measure is the EC50.

The inventors identified multispecific-multivalent polypeptides as described herein, comprising a first ISVD with improved functionality compared to the same polypeptide wherein the first ISVD is replaced by an ISVD of SEQ ID NO:1. In one embodiment, as exemplified herein (cf. Example 6), the multispecific-multivalent polypeptides of the present technology comprising an ISVD of the present technology are capable of activating human and/or non-human primate T cells to lyse a target cell with an improved EC50 value as compared to the same polypeptide wherein the first ISVD is replaced by an ISVD of T0170056G05, i.e. an ISVD of SEQ ID NO: 1

In one embodiment, the multispecific-multivalent polypeptide of the present technology causes a human T cell to lyse the target cell with a lower EC50 value than that of the same polypeptide wherein the first ISVD is replaced by an ISVD of SEQ ID NO: 1, said EC50 value as determined in a T cell mediated killing assay. For instance, the EC50 is determined as set out in the examples section.

In one embodiment, the multispecific-multivalent polypeptide of the present technology causes a non-human primate T cell to lyse the target cell with a lower EC50 value than that of the same polypeptide wherein the first ISVD is replaced by an ISVD of SEQ ID NO: 1, said EC50 value as determined in a T cell mediated killing assay. For instance, the EC50 is determined as set out in the examples section.

In one embodiment, the multispecific-multivalent polypeptides as described herein, comprising a first ISVD of the present technology, show improved binding to the constant domain of a human and/or of a non-human primate TCR, compared to the same polypeptide wherein the first ISVD is replaced by an ISVD of SEQ ID NO: 1, i.e. a polypeptide which comprises as first ISVD and ISVD with the CDR sequences of T0170056G05.

The binding characteristics of the multispecific-multivalent polypeptides of the present technology is discussed in more detail below (section 5.4; “specificity”).

In some embodiments, the multispecific-multivalent polypeptides of the present technology have an on rate constant (kₒₙ) for binding to the human TCR selected from the group consisting of at least about 10³ M⁻¹ s⁻¹, at least about 10⁴ M⁻¹ s⁻¹, and at least about 10⁵ M⁻¹ s⁻¹.

In some embodiments, the multispecific-multivalent polypeptides as described herein have a kₒₙ for binding to the non-human primate TCR selected from the group consisting of at least about 10³ M⁻¹ s⁻¹, at least about 10⁴ M⁻¹ s⁻¹, at least about 10⁵ M⁻¹ s⁻¹, and at least about 106 M⁻¹ s⁻¹.

In some embodiments, the multispecific-multivalent polypeptides of the present technology have an off rate constant (k_{off}) for binding to the human TCR selected from the group consisting of at most about 10⁻² s⁻¹, at most about 10⁻³ s⁻¹, and at most about 10⁻⁴ s⁻¹.

In some embodiments, the multispecific-multivalent polypeptides as described herein have a k_{off} for binding to the non-human primate TCR selected from the group consisting of at most about 10⁻¹ s⁻¹, at most about 10⁻² s⁻¹, at most about 10⁻³ s⁻¹, and at most about 10⁻⁴ s⁻¹.

In some embodiments, the multispecific-multivalent polypeptides of the present technology have an affinity (KD) for binding to the human TCR selected from the group consisting of at most about 10⁻⁶ M, at most about 10⁻⁷ M, at most about 10⁻⁸ M, and at most about 10⁻⁹ M.

In some embodiments, the multispecific-multivalent polypeptides of the present technology have a KD for binding to the non-human primate TCR selected from the group consisting of at most about 10⁻⁶ M, at most about 10⁻⁷ M, and at most about 10⁻⁸ M.

In one embodiment, the kₒₙ, k_{off}, or KD is measured by surface plasmon resonance (SPR). For instance, the kₒₙ, k_{off}, or KD is determined as set out in the examples section. In another embodiment, the kₒₙ, k_{off}, or KD is measured by bio-layer interferometry (BLI).

In one embodiment, the ISVDs of the present technology that specifically bind to the constant domain of the TCR and which are comprised in the polypeptides of the current technology, have improved binding characteristics compared to a corresponding ISVD which comprises the CDR sequences of T0170056G05 of SEQ ID NO: 1, i.e. a CDR1 of SEQ ID NO: 153, a CDR2 of SEQ ID NO: 209, and a CDR3 of SEQ ID NO: 223. As a result, and as exemplified herein, in one embodiment, the multispecific-multivalent polypeptides of the present technology show improved binding to the constant domain of a human and/or of a non-human primate TCR.

In one embodiment, the multispecific-multivalent polypeptides of the present technology binds to the human TCR with a lower KD than that of the same polypeptide wherein the first ISVD is replaced by an ISVD of SEQ ID NO: 1. In one embodiment, the multispecific-multivalent polypeptides of the present technology binds to the non-human primate TCR with a lower KD than that of the same polypeptide wherein the first ISVD is replaced by an ISVD of SEQ ID NO: 1.

It will be appreciated (as is also demonstrated in the Example section) that the first ISVD binding TCR and the second ISVD binding the antigen on a target cell can be positioned in any order in the polypeptide of the present technology. The terms “first ISVD”, “second ISVD”, “third ISVD”, etc., in this regard don't indicate the relative position of the ISVDs to each other. It is also not excluded that other binding units/building blocks such as additional ISVDs binding to additional antigens on a target cells, or binding to another target may be present in the polypeptide. Moreover, it does not exclude the possibility that other binding units/building blocks such as ISVDs can be placed in between. For instance, as described further below (see in particular, section 5.5 “(In vivo) half-life extension” below), the polypeptide can further comprise another ISVD binding to human serum albumin that can even be located between e.g. the “first ISVD” and “second ISVD”.

The inventors surprisingly found, as described in examples 6 and 7, that for both multispecific polypeptides which comprise the ISVD T017000624 either at C-terminal or at N-terminal position, improved potency in a T cell mediated killing assay and improved cross-reactivity for binding to human and non-human primate TCR could be observed.

Accordingly, in one embodiment, the first ISVD binding to TCR is positioned N-terminally from the second ISVD binding the antigen on a target cell. In another embodiment, the first ISVD is positioned at the N-terminus of the polypeptide. In another embodiment, the first ISVD binding to TCR is positioned C-terminally from the second ISVD binding the antigen on a target cell. In a specific embodiment, the first ISVD is positioned at the C-terminus of the polypeptide. In another specific embodiment, the first ISVD is not positioned at the C-terminus of the polypeptide.

In one embodiment, the polypeptide of the present technology comprises at least a first, at least a second, and at least a third ISVD, wherein said at least a first ISVD specifically binds to the constant domain of the T cell receptor (TCR); said at least a second ISVD specifically binds to a first antigen on a target cell, and said at least a third ISVD specifically binds to a second antigen on a target cell. The therapeutic activity of T cell engaging polypeptide can be improved by the simultaneous targeting of multiple tumour associated antigens. In one embodiment, said second antigen is different from said first antigen. In another embodiment, said second antigen is the same as the first antigen.

In such a multispecific-multivalent polypeptide of the present technology, comprising at least a first ISVD specifically binding to the constant domain of the TCR, and at least a second and third ISVD that specifically bind to the same antigen, the at least second and third ISVD can either bind to the same or to different epitopes of the antigen. In one embodiment, the second and third ISVD of the multispecific-multivalent polypeptide of the present technology, bind to the same epitope of the antigen. In another embodiment, the second and third ISVD thus bind to a different epitope of the antigen. Binding multiple epitopes on a single antigen can increase the affinity for binding to the antigen (avidity effect), especially if the different ISVDs are able to simultaneously bind to the different epitopes. In a specific embodiment, the second and third ISVD are able to simultaneously bind to two different epitopes on the same antigen molecule.

It will be appreciated (as is also demonstrated in the Example section) that the ISVD binding TCR and the ISVDs binding the first and second antigen on a target cell can be positioned in any order in the multispecific-multivalent polypeptide of the present technology.

More particularly, in one embodiment, the first ISVD that specifically binds TCR is positioned N-terminally from the second and third ISVD. In a specific embodiment, the first ISVD is positioned at the N-terminus of the polypeptide. In another specific embodiment, the first ISVD that specifically binds TCR is positioned N-terminally, the ISVD specifically binding the second antigen on a target cell is positioned centrally and the ISVD specifically binding the first antigen on a target cell is positioned C-terminally. In yet another specific embodiment, the first ISVD that specifically binds TCR is positioned N-terminally, the ISVD specifically binding the first antigen on a target cell is positioned centrally and the ISVD specifically binding the second antigen on a target cell is positioned C-terminally.

In another embodiment, the first ISVD specifically binding to TCR is positioned C-terminally from the second and third ISVD. In a specific embodiment, the first ISVD is positioned at the C-terminus of the polypeptide. In another specific embodiment, the first ISVD that specifically binds TCR is positioned C-terminally, the ISVD specifically binding the second antigen on a target cell is positioned centrally and the ISVD specifically binding the first antigen on a target cell is positioned N-terminally. In yet another specific embodiment, the first ISVD that specifically binds TCR is positioned C-terminally, the ISVD specifically binding the first antigen on a target cell is positioned centrally and the ISVD specifically binding the second antigen on a target cell is positioned N-terminally.

In yet another embodiment, the first ISVD that specifically binds TCR is positioned in between the second and the third ISVD. In a specific embodiment, the first ISVD that specifically binds TCR is positioned centrally, the ISVD specifically binding the second antigen on a target cell is positioned N-terminally and the ISVD specifically binding the first antigen on a target cell is positioned C-terminally. In yet another specific embodiment, the first ISVD that specifically binds TCR is positioned centrally, the ISVD specifically binding the first antigen on a target cell is positioned N-terminally and the ISVD specifically binding the second antigen on a target cell is positioned C-terminally.

Once again, it is not excluded that other binding units/building blocks such as additional ISVDs binding to additional antigens on a target cells, or binding to another target may be present in the polypeptide. Moreover, the possibility is not excluded that other binding units/building blocks such as ISVDs can be placed in between. For instance, as described further below (see in particular, section 5.5 “(In vivo) half-life extension” below), the polypeptide can further comprise another ISVD specifically binding to human serum albumin that can even be located between e.g. the “first ISVD” and “second ISVD”, the “first ISVD” and “third ISVD”, or the “second ISVD” and “third ISVD”.

The second ISVD of the polypeptide of the present technology specifically binds to an antigen on a target cell, preferably a cancer cell. A “target cell” as referred to herein, is a cell that presents a particular antigen on its surface. In one embodiment, the “target cell” is a cancer cell.

The membrane (also called plasma membrane or phospholipid bilayer) surrounds the cytoplasm of a cell, which is the outer boundary of the cell, i.e. the membrane is the surface of the cell. This membrane serves to separate and protect a cell from its surrounding environment and is made mostly from a double layer of phospholipids. Embedded within this membrane is a variety of protein molecules, such as channels, pumps and cellular receptors. Since the membrane is fluid, the protein molecules can travel within the membrane. The term “antigen on a target cell” as used herein denotes a molecule, which is displayed on the surface of a cell. In most cases, this molecule will be located in or on the plasma membrane of the cell such that at least part of this molecule remains accessible from outside the cell in tertiary form. A non-limiting example of a cell surface molecule, which is located in the plasma membrane, is a transmembrane protein comprising, in its tertiary conformation, regions of hydrophilicity and hydrophobicity. Here, at least one hydrophobic region allows the cell surface molecule to be embedded, or inserted in the hydrophobic plasma membrane of the cell while the hydrophilic regions extend on either side of the plasma membrane into the cytoplasm and extracellular space, respectively.

Said antigen can be any target on a cell, e.g. a tumour antigen. In one embodiment, said antigen is specific for said target cell, e.g. cancer cell, such as a tumour associated antigen (TAA) on said cancer cell.

The term “tumour antigen” as used herein may be understood as those antigens that are presented on tumour cells. These antigens can be presented on the cell surface with an extracellular part, which is often combined with a transmembrane and cytoplasmic part of the molecule. These antigens can sometimes be presented only by tumour cells and never by a normal or healthy cell. Tumour antigens can be exclusively expressed on tumour cells or might represent a tumour specific mutation compared to normal cells. In this case, they are called tumour-specific antigens. However, this will not be the case generally. More common are antigens that are presented by tumour cells and normal cells, and they are called “tumour-associated antigens (TAA)”. These tumour-associated antigens can be overexpressed on tumour cells compared to normal cells or are better accessible for antibody binding in tumour cells due to the less compact structure of the tumour tissue compared to normal tissue. TAA are preferably antigens that are expressed on cells of particular tumours, but that are preferably not expressed in normal cells. Often, TAA are antigens that are normally expressed in cells only at particular points in an organism's development (such as during fetal development) and that are being inappropriately expressed in the organism at the present point of development, or are antigens not expressed in normal tissues or cells of an organ now expressing the antigen.

In an embodiment, said antigen is present more abundantly on a cancer cell than on a normal cell. The antigen on a target cell is preferably a tumour associated antigen (TAA).

In an embodiment, said first antigen on a target cell is a tumour antigen, preferably a tumour associated antigen (TAA).

In an embodiment, said second antigen on a target cell is a tumour antigen, preferably a tumour associated antigen (TAA).

In one embodiment, said first antigen and second antigen are present on the same target cell. In another embodiment, said first antigen and second antigen are present on different target cells.

Contemplated TAAs include MART-1, carcinoembryonic antigen (“CEA”), gp100, MAGE-1, HER-2, CD20, Lewis^{Y} antigens, Melanoma-associated Chondroitin Sulfate Proteoglycan (MCSP), Epidermal Growth Factor Receptor (EGFR), Fibroblast Activation Protein (FAP), and CD19.

Cell surface antigens that are preferentially expressed on AML LSC compared with normal hematopoietic stem cells, and thus contemplated as TAA, include CD123, CD44, CLL-1, CD96, CD47, CD32, CXCR4, Tim-3 and CD25.

Other tumour-associated antigens suitable as an antigen on a target cell for binding by the second ISVD within the polypeptides of the present technology include: TAG-72, Ep-CAM, PSMA, PSA, glycolipids such as GD2 and GD3.

The TAA bound by the second ISVD of the polypeptides of the present technology include also hematopoietic differentiation antigens, i.e. glycoproteins usually associated with cluster differentiation (CD) grouping, such as CD4, CD5, CD19, CD20, CD22, CD36, CD45, CD52, CD69 and CD147; growth factor receptors, including HER2, ErbB3 and ErbB4; Cytokine receptors, including Interleukin-2 receptor gamma chain (CD132 antigen), Interleukin-10 receptor alpha chain (IL-10R-A), Interleukin-10 receptor beta chain (IL-10R-B), Interleukin-12 receptor beta-1 chain (IL-12R-beta1), Interleukin-12 receptor beta-2 chain (IL-12 receptor beta-2), Interleukin-13 receptor alpha-1 chain (IL-13R-alpha-1) (CD213a1 antigen), Interleukin-13 receptor alpha-2 chain (Interleukin-13 binding protein), Interleukin-17 receptor (IL-17 receptor), Interleukin-17B receptor (IL-17B receptor), Interleukin 21 receptor precursor (IL-21R), Interleukin-1 receptor type I (IL-1R-1) (CD121a), Interleukin-1 receptor type II (IL-1R-beta) (CDw121b), Interleukin-1 receptor antagonist protein (IL-1ra), Interleukin-2 receptor alpha chain (CD25 antigen), Interleukin-2 receptor beta chain (CD122 antigen), Interleukin-3 receptor alpha chain (IL-3R-alpha) (CD123 antigen); as well as others, such as CD30, IL23R, IGF-1R, IL5R, IgE, CD248 (endosialin), CD44v6, gpA33, Ron, Trop2, PSCA, claudin 6, claudin 18.2, CLEC12A, CD38, ephA2, c-Met, CD56, MUC16, EGFRvIII, AGS-16, CD27L, Nectin-4, SLITRK6, mesothelin, folate receptor, tissue factor, axl, Glypican-3, CA9, Cripto, CD138, CD37, MUC1, CD70, gastrin releasing peptide receptor, PAP, CEACAM5, CEACAM6, CXCR7, N-cadherin, FXYD2 gamma a, CD21, CD133, Na/K-ATPase, mIgM (membrane-bound IgM), mlgA (membrane-bound IgA), Mer, Tyro2, CD120, CD95, CA 195, DR5, DR6, DcR3 and CAIX.

Accordingly the present technology relates to a polypeptide as described herein, wherein said TAA(s) is (are independently) chosen from the group consisting of Melanoma-associated Chondroitin Sulfate Proteoglycan (MCSP), Epidermal Growth Factor Receptor (EGFR), Fibroblast Activation Protein (FAP), MART-1, carcinoembryonic antigen (“CEA”), gp100, MAGE-1, HER-2, Lewis^{Y} antigens, CD123, CD44, CLL-1, CD96, CD47, CD32, CXCR4, Tim-3, CD25, TAG-72, Ep-CAM, PSMA, PSA, GD2, GD3, CD4, CD5, CD19, CD20, CD22, CD36, CD45, CD52, CD147; growth factor receptors, including ErbB3 and ErbB4; Cytokine receptors, including Interleukin-2 receptor gamma chain (CD132 antigen), Interleukin-10 receptor alpha chain (IL-10R-A), Interleukin-10 receptor beta chain (IL-10R-B), Interleukin-12 receptor beta-1 chain (IL-12R-beta1), Interleukin-12 receptor beta-2 chain (IL-12 receptor beta-2), Interleukin-13 receptor alpha-1 chain (IL-13R-alpha-1) (CD213a1 antigen), Interleukin-13 receptor alpha-2 chain (Interleukin-13 binding protein), Interleukin-17 receptor (IL-17 receptor), Interleukin-17B receptor (IL-17B receptor), Interleukin 21 receptor precursor (IL-21R), Interleukin-1 receptor type I (IL-1R-1) (CD121a), Interleukin-1 receptor type II (IL-1R-beta) (CDw121b), Interleukin-1 receptor antagonist protein (IL-1ra), Interleukin-2 receptor alpha chain (CD25 antigen), Interleukin-2 receptor beta chain (CD122 antigen), Interleukin-3 receptor alpha chain (IL-3R-alpha) (CD123 antigen), CD30, IL23R, IGF-1R, IL5R, IgE, CD248 (endosialin), CD44v6, gpA33, Ron, Trop2, PSCA, claudin 6, claudin 18.2, CLEC12A, CD38, ephA2, c-Met, CD56, MUC16, EGFRvIII, AGS-16, CD27L, Nectin-4, SLITRK6, mesothelin, folate receptor, tissue factor, axl, Glypican-3, CA9, Cripto, CD138, CD37, MUC1, CD70, gastrin releasing peptide receptor, PAP, CEACAM5, CEACAM6, CXCR7, N-cadherin, FXYD2 gamma a, CD21, CD133, Na/K-ATPase, mIgM (membrane-bound IgM), mlgA (membrane-bound IgA), Mer, Tyro2, CD120, CD95, CA 195, DR5, DR6, DcR3 and CAIX, and related polymorphic variants and isoforms, preferably said TAA is CD20 (UniProt 11836), HER2 (Uniprot P04626), EGFR, or CEACAM, polymorphic variants and/or isoforms thereof.

In one embodiment, the multispecific-multivalent polypeptides of the current technology comprise a second ISVD that specifically binds to CD123 or Glypican-3.

Particular multispecific-multivalent polypeptides of the present technology, which comprise a second ISVD that specifically binds to CD123, are provided in Example 6. Their characterization is described in Example 7. Accordingly, in one specific embodiment, the multispecific-multivalent polypeptides of the present technology, comprise a first ISVD chosen from the group consisting of SEQ ID NOs: 46 to 50, 147, 150 to 152, 261 and 262, and a second ISVD is the amino acid sequence of SEQ ID NOs: 263. In a more specific embodiment, said first ISVD is chosen from the group consisting of SEQ ID NOs: 46, 150 to 152, 261 and 262, and said second ISVD consists of the amino acid sequence of SEQ ID NOs: 263. In one embodiment, the multispecific-multivalent polypeptide is chosen from the group of amino acid sequences of SEQ ID NO: 275 and 276.

In another embodiment, the multispecific-multivalent polypeptides of the current technology, comprise a second and a third ISVD that specifically bind to CD123 or Glypican-3.

Particular multispecific-multivalent polypeptides of the present technology, which comprise a second and a third ISVD that specifically binds to CD123, are provided in Example 12. Their characterization is described in Examples 13 and 14. Accordingly, in one specific embodiment, the present technology relates to multispecific-multivalent polypeptides, comprising a first ISVD chosen from the group consisting of SEQ ID NOs: 46, 150 to 152, 261 and 262, a second ISVD with the amino acid sequence of SEQ ID NOs: 263, and a third ISVD which consists of the amino acid sequence of SEQ ID NO: 265. In one embodiment, the multispecific-multivalent polypeptide is chosen from the group of amino acid sequences of SEQ ID NO: 283, 284, 288, 289 and 290.

The second building block, or ISVD in the polypeptide of the present technology has a high affinity for its antigen. The second building block, or ISVD may, for example, be directed against an antigenic determinant, epitope, part, domain, subunit or confirmation (where applicable) of said antigen on a target cell.

The target cell of the present technology relates in particular to mammalian cells, preferably to primate cells, and even more preferably to human cells. The target cell is preferably a hyperproliferative cell such as e.g. a cancer cell.

In one embodiment, the present technology provides polypeptides comprising the multispecific-multivalent polypeptides above and one or more other groups, residues, moieties or binding units, which provide the polypeptide with an increased half-life, as described in detail below (section 5.5; “(In vivo) half-life extension”). In one embodiment, the one or more other groups, residues, moieties or binding units, which provide the polypeptide with an increased half-life is an ISVD that can bind to human serum albumin.

In the polypeptides of the present technology, the two or more ISVDs, and the optionally one or more other groups, residues, moieties or binding units, which provide the polypeptide with an increased half-life, may be directly linked to each other (as for example described in WO 99/23221) and/or may be linked to each other via one or more suitable spacers or linkers, or any combination thereof. In one embodiment, the ISVDs part of the polypeptides of the present technology, are directly linked to each other or are linked via a linker.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, it should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the present technology, since each ISVD by itself forms a complete antigen-binding site).

For example, a linker may be a suitable amino acid sequence, and in particular amino acid sequences of between 1 and 50, preferably between 1 and 30, such as between 1 and 10 amino acid residues. Exemplary peptidic linkers are shown in Table A-5. A linker may be a hinge-like regions, such as the hinge regions of naturally occurring heavy chain antibodies or similar sequences (such as described in WO 94/04678). One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO: 323) motif (for example, having the formula (Gly-Gly-Gly-Gly-Ser)n in which n may be 1, 2, 3, 4, 5, 6, 7 or more). Some often used examples of such GS linkers are 9GS linkers (GGGGSGGGS, SEQ ID NO: 326) 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330.

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the present technology that the length, the degree of flexibility and/or other properties of the linker(s) used (although not critical, as it usually is for linkers used in ScFv fragments) may have some influence on the properties of the final polypeptide of the present technology, including but not limited to the affinity, specificity or avidity for the TCR, or for one or more of the other antigens. Based on the disclosure herein, the skilled person will be able to determine the optimal linker(s) for use in a specific polypeptide of the present technology, optionally after some limited routine experiments.

For example, in multivalent-multispecific polypeptides of the present technology that comprise building blocks, or ISVDs directed against a first and second target, the length and flexibility of the linker are preferably such that it allows each building block, or ISVD present in the polypeptide to bind to its cognate target, e.g. the antigenic determinant on each of the targets. Again, based on the disclosure herein, the skilled person will be able to determine the optimal linker(s) for use in a specific polypeptide of the present technology, optionally after some limited routine experiments.

It is also within the scope of the present technology that the linker(s) used confer one or more other favourable properties or functionality to the polypeptides of the present technology, and/or provide one or more sites for the formation of derivatives and/or for the attachment of functional groups (e.g. as described herein for the derivatives of the ISVDs, or polypeptides of the present technology). For example, linkers containing one or more charged amino acid residues can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the present technology, optionally after some limited routine experiments.

Finally, when two or more linkers are used in the polypeptides of the present technology, these linkers may be the same or different. Again, based on the disclosure herein, the skilled person will be able to determine the optimal linkers for use in a specific polypeptide of the present technology, optionally after some limited routine experiments.

Accordingly, the present technology relates to a polypeptide as described herein, wherein said first ISVD and/or said second ISVD and/or possibly said third ISVD and/or possibly said fourth ISVD binding to (human) serum albumin are linked via a linker.

In one embodiment, said linker is chosen from the group consisting of linkers of 3A, 5GS, 7GS, 8GS, 9GS, 10GS, 15GS, 18GS, 20GS, 25GS, 30GS, 35GS, 40GS, G1 hinge, 9GS-G1 hinge, a llama upper long hinge region, and a G3 hinge (SEQ ID NOs: 322 to 338).

Preferably, the multispecific-multivalent polypeptide of the present technology exhibits reduced binding by pre-existing antibodies in human serum. To this end, in one embodiment, the polypeptide has a valine (V) at amino acid position 11 and a leucine (L), alanine (A), or threonine (T) at amino acid position 89 (according to Kabat numbering) in at least one ISVD, but preferably in each ISVD. In a specific embodiment, the first ISVD of the present technology that specifically binds to the constant domain of the TCR, has a valine (V) at amino acid position 11 and a leucine (L) at amino acid position 89 (according to Kabat numbering). In another specific embodiment, the first ISVD of the present technology that specifically binds to the constant domain of the TCR, has a valine (V) at amino acid position 11 and an alanine (A) at amino acid position 89 (according to Kabat numbering).

In another embodiment, the polypeptide of the current technology comprises a C-terminal extension (X)n, in which n is 1 to 5, such as 1, 2, 3, 4 or 5, and in which X is a naturally occurring amino acid, preferably no cysteine. In one embodiment, the polypeptide has an extension of 1 to 5 (preferably naturally occurring) amino acids, such as a single alanine (A) extension, at the C-terminus of the C-terminal ISVD. The C-terminus of an ISVD is normally VTVSS (SEQ ID NO: 339). In another embodiment, the polypeptide has a lysine (K) or glutamine (Q) at position 110 (according to Kabat numbering) in at least one ISVD. In another embodiment, the ISVD has a lysine (K) or glutamine (Q) at position 112 (according to Kabat numbering) in at least on ISVD. In these embodiments, the C-terminus of the ISVD is VKVSS (SEQ ID NO: 340), VQVSS (SEQ ID NO: 341), VTVKS (SEQ ID NO: 342), VTVQS (SEQ ID NO: 343), VKVKS (SEQ ID NO: 344), VKVQS (SEQ ID NO: 345), VQVKS (SEQ ID NO: 346), or VQVQS (SEQ ID NO: 347), such that after addition of a single alanine the C-terminus of the polypeptide for example has the sequence VTVSSA (SEQ ID NO: 348), VKVSSA (SEQ ID NO: 349), VQVSSA (SEQ ID NO: 350), VTVKSA (SEQ ID NO: 351), VTVQSA (SEQ ID NO: 352), VKVKSA (SEQ ID NO: 353), VKVQSA (SEQ ID NO: 354), VQVKSA (SEQ ID NO: 355), or VQVQSA (SEQ ID NO: 356), preferably VTVSSA. In another embodiment, the polypeptide has a valine (V) at amino acid position 11 and a leucine (L) at amino acid position 89 (according to Kabat numbering) in each ISVD, optionally a lysine (K) or glutamine (Q) at position 110 (according to Kabat numbering) in at least one ISVD and has an extension of 1 to 5 (preferably naturally occurring) amino acids, such as a single alanine (A) extension, at the C-terminus of the C-terminal ISVD (such that the C-terminus of the polypeptide for example has the sequence VTVSSA, VKVSSA or VQVSSA, preferably VTVSSA). See e.g. WO2012/175741 and WO2015/173325 for further information in this regard.

### 5.3 Immunoglobulin Single Variable Domains

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain.

The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb); other single variable domains, or any suitable fragment of any one thereof.

In particular, the immunoglobulin single variable domain may be a immunoglobulin single variable domain Nanobody® (such as a V_{HH}, including a humanized V_{HH} or camelized V_{H}) or a suitable fragment thereof. Nanobody®, Nanobodies® and Nanoclone® are registered trademarks of Ablynx N.V.

“V_{HH} domains”, also known as V_{HH}S, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}S, reference is made to the review article by Muyldermans (Reviews in Molecular Biotechnology 74: 277-302, 2001). Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries e.g. by phage display.

The generation of immunoglobulin sequences has been described extensively in various publications, among which WO 94/04678, Hamers-Casterman et a. 1993 and Muyldermans et al. 2001 can be exemplified. In these methods, camelids are immunized with the target antigen in order to induce an immune response against said target antigen. The repertoire of V_{HH}S obtained from said immunization is further screened for V_{HH}S that bind the target antigen.

In these instances, the generation of antibodies requires purified antigen for immunization and/or screening. Antigens can be purified from natural sources, or in the course of recombinant production.

Immunization and/or screening for immunoglobulin sequences can be performed using peptide fragments of such antigens.

The present technology may use immunoglobulin sequences of different origin, comprising mouse, rat, rabbit, donkey, human and camelid immunoglobulin sequences. The present technology also includes fully human, humanized or chimeric sequences. For example, the present technology comprises camelid immunoglobulin sequences and humanized camelid immunoglobulin sequences, or camelized domain antibodies, e.g. camelized dAb as described by Ward et al (see for example WO 94/04678 and Davies and Riechmann (1994 and 1996)). Moreover, the present technology also uses fused immunoglobulin sequences, e.g. forming a multivalent and/or multispecific construct (for multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221), and immunoglobulin sequences comprising tags or other functional moieties, e.g. toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art (e.g. WO 2008/020079). Again, it should be noted that such humanized V_{HH}S can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein and the prior art (e.g. WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

It should be noted that one or more immunoglobulin sequences may be linked to each other and/or to other amino acid sequences (e.g. via disulphide bridges) to provide peptide constructs that may also be useful in the present technology (for example Fab′ fragments, F(ab′)2 fragments, scFv constructs, “diabodies” and other multispecific constructs). Reference is for example made to the review by Holliger and Hudson, Nat Biotechnol. 2005 September; 23(9):1126-36)). Generally, when a polypeptide is intended for administration to a subject (for example for prophylactic, therapeutic and/or diagnostic purposes), it preferably comprises an immunoglobulin sequence that does not occur naturally in said subject.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

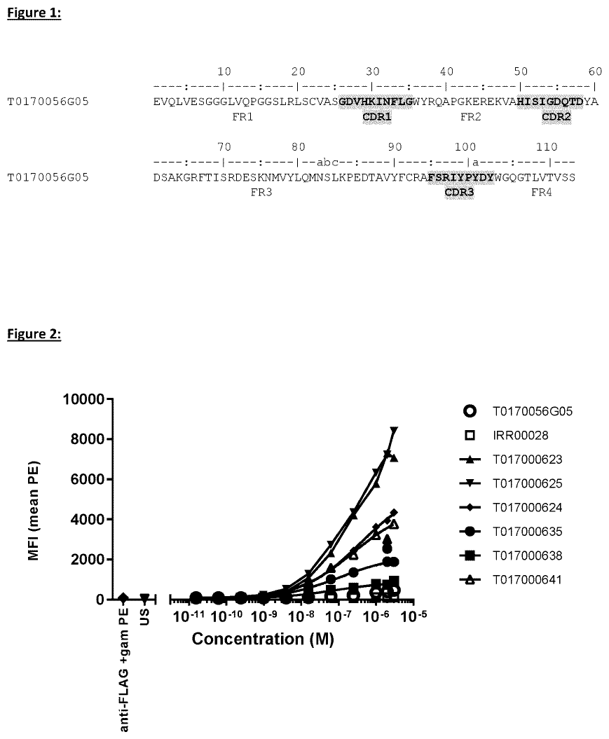

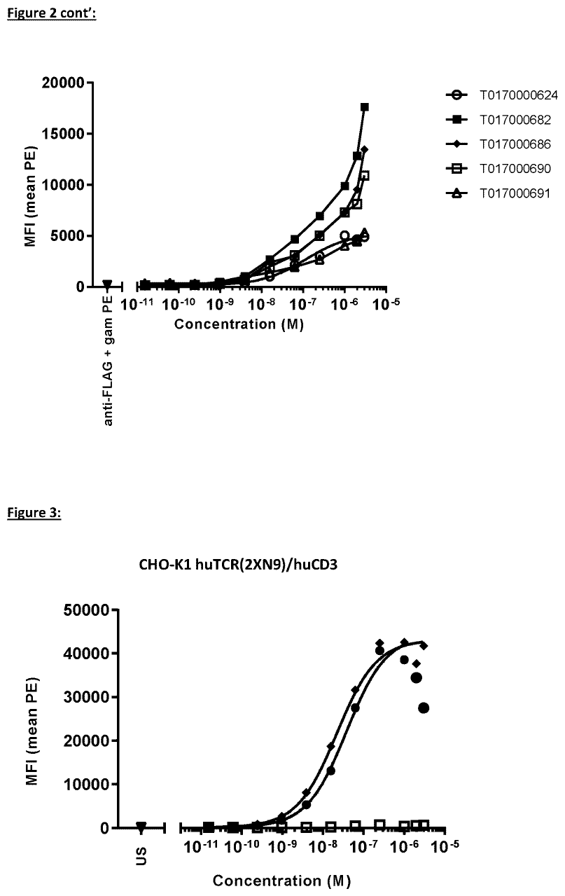

As further described in paragraph q) on pages 58 and 59 of WO 08/020079, the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, CDR sequences were determined according to the AbM definition as described in Kontermann and DQbel (Eds. 2010, Antibody Engineering, vol 2, Springer Verlag Heidelberg Berlin, Martin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are a suitable combination of immunoglobulin framework sequences or framework sequences that have been derived from immunoglobulin framework sequences, for example, by humanization or camelization. For example, the framework sequences may be framework sequences derived from a light chain variable domain (e.g. a V_{L}-sequence) and/or from a heavy chain variable domain (e.g. a V_{H}-sequence or V_{HH} sequence). In one aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the present technology may contain one or more of hallmark residues (as defined herein), such that the ISVD sequence is a V_{HH}, including a humanized V_{HH} or camelized V_{H}. Some non-limiting examples of (suitable combinations of) such framework sequences will become clear from the further disclosure herein.

Again, as generally described herein for the immunoglobulin sequences, it is also possible to use suitable fragments or combinations of fragments of any of the foregoing, such as fragments that contain one or more CDR sequences, suitably flanked by and/or linked via one or more framework sequences; for example, in the same order as these CDR's and framework sequences may occur in the full-sized immunoglobulin sequence from which the fragment has been derived.

However, it should be noted that the current technology is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, an ISVD may be a Nanobody® or a suitable fragment thereof. For a general description of Nanobodies, reference is made to the further description below, as well as to the prior art cited herein. In this respect, it should however be noted that this description and the prior art mainly described Nanobodies of the so-called “V_{H}3 class” (i.e.

Nanobodies with a high degree of sequence homology to human germline sequences of the V_{H}3 class such as DP-47, DP-51 or DP-29). It should however be noted that the present technology in its broadest sense can generally use any type of Nanobody, and for example also uses the Nanobodies belonging to the so-called “V_{H}4 class” (i.e. Nanobodies with a high degree of sequence homology to human germline sequences of the V_{H}4 class such as DP-78), as for example described in WO 2007/118670.

Generally, Nanobodies (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody can be defined as an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody can be an immunoglobulin sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table B-2 below.

TABLE B-2
Hallmark Residues in Nanobodies

| Position | Human V_{H}3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or |
| | | Q⁽³⁾. |
| 45⁽⁸⁾ | L | L⁽²⁾, R⁽³⁾, P, H, F, G, Q, S, E, T, Y, C, I, D, V; preferably |
| | | L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, |
| | | N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; preferably |
| | | P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

The present technology inter alia uses ISVDs that can specifically bind to the constant domain of a human and of a non-humane primate TCR present on a T cell.

In the context of the present technology, “binding to” a certain target molecule has the usual meaning in the art as understood in the context of antibodies and their respective antigens.

The monovalent-monospecific polypeptides of the present technology as described in detail above (section 5.2: “monovalent-monospecific polypeptides”) comprise or (essentially) consist of an ISVD specifically binding to the constant domain of a human and of a non-humane primate TCR present on a T cell. The multivalent-multispecific polypeptides of the present technology as described in detail above (section 5.2: “multispecific-multivalent polypeptides”) comprise an ISVD specifically binding to the constant domain of a human and of a non-humane primate TCR present on a T cell, and one or more ISVDs that specifically bind to an antigen on a target cell.

In one embodiment, the ISVD used in the polypeptides of the current technology specifically binds to the constant domain of a human and of a non-humane primate TCR, essentially consists of 4 framework regions and three complementarity determining regions (CDR1 to CDR3, respectively), wherein
(i) the amino acid sequence of CDR1 is Xaa Xaa Val His Xaa Xaa Xaa Xaa Xaa Xaa (SEQ ID NO: 295), wherein
   Xaa at position 1 is Gly, Trp, Gln, or Tyr,
   Xaa at position 2 is Asp, Glu, Tyr, Ser, or Ala,
   Xaa at position 5 is Lys, or Gln,
   Xaa at position 6 is lie, or Leu,
   Xaa at position 7 is Asn, or Phe,
   Xaa at position 8 is Phe, Val, or Tyr,
   Xaa at position 9 is Leu, Tyr, or Phe,
   Xaa at position 10 is Gly, Thr, or Arg;
(ii) the amino acid sequence of CDR2 is Xaa lie Ser lie Xaa Asp Xaa Xaa Asp (SEQ ID NO: 296), wherein
   Xaa at position 1 is His, or Val,
   Xaa at position 5 is Gly, or Ala,
   Xaa at position 7 is Gln, Val, or Glu,
   Xaa at position 8 is Thr, Ser, or Val; and
(iii) the amino acid sequence of CDR3 is Xaa Ser Xaa lie Xaa Pro Tyr Xaa Xaa (SEQ ID NO: 297), wherein
   Xaa at position 1 is Phe, Leu or Met,
   Xaa at position 3 is Arg, Lys, or Ser,
   Xaa at position 5 is Tyr, or Trp,
   Xaa at position 8 is Asp, or Gln,
   Xaa at position 9 is Tyr, or Trp,
wherein Xaa at position 1, 2, 5, 6, 7, 8, 9 and 10 of CDR1 are not simultaneously Gly, Asp, Lys, lie, Asn, Phe, Leu, and Gly, respectively;
wherein Xaa at position 1, 5, 7, and 8 of CDR2 are not simultaneously His, Gly, Gln, and Thr, respectively; and
wherein Xaa at position 1, 3, 5, 8, and 9 of CDR3 are not simultaneously Phe, Arg, Tyr, Asp, and Tyr, respectively.

When the first ISVD comprised in the polypeptides of the present technology is said to comprise a CDR1 of SEQ ID NO: 295, wherein Xaa at position 1, 2, 5, 6, 7, 8, 9 and 10 are not simultaneously Gly, Asp, Lys, lie, Asn, Phe, Leu, and Gly, respectively; a CDR2 of SEQ ID NO: 296, wherein Xaa at position 1, 5, 7, and 8 are not simultaneously His, Gly, Gln, and Thr, respectively; and a CDR3 of SEQ ID NO: 297, wherein Xaa at position 1, 3, 5, 8, and 9 of CDR3 are not simultaneously Phe, Arg, Tyr, Asp, and Tyr, respectively; it means that in said first ISVD the CDR1, CDR2, CDR3 are not simultaneously the amino acid sequences of SEQ ID NO's: 153, 209, 223, respectively.

As further exemplified herein, the introduction of these particular amino acid residues at these specific positions in a CDR results in improved binding of the ISVD to the constant domain of a human and/or a non-human primate TCR as compared to the binding of ISVD T01700056G05. The inventors have modified the CDRs of ISVD T0170056G05, as to obtain ISVDs with improved binding characteristics compared to a corresponding ISVD which comprises the non-modified CDRs of T0170056G05. In one particular embodiment, the mutations in the CDRs of the ISVD contained in the polypeptide of the present technology, result in improved binding to the constant domain of a human and/or a non-human primate TCR, when compared with an ISVD which comprises a CDR1 of SEQ ID NO: 153, a CDR2 of SEQ ID NO: 209, and a CDR3 of SEQ ID NO: 223 (i.e. the CDR sequences of T0170056G05), such as the ISVD of SEQ ID NO:2.

T0170056G05 was previously developed and described in international application with publication number WO2016180969A1. Based on sequence similarities and differences in CDR2 and CDR3, this ISVD was clustered with 103 other ISVDs, denominated as cluster A. An amino acid sequence alignment of these ISVDs belonging to cluster A is provided in Table A-1 of WO2016180969. None of these cluster A ISVDs have in their CDR1 at position 5 a Lys, or Gln, in combination with in their CDR2 a Ser at position 3, and a Gly or Ala at position 5. The ISVDs comprised in the polypeptides of the present technology are thus novel compared to the sequences described in WO2016180969.

The ISVDs of the current technology which are comprised in the polypeptides of the current technology specifically binds to the constant domain of a human and of a non-human primate TCR present on a T cell.

As used herein, the terms “TCR complex” or “at TCR-CD3 complex” refers to the T cell receptor complex presented on the surface of T cells (see Kuhns et al. 2006, Immunity 24: 133-139). The TCR complex is composed of six different type I single-spanning transmembrane proteins: the TCR-α and TCR-β chains that form the TCR heterodimer responsible for ligand recognition, and the non-covalently associated CD3γ, CD3δ, CD3ε and ζ chains, which bear cytoplasmic sequence motifs that are phosphorylated upon receptor activation and recruit a large number of signalling components. Both α and β chains of the T cell receptor consist of a constant domain and a variable domain.

The sequences of the CD3 chains from human and cynomolgus (cyno) origin are provided in Table A-6 (SEQ ID NOs': 300-303 for CD3 chains of human origin and SEQ ID NO's: 312 to 315 for the CD3 chains of cyno origin). The sequences of the TCR-α/p constant domains of human and cyno origin are also provided in Table A-6 (SEQ ID NO: 291 and 293 for the constant domain of TCR α from human and cyno origin, respectively; SEQ ID NO: 292 and 294 for the constant domain of TCR β from human and cyno origin, respectively). The origin of each of these sequences, as expressed by a UniProt or Genbank files identifier, is listed for each of the aforementioned sequences in Table A-6. In house sequencing confirmed that the amino acid sequences originally derived from rhesus origin, were identical to those from cyno origin.

In one embodiment, the ISVD comprised in the polypeptides of the present technology specifically binds to the constant domain of a human and of a non-human primate TCR present on a viable T cell. A viable T cell, is a T cell capable of exerting a biological activity, such as a cellular response selected from the group consisting of proliferation, differentiation, cytokine secretion, cytotoxic effector molecule release, cytotoxic activity, expression of activation markers and redirected target cell lysis.

In one embodiment, the ISVD comprised in the polypeptide of the present technology specifically binds to the constant domain of a human T cell receptor α (TCR-α) (SEQ ID NO: 291) and/or the constant domain of the human T cell receptor β (TCR-β) (SEQ ID NO: 292), or polymorphic variants or isoforms thereof.

In one embodiment, the non-human primate TCR is a macaque or rhesus TCR. In one embodiment, the macaque or rhesus TCR comprises the constant domain of a TCR-α of SEQ ID NO: 293 and/or of a TCR-β of SEQ ID NO: 294, or polymorphic variants or isoforms thereof.

Isoforms are alternative protein sequences that can be generated from the same gene by a single or by the combination of biological events such as alternative promoter usage, alternative splicing, alternative initiation and ribosomal frameshifting, all as known in the art.

The binding characteristics of the ISVDs part of the polypeptides of the present technology are discussed in more detail herein (section 5.2: “polypeptides of the present technology” and section 5.4: “specificity”). In some embodiments, the ISVD binds to the human TCR with the same or lower off rate constant (k_{off}) compared to an ISVD of SEQ ID NO: 2. In some embodiments, the ISVD binds to non-human primate TCR with the same or lower k_{off} compared to an ISVD of SEQ ID NO: 2. In one embodiment, the ISVD has improved cross-reactivity for binding to human and non-human primate TCR. Accordingly, in a particular embodiment, the ISVD part of the polypeptides of the present technology, has a k_{off} for binding to non-human primate TCR which is within 5-fold range of the k_{off} for binding to human TCR.

Examples of ISVDs which comprise CDR sequences with only one mutation as compared to the CDRs sequences of T01700056G05, and their binding characteristics are depicted in Tables 2, 3 and 4. ISVDs with mutations resulting in an improved off-rate on either human or cyno TCR, have been indicated with a plus in the column listing the effect. Reference is made to Table A-2, which lists the FR and CDR sequences of these ISVDs. Such CDR sequences that confer the ISVD with improved binding characteristics can be used in the ISVD of the current technology. Specific examples of such ISVDs that can be used in the polypeptides of the current technology are as described in the embodiments below.

In one embodiment, the ISVD that specifically binds to the constant domain of a human and of a non-human primate TCR comprises a CDR1 chosen from the group of amino acid sequences of SEQ ID NO's: 154 to 169.

In one embodiment, the ISVD that specifically binds to the constant domain of a human and of a non-human primate TCR comprises a CDR2 chosen from the group of amino acid sequences of SEQ ID NO's: 210 to 215.

In one embodiment, the ISVD that specifically binds to the constant domain of a human and of a non-human primate TCR comprises a CDR3 chosen from the group of amino acid sequences of SEQ ID NO's: 224 to 230.

In one particular embodiment, the ISVD that specifically binds to the constant domain of a human and of a non-human primate TCR comprises
(i) a CDR1 is chosen from the group of amino acid sequences of SEQ ID NO's: 153 to 169;
(ii) a CDR2 is chosen from the group of amino acid sequences of SEQ ID NO's: 209 to 215; and
(iii) a CDR3 is chosen from the group of amino acid sequences of SEQ ID NO's: 223 to 230, and
wherein CDR1, CDR2, CDR3 of said ISVD are not simultaneously the amino acid sequences of SEQ ID NO's: 153, 209 and 223, respectively.

In one embodiment, the ISVD that specifically binds to the constant domain of a human and of a non-human primate TCR comprises
(i) a CDR1 chosen from the group of amino acid sequences of SEQ ID NO's: 154 to 169;
(ii) a CDR2 consisting of the amino acid sequence HISIGDQTD (SEQ ID NO: 209); and
(iii) a CDR3 consisting of the amino acid sequence FSRIYPYDY (SEQ ID NO: 223).

In one embodiment, the ISVD that specifically binds to the constant domain of a human and of a non-human primate TCR comprises
(i) a CDR1 consisting of the amino acid sequence GDVHKINFLG (SEQ ID NO: 153);
(ii) a CDR2 chosen from the group of amino acid sequences of SEQ ID NO's: 210 to 215; and
(iii) a CDR3 consisting of the amino acid sequence FSRIYPYDY (SEQ ID NO: 223).

In one embodiment, the ISVD that specifically binds to the constant domain of a human and of a non-human primate TCR comprises
(i) a CDR1 consisting of the amino acid sequence GDVHKINFLG (SEQ ID NO: 153);
(ii) a CDR2 consisting of the amino acid sequence HISIGDQTD (SEQ ID NO: 209); and
(iii) a CDR3 chosen from the group of amino acid sequences of SEQ ID NO's: 224 to 230.

In a specific embodiment, the ISVD that specifically binds to the constant domain of a human and of a non-human primate TCR is chosen from the group of amino acid sequences of SEQ ID NO's: 1 to 20, 22 to 27, and 35 to 41.

As described in the second part of Example 4 and based on the screening data depicted in Tables 2, 3 and 4, the inventors selected a set of 12 single mutations divided over 9 positions in CDR1 and CDR3—as indicated in Table 5—for combination in order to further improve binding of the ISVD to human and/or non-human primate TCR. CDR sequences with such combinations of mutations can be used in the ISVD comprised in the polypeptides of the current technology.

Accordingly, in a more specific embodiment, the ISVD that specifically binds to the constant domain of a human and of a non-human primate TCR comprises 3 complementarity determining regions (CDR1 to CDR3, respectively), in which:
(i) the amino acid sequence of CDR1 is Xaa Xaa Val His Xaa Xaa Asn Phe Xaa Xaa (SEQ ID NO: 298), wherein
   Xaa at position 1 is Gly, Trp, or Tyr,
   Xaa at position 2 is Asp, Glu, Tyr, Ser, or Ala,
   Xaa at position 5 is Lys, or Gln,
   Xaa at position 6 is lie, or Leu,
   Xaa at position 9 is Leu, or Tyr,
   Xaa at position 10 is Gly, or Thr,
(ii) the amino acid sequence of CDR2 is His lie Ser lie Gly Asp Gln Thr Asp (SEQ ID NO: 209), and
(iii) the amino acid sequence of CDR3 is Xaa Ser Xaa lie Xaa Pro Tyr Asp Xaa (SEQ ID NO: 299), wherein
   Xaa at position 1 is Phe, Leu or Met,
   Xaa at position 3 is Arg, or Lys,
   Xaa at position 5 is Tyr, or Trp, and
   Xaa at position 9 is Tyr, or Trp, and
wherein CDR1, CDR2, CDR3 of said ISVD are not simultaneously the amino acid sequences of SEQ ID NO's: 153, 209 and 223, respectively.

The mutations selected by the inventors for further combination, are those mutations present in the CDR1 with the amino acid sequences of SEQ ID NO's: 154 to 163 as compared to the CDR1 of T017000141, and the mutations present in the CDR3 with the amino acid sequences of SEQ ID NO's: 224 to 228 as compared to the CDR3 of TO17000141. These particular CDR1 and/or CDR3 sequences that confer the ISVD with improved binding characteristics, can be used in the ISVD of the current technology. Specific examples of such ISVDs that can be used in the polypeptides of the current technology are as described in the embodiments below.

In one embodiment, the ISVD that specifically binds to the constant domain of a human and of a non-human primate TCR comprises a CDR1 chosen from the group of amino acid sequences of SEQ ID NO's: 154 to 163. In a specific embodiment, CDR1 is chosen from the group of amino acid sequences of SEQ ID NO's: 154 to 158. In yet another specific embodiment, CDR1 is chosen from the group of amino acid sequences of SEQ ID NO's: 154 and 155.

In one embodiment, the ISVD that specifically binds to the constant domain of a human and of a non-human primate TCR comprises a CDR3 chosen from the group of amino acid sequences of SEQ ID NO's: 224 to 228. In a specific embodiment, CDR3 is chosen from the group of amino acid sequences of SEQ ID NO's: 224 to 226. In yet another specific embodiment, CDR3 is chosen from the group of amino acid sequences of SEQ ID NO's: 224 and 225.

In one particular embodiment, the ISVD that specifically binds to the constant domain of a human and of a non-human primate TCR comprises:
(i) a CDR1 chosen from the group of amino acid sequences of SEQ ID NO's: 153 to 163;
(ii) a CDR2 with the amino acid sequences of SEQ ID NO's: 209; and
(iii) a CDR3 chosen from the group of amino acid sequences of SEQ ID NO's: 223 to 228,
wherein CDR1, CDR2, CDR3 of said ISVD are not simultaneously the amino acid sequences of SEQ ID NO's: 153, 209 and 223, respectively.

In another particular embodiment, the ISVD that specifically binds to the constant domain of a human and of a non-human primate TCR comprises:
(i) a CDR1 chosen from the group of amino acid sequences of SEQ ID NO's: 153 to 158;
(ii) a CDR2 with the amino acid sequences of SEQ ID NO's: 209; and
(iii) a CDR3 chosen from the group of amino acid sequences of SEQ ID NO's: 223 to 226,
wherein CDR1, CDR2, CDR3 of said ISVD are not simultaneously the amino acid sequences of SEQ ID NO's: 153, 209 and 223, respectively.

In yet another particular embodiment, the ISVD that specifically binds to the constant domain of a human and of a non-human primate TCR comprises:
(i) a CDR1 chosen from the group of amino acid sequences of SEQ ID NO's: 153 to 155;
(ii) a CDR2 with the amino acid sequences of SEQ ID NO's: 209; and
(iii) a CDR3 chosen from the group of amino acid sequences of SEQ ID NO's: 223 to 225,
wherein CDR1, CDR2, CDR3 of said ISVD are not simultaneously the amino acid sequences of SEQ ID NO's: 153, 209 and 223, respectively.

The random combination of the set of 12 single mutations divided over 9 positions in CDR1 and CDR3, resulted in the identification of CDR1 and CDR3 sequences with more than one mutation, which in combination conferred the resulting ISVD with improved binding properties as compared to the reference ISVD T017000141 with non-mutated CDRs. The CDR1 and CDR3 sequences comprised in these improved ISVDs, were the CDR1 sequences with any of SEQ ID NO's: 171 to 207 and the CDR3 sequences with any of SEQ ID NO's: 235 to 247. CDR sequences with such combinations of mutations can be used in the ISVD comprised in the polypeptides of the current technology. Specific examples of such ISVDs that can be used in the polypeptides of the current technology are as described in the embodiments below.

In one embodiment, the ISVD that specifically binds to the constant domain of a human and of a non-human primate TCR comprises a CDR1 chosen from the group of amino acid sequences of SEQ ID NO's: 171 to 207.

In one embodiment, the ISVD that specifically binds to the constant domain of a human and of a non-human primate TCR comprises CDR3 is chosen from the group of amino acid sequences of SEQ ID NO's: 235 to 247.

In one particular embodiment, the ISVD that specifically binds to the constant domain of a human and of a non-human primate TCR comprises:
(i) a CDR1 chosen from the group of amino acid sequences of SEQ ID NO's: 153, and 171 to 207;
(ii) a CDR2 with the amino acid sequences of SEQ ID NO's: 209; and
(iii) a CDR3 chosen from the group of amino acid sequences of SEQ ID NO's: 223, and 235 to 247,
wherein CDR1, CDR2, CDR3 of said ISVD are not simultaneously the amino acid sequences of SEQ ID NO's: 153, 209 and 223, respectively.

In a specific embodiment, the ISVD that specifically binds to the constant domain of a human and of a non-human primate TCR is chosen from the group of amino acid sequences of SEQ ID NO's: 46 to 147, 150 to 152, 261 and 262.

Contemplated CDR sequences for use in the ISVDs of the present technology include those that confer an ISVD with improved cross-reactivity for binding to human and non-human primate TCR. Particularly advantageous CDR sequences are those that provide the ISVD with a k_{off} for binding to non-human primate TCR which is within 5-fold range of the k_{off} for binding to human TCR.

Accordingly, in a particular embodiment, the ISVD that specifically binds to the constant domain of a human and of a non-human primate TCR comprises a CDR1 chosen from the group of amino acid sequences of SEQ ID NO's: 154, 161, 171 to 175, and 177 to 191.

In another particular embodiment, CDR3 is chosen from the group of amino acid sequences of SEQ ID NO's: 226, 227, and 235 to 243.

In one particular embodiment, the ISVD that specifically binds to the constant domain of a human and of a non-human primate TCR comprises:
(i) a CDR1 chosen from the group of amino acid sequences of SEQ ID NO's: 153, 154, 161, 171 to 175, and 177 to 191;
(ii) a CDR2 with the amino acid sequences of SEQ ID NO: 209; and
(iii) a CDR3 chosen from the group of amino acid sequences of SEQ ID NO's: 223, 226, 227, and 235 to 243,
wherein CDR1, CDR2, CDR3 of said ISVD are not simultaneously the amino acid sequences of SEQ ID NO's: 153, 209 and 223, respectively.

Contemplated CDR sequences for use in the ISVDs of the present technology include the CDR sequences of T017000623, T017000624, T017000625, T017000635, T017000638, and T017000641. Reference is made to Table A-2, which lists the FR and CDR sequences of these ISVDs. Specific examples of ISVDs comprising these CDR sequences and that can be used in the polypeptides of the current technology are as described in the embodiments below.

In one embodiment, the ISVD that specifically binds to the constant domain of a human and of a non-human primate TCR comprises a CDR1 chosen from the group of amino acid sequences of SEQ ID NO's: 171 to 175.

In one embodiment, the ISVD that specifically binds to the constant domain of a human and of a non-human primate TCR comprises a CDR3 is chosen from the group of amino acid sequences of SEQ ID NO's: 235 and 236.

In one particular embodiment, the ISVD that specifically binds to the constant domain of a human and of a non-human primate TCR comprises:
(i) a CDR1 chosen from the group of amino acid sequences of SEQ ID NO's: 153, and 171 to 175;
(ii) a CDR2 with the amino acid sequences of SEQ ID NO: 209; and
(iii) a CDR3 chosen from the group of amino acid sequences of SEQ ID NO's: 223, 235 and 236,
wherein CDR1, CDR2, CDR3 of said ISVD are not simultaneously the amino acid sequences of SEQ ID NO's: 153, 209 and 223, respectively.

In a specific embodiment, the ISVD that specifically binds to the constant domain of a human and of a non-human primate TCR is chosen from the group of amino acid sequences of SEQ ID NO's: 46 to 50, 147 and 150 to 152, 261 and 262.

Contemplated CDR sequences for use in the ISVDs of the present technology are the CDR sequences of T017000624.

Accordingly, in a particular embodiment, the ISVD comprised in the polypeptide of the present technology, comprises a CDR1 consisting of the amino acid sequence of GYVHKINFYG (SEQ ID NO: 171).

In another particular embodiment, the ISVD comprised in the polypeptide of the present technology, comprises a CDR3 consisting of the amino acid sequence of LSRIWPYDY (SEQ ID NO: 235).

Contemplated combinations of CDR sequences for use in the ISVDs of the present technology, include those combinations of CDRs as indicated for T017000623, T017000624, T017000625, T017000635, T017000638, and T017000641 in Table A-2. Specific examples of ISVDs comprising these combinations of CDR sequences and that can be used in the polypeptides of the current technology are as described below.

In one embodiment, the ISVD that specifically binds to the constant domain of a human and of a non-human primate TCR comprises 3 complementarity determining regions (CDR1 to CDR3, respectively), in which:

a) CDR1 consists of the amino acid sequence of
(SEQ ID NO: 171)
GYVHKINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
b) CDR1 consists of the amino acid sequence of
(SEQ ID NO: 172)
WDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
c)
CDR1 consists of the amino acid sequence of
(SEQ ID NO: 173)
YDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
d) CDR1 consists of the amino acid sequence of
(SEQ ID NO: 174)
WDVHQINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
e) CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
f)
CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 236)
LSKIWPYDY.

A particular combination of CDR sequences for use in the ISVDs of the present technology are those of T017000624. Accordingly, in a particular embodiment, the ISVD comprised in the polypeptide of the present technology, comprises 3 complementarity determining regions (CDR1 to CDR3, respectively), in which: CDR1 consists of the amino acid sequence of GYVHKINFYG (SEQ ID NO: 171), CDR2 consists of the amino acid sequence of HISIGDQTD (SEQ ID NO: 209), and CDR3 consists of the amino acid sequence of LSRIWPYDY (SEQ ID NO: 235).

Particular examples of ISVDs that specifically bind to the constant domain of a human and of a non-human primate TCR have one or more (and preferably all) framework regions as indicated for the ISVDs in Table A-2 (in addition to the CDRs as defined herein above).

Particular ISVDs have the full amino acid sequence of SEQ ID NOs: 1 to 152, 261 or 262 (see Table-1).

In a specific embodiment, the ISVD that specifically binds to the constant domain of a human and of a non-human primate TCR is thus chosen from the group of amino acid sequences of SEQ ID NO's: 46, 150 to 152, 261 and 262.

In one embodiment, the ISVD for use in the polypeptides of the present technology has at least 80%, more preferably 90%, even more preferably 95% sequence identity with at least one of the amino acid sequences of SEQ ID NO's: 1 to 152, 261 or 262 in which for the purposes of determining the degree of sequence identity, the amino acid residues that form the CDR sequences are disregarded.

In one specific embodiment, the amino acid residue at position 73 according to Kabat numbering is not asparagine. In one specific embodiment, the amino acid residue at position 73 according to Kabat numbering is glutamic acid.

In one embodiment, the ISVD for use in the polypeptides of the present technology comprises 4 framework regions (FR1 to FR4, respectively), in which
(i) FR1 is chosen from the group of amino acid sequences of SEQ ID NO's: 248 to 250,
(ii) FR2 is the amino acid sequence of SEQ ID NO: 251,
(iii) FR3 is chosen from the group of amino acid sequences of SEQ ID NO's: 252 to 259, and
(v) FR4 is the amino acid sequence of SEQ ID NO: 260.

In particular, the framework sequences present in the first ISVD used in the polypeptide of the present technology may be the framework sequences of the ISVD T0170056G05.

Accordingly, the ISVD for use in the polypeptides of the present technology comprises 4 framework regions (FR1 to FR4, respectively), in which FR1 is the amino acid sequence of SEQ ID NO: 248, FR2 is the amino acid sequence of SEQ ID NO: 251, FR3 is the amino acid sequence of SEQ ID NO: 252, and FR4 is the amino acid sequence of SEQ ID NO: 260.

Alternatively, the framework sequences present in the first ISVD used in the polypeptide of the present technology may have derived from T0170056G05, but have been further optimised, such as partially or fully humanized or camelized. Alternatively, the framework sequences may have been optimised to avoid the occurrence of post-translation modification during production of the polypeptide, or to reduce the immunogenic profile of said first ISVD. Accordingly, in one embodiment, the ISVD for use in the polypeptides of the present technology comprises 4 framework regions (FR1 to FR4, respectively), in which FR1 is chosen from the group of amino acid sequences of SEQ ID NO: 249 and 250, FR2 is the amino acid sequence of SEQ ID NO: 251, FR3 is chosen from the group of amino acid sequences of SEQ ID NO: 253-259, and FR4 is the amino acid sequence of SEQ ID NO: 260.

In a particular embodiment, the FR3 of the first ISVD is the amino acid sequence of SEQ ID NO: 258. As disclosed in Example 18, ISVD T017000680 which comprises the FR3 of SEQ ID NO: 258 (reference is hereto made to Table A-2), exhibited the best immunogenicity profile in a Dendritic Cell-T cell proliferation assay.

In case the first ISVD is placed at the N-terminal position of the multispecific-multivalent polypeptide of the present technology, preferably the amino acid residue at position 1 according to Kabat numbering in said first ISVD is aspartic acid, as to avoid potential pyroglutamate formation.

In one embodiment, the ISVD for use in the polypeptides of the present technology thus comprises 4 framework regions (FR1 to FR4, respectively), in which FR1 is the amino acid sequence of SEQ ID NO: 249, FR2 is the amino acid sequence of SEQ ID NO: 251, FR3 is the amino acid sequence of SEQ ID NO: 258, and FR4 is the amino acid sequence of SEQ ID NO: 260. In another embodiment, the ISVD comprises a FR1 with the amino acid sequences of SEQ ID NO: 250, a FR2 of the amino acid sequence of SEQ ID NO: 251, a FR3 of the amino acid sequences of SEQ ID NO: 258, and a FR4 of the amino acid sequence of SEQ ID NO: 260.

Specific examples of ISVDs that specifically binds to the constant domain of a human and of a non-human primate TCR and that may be used in the polypeptides of the present technology, are the ISVDs with SEQ ID NO's: 1 to 20, 22 to 27, 35 to 41, 46 to 147, 150 to 152 and 261 to 262. In one embodiment, the ISVD for use in the polypeptides of the present technology is thus chosen from the group of amino acid sequences of SEQ ID NO's: 1 to 20, 22 to 27, 35 to 41, 46 to 147, 150 to 152 and 261 to 262.

A particular ISVD for use in the polypeptides of the present technology is the ISVD with the full amino acid sequence of T017000680 or T017000697. Accordingly, in a particular embodiment, the ISVD for use in the polypeptides of the present technology is thus chosen from the group of amino acid sequences of SEQ ID NO's: 151 and 261.

### 5.4 Specificity

The terms “specificity”, “binding specifically”, “specifically binding”, or “specific binding” refer to the number of different target molecules, such as antigens or a constant domain of the TCR, from the same organism to which a particular binding unit, such as an ISVD, can bind with sufficiently high affinity (see below). Binding units, such as ISVDs, preferably specifically bind to their designated targets.

Specific binding to a certain target from a certain species does not exclude that the binding unit can also specifically bind to the analogous target from a different species. For example, specific binding to a constant domain of the human TCR does not exclude that the binding unit (or a polypeptide comprising the same) can also specifically bind to a constant domain of the TCR from non-humane primates. Likewise, for example, specific binding to an antigen on a target cell, such as human CD123, does not exclude that the binding unit (or a polypeptide comprising the same) can also specifically bind to CD123 from cynomolgus monkeys (“cyno”).

An amino acid sequence such as e.g., an ISVD or polypeptide according to the present technology is said to be “cross-reactive” for two different antigens or antigenic determinants (such as TCR from two different species of mammal, such as human TCR and cynomolgus monkey TCR) if it is specific for (as defined herein) both these different antigens or antigenic determinants.

The specificity/selectivity of a binding unit or polypeptide can be determined based on affinity. The affinity denotes the strength or stability of a molecular interaction. The affinity is commonly indicated by the KD, or dissociation constant, which has units of mol/liter (or M). The affinity can also be expressed as an association constant, KA, which equals 1/KD and has units of (mol/liter)⁻¹ (or M⁻¹). The affinity is a measure for the binding strength between a moiety and a binding site on the target molecule: the lower the value of the KD, the stronger the binding strength between a target molecule and a targeting moiety.

The KD can also be expressed as the ratio of the dissociation rate constant of a complex, denoted as k_{off}, to the rate of its association, denoted kₒₙ(so that K_{D}=k_{off}/kₒₙ and K_{A}=kₒₙ/k_{off}). The “k_{off}”, also referred to herein as “off rate constant”, has units s⁻¹ (where s is the SI unit notation of second). The “kₒₙ”, also referred to herein as “on rate constant”, has units M⁻¹ s⁻¹. The on-rate constant may vary between 10² M⁻¹ s⁻¹ to about 10⁷ M⁻¹ s⁻¹, approaching the diffusion-limited association rate constant for biomolecular interactions. The off rate constant is related to the half-life of a given molecular interaction by the relation t_{1/2}=ln(2)/k_{off}. The off rate constant may vary between 10⁻⁶ s⁻¹ (near irreversible complex with a t_{1/2} of multiple days) to 1 s⁻¹ (t_{1/2}=0.69 s). The lower the value of the k_{off}, the longer it takes for the targeting moiety to dissociate from the target molecule.

Alternatively, the specificity/selectivity of binding unit or polypeptide can be determined based on the off rate constant k_{off}. A mutation in an amino sequence resulting in lowered off rate for binding to a target molecule, will dissociate slower from its target molecule compared to the non-modified amino sequence.

When an ISVD or polypeptide with CDR mutations is said to have improved binding characteristics or properties, this indicates that the ISVD or polypeptide with mutated CDRs has a lower KD or a lower k_{off} compared to a reference ISVD or polypeptide with non-mutated CDRs. In other words, the CDR mutations introduced in the ISVD of polypeptide result in improved binding characteristics, such as the lowering of the KD or the k_{off}.

Accordingly, improved binding may mean that—using the same measurement method, e.g.

SPR, or BLI—an ISVD (or polypeptide comprising the same) binds to the constant domain of TCR with a lower KD value or k_{off} as compared to the ISVD consisting of the amino acid of SEQ ID NO: 2 (or polypeptide comprising the same).

As further exemplified herein, in one embodiment, the ISVDs comprised in the polypeptides of the current technology, have improved binding characteristics compared to a corresponding ISVD which comprises the CDR sequences of T0170056G05, i.e. a CDR1 of SEQ ID NO: 153, a CDR2 of SEQ ID NO: 209, and a CDR3 of SEQ ID NO: 223, such as the ISVD T017000141 with SEQ ID NO:2.

In some embodiments, the polypeptides of the present technology comprise an ISVD that specifically binds to the constant domain of a human TCR with the same or lower off rate constant (k_{off}) compared to an ISVD of SEQ ID NO: 2. In one embodiment, the binding affinity of the ISVD is compared to that of an ISVD of SEQ ID NO:2, wherein the binding affinity is measured using the same method, such as SPR or BLI.

In some other embodiments, the ISVD comprised in the polypeptide binds to the constant domain of a non-human primate TCR with the same or lower k_{off} compared to an ISVD of SEQ ID NO: 2. In one embodiment, the binding affinity of the ISVD is compared to that of an ISVD of SEQ ID NO:2, wherein the binding affinity is measured using the same method, such as SPR or BLI.

In one embodiment, the ISVD comprised in the polypeptides of the present technology has a k_{off} for binding to the constant domain of a human TCR selected from the group consisting of at most about 10⁻³ s⁻¹, at most about 10⁻⁴ s⁻¹, and at most about 10⁻⁵ s⁻¹. In one embodiment, the k_{off} is measured by surface plasmon resonance (SPR). For instance, the k_{off} is determined as set out in the examples section. In another embodiment, the k_{off} is measured by bio-layer interferometry (BLI).

In one embodiment, the ISVD comprised in the polypeptides of the present technology has a k_{off} for binding to the constant domain of a non-human primate TCR selected from the group consisting of at most about 10⁻² s⁻¹, at most about 10⁻³ s⁻¹, and at most about 10⁻⁴ s⁻¹. In one embodiment, the k_{off} is measured by SPR. For instance, the k_{off} is determined as set out in the examples section. In another embodiment, the k_{off} is measured by BLI.

As further exemplified herein, the inventors identified specific amino acid residues at specified position in the CDRs, contributing to improved cross-reactivity for binding to human and non-human primate TCR.

When an ISVD is said to have “improved cross-reactivity for binding to human and non-human primate TCR” or “improved human/non-human primate cross-reactivity” compared to another ISVD, it means that for said ISVD the ratio of the binding activity (such as expressed in terms of KD or k_{off}) for human TCR and for non-human primate TCR is lower than that same ratio calculated for the other ISVD in the same assay.

In one embodiment, the ISVD part of the polypeptides of the present technology, has a k_{off} for binding to non-human primate TCR which is within 5-fold range of the k_{off} for binding to human TCR.

When it is said that an ISVD or polypeptide has a k_{off} for binding to a first antigen (such as non-human primate TCR) which is within 5-fold range of the k_{off} for binding to another antigen (such as human TCR), this means that the k_{off} with which said ISVD or polypeptide binds to the first antigen is not more than 5 times different than the k_{off} with which said ISVD or polypeptide binds to the second antigen. In other words, the ratio of the k_{off} for binding to the first antigen and the k_{off} for binding to the second antigen, is between 0.2 and 5. For example, when an ISVD which has a k_{off} for binding to non-human primate TCR which is within 5-fold range of the k_{off} for binding to human TCR, the ratio of the k_{off} for binding to non-human primate TCR and k_{off} for binding to human TCR, is between 0.2 and 5.

Accordingly, in one embodiment, the ISVD part of the polypeptides of the present technology, has a k_{off}(non-human primate TCR)/k_{off}(human TCR), defined as the ratio of the k_{off} for binding to non-human primate TCR and k_{off} for binding to human TCR, between 0.2 and 5.

Alternatively, as in Example 4 and 5, the difference in k_{off} for binding to a first antigen (such as non-human primate TCR) and a second antigen (such as human TCR) can be calculated by dividing the highest k_{off} value by the lowest k_{off} value. For example, when an ISVD or polypeptide has a k_{off} for binding to non-human primate TCR that is lower than its k_{off} for binding to human primate TCR, the k_{off} for binding to human TCR will be divided by the k_{off} for binding to non-human primate TCR. In the opposite case, when an ISVD or polypeptide has a k_{off} for binding to non-human primate TCR that is higher than its k_{off} for binding to human primate TCR, the k_{off} for binding to non-human primate TCR will be divided by the k_{off} for binding to human TCR. This calculation method will result in a value which is 1 or higher. A high value is reflective of a big difference in k_{off} for binding to human and non-human primate TCR, while a low value is indicative for a small difference in k_{off} for binding to human and non-human primate TCR. When the calculated value for an ISVD or polypeptide is between 1 and 5, it means that the ISVD or polypeptide has a k_{off} for binding to non-human primate TCR which is within 5-fold range of its k_{off} for binding to human TCR. The value obtained by this calculation is indicated in the Examples as the “gap in off-rate human vs cyno”.

This alternative calculation method allows for easy comparison of species cross-reactivity of different ISVDs variants. When, upon introduction of one or more mutation(s) in a CDR, the calculated value for the ISVD is lower than the value calculated for the reference ISVD without any of these mutations in its CDRs, it indicates that the mutated ISVD has improved cross-reactivity for binding to human and non-human primate TCR, also referred to interchangeably herein as “improved human/non-human primate cross-reactivity” or a “lower gap for human/non-human primate cross-reactivity”, or more specifically “improved human/cyno cross-reactivity” or a “lower gap for human/cyno cross-reactivity”. In this case, the mutations introduced in the CDRs of the ISVD contribute to improved human/cyno cross-reactivity. Such mutations and the resulting CDRs are particularly advantageous for use in the ISVDs and polypeptides of the present technology. Particularly advantageous are those CDR sequences comprised in an ISVD or polypeptide with a k_{off} for binding to non-human primate TCR which is within 5-fold range of its k_{off} for binding to human TCR. Such ISVDs or polypeptides are also referred to herein as having a gap for human/cyno cross-reactivity of 5-fold or lower.

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (K_{A}), by means of the relationship [KD=₁/K_{A}].

The affinity of a molecular interaction between two molecules can be measured via different techniques known per se, such as the well-known surface plasmon resonance (SPR) biosensor technique (see for example Ober et al. 2001, Intern. Immunology 13: 1551-1559). The term “surface plasmon resonance” (“SPR”), as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values.

This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991 Biotechniques 11: 620-627), Johnsson et al. (1995, J. Mol. Recognit. 8: 125-131), and Johnnson et al. (1991, Anal. Biochem. 198: 268-277). Alternatively, SPR can be performed using the well-known ProteOn™ system (Bio-Rad Laboratories Inc).

Another well-known biosensor technique to determine affinities of biomolecular interactions is bio-layer interferometry (BLI) (see for example Abdiche et al. 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5: 1765-74).

Any KD value greater than 10⁻⁴ mol/liter (or any K_{A} value lower than 10⁴ liters/mol) is generally considered to indicate non-specific binding.

The KD for biological interactions, such as the binding of immunoglobulin sequences to an antigen, which are considered specific are typically in the range of 10⁻¹ moles/liter (10000 nM or 10 μM) to 10⁻¹2 moles/liter (0.001 nM or 1 μM) or less.

An ISVD or polypeptide is said to be “specific for” a first target or antigen compared to a second target or antigen when it binds to the first antigen with an affinity/avidity (as described above, and suitably expressed as a K_{D} value, K_{A} value, k_{off} rate and/or kₒₙ rate) that is at least 10 times, such as at least 100 times, and preferably at least 1000 times, and up to 10.000 times or more better than the affinity with which said ISVD or polypeptide binds to the second target or polypeptide. For example, the ISVD or polypeptide may bind to the target or antigen with a K_{D} value that is at least 10 times less, such as at least 100 times less, and preferably at least 1000 times less, such as 10.000 times less or even less than that, than the K_{D} with which said ISVD or polypeptide binds to the second target or polypeptide.

Preferably, when an immunoglobulin single variable domain or polypeptide is “specific for” a first target or antigen compared to a second target or antigen, it is directed against (as defined herein) said first target or antigen, but not directed against said second target or antigen.

Binding affinities of the multispecific-multivalent polypeptides of the present technology have been tested, as described in Example 7.

In some embodiments, the multispecific-multivalent polypeptides of the present technology have an on rate constant (kₒₙ) for binding to the human TCR selected from the group consisting of at least about 10³ M⁻¹ s⁻¹, at least about 10⁴ M⁻¹ s⁻¹, and at least about 10⁵ M⁻¹ s⁻¹.

In one embodiment, the kₒₙ is at least about 10⁴ M⁻¹ s⁻¹. In another embodiment, the kₒₙ is between 10⁴ M⁻¹s⁻¹ and 10⁵ M⁻¹ s⁻¹.

In some embodiments, the multispecific-multivalent polypeptides as described herein have a kₒₙ for binding to the non-human primate TCR selected from the group consisting of at least about 10³ M⁻¹s⁻¹, at least about 10⁴ M⁻¹ s⁻¹, and at least about 10⁵ M⁻¹ s⁻¹. In one embodiment, the kₒₙ is at least about 10⁴ M⁻¹ s⁻¹. In another embodiment, the kₒₙ is between 10⁴ M⁻¹ s⁻¹ and 10⁵ M⁻¹ s⁻¹.

In some embodiments, the multispecific-multivalent polypeptides of the present technology have a k_{off} for binding to the human TCR selected from the group consisting of at most about 10⁻² s⁻¹, at most about 10⁻³ s⁻¹, and at most about 10⁻⁴ s⁻¹. In one embodiment, the k_{off} is at most about 10⁻³ s⁻¹. In another embodiment, the k_{off} is between 10⁻³ s⁻¹ and 10⁻⁴ s⁻¹.

In some embodiments, the multispecific-multivalent polypeptides as described herein have a k_{off} for binding to the non-human primate TCR selected from the group consisting of at most about 10⁻¹ s⁻¹, at most about 10⁻² s⁻¹, at most about 10⁻³ s⁻¹, and at most about 10⁻⁴ s⁻¹. In one embodiment, the k_{off} is at most about 10⁻² s⁻¹. In another embodiment, the k_{off} is between 10⁻² s-land 10⁻³ s⁻¹.

In some embodiments, the multispecific-multivalent polypeptides of the present technology have an affinity (KD) for binding to the human TCR selected from the group consisting of at most about 10⁻⁶ M, at most about 10⁻⁷ M, at most about 10⁻⁸ M, and at most about 10⁻⁹ M. In one embodiment, the KD is at most about 10⁻⁸ M. In another embodiment, the KD is between 10⁻⁹ M and 10⁻⁸ M.

In some embodiments, the multispecific-multivalent polypeptides of the present technology have a KD for binding to the non-human primate TCR selected from the group consisting of at most about 10⁻⁶ M, at most about 10⁻⁷ M, and at most about 10⁻⁸ M. In one embodiment, the KD is at most about 10⁻⁷ M. In another embodiment, the KD is between 10⁻⁸ M and 10⁻⁷ M.

In one embodiment, the ISVDs of the present technology that bind to the constant domain of the TCR and which are comprised in the polypeptides of the current technology, have improved binding characteristics compared to a corresponding ISVD which comprises the CDR sequences of T0170056G05 of SEQ ID NO: 1, i.e. a CDR1 of SEQ ID NO: 153, a CDR2 of SEQ ID NO: 209, and a CDR3 of SEQ ID NO: 223. As a result, and as exemplified herein, the multispecific-multivalent polypeptides of the present technology, in one embodiment, show improved binding to the constant domain of a human and/or of a non-human primate TCR.

In one embodiment, the multispecific-multivalent polypeptides of the present technology binds to the human TCR with a lower KD than that of the same polypeptide wherein the first ISVD is replaced by an ISVD of SEQ ID NO: 1. In one embodiment, the multispecific-multivalent polypeptides of the present technology binds to the non-human primate TCR with a lower KD than that of the same polypeptide wherein the first ISVD is replaced by an ISVD of SEQ ID NO: 1. The KD of the multispecific-multivalent polypeptides of the present technology and of the same polypeptide wherein the first ISVD is replaced by an ISVD of SEQ ID NO: 1, may be measured with the same method. In one embodiment, the KD of the multispecific-multivalent polypeptides of the present technology for binding to TCR is thus compared to that of the same polypeptide wherein the first ISVD is replaced by an ISVD of SEQ ID NO: 1, wherein the KD is measured using the same method.

Affinity of the multispecific-multivalent polypeptides of the present technology can be measured via different techniques as described herein above. In one embodiment, the kₒₙ, k_{off}, or KD, of the multispecific-multivalent polypeptides of the current technology is measured by SPR. For instance, the kₒₙ is determined as set out in the examples section. In another embodiment, the kₒₙ, k_{off}, or KD, of said polypeptides is measured by BLI.

### 5.5 (In Vivo) Half-Life Extension

The polypeptide of the present technology may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide has an increased half-life in a mammal, such as a human subject, after administration. Half-life can be expressed for example as t1/2beta.

The type of groups, residues, moieties or binding units is not generally restricted and may for example be chosen from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be chosen from the group consisting of binding units that can bind to serum albumin, such as human serum albumin, or a serum immunoglobulin, such as IgG. In one embodiment, said one or more other groups, residues, moieties or binding units that provide the polypeptide with increased half-life is a binding unit that can specifically bind to human serum albumin. In one embodiment, the binding unit is an ISVD.

For example, WO 04/041865 describes Nanobodies® binding to serum albumin (and in particular against human serum albumin) that can be linked to other proteins (such as one or more other Nanobodies binding to a desired target) in order to increase the half-life of said protein.

The international application WO 06/122787 describes a number of Nanobodies® against (human) serum albumin. These Nanobodies® include the Nanobody® called Alb-1 (SEQ ID NO: 52 in WO 06/122787) and humanized variants thereof, such as Alb-8 (SEQ ID NO: 62 in WO 06/122787). Again, these can be used to extend the half-life of therapeutic proteins and polypeptide and other therapeutic entities or moieties.

Moreover, WO2012/175400 describes a further improved version of Alb-1, called Alb-23 (SEQ ID NO: 1 in WO2012/175400).

In one embodiment, the polypeptide comprises an ISVD that can bind to human serum albumin selected from Alb-1, Alb-3, Alb-4, Alb-5, Alb-6, Alb-7, Alb-8, Alb-9, Alb-10 (respectively SEQ ID NOs: 52, 50, 57-64 in WO 06/122787) and Alb-23. In one embodiment, the ISVD that can bind to human serum albumin is Alb-8 or Alb-23 or its variants, as shown in pages 7-9 of WO2012/175400 and the albumin binders described in WO 2012/175741, WO2015/173325, WO2017/080850, WO2017/085172, WO2018/104444, WO2018/134235, WO2018/134234.

In one embodiment, the ISVD that can bind to human serum albumin is selected from the ISVDs shown in Table A-4 with SEQ ID NOs: 361 to 378.

In one embodiment, the binding unit that provides the polypeptide with increased (in vivo) half-life is an ISVD that can bind to human serum albumin, which essentially consists of 4 framework regions (FR1 to FR4, respectively) and 3 complementarity determining regions (CDR1 to CDR3, respectively), wherein
i. the amino acid sequence of CDR1 is GFTFRSFGMS (SEQ ID NO: 357), GFTFSSFGMS (SEQ ID NO: 358), an amino acid sequence with 2 or 1 amino acid difference with SEQ ID NO: 357, or an amino acid sequence with 2 or 1 amino acid difference with SEQ ID NO 358;
ii. the amino acid sequence of CDR2 is SISGSGSDTL (SEQ ID NO: 359), or an amino acid sequence with 2 or 1 amino acid difference with SEQ ID NO 359; and
iii. the amino acid sequence of CDR3 is GGSLSR (SEQ ID NO: 360), or an amino acid sequence with 2 or 1 amino acid difference with SEQ ID NO 360;

When such an ISVD binding to human serum albumin has 2 or 1 amino acid difference in at least one CDR relative to a corresponding reference CDR sequence, the ISVD has at least half the binding affinity, or at least the same binding affinity to human serum albumin compared to the binding affinity of SEQ ID NO: 375, wherein the binding affinity is measured using the same method, such as SPR.

In one embodiment, the ISVD that binds to human serum albumin essentially consists of 4 framework regions (FR1 to FR4, respectively) and 3 complementarity determining regions (CDR1 to CDR3, respectively), wherein the amino acid sequence of CDR1 is GFTFRSFGMS (SEQ ID NO: 357) or GFTFSSFGMS (SEQ ID NO: 358), the amino acid sequence of CDR2 is SISGSGSDTL (SEQ ID NO: 359), and the amino acid sequence of CDR3 is GGSLSR (SEQ ID NO: 360).

Also, in one embodiment, the amino acid sequence of an ISVD binding to human serum albumin may have a sequence identity of more than 90%, such as more than 95% or more than 99%, with SEQ ID NO: 375 or SEQ ID NO: 361, respectively, in which for the purposes of determining the degree of sequence identity, the amino acid residues that form the CDR sequences are disregarded. In one embodiment, the ISVD binding to human serum albumin has the amino acid sequence of SEQ ID NO: 375.

In one embodiment, when such an ISVD binding to human serum albumin has a C-terminal position it exhibits a C-terminal extension, such as a C-terminal alanine (A) or glycine (G) extension. In one embodiment such an ISVD is selected from SEQ ID NOs: 362, 364, 366, 368, 370, 372, 374, 376 and 378 (see Table A-4 below). In another embodiment, the ISVD binding to human serum albumin has another position than the C-terminal position (i.e. is not the C-terminal ISVD of the polypeptide of the present technology). In one embodiment such and ISVD is selected from SEQ ID NOs: 361, 363, 365, 367, 369, 371, 373, 375 and 377 (see Table A-4 below).

### 5.6 Nucleic Acid Molecules

Also provided is a nucleic acid molecule encoding the polypeptide of the present technology.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A host or host cell transformed or transfected with a nucleic acid encoding the polypeptide of the present technology is also encompassed by the present technology.

A nucleic acid may be for example DNA, RNA, or a hybrid thereof, and may also comprise (e.g. chemically) modified nucleotides, like PNA. It can be single- or double-stranded. In one embodiment, it is in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly)peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### 5.7 Vectors

Also provided is a vector comprising the nucleic acid molecule encoding the polypeptide of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

In some embodiments, vectors generally comprise at least one nucleic acid that is optionally linked to one or more regulatory elements, such as for example one or more suitable promoter(s), enhancer(s), terminator(s), etc.). In one embodiment, the vector is an expression vector, i.e. a vector suitable for expressing an encoded polypeptide or construct under suitable conditions, e.g. when the vector is introduced into a (e.g. human) cell. For DNA-based vectors, this usually includes the presence of elements for transcription (e.g. a promoter and a polyA signal) and translation (e.g. Kozak sequence).

In one embodiment, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

In one embodiment, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

Some preferred, but non-limiting promoters for use with particular host cells include:
for expression in E. coli: lac promoter (and derivatives thereof such as the lacUV5 promoter); arabinose promoter; left—(PL) and rightward (PR) promoter of phage lambda; promoter of the trp operon; hybrid lac/trp promoters (tac and trc); T7-promoter (more specifically that of T7-phage gene 10) and other T-phage promoters; promoter of the Tn10 tetracycline resistance gene; engineered variants of the above promoters that include one or more copies of an extraneous regulatory operator sequence;
for expression in S. cerevisiae: constitutive: ADH1 (alcohol dehydrogenase 1), ENO (enolase), CYC1 (cytochrome c iso-1), GAPDH (glyceraldehydes-3-phosphate dehydrogenase), PGK1 (phosphoglycerate kinase), PYK1 (pyruvate kinase); regulated: GAL1,10,7 (galactose metabolic enzymes), ADH2 (alcohol dehydrogenase 2), PHO5 (acid phosphatase), CUP1 (copper metallothionein); heterologous: CaMV (cauliflower mosaic virus 35S promoter);
for expression in Pichia pastoris: the AOX1 promoter (alcohol oxidase 1);
for expression in mammalian cells: human cytomegalovirus (hCMV) immediate early enhancer/promoter; human cytomegalovirus (hCMV) immediate early promoter variant that contains two tetracycline operator sequences such that the promoter can be regulated by the Tet repressor; Herpes Simplex Virus thymidine kinase (TK) promoter; Rous Sarcoma Virus long terminal repeat (RSV LTR) enhancer/promoter; elongation factor 1α (hEF-1α) promoter from human, chimpanzee, mouse or rat; the SV40 early promoter; HIV-1 long terminal repeat promoter; β-actin promoter.

Some preferred, but non-limiting vectors for use with particular host cells include:
vectors for expression in mammalian cells: pMAMneo (Clontech), pcDNA3 (Invitrogen), pMC1neo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565), as well as viral-based expression systems, such as those based on adenovirus;
vectors for expression in bacterial cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacIl (Invitrogen) and other baculovirus vectors;
vectors for expression in plants or plant cells: for example vectors based on cauliflower mosaic virus or tobacco mosaic virus, suitable strains of Agrobacterium, or Ti-plasmid based vectors.

Some preferred, but non-limiting secretory sequences for use with particular host cells include:
for use in bacterial cells such as E. coli: PeIB, Bla, OmpA, OmpC, OmpF, OmpT, StIl, PhoA, PhoE, MalE, Lpp, LamB, and the like; TAT signal peptide, hemolysin C-terminal secretion signal;
for use in yeast: α-mating factor prepro-sequence, phosphatase (phol), invertase (Suc), etc.;
for use in mammalian cells: indigenous signal in case the target protein is of eukaryotic origin; murine Ig K-chain V-J2-C signal peptide; etc.

### 5.8 Host Organisms

The present technology also pertains to host cells or host organisms comprising the polypeptide of the present technology. In one embodiment, the host cell is transformed or transfected with the nucleic acid encoding the polypeptide of the present technology, or the vector comprising the nucleic acid molecule encoding the polypeptide of the present technology.

The host is preferably a non-human host. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al. 1998 (Res. Immunol. 149: 589-99); Riechmann and Muyldermans 1999 (J. Immunol. Met. 231: 25-38); van der Linden 2000 (J. Biotechnol. 80: 261-70); Joosten et al. 2003 (Microb. Cell Fact. 2: 1); Joosten et al. 2005 (Appl. Microbiol. Biotechnol. 66: 384-92); and the further references cited herein.

Suitable techniques for transforming a host or host cell of the present technology will be clear to the skilled person and may depend on the intended host cell/host organism and the nucleic acid or vector to be used. Reference is again made to the handbooks and patent applications mentioned above.

After transformation, a step for detecting and selecting those host cells or host organisms that have been successfully transformed with the nucleic acid/vector of the present technology may be performed. This may for instance be a selection step based on a selectable marker present in the vector of the current technology or a step involving the detection of the polypeptide of the present technology, e.g. using specific antibodies.

The transformed host cell (which may be in the form or a stable cell line) or host organisms (which may be in the form of a stable mutant line or strain) form further aspects of the present technology.

Preferably, these host cells or host organisms are such that they express, or are (at least) capable of expressing (e.g. under suitable conditions), an ISVD or polypeptide of the current technology (and in case of a host organism: in at least one cell, part, tissue or organ thereof). The present technology also includes further generations, progeny and/or offspring of the host cell or host organism of the present technology, for instance obtained by cell division or by sexual or asexual reproduction.

Accordingly, in another aspect, the technology relates to a host or host cell that expresses (or that under suitable circumstances is capable of expressing) an ISVD or polypeptide of the current technology; and/or that contains a nucleic acid or vector encoding the same. Some preferred but non-limiting examples of such hosts or host cells can be as generally described in WO 04/041867, WO 04/041865 or WO 09/068627. For example, ISVDs and polypeptides of the current technology may with advantage be expressed, produced or manufactured in a yeast strain, such as a strain of Pichia pastoris. Reference is also made to WO 04/25591, WO 10/125187, WO 11/003622, and WO 12/056000 which also describes the expression/production in Pichia and other hosts/host cells of immunoglobulin single variable domains and polypeptides comprising the same.

### 5.9 Process

The present technology also provides a process for producing the polypeptide of the present technology. The process may comprise transforming/transfecting a host cell or host organism with a nucleic acid encoding the polypeptide, or with a vector comprising the nucleic acid encoding the polypeptide, expressing the polypeptide in the host, optionally followed by one or more isolation and/or purification steps. In one embodiment, the method comprises culturing a host cell transformed or transfected with a nucleic acid encoding the polypeptide, or with a vector comprising the nucleic acid encoding the polypeptide, under conditions allowing the expression of the polypeptide and recovering the produced polypeptide from the culture.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

To produce/obtain expression of the polypeptides of the present technology, the transformed host cell or transformed host organism may generally be kept, maintained and/or cultured under conditions such that the (desired) ISVD, or polypeptide of the present technology is expressed/produced. Suitable conditions will be clear to the skilled person and will usually depend upon the host cell/host organism used, as well as on the regulatory elements that control the expression of the (relevant) nucleotide sequence of the present technology.

Generally, suitable conditions may include the use of a suitable medium, the presence of a suitable source of food and/or suitable nutrients, the use of a suitable temperature, and optionally the presence of a suitable inducing factor or compound (e.g., when the nucleotide sequences of the present technology are under the control of an inducible promoter); all of which may be selected by the skilled person. Again, under such conditions, the ISVDs, or polypeptides of the present technology may be expressed in a constitutive manner, in a transient manner, or only when suitably induced.

It will also be clear to the skilled person that the polypeptide of the present technology may (first) be generated in an immature form (as mentioned above), which may then be subjected to post-translational modification, depending on the host cell/host organism used. Also, the polypeptide of the current technology may be glycosylated, again depending on the host cell/host organism used.

The polypeptide of the present technology may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g., using a specific, cleavable amino acid sequence fused with the ISVD, or polypeptide of the present technology) and/or preparative immunological techniques (i.e. using antibodies against the polypeptide or construct to be isolated).

### 5.10 Compositions

The current technology also provides a composition comprising the polypeptide of the present technology, the nucleic acid encoding a polypeptide of the current technology, or the vector comprising such a nucleic acid. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Generally, the ISVDs, or polypeptides present technology can be formulated and administered in any suitable manner known per se. Reference is for example made to the general background art cited above (and in particular to WO 04/041862, WO 04/041863, WO 04/041865, WO 04/041867 and WO 08/020079) as well as to the standard handbooks, such as Remington's Pharmaceutical Sciences, 18th Ed., Mack Publishing Company, USA (1990), Remington, the Science and Practice of Pharmacy, 21st Ed., Lippincott Williams and Wilkins (2005); or the Handbook of Therapeutic Antibodies (S. Dubel, Ed.), Wiley, Weinheim, 2007 (see for example pages 252-255).

The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In another embodiment, kits are provided comprising a polypeptide of the present technology, a nucleic acid molecule of the present technology, a vector of the present technology, or a host cell of the present technology. The kit may comprise one or more vials containing the binding molecule and instructions for use. The kit may also contain means for administering the binding molecule of the present technology, such as a syringe, pump, infuser or the like.

### 5.11 Uses of the Polypeptide

The present technology further relates to applications and uses of the polypeptides, and compositions described herein, as well as to methods for the prevention and/or treatment of diseases. Some preferred but non-limiting applications and uses will become clear from the further description herein.

The multispecific-multivalent polypeptides and compositions of the present technology can generally be used to activate T cells at (the site of) the antigen-expressing target cells; such as to lyse these antigen-expressing target cells. The simultaneous binding by the multispecific-multivalent polypeptides of the present technology to TCR on T cells and to an antigen on target cells induces the activation of the cells and the subsequent lysis (killing) of the antigen expressing target cells. When not bound to the antigen expressing target cells, the polypeptides of the present technology show hardly any T cell activation. As such, target-independent lysis (i.e., lysis of cells without antigen expression) by the polypeptides of the present technology is minimal.

This killing of antigen expressing target cells can be advantageous in diseases or conditions in which the presence of these antigen expressing target cells is abundant and/or not desired.

Accordingly, in one embodiment, the present technology provides a polypeptide of the present technology, or a composition of the present technology, for use as a medicament.

In another embodiment, the present technology provides a polypeptide of the present technology, or a composition of the present technology, for use in treating a subject in need thereof.

In a further embodiment, the present technology relates to a polypeptide of the present technology, or a composition of the present technology, for use in the prevention, treatment or amelioration of a disease selected from the group consisting of a proliferative disease, an inflammatory disease, an infectious disease and an autoimmune disease.

In one embodiment, the present technology provides methods for delivering a prophylactic or therapeutic polypeptide to a specific location, tissue or cell type in the body, the method comprising the step of administering to a subject a polypeptide of the present technology or a composition of the present technology.

The present technology, in one embodiment, also relates to a method for treating a subject in need thereof, the method comprising the step of administering to a subject a polypeptide of the present technology, or a composition of the present technology.

The present technology also relates to a method for the treatment or amelioration of a disease selected from the group consisting of a proliferative disease, an inflammatory disease, an infectious disease and an autoimmune disease, comprising the step of administering to a subject in need thereof a polypeptide of the present technology or a composition of the present technology.

The present technology also relates to the use of a polypeptide of the present technology, or a composition of the present technology, for the manufacture of a medicament.

In a further embodiment, the present technology relates to the use of a polypeptide of the present technology, or a composition of the present technology, for the manufacture of a medicament for use in the prevention, treatment or amelioration of a disease selected from the group consisting of a proliferative disease, an inflammatory disease, an infectious disease and an autoimmune disease.

In one embodiment of the present technology, the treatment is a combination treatment.

The proliferative disease can be any proliferative disease prevented, treated and/or ameliorated by killing of antigen expressing target cells. In one embodiment, said proliferative disease is cancer.

In one embodiment, said cancer is chosen from the group consisting of carcinomas, gliomas, mesotheliomas, melanomas, lymphomas, leukemias, adenocarcinomas: breast cancer, ovarian cancer, cervical cancer, glioblastoma, multiple myeloma (including monoclonal gammopathy of undetermined significance, asymptomatic and symptomatic myeloma), prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease or AIDS-associated primary effusion lymphoma, neuroectodermal tumors, rhabdomyosarcoma; as well as any metastasis of any of the above cancers.

In a specific embodiment, the proliferative disease is a proliferative disease that can prevented, treated and/or ameliorated by killing of CD123 expressing cells. In one specific embodiment, said proliferative disease is cancer. Examples of cancers associated with CD123 overexpression will be clear to the skilled person based on the disclosure herein, and for example include (without being limiting) the following cancers: lymphomas (including Burkitt's lymphoma, Hodgkin's lymphoma and non-Hodgkin's lymphoma), leukemias (including acute myeloid leukemia, chronic myeloid leukemia, acute B lymphoblastic leukemia, chronic lymphocytic leukemia and hairy cell leukemia), myelodysplastic syndrome, blastic plasmacytoid dendritic cell neoplasm, systemic mastocytosis and multiple myeloma.

Accordingly, in a specific embodiment, said cancer is chosen from the group consisting of lymphomas (including Burkitt's lymphoma, Hodgkin's lymphoma and non-Hodgkin's lymphoma), leukemias (including acute myeloid leukemia, chronic myeloid leukemia, acute B lymphoblastic leukemia, chronic lymphocytic leukemia and hairy cell leukemia), myelodysplastic syndrome, blastic plasmacytoid dendritic cell neoplasm, systemic mastocytosis and multiple myeloma.

The inflammatory disease can be any inflammatory disease prevented, treated and/or ameliorated by killing of antigen expressing target cells.

In a specific embodiment, the inflammatory disease is a inflammatory disease that can prevented, treated and/or ameliorated by killing of CD123 expressing cells. In one specific embodiment, the inflammatory condition is chosen from the group consisting of Autoimmune Lupus (SLE), allergy, asthma and rheumatoid arthritis.

A “subject” as referred to in the context of the present technology can be any animal, preferably a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal, but preferably is a human subject.

As will be clear to the skilled person, the subject to be treated will in particular be a person suffering from, or at risk of the diseases and conditions mentioned herein.

As used herein, the terms “treat”, “treatment” and “treating” in the context of administering (a) therapy(ies) to a subject refer to the reduction or amelioration of the progression, severity, and/or duration of a disorder associated with a hyperproliferative cell disorder, e.g., cancer, and/or the amelioration of one or more symptoms thereof resulting from the administration of one or more therapies (including, but not limited to, the administration of one or more prophylactic or therapeutic agents). In specific embodiments, the terms “treat”, “treatment” and “treating” in the context of administering (a) therapy(ies) to a subject refer to the reduction or amelioration of the progression, severity, and/or duration of a hyperproliferative cell disorder, e.g., cancer, refers to a reduction in cancer cells by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline). In other embodiments, the terms “treat”, “treatment” and “treating” in the context of administering (a) therapy(ies) to a subject refer to the reduction or amelioration of the progression, severity, and/or duration of a hyperproliferative cell disorder, e.g., cancer, refers to no change in cancer cell number, a reduction in hospitalization time, a reduction in mortality, or an increase in survival time of the subject with cancer.

As used herein, the term “therapy” refers to any protocol, method and/or agent that can be used in the treatment, prevention and/or management of a hyperproliferative cell disorder, e.g., cancer. In certain embodiments, the terms “therapies” and “therapy” refer to a biological therapy, supportive therapy, and/or other therapies useful in the treatment, prevention and/or management of a hyperproliferative cell disorder, e.g., cancer, or one or more symptoms thereof known to one of skill in the art such as medical personnel.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Usually, in the above method, a single ISVD, or polypeptide of the present technology will be used. It is however within the scope of the present technology to use two or more ISVDs, or polypeptides of the present technology in combination.

The polypeptides of the present technology may also be used in combination with one or more further pharmaceutically active compounds or principles, i.e. as a combined treatment regimen, which may or may not lead to a synergistic effect. The clinician will be able to select such further compounds or principles, as well as a suitable combined treatment regimen, based on the factors cited above and his expert judgement.

In particular, the polypeptides of the present technology may be used in combination with other pharmaceutically active compounds or principles that are or can be used for the prevention and/or treatment of the hyperproliferative cell disorder, e.g., cancer, disease and/or disorder cited herein, as a result of which a synergistic effect may or may not be obtained. Examples of such compounds and principles, as well as routes, methods and pharmaceutical formulations or compositions for administering them will be clear to the clinician.

The polypeptides and compositions of the present technology can be used for the prevention and/or treatment of diseases and disorders of the present technology (herein also “diseases and disorders of the present technology”) which include, but are not limited to cancer. The term “cancer” refers to the pathological condition in mammals that is typically characterized by dysregulated cellular proliferation or survival. Examples of cancer include, but are not limited to, carcinomas, gliomas, mesotheliomas, melanomas, lymphomas, leukemias, adenocarcinomas: breast cancer, ovarian cancer, cervical cancer, glioblastoma, multiple myeloma (including monoclonal gammopathy of undetermined significance, asymptomatic and symptomatic myeloma), prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease or AIDS-associated primary effusion lymphoma, neuroectodermal tumors, rhabdomyosarcoma (see e.g., Cancer, Principles and practice (DeVita et al. eds 1997) for additional cancers); as well as any metastasis of any of the above cancers.

Substances (including polypeptides, nucleic acid molecules and vectors) or compositions may be administered to a subject by any suitable route of administration, for example by enteral (such as oral or rectal) or parenteral (such as epicutaneous, sublingual, buccal, nasal, intra-articular, intradermal, intramuscular, intraperitoneal, intravenous, subcutaneous, transdermal, or transmucosal) administration. Parenteral administration, such as intramuscular, subcutaneous or intradermal, administration is preferred.

An effective amount of a polypeptide, a nucleic acid molecule or vector as described, or a composition comprising the polypeptide, nucleic acid molecule or vector can be administered to a subject in order to provide the intended treatment results.

One or more doses can be administered. If more than one dose is administered, the doses can be administered in suitable intervals in order to maximize the effect of the polypeptide, composition, nucleic acid molecule or vector.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Usually, in the above method, a single ISVD, or polypeptide of the present technology will be used. It is however within the scope of the present technology to use two or more ISVDs, or polypeptides of the present technology in combination.

The polypeptides of the present technology may also be used in combination with one or more further pharmaceutically active compounds or principles, i.e. as a combined treatment regimen, which may or may not lead to a synergistic effect. The clinician will be able to select such further compounds or principles, as well as a suitable combined treatment regimen, based on the factors cited above and his expert judgement.

In particular, the polypeptides of the present technology may be used in combination with other pharmaceutically active compounds or principles that are or can be used for the prevention and/or treatment of the hyperproliferative cell disorder, e.g., cancer, disease and/or disorder cited herein, as a result of which a synergistic effect may or may not be obtained. Examples of such compounds and principles, as well as routes, methods and pharmaceutical formulations or compositions for administering them will be clear to the clinician.

TABLE A-1
Sequences of monovalent ISVDs binding to the constant domain of the TCR (″ID″ refers
to the SEQ ID NO as used herein)

| Name | ID | Amino acid sequence |
|---|---|---|
| T0170056G05 | 1 | EVQLVESGGGLVQPGGSLRLSCVASGDVHKINFLGWYRQAPGKEREKVAHISIGDQ |
| | | TDYADSAKGRFTISRDESKNMVYLQMNSLKPEDTAVYFCRAFSRIYPYDYWGQGTLV |
| | | TVSS |
| T017000141 | 2 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000351 | 2 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000343 | 3 | EVQLVESGGGVVQPGGSLRLSCVASGDVHKINFLGWYRQAPGKEREKVAHISIGDQ |
| | | TDYADSAKGRFTISRDESKNTVYLQMNSLRPEDTALYFCRAFSRIYPYDYWGQGTLVT |
| | | VSS |
| T017000345 | 4 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYYCRAFSRIYPYDYWGQGTLV |
| | | TVSS |
| T017000164 | 5 | EVQLVESGGGVVQPGGSLRLSCVASGYVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000166 | 6 | EVQLVESGGGVVQPGGSLRLSCVASGDVHKINFYGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000161 | 7 | EVQLVESGGGVVQPGGSLRLSCVASGEVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000177 | 8 | EVQLVESGGGVVQPGGSLRLSCVASGSVHKINFLGWYRQAPGKEREKVAHISIGDQT |
| | | DYADSAKGRFTISRDESKNMVYLQMNSLRPEDTALYFCRAFSRIYPYDYWGQGTLVT |
| | | VSS |
| T017000188 | 9 | EVQLVESGGGVVQPGGSLRLSCVASGAVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000174 | 10 | EVQLVESGGGVVQPGGSLRLSCVASWDVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000208 | 11 | EVQLVESGGGVVQPGGSLRLSCVASYDVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000163 | 12 | EVQLVESGGGVVQPGGSLRLSCVASGDVHQINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000745 | 12 | EVQLVESGGGVVQPGGSLRLSCVASGDVHQINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000176 | 13 | EVQLVESGGGVVQPGGSLRLSCVASGDVHKLNFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000179 | 14 | EVQLVESGGGVVQPGGSLRLSCVASGDVHKINFLTWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000180 | 15 | EVQLVESGGGVVQPGGSLRLSCVASQDVHKINFLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000196 | 16 | EVQLVESGGGVVQPGGSLRLSCVASGDVHKIFFLGWYRQAPGKEREKVAHISIGDQT |
| | | DYADSAKGRFTISRDESKNMVYLQMNSLRPEDTALYFCRAFSRIYPYDYWGQGTLVT |
| | | VSS |
| T017000170 | 17 | EVQLVESGGGVVQPGGSLRLSCVASGDVHKINYLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000173 | 18 | EVQLVESGGGVVQPGGSLRLSCVASGDVHKINVLGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000185 | 19 | EVQLVESGGGVVQPGGSLRLSCVASGDVHKINFFGWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000200 | 20 | EVQLVESGGGVVQPGGSLRLSCVASGDVHKINFLRWYRQAPGKEREKVAHISIGDQ |
| | | TDYADSAKGRFTISRDESKNMVYLQMNSLRPEDTALYFCRAFSRIYPYDYWGQGTLV |
| | | TVSS |
| T017000190 | 21 | EVQLVESGGGVVQPGGSLRLSCVASGDVHKINFLSWYRQAPGKEREKVAHISIGDQT |
| | | DYADSAKGRFTISRDESKNMVYLQMNSLRPEDTALYFCRAFSRIYPYDYWGQGTLVT |
| | | VSS |
| T017000231 | 22 | EVQLVESGGGVVQPGGSLRLSCVASGDVHKINFLGWYRQAPGKEREKVAVISIGDQ |
| | | TDYADSAKGRFTISRDESKNMVYLQMNSLRPEDTALYFCRAFSRIYPYDYWGQGTLV |
| | | TVSS |
| T017000245 | 23 | EVQLVESGGGVVQPGGSLRLSCVASGDVHKINFLGWYRQAPGKEREKVAHISIADQ |
| | | TDYADSAKGRFTISRDESKNMVYLQMNSLRPEDTALYFCRAFSRIYPYDYWGQGTLV |
| | | TVSS |
| T017000238 | 24 | EVQLVESGGGVVQPGGSLRLSCVASGDVHKINFLGWYRQAPGKEREKVAHISIGDET |
| | | DYADSAKGRFTISRDESKNMVYLQMNSLRPEDTALYFCRAFSRIYPYDYWGQGTLVT |
| | | VSS |
| T017000249 | 25 | EVQLVESGGGVVQPGGSLRLSCVASGDVHKINFLGWYRQAPGKEREKVAHISIGDV |
| | | TDYADSAKGRFTISRDESKNMVYLQMNSLRPEDTALYFCRAFSRIYPYDYWGQGTLV |
| | | TVSS |
| T017000232 | 26 | EVQLVESGGGVVQPGGSLRLSCVASGDVHKINFLGWYRQAPGKEREKVAHISIGDQ |
| | | VDYADSAKGRFTISRDESKNMVYLQMNSLRPEDTALYFCRAFSRIYPYDYWGQGTLV |
| | | TVSS |
| T017000233 | 27 | EVQLVESGGGVVQPGGSLRLSCVASGDVHKINFLGWYRQAPGKEREKVAHISIGDQ |
| | | SDYADSAKGRFTISRDESKNMVYLQMNSLRPEDTALYFCRAFSRIYPYDYWGQGTLV |
| | | TVSS |
| T017000234 | 28 | EVQLVESGGGVVQPGGSLRLSCVASGDVHKINFLGWYRQAPGKEREKVAMISIGDQ |
| | | TDYADSAKGRFTISRDESKNMVYLQMNSLRPEDTALYFCRAFSRIYPYDYWGQGTLV |
| | | TVSS |
| T017000258 | 29 | EVQLVESGGGVVQPGGSLRLSCVASGDVHKINFLGWYRQAPGKEREKVAHISIGEQ |
| | | TDYADSAKGRFTISRDESKNMVYLQMNSLRPEDTALYFCRAFSRIYPYDYWGQGTLV |
| | | TVSS |
| T017000235 | 30 | EVQLVESGGGVVQPGGSLRLSCVASGDVHKINFLGWYRQAPGKEREKVAHISIGDH |
| | | TDYADSAKGRFTISRDESKNMVYLQMNSLRPEDTALYFCRAFSRIYPYDYWGQGTLV |
| | | TVSS |
| T017000250 | 31 | EVQLVESGGGVVQPGGSLRLSCVASGDVHKINFLGWYRQAPGKEREKVAHISIGDK |
| | | TDYADSAKGRFTISRDESKNMVYLQMNSLRPEDTALYFCRAFSRIYPYDYWGQGTLV |
| | | TVSS |
| T017000254 | 32 | EVQLVESGGGVVQPGGSLRLSCVASGDVHKINFLGWYRQAPGKEREKVAHISIGDR |
| | | TDYADSAKGRFTISRDESKNMVYLQMNSLRPEDTALYFCRAFSRIYPYDYWGQGTLV |
| | | TVSS |
| T017000223 | 33 | EVQLVESGGGVVQPGGSLRLSCVASGDVHKINFLGWYRQAPGKEREKVAHISIGDQ |
| | | ADYADSAKGRFTISRDESKNMVYLQMNSLRPEDTALYFCRAFSRIYPYDYWGQGTLV |
| | | TVSS |
| T017000241 | 34 | EVQLVESGGGVVQPGGSLRLSCVASGDVHKINFLGWYRQAPGKEREKVAHISIGDQ |
| | | TNYADSAKGRFTISRDESKNMVYLQMNSLRPEDTALYFCRAFSRIYPYDYWGQGTLV |
| | | TVSS |
| T017000312 | 35 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRALSRIYPYDYWGQGTLV |
| | | TVSS |
| T017000291 | 36 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIWPYDYWGQGTL |
| | | VTVSS |
| T017000281 | 37 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000290 | 38 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSKIYPYDYWGQGTLV |
| | | TVSS |
| T017000294 | 39 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYDWWGQGTL |
| | | VTVSS |
| T017000283 | 40 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSSIYPYDYWGQGTLV |
| | | TVSS |
| T017000287 | 41 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYQYWGQGTLV |
| | | TVSS |
| T017000278 | 42 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYSYWGQGTLV |
| | | TVSS |
| T017000285 | 43 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYLYWGQGTLV |
| | | TVSS |
| T017000301 | 44 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYAYWGQGTLV |
| | | TVSS |
| T017000302 | 45 | EVQLVESGGGVVQPGGSLRLSCVASGDVHKINFLGWYRQAPGKEREKVAHISIGDQ |
| | | TDYADSAKGRFTISRDESKNMVYLQMNSLRPEDTALYFCRAFSRIYPYTYWGQGTLV |
| | | TVSS |
| T017000624 | 46 | EVQLVESGGGVVQPGGSLRLSCVASGYVHKINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000623 | 47 | EVQLVESGGGVVQPGGSLRLSCVASWDVHKINFYGWYRQAPGKEREKVAHISIGDQ |
| | | TDYADSAKGRFTISRDESKNTVYLQMNSLRPEDTALYYCRALSKIWPYDYWGQGTLV |
| | | TVSS |
| T017000625 | 48 | EVQLVESGGGVVQPGGSLRLSCVASYDVHKINFYGWYRQAPGKEREKVAHISIGDQ |
| | | TDYADSAKGRFTISRDESKNTVYLQMNSLRPEDTALYYCRALSKIWPYDYWGQGTLV |
| | | TVSS |
| T017000635 | 49 | EVQLVESGGGVVQPGGSLRLSCVASWDVHQINFYGWYRQAPGKEREKVAHISIGD |
| | | QTDYADSAKGRFTISRDESKNTVYLQMNSLRPEDTALYYCRALSRIWPYDYWGQGT |
| | | LVTVSS |
| T017000641 | 50 | EVQLVESGGGVVQPGGSLRLSCVASWDVHKINFYTWYRQAPGKEREKVAHISIGDQ |
| | | TDYADSAKGRFTISRDESKNTVYLQMNSLRPEDTALYYCRALSKIWPYDYWGQGTLV |
| | | TVSS |
| T017000687 | 51 | EVQLVESGGGVVQPGGSLRLSCVASGSVHQINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000613 | 52 | EVQLVESGGGVVQPGGSLRLSCVASWDVHKINFYGWYRQAPGKEREKVAHISIGDQ |
| | | TDYADSAKGRFTISRDESKNMVYLQMNSLRPEDTALYFCRALSKIWPYDYWGQGTL |
| | | VTVSS |
| T017000615 | 53 | EVQLVESGGGVVQPGGSLRLSCVASYDVHKINFYGWYRQAPGKEREKVAHISIGDQ |
| | | TDYADSAKGRFTISRDESKNMVYLQMNSLRPEDTALYFCRALSKIWPYDYWGQGTL |
| | | VTVSS |
| T017000505 | 54 | EVQLVESGGGVVQPGGSLRLSCVASGDVHQLNFLGWYRQAPGKEREKVAHISIGDQ |
| | | TDYADSAKGRFTISRDESKNMVYLQMNSLRPEDTALYFCRAFSKIYPYDYWGQGTLV |
| | | TVSS |
| T017000362 | 55 | EVQLVESGGGVVQPGGSLRLSCVASYDVHKINFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000440 | 56 | EVQLVESGGGVVQPGGSLRLSCVASWDVHKLNFYGWYRQAPGKEREKVAHISIGD |
| | | QTDYADSAKGRFTISRDESKNMVYLQMNSLRPEDTALYFCRAMSKIWPYDYWGQG |
| | | TLVTVSS |
| T017000378 | 57 | EVQLVESGGGVVQPGGSLRLSCVASWDVHKINFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000608 | 58 | EVQLVESGGGVVQPGGSLRLSCVASWDVHKINFYGWYRQAPGKEREKVAHISIGDQ |
| | | TDYADSAKGRFTISRDESKNMVYLQMNSLRPEDTALYFCRAFSKIWPYDYWGQGTL |
| | | VTVSS |
| T017000374 | 59 | EVQLVESGGGVVQPGGSLRLSCVASYDVHQINFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000607 | 60 | EVQLVESGGGVVQPGGSLRLSCVASWDVHQINFYGWYRQAPGKEREKVAHISIGD |
| | | QTDYADSAKGRFTISRDESKNMVYLQMNSLRPEDTALYFCRAFSKIWPYDYWGQGT |
| | | LVTVSS |
| T017000602 | 61 | EVQLVESGGGVVQPGGSLRLSCVASWDVHQINFLGWYRQAPGKEREKVAHISIGD |
| | | QTDYADSAKGRFTISRDESKNMVYLQMNSLRPEDTALYFCRAFSKIWPYDYWGQGT |
| | | LVTVSS |
| T017000359 | 62 | EVQLVESGGGVVQPGGSLRLSCVASWDVHQINFYGWYRQAPGKEREKVAHISIGD |
| | | QTDYADSAKGRFTISRDESKNMVYLQMNSLRPEDTALYFCRAFSKIYPYDYWGQGTL |
| | | VTVSS |
| T017000740 | 63 | EVQLVESGGGVVQPGGSLRLSCVASWDVHQLNFYGWYRQAPGKEREKVAHISIGD |
| | | QTDYADSAKGRFTISRDESKNMVYLQMNSLRPEDTALYFCRAMSKIYPYDYWGQGT |
| | | LVTVSS |
| T017000554 | 64 | EVQLVESGGGVVQPGGSLRLSCVASYDVHQINFLGWYRQAPGKEREKVAHISIGDQ |
| | | TDYADSAKGRFTISRDESKNMVYLQMNSLRPEDTALYFCRAMSKIYPYDYWGQGTL |
| | | VTVSS |
| T017000413 | 65 | EVQLVESGGGVVQPGGSLRLSCVASWDVHQLNFYGWYRQAPGKEREKVAHISIGD |
| | | QTDYADSAKGRFTISRDESKNMVYLQMNSLRPEDTALYFCRAMSKIWPYDYWGQG |
| | | TLVTVSS |
| T017000379 | 66 | EVQLVESGGGVVQPGGSLRLSCVASWDVHQINFYGWYRQAPGKEREKVAHISIGD |
| | | QTDYADSAKGRFTISRDESKNMVYLQMNSLRPEDTALYFCRAMSKIWPYDYWGQG |
| | | TLVTVSS |
| T017000367 | 67 | EVQLVESGGGVVQPGGSLRLSCVASGYVHKINFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000390 | 68 | EVQLVESGGGVVQPGGSLRLSCVASWDVHQINFLGWYRQAPGKEREKVAHISIGD |
| | | QTDYADSAKGRFTISRDESKNMVYLQMNSLRPEDTALYFCRAMSKIWPYDYWGQG |
| | | TLVTVSS |
| T017000443 | 69 | EVQLVESGGGVVQPGGSLRLSCVASWDVHQINFYGWYRQAPGKEREKVAHISIGD |
| | | QTDYADSAKGRFTISRDESKNMVYLQMNSLRPEDTALYFCRAMSKIYPYDYWGQGT |
| | | LVTVSS |
| T017000776 | 70 | EVQLVESGGGVVQPGGSLRLSCVASWSVHQINFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000371 | 71 | EVQLVESGGGVVQPGGSLRLSCVASGYVHKINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000759 | 72 | EVQLVESGGGVVQPGGSLRLSCVASGSVHKINFYGWYRQAPGKEREKVAHISIGDQ |
| | | TDYADSAKGRFTISRDESKNMVYLQMNSLRPEDTALYFCRAFSKIWPYDWWGQGT |
| | | LVTVSS |
| T017000539 | 73 | EVQLVESGGGVVQPGGSLRLSCVASGYVHKINFYGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000499 | 74 | EVQLVESGGGVVQPGGSLRLSCVASGYVHKLNFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000538 | 75 | EVQLVESGGGVVQPGGSLRLSCVASGYVHKLNFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000410 | 76 | EVQLVESGGGVVQPGGSLRLSCVASGYVHKLNFYGWYRQAPGKEREKVAHISIGDQ |
| | | TDYADSAKGRFTISRDESKNMVYLQMNSLRPEDTALYFCRAFSRIWPYDWWGQGT |
| | | LVTVSS |
| T017000566 | 77 | EVQLVESGGGVVQPGGSLRLSCVASGYVHKLNFLGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000589 | 78 | EVQLVESGGGVVQPGGSLRLSCVASGYVHKLNFYGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000397 | 79 | EVQLVESGGGVVQPGGSLRLSCVASGSVHQLNFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000757 | 80 | EVQLVESGGGVVQPGGSLRLSCVASGAVHKLNFLGWYRQAPGKEREKVAHISIGDQ |
| | | TDYADSAKGRFTISRDESKNMVYLQMNSLRPEDTALYFCRAFSKIWPYDWWGQGT |
| | | LVTVSS |
| T017000372 | 81 | EVQLVESGGGVVQPGGSLRLSCVASGYVHKINFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000423 | 82 | EVQLVESGGGVVQPGGSLRLSCVASGSVHQINFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000521 | 83 | EVQLVESGGGVVQPGGSLRLSCVASGSVHQLNFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000370 | 84 | EVQLVESGGGVVQPGGSLRLSCVASGAVHQINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000383 | 85 | EVQLVESGGGVVQPGGSLRLSCVASGDVHQINFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000364 | 86 | EVQLVESGGGVVQPGGSLRLSCVASGAVHQINFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000442 | 87 | EVQLVESGGGVVQPGGSLRLSCVASGYVHKLNFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000461 | 88 | EVQLVESGGGVVQPGGSLRLSCVASGDVHQINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000384 | 89 | EVQLVESGGGVVQPGGSLRLSCVASGAVHQLNFYGWYRQAPGKEREKVAHISIGD |
| | | QTDYADSAKGRFTISRDESKNMVYLQMNSLRPEDTALYFCRAMSRIWPYDYWGQG |
| | | TLVTVSS |
| T017000593 | 90 | EVQLVESGGGVVQPGGSLRLSCVASGAVHKLNFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000373 | 91 | EVQLVESGGGVVQPGGSLRLSCVASGSVHKLNFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000614 | 92 | EVQLVESGGGVVQPGGSLRLSCVASGYVHKINFYGWYRQAPGKEREKVAHISIGDQ |
| | | TDYADSAKGRFTISRDESKNMVYLQMNSLRPEDTALYFCRALSRIWPYDYWGQGTL |
| | | VTVSS |
| T017000509 | 93 | EVQLVESGGGVVQPGGSLRLSCVASGAVHKINFLGWYRQAPGKEREKVAHISIGDQ |
| | | TDYADSAKGRFTISRDESKNMVYLQMNSLRPEDTALYFCRAMSKIWPYDWWGQG |
| | | TLVTVSS |
| T017000361 | 94 | EVQLVESGGGVVQPGGSLRLSCVASGSVHKINFLGWYRQAPGKEREKVAHISIGDQT |
| | | DYADSAKGRFTISRDESKNMVYLQMNSLRPEDTALYFCRAMSRIWPYDYWGQGTL |
| | | VTVSS |
| T017000498 | 95 | EVQLVESGGGVVQPGGSLRLSCVASGAVHKINFYGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000408 | 96 | EVQLVESGGGVVQPGGSLRLSCVASGSVHKINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000432 | 97 | EVQLVESGGGVVQPGGSLRLSCVASGSVHKLNFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000381 | 98 | EVQLVESGGGVVQPGGSLRLSCVASGAVHKINFLGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000754 | 99 | EVQLVESGGGVVQPGGSLRLSCVASGYVHQINFLGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000515 | 100 | EVQLVESGGGVVQPGGSLRLSCVASGAVHKINFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000360 | 101 | EVQLVESGGGVVQPGGSLRLSCVASWSVHQLNFYGWYRQAPGKEREKVAHISIGD |
| | | QTDYADSAKGRFTISRDESKNMVYLQMNSLRPEDTALYFCRAMSKIYPYDWWGQG |
| | | TLVTVSS |
| T017000588 | 102 | EVQLVESGGGVVQPGGSLRLSCVASGAVHKINFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000401 | 103 | EVQLVESGGGVVQPGGSLRLSCVASGSVHKINFYGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000357 | 104 | EVQLVESGGGVVQPGGSLRLSCVASGYVHQLNFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000354 | 105 | EVQLVESGGGVVQPGGSLRLSCVASGAVHKINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000547 | 106 | EVQLVESGGGVVQPGGSLRLSCVASGAVHKLNFYGWYRQAPGKEREKVAHISIGDQ |
| | | TDYADSAKGRFTISRDESKNMVYLQMNSLRPEDTALYFCRAFSRIWPYDYWGQGTL |
| | | VTVSS |
| T017000531 | 107 | EVQLVESGGGVVQPGGSLRLSCVASGSVHKINFLGWYRQAPGKEREKVAHISIGDQT |
| | | DYADSAKGRFTISRDESKNMVYLQMNSLRPEDTALYFCRAMSRIWPYDWWGQGT |
| | | LVTVSS |
| T017000391 | 108 | EVQLVESGGGVVQPGGSLRLSCVASGAVHKINFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000356 | 109 | EVQLVESGGGVVQPGGSLRLSCVASGSVHKLNFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000406 | 110 | EVQLVESGGGVVQPGGSLRLSCVASGYVHQINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000468 | 111 | EVQLVESGGGVVQPGGSLRLSCVASGSVHKINFYGWYRQAPGKEREKVAHISIGDQ |
| | | TDYADSAKGRFTISRDESKNMVYLQMNSLRPEDTALYFCRAFSRIWPYDYWGQGTL |
| | | VTVSS |
| T017000358 | 112 | EVQLVESGGGVVQPGGSLRLSCVASGAVHKLNFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000685 | 113 | EVQLVESGGGVVQPGGSLRLSCVASGEVHQINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000369 | 114 | EVQLVESGGGVVQPGGSLRLSCVASGSVHKLNFYGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000363 | 115 | EVQLVESGGGVVQPGGSLRLSCVASGSVHKINFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000450 | 116 | EVQLVESGGGVVQPGGSLRLSCVASGYVHQINFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000530 | 117 | EVQLVESGGGVVQPGGSLRLSCVASGAVHKLNFLGWYRQAPGKEREKVAHISIGDQ |
| | | TDYADSAKDRFTISRDESKNMVYLQMNSLRPEDTALYFCRAMSRIYPYDYWGQGTL |
| | | VTVSS |
| T017000473 | 118 | EVQLVESGGGVVQPGGSLRLSCVASGYVHQINFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000418 | 119 | EVQLVESGGGVVQPGGSLRLSCVASGAVHKLNFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000611 | 120 | EVQLVESGGGVVQPGGSLRLSCVASGDVHQINFYGWYRQAPGKEREKVAHISIGDQ |
| | | TDYADSAKGRFTISRDESKNMVYLQMNSLRPEDTALYFCRAFSKIYPYDYWGQGTLV |
| | | TVSS |
| T017000386 | 121 | EVQLVESGGGVVQPGGSLRLSCVASGSVHKLNFLGWYRQAPGKEREKVAHISIGDQ |
| | | TDYADSAKGRFTISRDESKNMVYLQMNSLRPEDTALYFCRAFSRIWPYDWWGQGT |
| | | LVTVSS |
| T017000382 | 122 | EVQLVESGGGVVQPGGSLRLSCVASGSVHKINFYGWYRQAPGKEREKVAHISIGDQ |
| | | TDYADSAKGRFTISRDESKNMVYLQMNSLRPEDTALYFCRAMSRIYPYDWWGQGT |
| | | LVTVSS |
| T017000419 | 123 | EVQLVESGGGVVQPGGSLRLSCVASGAVHKLNFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000774 | 124 | EVQLVESGGGVVQPGGSLRLSCVASGDVHQINFYGWYRQAPGKEREKVAHISIGDQ |
| | | TDYADSAKGRFTISRDESKNMVYLQMNSLRPEDTALYFCRAMSKIWPYDYWGQGT |
| | | LVTVSS |
| T017000686 | 125 | EVQLVESGGGVVQPGGSLRLSCVASGSVHKINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000569 | 126 | EVQLVESGGGVVQPGGSLRLSCVASGDVHKLNFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000365 | 127 | EVQLVESGGGVVQPGGSLRLSCVASGAVHKLNFLGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000434 | 128 | EVQLVESGGGVVQPGGSLRLSCVASGSVHKLNFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000612 | 129 | EVQLVESGGGVVQPGGSLRLSCVASGDVHQLNFLGWYRQAPGKEREKVAHISIGDQ |
| | | TDYADSAKGRFTISRDESKNMVYLQMNSLRPEDTALYFCRALSKIYPYDYWGQGTLV |
| | | TVSS |
| T017000610 | 130 | EVQLVESGGGVVQPGGSLRLSCVASGDVHQINFLGWYRQAPGKEREKVAHISIGDQ |
| | | TDYADSAKGRFTISRDESKNMVYLQMNSLRPEDTALYFCRALSKIYPYDYWGQGTLV |
| | | TVSS |
| T017000682 | 131 | EVQLVESGGGVVQPGGSLRLSCVASGAVHKINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000766 | 132 | EVQLVESGGGVVQPGGSLRLSCVASGYVHKINFYGWYRQAPGKEREKVAHISIGDQ |
| | | TDYADSAKGRFTISRDESKNMVYLQMNSLRPEDTALYFCRAMSKIYPYDYWGQGTL |
| | | VTVSS |
| T017000690 | 133 | EVQLVESGGGVVQPGGSLRLSCVASWEVHKINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000750 | 134 | EVQLVESGGGVVQPGGSLRLSCVASYAVHQINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000742 | 135 | EVQLVESGGGVVQPGGSLRLSCVASGYVHQINFYGWYRQAPGKEREKVAHISIGDQ |
| | | TDYADSAKGRFTISRDESKNMVYLQMNSLRPEDTALYFCRAMSRIWPYDWWGQG |
| | | TLVTVSS |
| T017000577 | 136 | EVQLVESGGGVVQPGGSLRLSCVASWDVHQINFLGWYRQAPGKEREKVAHISIGD |
| | | QTDYADSAKGRFTISRDESKNMVYLQMNSLRPEDTALYFCRAMSRIYPYDYWGQGT |
| | | LVTVSS |
| T017000609 | 137 | EVQLVESGGGVVQPGGSLRLSCVASGDVHQINFLGWYRQAPGKEREKVAHISIGDQ |
| | | TDYADSAKGRFTISRDESKNMVYLQMNSLRPEDTALYFCRAFSKIYPYDYWGQGTLV |
| | | TVSS |
| T017000355 | 138 | EVQLVESGGGVVQPGGSLRLSCVASWDVHQLNFYGWYRQAPGKEREKVAHISIGD |
| | | QTDYADSAKGRFTISRDESKNMVYLQMNSLRPEDTALYFCRAMSRIWPYDYWGQG |
| | | TLVTVSS |
| T017000403 | 139 | EVQLVESGGGVVQPGGSLRLSCVASWDVHQINFLGWYRQAPGKEREKVAHISIGD |
| | | QTDYADSAKGRFTISRDESKNMVYLQMNSLRPEDTALYFCRAMSRIWPYDYWGQG |
| | | TLVTVSS |
| T017000578 | 140 | EVQLVESGGGVVQPGGSLRLSCVASYDVHQLNFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000415 | 141 | EVQLVESGGGVVQPGGSLRLSCVASWDVHQINFYGWYRQAPGKEREKVAHISIGD |
| | | QTDYADSAKGRFTISRDESKNMVYLQMNSLRPEDTALYFCRAMSRIWPYDYWGQG |
| | | TLVTVSS |
| T017000368 | 142 | EVQLVESGGGVVQPGGSLRLSCVASWDVHQINFYGWYRQAPGKEREKVAHISIGD |
| | | QTDYADSAKGRFTISRDESKNMVYLQMNSLRPEDTALYFCRAMSRIYPYDYWGQGT |
| | | LVTVSS |
| T017000398 | 143 | EVQLVESGGGVVQPGGSLRLSCVASYDVHQINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000377 | 144 | EVQLVESGGGVVQPGGSLRLSCVASWDVHKINFYGWYRQAPGKEREKVAHISIGDQ |
| | | TDYADSAKGRFTISRDESKNMVYLQMNSLRPEDTALYFCRAMSRIYPYDYWGQGTL |
| | | VTVSS |
| T017000411 | 145 | EVQLVESGGGVVQPGGSLRLSCVASWDVHKINFYGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000691 | 146 | EVQLVESGGGVVQPGGSLRLSCVASWEVHQINFYGWYRQAPGKEREKVAHISIGD |
| | | QTDYADSAKGRFTISRDESKNTVYLQMNSLRPEDTALYYCRALSRIWPYDYWGQGT |
| | | LVTVSS |
| T017000638 | 147 | EVQLVESGGGVVQPGGSLRLSCVASWDVHKINFYTWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000550 | 148 | EVQLVESGGGVVQPGGSLRLSCVASGSVHQINFLGWYRQAPGKEREKVAHISIGDQ |
| | | TDYADSAKGRFTISRDESKNMVYLQMNSLRPEDTALYFCRAMSRIYPYDWWGQGT |
| | | LVTVSS |
| T017000518 | 149 | EVQLVESGGGVVQPGGSLRLSCVASYSVHQLNFLGWYRQAPGKEREKVAHISIGDQ |
| | | TDYADSAKGRFTISRDESKNMVYLQMNSLRPEDTALYFCRAMSRIWPYDYWGQGT |
| | | LVTVSS |
| T017000679 | 150 | DVQLVESGGGVVQPGGSLRLSCVASGYVHKINFYGWYRQAPGKEREKVAHISIGDQ |
| | | TDYADSAKGRFTISRDESKNTVYLQMNSLRPEDTALYYCRALSRIWPYDYWGQGTLV |
| | | TVSS |
| T017000680 | 151 | DVQLVESGGGVVQPGGSLRLSCVASGYVHKINFYGWYRQAPGKEREKVAHISIGDQ |
| | | TDYADSAKGRFTISRDESKNTVYLQMNSLRPEDTAAYYCRALSRIWPYDYWGQGTL |
| | | VTVSS |
| T017000681 | 152 | DVQLVESGGGVVQPGGSLRLSCVASGYVHKINFYGWYRQAPGKEREKVAHISIGDQ |
| | | TDYADSAKGRFTISRDESKNTVYLQMNSLRPEDTATYYCRALSRIWPYDYWGQGTLV |
| | | TVSS |
| T017000697 | 261 | EVQLVESGGGVVQPGGSLRLSCVASGYVHKINFYGWYRQAPGKEREKVAHISIGDQ |
| | | TDYADSAKGRFTISRDESKNTVYLQMNSLRPEDTAAYYCRALSRIWPYDYWGQGTL |
| | | VTVSS |
| T017000681 | 262 | EVQLVESGGGVVQPGGSLRLSCVASGYVHKINFYGWYRQAPGKEREKVAHISIGDQ |
| (D1E)* | | TDYADSAKGRFTISRDESKNTVYLQMNSLRPEDTATYYCRALSRIWPYDYWGQGTLV |
| | | TVSS |

*() indicates the amino acid substitutions introduced into the ISVD (e.g.: T017000681(D1E) means that ISVD T017000681 (SEQ ID NO: 152) contains a D1E substitution).

TABLE A-2
Sequences for CDRs according to AbM definition and frameworks, plus specific combinations as provided in formula I,
namely FR1-CDR1-FR2-CDR2-FR3-CDR3-FR4. (″ID″ refers to the given SEQ ID NO) The first column refers to the SEQ ID NO of the
complete ISVD, i.e. FR1-CDR1-FR2-CDR2-FR3-CDR3-FR4

| ID | Name | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 1 | T0170056G05 | 248 | EVQLVESGGGLVQ | 153 | GDVHKI | 251 | WYRQAPG | 209 | HISIG | 252 | YADSAKGRFTISRDESK | 223 | FSRIYPY | 260 | WGQGT |
| | | | PGGSLRLSCVAS | | NFLG | | KEREKVA | | DQTD | | NMVYLQMNSLKPEDT | | DY | | LVTVSS |
| | | | | | | | | | | | AVYFCRA | | | | |
| 2 | T017000141 | 249 | EVQLVESGGGVV | 153 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 2 | T017000351 | 249 | EVQLVESGGGVV | 153 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 3 | T017000343 | 249 | EVQLVESGGGVV | 153 | GDVHKI | 251 | WYRQAPG | 209 | HISIG | 254 | YADSAKGRFTISRDESK | 223 | FSRIYPY | 260 | WGQGT |
| | | | QPGGSLRLSCVAS | | NFLG | | KEREKVA | | DQTD | | NTVYLQMNSLRPEDTA | | DY | | LVTVSS |
| | | | | | | | | | | | LYFCRA | | | | |
| 4 | T017000345 | 249 | EVQLVESGGGVV | 153 | GDVHKI | 251 | WYRQAPG | 209 | HISIG | 255 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYYCRA | | | | |
| 5 | T017000164 | 249 | EVQLVESGGGVV | 154 | GYVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 6 | T017000166 | 249 | EVQLVESGGGVV | 155 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFYG | | KEREKVA | | DQTD | | NMVYLQMNSLRPEDT | | DY | | LVTVSS |
| | | | | | | | | | | | ALYFCRA | | | | |
| 7 | T017000161 | 249 | EVQLVESGGGVV | 156 | GEVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 8 | T017000177 | 249 | EVQLVESGGGVV | 157 | GSVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 9 | T017000188 | 249 | EVQLVESGGGVV | 158 | GAVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 10 | T017000174 | 249 | EVQLVESGGGVV | 159 | WDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 11 | T017000208 | 249 | EVQLVESGGGVV | 160 | YDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 12 | T017000163 | 249 | EVQLVESGGGVV | 161 | GDVHQI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 12 | T017000745 | 249 | EVQLVESGGGVV | 161 | GDVHQI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 13 | T017000176 | 249 | EVQLVESGGGVV | 162 | GDVHKL | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 14 | T017000179 | 249 | EVQLVESGGGVV | 163 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLT | | KEREKVA | | DQTD | | NMVYLQMNSLRPEDT | | DY | | LVTVSS |
| | | | | | | | | | | | ALYFCRA | | | | |
| 15 | T017000180 | 249 | EVQLVESGGGVV | 164 | QDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 16 | T017000196 | 249 | EVQLVESGGGVV | 165 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | FFLG | | KEREKVA | | DQTD | | NMVYLQMNSLRPEDT | | DY | | LVTVSS |
| | | | | | | | | | | | ALYFCRA | | | | |
| 17 | T017000170 | 249 | EVQLVESGGGVV | 166 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NYLG | | KEREKVA | | DQTD | | NMVYLQMNSLRPEDT | | DY | | LVTVSS |
| | | | | | | | | | | | ALYFCRA | | | | |
| 18 | T017000173 | 249 | EVQLVESGGGVV | 167 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NVLG | | KEREKVA | | DQTD | | NMVYLQMNSLRPEDT | | DY | | LVTVSS |
| | | | | | | | | | | | ALYFCRA | | | | |
| 19 | T017000185 | 249 | EVQLVESGGGVV | 168 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFFG | | KEREKVA | | DQTD | | NMVYLQMNSLRPEDT | | DY | | LVTVSS |
| | | | | | | | | | | | ALYFCRA | | | | |
| 20 | T017000200 | 249 | EVQLVESGGGVV | 169 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLR | | KEREKVA | | DQTD | | NMVYLQMNSLRPEDT | | DY | | LVTVSS |
| | | | | | | | | | | | ALYFCRA | | | | |
| 21 | T017000190 | 249 | EVQLVESGGGVV | 170 | GDVHKI | 251 | WYRQAPG | 209 | HISIG | 253 | YADSAKGRFTISRDESK | 223 | FSRIYPY | 260 | WGQGT |
| | | | QPGGSLRLSCVAS | | NFLS | | KEREKVA | | DQTD | | NMVYLQMNSLRPEDT | | DY | | LVTVSS |
| | | | | | | | | | | | ALYFCRA | | | | |
| 22 | T017000231 | 249 | EVQLVESGGGVV | 153 | GDVHKI | 251 | WYRQAPG | 210 | VISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 23 | T017000245 | 249 | EVQLVESGGGVV | 153 | GDVHKI | 251 | WYRQAPG | 211 | HISIA | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 24 | T017000238 | 249 | EVQLVESGGGVV | 153 | GDVHKI | 251 | WYRQAPG | 212 | HISIG | 253 | YADSAKGRFTISRDESK | 223 | FSRIYPY | 260 | WGQGT |
| | | | QPGGSLRLSCVAS | | NFLG | | KEREKVA | | DETD | | NMVYLQMNSLRPEDT | | DY | | LVTVSS |
| | | | | | | | | | | | ALYFCRA | | | | |
| 25 | T017000249 | 249 | EVQLVESGGGVV | 153 | GDVHKI | 251 | WYRQAPG | 213 | HISIG | 253 | YADSAKGRFTISRDESK | 223 | FSRIYPY | 260 | WGQGT |
| | | | QPGGSLRLSCVAS | | NFLG | | KEREKVA | | DVTD | | NMVYLQMNSLRPEDT | | DY | | LVTVSS |
| | | | | | | | | | | | ALYFCRA | | | | |
| 26 | T017000232 | 249 | EVQLVESGGGVV | 153 | GDVHKI | 251 | WYRQAPG | 214 | HISIG | 253 | YADSAKGRFTISRDESK | 223 | FSRIYPY | 260 | WGQGT |
| | | | QPGGSLRLSCVAS | | NFLG | | KEREKVA | | DQVD | | NMVYLQMNSLRPEDT | | DY | | LVTVSS |
| | | | | | | | | | | | ALYFCRA | | | | |
| 27 | T017000233 | 249 | EVQLVESGGGVV | 153 | GDVHKI | 251 | WYRQAPG | 215 | HISIG | 253 | YADSAKGRFTISRDESK | 223 | FSRIYPY | 260 | WGQGT |
| | | | QPGGSLRLSCVAS | | NFLG | | KEREKVA | | DQSD | | NMVYLQMNSLRPEDT | | DY | | LVTVSS |
| | | | | | | | | | | | ALYFCRA | | | | |
| 28 | T017000234 | 249 | EVQLVESGGGVV | 153 | GDVHKI | 251 | WYRQAPG | 216 | MISIG | 253 | YADSAKGRFTISRDESK | 223 | FSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 29 | T017000258 | 249 | EVQLVESGGGVV | 153 | GDVHKI | 251 | WYRQAPG | 217 | HISIGE | 253 | YADSAKGRFTISRDESK | 223 | FSRIYPY | 260 | WGQGT |
| | | | QPGGSLRLSCVAS | | NFLG | | KEREKVA | | QTD | | NMVYLQMNSLRPEDT | | DY | | LVTVSS |
| | | | | | | | | | | | ALYFCRA | | | | |
| 30 | T017000235 | 249 | EVQLVESGGGVV | 153 | GDVHKI | 251 | WYRQAPG | 218 | HISIG | 253 | YADSAKGRFTISRDESK | 223 | FSRIYPY | 260 | WGQGT |
| | | | QPGGSLRLSCVAS | | NFLG | | KEREKVA | | DHTD | | NMVYLQMNSLRPEDT | | DY | | LVTVSS |
| | | | | | | | | | | | ALYFCRA | | | | |
| 31 | T017000250 | 249 | EVQLVESGGGVV | 153 | GDVHKI | 251 | WYRQAPG | 219 | HISIG | 253 | YADSAKGRFTISRDESK | 223 | FSRIYPY | 260 | WGQGT |
| | | | QPGGSLRLSCVAS | | NFLG | | KEREKVA | | DKTD | | NMVYLQMNSLRPEDT | | DY | | LVTVSS |
| | | | | | | | | | | | ALYFCRA | | | | |
| 32 | T017000254 | 249 | EVQLVESGGGVV | 153 | GDVHKI | 251 | WYRQAPG | 220 | HISIG | 253 | YADSAKGRFTISRDESK | 223 | FSRIYPY | 260 | WGQGT |
| | | | QPGGSLRLSCVAS | | NFLG | | KEREKVA | | DRTD | | NMVYLQMNSLRPEDT | | DY | | LVTVSS |
| | | | | | | | | | | | ALYFCRA | | | | |
| 33 | T017000223 | 249 | EVQLVESGGGVV | 153 | GDVHKI | 251 | WYRQAPG | 221 | HISIG | 253 | YADSAKGRFTISRDESK | 223 | FSRIYPY | 260 | WGQGT |
| | | | QPGGSLRLSCVAS | | NFLG | | KEREKVA | | DQAD | | NMVYLQMNSLRPEDT | | DY | | LVTVSS |
| | | | | | | | | | | | ALYFCRA | | | | |
| 34 | T017000241 | 249 | EVQLVESGGGVV | 153 | GDVHKI | 251 | WYRQAPG | 222 | HISIG | 253 | YADSAKGRFTISRDESK | 223 | FSRIYPY | 260 | WGQGT |
| | | | QPGGSLRLSCVAS | | NFLG | | KEREKVA | | DQTN | | NMVYLQMNSLRPEDT | | DY | | LVTVSS |
| | | | | | | | | | | | ALYFCRA | | | | |
| 35 | T017000312 | 249 | EVQLVESGGGVV | 153 | GDVHKI | 251 | WYRQAPG | 209 | HISIG | 253 | YADSAKGRFTISRDESK | 224 | LSRIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 36 | T017000291 | 249 | EVQLVESGGGVV | 153 | GDVHKI | 251 | WYRQAPG | 209 | HISIG | 253 | YADSAKGRFTISRDESK | 225 | FSRIWP | 260 | WGQGT |
| | | | QPGGSLRLSCVAS | | NFLG | | KEREKVA | | DQTD | | NMVYLQMNSLRPEDT | | YDY | | LVTVSS |
| | | | | | | | | | | | ALYFCRA | | | | |
| 37 | T017000281 | 249 | EVQLVESGGGVV | 153 | GDVHKI | 251 | WYRQAPG | 209 | HISIG | 253 | YADSAKGRFTISRDESK | 226 | MSRIYP | 260 | WGQGT |
| | | | QPGGSLRLSCVAS | | NFLG | | KEREKVA | | DQTD | | NMVYLQMNSLRPEDT | | YDY | | LVTVSS |
| | | | | | | | | | | | ALYFCRA | | | | |
| 38 | T017000290 | 249 | EVQLVESGGGVV | 153 | GDVHKI | 251 | WYRQAPG | 209 | HISIG | 253 | YADSAKGRFTISRDESK | 227 | FSKIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 39 | T017000294 | 249 | EVQLVESGGGVV | 153 | GDVHKI | 251 | WYRQAPG | 209 | HISIG | 253 | YADSAKGRFTISRDESK | 228 | FSRIYPY | 260 | WGQGT |
| | | | QPGGSLRLSCVAS | | NFLG | | KEREKVA | | DQTD | | NMVYLQMNSLRPEDT | | DW | | LVTVSS |
| | | | | | | | | | | | ALYFCRA | | | | |
| 40 | T017000283 | 249 | EVQLVESGGGVV | 153 | GDVHKI | 251 | WYRQAPG | 209 | HISIG | 253 | YADSAKGRFTISRDESK | 229 | FSSIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 41 | T017000287 | 249 | EVQLVESGGGVV | 153 | GDVHKI | 251 | WYRQAPG | 209 | HISIG | 253 | YADSAKGRFTISRDESK | 230 | FSRIYPY | 260 | WGQGT |
| | | | QPGGSLRLSCVAS | | NFLG | | KEREKVA | | DQTD | | NMVYLQMNSLRPEDT | | QY | | LVTVSS |
| | | | | | | | | | | | ALYFCRA | | | | |
| 42 | T017000278 | 249 | EVQLVESGGGVV | 153 | GDVHKI | 251 | WYRQAPG | 209 | HISIG | 253 | YADSAKGRFTISRDESK | 231 | FSRIYPY | 260 | WGQGT |
| | | | QPGGSLRLSCVAS | | NFLG | | KEREKVA | | DQTD | | NMVYLQMNSLRPEDT | | SY | | LVTVSS |
| | | | | | | | | | | | ALYFCRA | | | | |
| 43 | T017000285 | 249 | EVQLVESGGGVV | 153 | GDVHKI | 251 | WYRQAPG | 209 | HISIG | 253 | YADSAKGRFTISRDESK | 232 | FSRIYPY | 260 | WGQGT |
| | | | QPGGSLRLSCVAS | | NFLG | | KEREKVA | | DQTD | | NMVYLQMNSLRPEDT | | LY | | LVTVSS |
| | | | | | | | | | | | ALYFCRA | | | | |
| 44 | T017000301 | 249 | EVQLVESGGGVV | 153 | GDVHKI | 251 | WYRQAPG | 209 | HISIG | 253 | YADSAKGRFTISRDESK | 233 | FSRIYPY | 260 | WGQGT |
| | | | QPGGSLRLSCVAS | | NFLG | | KEREKVA | | DQTD | | NMVYLQMNSLRPEDT | | AY | | LVTVSS |
| | | | | | | | | | | | ALYFCRA | | | | |
| 45 | T017000302 | 249 | EVQLVESGGGVV | 153 | GDVHKI | 251 | WYRQAPG | 209 | HISIG | 253 | YADSAKGRFTISRDESK | 234 | FSRIYPY | 260 | WGQGT |
| | | | QPGGSLRLSCVAS | | NFLG | | KEREKVA | | DQTD | | NMVYLQMNSLRPEDT | | TY | | LVTVSS |
| | | | | | | | | | | | ALYFCRA | | | | |
| 46 | T017000624 | 249 | EVQLVESGGGVV | 171 | GYVHKI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 47 | T017000623 | 249 | EVQLVESGGGVV | 172 | WDVHKI | 251 | WYRQAPG | 209 | HISIG | 256 | YADSAKGRFTISRDESK | 236 | LSKIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 48 | T017000625 | 249 | EVQLVESGGGVV | 173 | YDVHKI | 251 | WYRQAPG | 209 | HISIG | 256 | YADSAKGRFTISRDESK | 236 | LSKIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 49 | T017000635 | 249 | EVQLVESGGGVV | 174 | WDVHQ | 251 | WYRQAPG | 209 | HISIG | 256 | YADSAKGRFTISRDESK | 235 | LSRIWP | 260 | WGQGT |
| | | | QPGGSLRLSCVAS | | INFYG | | KEREKVA | | DQTD | | NTVYLQMNSLRPEDTA | | YDY | | LVTVSS |
| | | | | | | | | | | | LYYCRA | | | | |
| 50 | T017000641 | 249 | EVQLVESGGGVV | 175 | WDVHKI | 251 | WYRQAPG | 209 | HISIG | 256 | YADSAKGRFTISRDESK | 236 | LSKIWP | 260 | WGQGT |
| | | | QPGGSLRLSCVAS | | NFYT | | KEREKVA | | DQTD | | NTVYLQMNSLRPEDTA | | YDY | | LVTVSS |
| | | | | | | | | | | | LYYCRA | | | | |
| 51 | T017000687 | 249 | EVQLVESGGGVV | 176 | GSVHQI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 52 | T017000613 | 249 | EVQLVESGGGVV | 172 | WDVHKI | 251 | WYRQAPG | 209 | HISIG | 253 | YADSAKGRFTISRDESK | 236 | LSKIWP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 53 | T017000615 | 249 | EVQLVESGGGVV | 173 | YDVHKI | 251 | WYRQAPG | 209 | HISIG | 253 | YADSAKGRFTISRDESK | 236 | LSKIWP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 54 | T017000505 | 249 | EVQLVESGGGVV | 177 | GDVHQ | 251 | WYRQAPG | 209 | HISIG | 253 | YADSAKGRFTISRDESK | 227 | FSKIYPY | 260 | WGQGT |
| | | | QPGGSLRLSCVAS | | LNFLG | | KEREKVA | | DQTD | | NMVYLQMNSLRPEDT | | DY | | LVTVSS |
| | | | | | | | | | | | ALYFCRA | | | | |
| 55 | T017000362 | 249 | EVQLVESGGGVV | 173 | YDVHKI | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 56 | T017000440 | 249 | EVQLVESGGGVV | 178 | WDVHK | 251 | WYRQAPG | 209 | HISIG | 253 | YADSAKGRFTISRDESK | 237 | MSKIW | 260 | WGQGT |
| | | | QPGGSLRLSCVAS | | LNFYG | | KEREKVA | | DQTD | | NMVYLQMNSLRPEDT | | PYDY | | LVTVSS |
| | | | | | | | | | | | ALYFCRA | | | | |
| 57 | T017000378 | 249 | EVQLVESGGGVV | 172 | WDVHKI | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 58 | T017000608 | 249 | EVQLVESGGGVV | 172 | WDVHKI | 251 | WYRQAPG | 209 | HISIG | 253 | YADSAKGRFTISRDESK | 238 | FSKIWP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 59 | T017000374 | 249 | EVQLVESGGGVV | 179 | YDVHQI | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 60 | T017000607 | 249 | EVQLVESGGGVV | 174 | WDVHQ | 251 | WYRQAPG | 209 | HISIG | 253 | YADSAKGRFTISRDESK | 238 | FSKIWP | 260 | WGQGT |
| | | | QPGGSLRLSCVAS | | INFYG | | KEREKVA | | DQTD | | NMVYLQMNSLRPEDT | | YDY | | LVTVSS |
| | | | | | | | | | | | ALYFCRA | | | | |
| 61 | T017000602 | 249 | EVQLVESGGGVV | 180 | WDVHQ | 251 | WYRQAPG | 209 | HISIG | 253 | YADSAKGRFTISRDESK | 238 | FSKIWP | 260 | WGQGT |
| | | | QPGGSLRLSCVAS | | INFLG | | KEREKVA | | DQTD | | NMVYLQMNSLRPEDT | | YDY | | LVTVSS |
| | | | | | | | | | | | ALYFCRA | | | | |
| 62 | T017000359 | 249 | EVQLVESGGGVV | 174 | WDVHQ | 251 | WYRQAPG | 209 | HISIG | 253 | YADSAKGRFTISRDESK | 227 | FSKIYPY | 260 | WGQGT |
| | | | QPGGSLRLSCVAS | | INFYG | | KEREKVA | | DQTD | | NMVYLQMNSLRPEDT | | DY | | LVTVSS |
| | | | | | | | | | | | ALYFCRA | | | | |
| 63 | T017000740 | 249 | EVQLVESGGGVV | 181 | WDVHQ | 251 | WYRQAPG | 209 | HISIG | 253 | YADSAKGRFTISRDESK | 239 | MSKIYP | 260 | WGQGT |
| | | | QPGGSLRLSCVAS | | LNFYG | | KEREKVA | | DQTD | | NMVYLQMNSLRPEDT | | YDY | | LVTVSS |
| | | | | | | | | | | | ALYFCRA | | | | |
| 64 | T017000554 | 249 | EVQLVESGGGVV | 182 | YDVHQI | 251 | WYRQAPG | 209 | HISIG | 253 | YADSAKGRFTISRDESK | 239 | MSKIYP | 260 | WGQGT |
| | | | QPGGSLRLSCVAS | | NFLG | | KEREKVA | | DQTD | | NMVYLQMNSLRPEDT | | YDY | | LVTVSS |
| | | | | | | | | | | | ALYFCRA | | | | |
| 65 | T017000413 | 249 | EVQLVESGGGVV | 181 | WDVHQ | 251 | WYRQAPG | 209 | HISIG | 253 | YADSAKGRFTISRDESK | 237 | MSKIW | 260 | WGQGT |
| | | | QPGGSLRLSCVAS | | LNFYG | | KEREKVA | | DQTD | | NMVYLQMNSLRPEDT | | PYDY | | LVTVSS |
| | | | | | | | | | | | ALYFCRA | | | | |
| 66 | T017000379 | 249 | EVQLVESGGGVV | 174 | WDVHQ | 251 | WYRQAPG | 209 | HISIG | 253 | YADSAKGRFTISRDESK | 237 | MSKIW | 260 | WGQGT |
| | | | QPGGSLRLSCVAS | | INFYG | | KEREKVA | | DQTD | | NMVYLQMNSLRPEDT | | PYDY | | LVTVSS |
| | | | | | | | | | | | ALYFCRA | | | | |
| 67 | T017000367 | 249 | EVQLVESGGGVV | 154 | GYVHKI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 68 | T017000390 | 249 | EVQLVESGGGVV | 180 | WDVHQ | 251 | WYRQAPG | 209 | HISIG | 253 | YADSAKGRFTISRDESK | 237 | MSKIW | 260 | WGQGT |
| | | | QPGGSLRLSCVAS | | INFLG | | KEREKVA | | DQTD | | NMVYLQMNSLRPEDT | | PYDY | | LVTVSS |
| | | | | | | | | | | | ALYFCRA | | | | |
| 69 | T017000443 | 249 | EVQLVESGGGVV | 174 | WDVHQ | 251 | WYRQAPG | 209 | HISIG | 253 | YADSAKGRFTISRDESK | 239 | MSKIYP | 260 | WGQGT |
| | | | QPGGSLRLSCVAS | | INFYG | | KEREKVA | | DQTD | | NMVYLQMNSLRPEDT | | YDY | | LVTVSS |
| | | | | | | | | | | | ALYFCRA | | | | |
| 70 | T017000776 | 249 | EVQLVESGGGVV | 183 | WSVHQI | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 71 | T017000371 | 249 | EVQLVESGGGVV | 171 | GYVHKI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 72 | T017000759 | 249 | EVQLVESGGGVV | 184 | GSVHKI | 251 | WYRQAPG | 209 | HISIG | 253 | YADSAKGRFTISRDESK | 241 | FSKIWP | 26 | WGQGT |
| | | | QPGGSLRLSCVAS | | NFYG | | KEREKVA | | DQTD | | NMVYLQMNSLRPEDT | | YDW | | LVTVSS |
| | | | | | | | | | | | ALYFCRA | | | | |
| 73 | T017000539 | 249 | EVQLVESGGGVV | 171 | GYVHKI | 251 | WYRQAPG | 209 | HISIG | 253 | YADSAKGRFTISRDESK | 242 | MSRIW | 260 | WGQGT |
| | | | QPGGSLRLSCVAS | | NFYG | | KEREKVA | | DQTD | | NMVYLQMNSLRPEDT | | PYDW | | LVTVSS |
| | | | | | | | | | | | ALYFCRA | | | | |
| 74 | T017000499 | 249 | EVQLVESGGGVV | 185 | GYVHKL | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 75 | T017000538 | 249 | EVQLVESGGGVV | 186 | GYVHKL | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 76 | T017000410 | 249 | EVQLVESGGGVV | 186 | GYVHKL | 251 | WYRQAPG | 209 | HISIG | 253 | YADSAKGRFTISRDESK | 243 | FSRIWP | 260 | WGQGT |
| | | | QPGGSLRLSCVAS | | NFYG | | KEREKVA | | DQTD | | NMVYLQMNSLRPEDT | | YDW | | LVTVSS |
| | | | | | | | | | | | ALYFCRA | | | | |
| 77 | T017000566 | 249 | EVQLVESGGGVV | 185 | GYVHKL | 251 | WYRQAPG | 209 | HISIG | 253 | YADSAKGRFTISRDESK | 242 | MSRIW | 260 | WGQGT |
| | | | QPGGSLRLSCVAS | | NFLG | | KEREKVA | | DQTD | | NMVYLQMNSLRPEDT | | PYDW | | LVTVSS |
| | | | | | | | | | | | ALYFCRA | | | | |
| 78 | T017000589 | 249 | EVQLVESGGGVV | 186 | GYVHKL | 251 | WYRQAPG | 209 | HISIG | 253 | YADSAKGRFTISRDESK | 242 | MSRIW | 260 | WGQGT |
| | | | QPGGSLRLSCVAS | | NFYG | | KEREKVA | | DQTD | | NMVYLQMNSLRPEDT | | PYDW | | LVTVSS |
| | | | | | | | | | | | ALYFCRA | | | | |
| 79 | T017000397 | 249 | EVQLVESGGGVV | 187 | GSVHQL | 251 | WYRQAPG | 209 | HISIG | 253 | YADSAKGRFTISRDESK | 240 | MSRIW | 26 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 80 | T017000757 | 249 | EVQLVESGGGVV | 188 | GAVHKL | 251 | WYRQAPG | 209 | HISIG | 253 | YADSAKGRFTISRDESK | 241 | FSKIWP | 260 | WGQGT |
| | | | QPGGSLRLSCVAS | | NFLG | | KEREKVA | | DQTD | | NMVYLQMNSLRPEDT | | YDW | | LVTVSS |
| | | | | | | | | | | | ALYFCRA | | | | |
| 81 | T017000372 | 249 | EVQLVESGGGVV | 171 | GYVHKI | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 82 | T017000423 | 249 | EVQLVESGGGVV | 189 | GSVHQI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 83 | T017000521 | 249 | EVQLVESGGGVV | 190 | GSVHQL | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 84 | T017000370 | 249 | EVQLVESGGGVV | 191 | GAVHQI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 85 | T017000383 | 249 | EVQLVESGGGVV | 161 | GDVHQI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 86 | T017000364 | 249 | EVQLVESGGGVV | 192 | GAVHQI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 87 | T017000442 | 249 | EVQLVESGGGVV | 186 | GYVHKL | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 88 | T017000461 | 249 | EVQLVESGGGVV | 193 | GDVHQI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 89 | T017000384 | 249 | EVQLVESGGGVV | 194 | GAVHQ | 251 | WYRQAPG | 209 | HISIG | 253 | YADSAKGRFTISRDESK | 240 | MSRIW | 260 | WGQGT |
| | | | QPGGSLRLSCVAS | | LNFYG | | KEREKVA | | DQTD | | NMVYLQMNSLRPEDT | | PYDY | | LVTVSS |
| | | | | | | | | | | | ALYFCRA | | | | |
| 90 | T017000593 | 249 | EVQLVESGGGVV | 195 | GAVHKL | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 91 | T017000373 | 249 | EVQLVESGGGVV | 196 | GSVHKL | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 92 | T017000614 | 249 | EVQLVESGGGVV | 171 | GYVHKI | 251 | WYRQAPG | 209 | HISIG | 253 | YADSAKGRFTISRDESK | 235 | LSRIWP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 93 | T017000509 | 249 | \|EVQLVESGGGVV | 158 | GAVHKI | 251 | WYRQAPG | 209 | HISIG | 253 | YADSAKGRFTISRDESK | 244 | MSKIW | 260 | WGQGT |
| | | | QPGGSLRLSCVAS | | NFLG | | KEREKVA | | DQTD | | NMVYLQMNSLRPEDT | | PYDW | | LVTVSS |
| | | | | | | | | | | | ALYFCRA | | | | |
| 94 | T017000361 | 249 | \|EVQLVESGGGVV | 157 | GSVHKI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 95 | T017000498 | 249 | EVQLVESGGGVV | 197 | GAVHKI | 251 | WYRQAPG | 209 | HISIG | 253 | YADSAKGRFTISRDESK | 242 | MSRIW | 260 | WGQGT |
| | | | QPGGSLRLSCVAS | | NFYG | | KEREKVA | | DQTD | | NMVYLQMNSLRPEDT | | PYDW | | LVTVSS |
| | | | | | | | | | | | ALYFCRA | | | | |
| 96 | T017000408 | 249 | EVQLVESGGGVV | 184 | GSVHKI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 97 | T017000432 | 249 | EVQLVESGGGVV | 198 | GSVHKL | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 98 | T017000381 | 249 | EVQLVESGGGVV | 158 | GAVHKI | 251 | WYRQAPG | 209 | HISIG | 253 | YADSAKGRFTISRDESK | 226 | MSRIYP | 260 | WGQGT |
| | | | QPGGSLRLSCVAS | | NFLG | | KEREKVA | | DQTD | | NMVYLQMNSLRPEDT | | YDY | | LVTVSS |
| | | | | | | | | | | | ALYFCRA | | | | |
| 99 | T017000754 | 249 | EVQLVESGGGVV | 199 | GYVHQI | 251 | WYRQAPG | 209 | HISIG | 253 | YADSAKGRFTISRDESK | 237 | MSKIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 100 | T017000515 | 249 | EVQLVESGGGVV | 158 | GAVHKI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 101 | T017000360 | 249 | EVQLVESGGGVV | 200 | WSVHQ | 251 | WYRQAPG | 209 | HISIG | 253 | YADSAKGRFTISRDESK | 245 | MSKIYP | 260 | WGQGT |
| | | | QPGGSLRLSCVAS | | LNFYG | | KEREKVA | | DQTD | | NMVYLQMNSLRPEDT | | YDW | | LVTVSS |
| | | | | | | | | | | | ALYFCRA | | | | |
| 102 | T017000588 | 249 | EVQLVESGGGVV | 197 | GAVHKI | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 103 | T017000401 | 249 | EVQLVESGGGVV | 184 | GSVHKI | 251 | WYRQAPG | 209 | HISIG | 253 | YADSAKGRFTISRDESK | 242 | MSRIW | 260 | WGQGT |
| | | | QPGGSLRLSCVAS | | NFYG | | KEREKVA | | DQTD | | NMVYLQMNSLRPEDT | | PYDW | | LVTVSS |
| | | | | | | | | | | | ALYFCRA | | | | |
| 104 | T017000357 | 249 | EVQLVESGGGVV | 201 | GYVHQL | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 105 | T017000354 | 249 | EVQLVESGGGVV | 197 | GAVHKI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 106 | T017000547 | 249 | EVQLVESGGGVV | 195 | GAVHKL | 251 | WYRQAPG | 209 | HISIG | 253 | YADSAKGRFTISRDESK | 225 | FSRIWP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 107 | T017000531 | 249 | EVQLVESGGGVV | 157 | GSVHKI | 251 | WYRQAPG | 209 | HISIG | 253 | YADSAKGRFTISRDESK | 242 | MSRIW | 260 | WGQGT |
| | | | QPGGSLRLSCVAS | | NFLG | | KEREKVA | | DQTD | | NMVYLQMNSLRPEDT | | PYDW | | LVTVSS |
| | | | | | | | | | | | ALYFCRA | | | | |
| 108 | T017000391 | 24 | EVQLVESGGGVV | 197 | GAVHKI | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 109 | T017000356 | 249 | EVQLVESGGGVV | 198 | GSVHKL | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 110 | T017000406 | 249 | EVQLVESGGGVV | 202 | GYVHQI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 111 | T017000468 | 249 | EVQLVESGGGVV | 184 | GSVHKI | 251 | WYRQAPG | 209 | HISIG | 253 | YADSAKGRFTISRDESK | 225 | FSRIWP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 112 | T017000358 | 249 | EVQLVESGGGVV | 188 | GAVHKL | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 113 | T017000685 | 249 | EVQLVESGGGVV | 203 | GEVHQI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 114 | T017000369 | 249 | EVQLVESGGGVV | 198 | GSVHKL | 251 | WYRQAPG | 209 | HISIG | 253 | YADSAKGRFTISRDESK | 242 | MSRIW | 260 | WGQGT |
| | | | QPGGSLRLSCVAS | | NFYG | | KEREKVA | | DQTD | | NMVYLQMNSLRPEDT | | PYDW | | LVTVSS |
| | | | | | | | | | | | ALYFCRA | | | | |
| 115 | T017000363 | 249 | EVQLVESGGGVV | 184 | GSVHKI | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 116 | T017000450 | 249 | EVQLVESGGGVV | 199 | GYVHQI | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 117 | T017000530 | 249 | EVQLVESGGGVV | 188 | GAVHKL | 251 | WYRQAPG | 209 | HISIG | 257 | YADSAKDRFTISRDESK | 226 | MSRIYP | 260 | WGQGT |
| | | | QPGGSLRLSCVAS | | NFLG | | KEREKVA | | DQTD | | NMVYLQMNSLRPEDT | | YDY | | LVTVSS |
| | | | | | | | | | | | ALYFCRA | | | | |
| 118 | T017000473 | 249 | EVQLVESGGGVV | 202 | GYVHQI | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 119 | T017000418 | 249 | EVQLVESGGGVV | 195 | GAVHKL | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 120 | T017000611 | 249 | EVQLVESGGGVV | 193 | GDVHQI | 251 | WYRQAPG | 209 | HISIG | 253 | YADSAKGRFTISRDESK | 227 | FSKIYPY | 260 | WGQGT |
| | | | QPGGSLRLSCVAS | | NFYG | | KEREKVA | | DQTD | | NMVYLQMNSLRPEDT | | DY | | LVTVSS |
| | | | | | | | | | | | ALYFCRA | | | | |
| 121 | T017000386 | 249 | EVQLVESGGGVV | 196 | GSVHKL | 251 | WYRQAPG | 209 | HISIG | 253 | YADSAKGRFTISRDESK | 243 | FSRIWP | 260 | WGQGT |
| | | | QPGGSLRLSCVAS | | NFLG | | KEREKVA | | DQTD | | NMVYLQMNSLRPEDT | | YDW | | LVTVSS |
| | | | | | | | | | | | ALYFCRA | | | | |
| 122 | T017000382 | 249 | EVQLVESGGGVV | 184 | GSVHKI | 251 | WYRQAPG | 209 | HISIG | 253 | YADSAKGRFTISRDESK | 246 | MSRIYP | 260 | WGQGT |
| | | | QPGGSLRLSCVAS | | NFYG | | KEREKVA | | DQTD | | NMVYLQMNSLRPEDT | | YDW | | LVTVSS |
| | | | | | | | | | | | ALYFCRA | | | | |
| 123 | T017000419 | 249 | EVQLVESGGGVV | 195 | GAVHKL | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 124 | T017000774 | 249 | EVQLVESGGGVV | 193 | GDVHQI | 251 | WYRQAPG | 209 | HISIG | 253 | YADSAKGRFTISRDESK | 237 | MSKIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 125 | T017000686 | 249 | EVQLVESGGGVV | 184 | GSVHKI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 126 | T017000569 | 249 | EVQLVESGGGVV | 162 | GDVHKL | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 127 | T017000365 | 249 | EVQLVESGGGVV | 188 | GAVHKL | 251 | WYRQAPG | 209 | HISIG | 253 | YADSAKGRFTISRDESK | 242 | MSRIW | 260 | WGQGT |
| | | | QPGGSLRLSCVAS | | NFLG | | KEREKVA | | DQTD | | NMVYLQMNSLRPEDT | | PYDW | | LVTVSS |
| | | | | | | | | | | | ALYFCRA | | | | |
| 128 | T017000434 | 249 | EVQLVESGGGVV | 198 | GSVHKL | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 129 | T017000612 | 249 | EVQLVESGGGVV | 177 | GDVHQ | 251 | WYRQAPG | 209 | HISIG | 253 | YADSAKGRFTISRDESK | 247 | LSKIYPY | 260 | WGQGT |
| | | | QPGGSLRLSCVAS | | LNFLG | | KEREKVA | | DQTD | | NMVYLQMNSLRPEDT | | DY | | LVTVSS |
| | | | | | | | | | | | ALYFCRA | | | | |
| 130 | T017000610 | 249 | EVQLVESGGGVV | 161 | GDVHQI | 251 | WYRQAPG | 209 | HISIG | 253 | YADSAKGRFTISRDESK | 247 | LSKIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 131 | T017000682 | 249 | EVQLVESGGGVV | 197 | GAVHKI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 132 | T017000766 | 249 | EVQLVESGGGVV | 171 | GYVHKI | 251 | WYRQAPG | 209 | HISIG | 253 | YADSAKGRFTISRDESK | 239 | MSKIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 133 | T017000690 | 249 | EVQLVESGGGVV | 204 | WEVHKI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 134 | T017000750 | 249 | EVQLVESGGGVV | 205 | YAVHQI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 135 | T017000742 | 249 | EVQLVESGGGVV | 202 | GYVHQI | 251 | WYRQAPG | 209 | HISIG | 253 | YADSAKGRFTISRDESK | 242 | MSRIW | 260 | WGQGT |
| | | | QPGGSLRLSCVAS | | NFYG | | KEREKVA | | DQTD | | NMVYLQMNSLRPEDT | | PYDW | | LVTVSS |
| | | | | | | | | | | | ALYFCRA | | | | |
| 136 | T017000577 | 249 | EVQLVESGGGVV | 180 | WDVHQ | 251 | WYRQAPG | 209 | HISIG | 253 | YADSAKGRFTISRDESK | 226 | MSRIYP | 260 | WGQGT |
| | | | QPGGSLRLSCVAS | | INFLG | | KEREKVA | | DQTD | | NMVYLQMNSLRPEDT | | YDY | | LVTVSS |
| | | | | | | | | | | | ALYFCRA | | | | |
| 137 | T017000609 | 249 | EVQLVESGGGVV | 161 | GDVHQI | 251 | WYRQAPG | 209 | HISIG | 253 | YADSAKGRFTISRDESK | 227 | FSKIYPY | 260 | WGQGT |
| | | | QPGGSLRLSCVAS | | NFLG | | KEREKVA | | DQTD | | NMVYLQMNSLRPEDT | | DY | | LVTVSS |
| | | | | | | | | | | | ALYFCRA | | | | |
| 138 | T017000355 | 249 | EVQLVESGGGVV | 181 | WDVHQ | 251 | WYRQAPG | 209 | HISIG | 253 | YADSAKGRFTISRDESK | 240 | MSRIW | 260 | WGQGT |
| | | | QPGGSLRLSCVAS | | LNFYG | | KEREKVA | | DQTD | | NMVYLQMNSLRPEDT | | PYDY | | LVTVSS |
| | | | | | | | | | | | ALYFCRA | | | | |
| 139 | T017000403 | 249 | EVQLVESGGGVV | 180 | WDVHQ | 251 | WYRQAPG | 209 | HISIG | 253 | YADSAKGRFTISRDESK | 240 | MSRIW | 260 | WGQGT |
| | | | QPGGSLRLSCVAS | | INFLG | | KEREKVA | | DQTD | | NMVYLQMNSLRPEDT | | PYDY | | LVTVSS |
| | | | | | | | | | | | ALYFCRA | | | | |
| 140 | T017000578 | 249 | EVQLVESGGGVV | 206 | YDVHQL | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 141 | T017000415 | 249 | EVQLVESGGGVV | 174 | WDVHQ | 251 | WYRQAPG | 209 | HISIG | 253 | YADSAKGRFTISRDESK | 240 | MSRIW | 260 | WGQGT |
| | | | QPGGSLRLSCVAS | | INFYG | | KEREKVA | | DQTD | | NMVYLQMNSLRPEDT | | PYDY | | LVTVSS |
| | | | | | | | | | | | ALYFCRA | | | | |
| 142 | T017000368 | 249 | EVQLVESGGGVV | 174 | WDVHQ | 251 | WYRQAPG | 209 | HISIG | 253 | YADSAKGRFTISRDESK | 226 | MSRIYP | 260 | WGQGT |
| | | | QPGGSLRLSCVAS | | INFYG | | KEREKVA | | DQTD | | NMVYLQMNSLRPEDT | | YDY | | LVTVSS |
| | | | | | | | | | | | ALYFCRA | | | | |
| 143 | T017000398 | 249 | EVQLVESGGGVV | 179 | YDVHQI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 144 | T017000377 | 249 | EVQLVESGGGVV | 172 | WDVHKI | 251 | WYRQAPG | 209 | HISIG | 253 | YADSAKGRFTISRDESK | 226 | MSRIYP | 260 | WGQGT |
| | | | QPGGSLRLSCVAS | | NFYG | | KEREKVA | | DQTD | | NMVYLQMNSLRPEDT | | YDY | | LVTVSS |
| | | | | | | | | | | | ALYFCRA | | | | |
| 145 | T017000411 | 249 | EVQLVESGGGVV | 172 | WDVHKI | 251 | WYRQAPG | 209 | HISIG | 253 | YADSAKGRFTISRDESK | 240 | MSRIW | 260 | WGQGT |
| | | | QPGGSLRLSCVAS | | NFYG | | KEREKVA | | DQTD | | NMVYLQMNSLRPEDT | | PYDY | | LVTVSS |
| | | | | | | | | | | | ALYFCRA | | | | |
| 146 | T017000691 | 249 | EVQLVESGGGVV | 207 | WEVHQ | 251 | WYRQAPG | 209 | HISIG | 256 | YADSAKGRFTISRDESK | 235 | LSRIWP | 260 | WGQGT |
| | | | QPGGSLRLSCVAS | | INFYG | | KEREKVA | | DQTD | | NTVYLQMNSLRPEDTA | | YDY | | LVTVSS |
| | | | | | | | | | | | LYYCRA | | | | |
| 147 | T017000638 | 249 | EVQLVESGGGVV | 175 | WDVHKI | 251 | WYRQAPG | 209 | HISIG | 256 | YADSAKGRFTISRDESK | 235 | LSRIWP | 260 | WGQGT |
| | | | QPGGSLRLSCVAS | | NFYT | | KEREKVA | | DQTD | | NTVYLQMNSLRPEDTA | | YDY | | LVTVSS |
| | | | | | | | | | | | LYYCRA | | | | |
| 148 | T017000550 | 249 | EVQLVESGGGVV | 189 | GSVHQI | 251 | WYRQAPG | 209 | HISIG | 253 | YADSAKGRFTISRDESK | 246 | MSRIYP | 260 | WGQGT |
| | | | QPGGSLRLSCVAS | | NFLG | | KEREKVA | | DQTD | | NMVYLQMNSLRPEDT | | YDW | | LVTVSS |
| | | | | | | | | | | | ALYFCRA | | | | |
| 149 | T017000518 | 249 | EVQLVESGGGVV | 208 | YSVHQL | 251 | WYRQAPG | 209 | HISIG | 253 | YADSAKGRFTISRDESK | 240 | MSRIW | 260 | WGQGT |
| | | | QPGGSLRLSCVAS | | NFLG | | KEREKVA | | DQTD | | NMVYLQMNSLRPEDT | | PYDY | | LVTVSS |
| | | | | | | | | | | | ALYFCRA | | | | |
| 150 | T017000679 | 250 | DVQLVESGGGVV | 171 | GYVHKI | 251 | WYRQAPG | 209 | HISIG | 256 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | LYYCRA | | | | |
| 151 | T017000680 | 250 | DVQLVESGGGVV | 171 | GYVHKI | 251 | WYRQAPG | 209 | HISIG | 258 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | AYYCRA | | | | |
| 152 | T017000681 | 250 | DVQLVESGGGVV | 171 | GYVHKI | 251 | WYRQAPG | 209 | HISIG | 259 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | TYYCRA | | | | |
| 261 | T017000697 | 249 | EVQLVESGGGVV | 171 | GYVHKI | 251 | WYRQAPG | 209 | HISIG | 258 | YADSAKGRFTISRDESK | 235 | LSRIWP | 260 | WGQGT |
| | | | QPGGSLRLSCVAS | | NFYG | | KEREKVA | | DQTD | | NTVYLQMNSLRPEDTA | | YDY | | LVTVSS |
| | | | | | | | | | | | AYYCRA | | | | |
| 262 | T017000681 | 249 | EVQLVESGGGVV | 171 | GYVHKI | 251 | WYRQAPG | 209 | HISIG | 259 | YADSAKGRFTISRDESK | 235 | LSRIWP | 260 | WGQGT |
| | (D1E) | | QPGGSLRLSCVAS | | NFYG | | KEREKVA | | DQTD | | NTVYLQMNSLRPEDTA | | YDY | | LVTVSS |
| | | | | | | | | | | | TYYCRA | | | | |

TABLE A-3
Amino acid sequences of ISVD formats (″ID″ refers to the given SEQ ID NO)

| Name | ID | Amino acid sequence |
|---|---|---|
| T017000128 | 268 | DVQLVESGGGLVQPGGSLRLSCVASGDVHKINFLGWYRQAPGKEREKVAHISIGDQTDYADSAKGRFTISRDESKNMVYLQMNSLKPEDTAVYFCR |
| | | AFSRIYPYDYWGQGTLVTVSSGGGGSGGGGSGGGGSGGGGSGGGGSGGGGSGGGGSEVQLVESGGGLVQAGGSLRLSCAASGITSKINDMGW |
| | | YRQTPGNYREWVASITATGTTNYRDSVKGRFTISRDNAKSTVYLQMNSLKPEDTTVYYCNTFPPISNFWGQGTLVTVSSA |
| T017000131 | 269 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGLVQPGGSLRLSCVASGDVHKINFLGWYR |
| | | QAPGKEREKVAHISIGDQTDYADSAKGRFTISRDESKNMVYLQMNSLKPEDTAVYFCRAFSRIYPYDYWGQGTLVTVSSA |
| T017000139 | 270 | DVQLVESGGGLVQPGGSLRLSCVASGDVHKINFLGWYRQAPGKEREKVAHISIGDQTDYADSAKGRFTISRDESKNMVYLQMNSLKPEDTAVYFCR |
| | | AFSRIYPYDYWGQGTLVTVSSGGGGSGGGGSGGGGSGGGGSGGGGSGGGGSGGGGSEVQLVESGGGLVQAGGPLRLSCAASGRTFSSYVMGW |
| | | FRQAPGKEREFVAAIYWSNGKTQYTDSVKGRFTISGDNAKNTVYLQMNSLNPEDTAVYYCVADKDETGFRTLPIAYDYWGQGTLVTVSSGGGGSG |
| | | GGGSGGGGSGGGGSGGGGSGGGGSGGGGSEVQLVESGGGLVQAGGSLRLSCAASGITSKINDMGWYRQTPGNYREWVASITATGTTNYRDSV |
| | | KGRFTISRDNAKSTVYLQMNSLKPEDTTVYYCNTFPPISNFWGQGTLVTVSSA |
| T017000627 | 271 | DVQLVESGGGVVQPGGSLRLSCVASWDVHKINFYGWYRQAPGKEREKVAHISIGDQTDYADSAKGRFTISRDESKNTVYLQMNSLRPEDTALYYCR |
| | | ALSKIWPYDYWGQGTLVTVSSGGGGSGGGGSGGGGSGGGGSGGGGSGGGGSGGGGSEVQLVESGGGLVQAGGSLRLSCAASGITSKINDMGW |
| | | YRQTPGNYREWVASITATGTTNYRDSVKGRFTISRDNAKSTVYLQMNSLKPEDTTVYYCNTFPPISNFWGQGTLVTVSSA |
| T017000628 | 272 | DVQLVESGGGVVQPGGSLRLSCVASYDVHKINFYGWYRQAPGKEREKVAHISIGDQTDYADSAKGRFTISRDESKNTVYLQMNSLRPEDTALYYCR |
| | | ALSKIWPYDYWGQGTLVTVSSGGGGSGGGGSGGGGSGGGGSGGGGSGGGGSGGGGSEVQLVESGGGLVQAGGSLRLSCAASGITSKINDMGW |
| | | YRQTPGNYREWVASITATGTTNYRDSVKGRFTISRDNAKSTVYLQMNSLKPEDTTVYYCNTFPPISNFWGQGTLVTVSSA |
| T017000630 | 273 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGVVQPGGSLRLSCVASWDVHKINFYGWYR |
| | | QAPGKEREKVAHISIGDQTDYADSAKGRFTISRDESKNTVYLQMNSLRPEDTALYYCRALSKIWPYDYWGQGTLVTVSSA |
| T017000631 | 274 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGGGGGSGGGGGGGGSGGGGSGGGGSEVQLVESGGGVVQPGGSLRLSCVASYDVHKINFYGWYR |
| | | QAPGKEREKVAHISIGDQTDYADSAKGRFTISRDESKNTVYLQMNSLRPEDTALYYCRALSKIWPYDYWGQGTLVTVSSA |
| T017000633 | 275 | DVQLVESGGGVVQPGGSLRLSCVASGYVHKINFYGWYRQAPGKEREKVAHISIGDQTDYADSAKGRFTISRDESKNTVYLQMNSLRPEDTALYYCR |
| | | ALSRIWPYDYWGQGTLVTVSSGGGGSGGGGSGGGGSGGGGSGGGGSGGGGSGGGGSEVQLVESGGGLVQAGGSLRLSCAASGITSKINDMGW |
| | | YRQTPGNYREWVASITATGTTNYRDSVKGRFTISRDNAKSTVYLQMNSLKPEDTTVYYCNTFPPISNFWGQGTLVTVSSA |
| T017000634 | 276 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGVVQPGGSLRLSCVASGYVHKINFYGWYR |
| | | QAPGKEREKVAHISIGDQTDYADSAKGRFTISRDESKNTVYLQMNSLRPEDTALYYCRALSRIWPYDYWGQGTLVTVSSA |
| T017000636 | 277 | DVQLVESGGGVVQPGGSLRLSCVASWDVHQINFYGWYRQAPGKEREKVAHISIGDQTDYADSAKGRFTISRDESKNTVYLQMNSLRPEDTALYYC |
| | | RALSRIWPYDYWGQGTLVTVSSGGGGSGGGGSGGGGSGGGGSGGGGSGGGGSGGGGSEVQLVESGGGLVQAGGSLRLSCAASGITSKINDMG |
| | | WYRQTPGNYREWVASITATGTTNYRDSVKGRFTISRDNAKSTVYLQMNSLKPEDTTVYYCNTFPPISNFWGQGTLVTVSSA |
| T017000637 | 278 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGGGGGSGGGGSGGGGSGGGGSGGGGSGGGGSEVQLVESGGGVVQPGGSLRLSCVASWDVHQINFYGWY |
| | | RQAPGKEREKVAHISIGDQTDYADSAKGRFTISRDESKNTVYLQMNSLRPEDTALYYCRALSRIWPYDYWGQGTLVTVSSA |
| T017000639 | 279 | DVQLVESGGGVVQPGGSLRLSCVASWDVHKINFYTWYRQAPGKEREKVAHISIGDQTDYADSAKGRFTISRDESKNTVYLQMNSLRPEDTALYYCR |
| | | ALSRIWPYDYWGQGTLVTVSSGGGGSGGGGSGGGGSGGGGSGGGGSGGGGSGGGGSEVQLVESGGGLVQAGGSLRLSCAASGITSKINDMGW |
| | | YRQTPGNYREWVASITATGTTNYRDSVKGRFTISRDNAKSTVYLQMNSLKPEDTTVYYCNTFPPISNFWGQGTLVTVSSA |
| T017000640 | 280 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGVVQPGGSLRLSCVASWDVHKINFYTWYR |
| | | QAPGKEREKVAHISIGDQTDYADSAKGRFTISRDESKNTVYLQMNSLRPEDTALYYCRALSRIWPYDYWGQGTLVTVSSA |
| T017000642 | 281 | DVQLVESGGGVVQPGGSLRLSCVASWDVHKINFYTWYRQAPGKEREKVAHISIGDQTDYADSAKGRFTISRDESKNTVYLQMNSLRPEDTALYYCR |
| | | ALSKIWPYDYWGQGTLVTVSSGGGGSGGGGGGGGSGGGGSGGGGSGGGGSGGGGSEVQLVESGGGLVQAGGSLRLSCAASGITSKINDMGW |
| | | YRQTPGNYREWVASITATGTTNYRDSVKGRFTISRDNAKSTVYLQMNSLKPEDTTVYYCNTFPPISNFWGQGTLVTVSSA |
| T017000643 | 282 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGVVQPGGSLRLSCVASWDVHKINFYTWYR |
| | | QAPGKEREKVAHISIGDQTDYADSAKGRFTISRDESKNTVYLQMNSLRPEDTALYYCRALSKIWPYDYWGQGTLVTVSSA |
| T017000670 | 283 | DVQLVESGGGVVQPGGSLRLSCVASGYVHKINFYGWYRQAPGKEREKVAHISIGDQTDYADSAKGRFTISRDESKNTVYLQMNSLRPEDTALYYCR |
| | | ALSRIWPYDYWGQGTLVTVSSGGGGSGGGGSGGGGSGGGGSGGGGSGGGGSGGGGSEVQLVESGGGLVQAGGPLRLSCAASGRTFSSYVMG |
| | | WFRQAPGKEREFVAAIYWSNGKTQYTDSVKGRFTISGDNAKNTVYLQMNSLNPEDTAVYYCVADKDETGFRTLPIAYDYWGQGTLVTVSSGGGGS |
| | | GGGGSGGGGSGGGGSGGGGSGGGGSGGGGSEVQLVESGGGLVQAGGSLRLSCAASGITSKINDMGWYRQTPGNYREWVASITATGTTNYRDS |
| | | VKGRFTISRDNAKSTVYLQMNSLKPEDTTVYYCNTFPPISNFWGQGTLVTVSSA |
| T017000671 | 284 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGVVQPGGSLRLSCVASGYVHKINFYGWYR |
| | | QAPGKEREKVAHISIGDQTDYADSAKGRFTISRDESKNTVYLQMNSLRPEDTALYYCRALSRIWPYDYWGQGTLVTVSSGGGGSGGGGSGGGGSG |
| | | GGGSGGGGSGGGGSGGGGSEVQLVESGGGLVQAGGPLRLSCAASGRTFSSYVMGWFRQAPGKEREFVAAIYWSNGKTQYTDSVKGRFTISGDN |
| | | AKNTVYLQMNSLNPEDTAVYYCVADKDETGFRTLPIAYDYWGQGTLVTVSSA |
| T017000672 | 285 | DVQLVESGGGVVQPGGSLRLSCVASGYVHKINFYGWYRQAPGKEREKVAHISIGDQTDYADSAKGRFTISRDESKNTVYLQMNSLRPEDTALYYCR |
| | | ALSRIWPYDYWGQGTLVTVSSGGGGSGGGGSGGGGSGGGGSGGGGSGGGGSGGGGSEVQLVESGGGLVQAGDSLRLSCAASGRTFSSYAMG |
| | | WFRQAPGKEREFVAAISWSDGSTYYADSVKGRFTISRDNAKNTVYLQMNSLKPEDTAVYYCAADLTSTNPGSYIYIWAYDYWGQGTLVTVSSGGG |
| | | GSGGGGSGGGGSGGGGSGGGGSGGGGSGGGGSEVQLVESGGGLVQAGDSLRLSCAASGRTFSSYAMGWFRQAPGKEREFVAAISWSDGSTYY |
| | | ADSVKGRFTISRDNAKNTVYLQMNSLKPEDTAVYYCAADLTSTNPGSYIYIWAYDYWGQGTLVTVSSA |
| T017000673 | 286 | DVQLVESGGGLVQAGDSLRLSCAASGRTFSSYAMGWFRQAPGKEREFVAAISWSDGSTYYADSVKGRFTISRDNAKNTVYLQMNSLKPEDTAVYY |
| | | CAADLTSTNPGSYIYIWAYDYWGQGTLVTVSSGGGGSGGGGSGGGGSGGGGSGGGGSGGGGSGGGGSEVQLVESGGGVVQPGGSLRLSCVAS |
| | | GYVHKINFYGWYRQAPGKEREKVAHISIGDQTDYADSAKGRFTISRDESKNTVYLQMNSLRPEDTALYYCRALSRIWPYDYWGQGTLVTVSSGGGG |
| | | SGGGGSGGGGSGGGGSGGGGSGGGGSGGGGSEVQLVESGGGLVQAGDSLRLSCAASGRTFSSYAMGWFRQAPGKEREFVAAISWSDGSTYYA |
| | | DSVKGRFTISRDNAKNTVYLQMNSLKPEDTAVYYCAADLTSTNPGSYIYIWAYDYWGQGTLVTVSSA |
| T017000674 | 287 | DVQLVESGGGLVQAGDSLRLSCAASGRTFSSYAMGWFRQAPGKEREFVAAISWSDGSTYYADSVKGRFTISRDNAKNTVYLQMNSLKPEDTAVYY |
| | | CAADLTSTNPGSYIYIWAYDYWGQGTLVTVSSGGGGGGGGSGGGGSGGGGSGGGGSGGGGGGGGSEVQLVESGGGLVQAGDSLRLSCAAS |
| | | GRTFSSYAMGWFRQAPGKEREFVAAISWSDGSTYYADSVKGRFTISRDNAKNTVYLQMNSLKPEDTAVYYCAADLTSTNPGSYIYIWAYDYWGQG |
| | | TLVTVSSGGGGSGGGGSGGGGSGGGGSGGGGSGGGGSGGGGSEVQLVESGGGVVQPGGSLRLSCVASGYVHKINFYGWYRQAPGKEREKVAHI |
| | | SIGDQTDYADSAKGRFTISRDESKNTVYLQMNSLRPEDTALYYCRALSRIWPYDYWGQGTLVTVSSA |
| T017000675 | 288 | DVQLVESGGGLVQAGGPLRLSCAASGRTFSSYVMGWFRQAPGKEREFVAAIYWSNGKTQYTDSVKGRFTISGDNAKNTVYLQMNSLNPEDTAVY |
| | | YCVADKDETGFRTLPIAYDYWGQGTLVTVSSGGGGSGGGGSGGGGSGGGGGGGGSGGGGSGGGGSEVQLVESGGGLVQAGGSLRLSCAASGI |
| | | TSKINDMGWYRQTPGNYREWVASITATGTTNYRDSVKGRFTISRDNAKSTVYLQMNSLKPEDTTVYYCNTFPPISNFWGQGTLVTVSSGGGGSGG |
| | | GGSGGGGSGGGGSGGGGSGGGGSGGGGSEVQLVESGGGVVQPGGSLRLSCVASGYVHKINFYGWYRQAPGKEREKVAHISIGDQTDYADSAKG |
| | | RFTISRDESKNTVYLQMNSLRPEDTALYYCRALSRIWPYDYWGQGTLVTVSSA |
| T017000676 | 289 | DVQLVESGGGLVQAGGSLRLSCAASGITSKINDMGWYRQTPGNYREWVASITATGTTNYRDSVKGRFTISRDNAKSTVYLQMNSLKPEDTTVYYCN |
| | | TFPPISNFWGQGTLVTVSSGGGGSGGGGSGGGGSGGGGSGGGGSGGGGSGGGGSEVQLVESGGGLVQAGGPLRLSCAASGRTFSSYVMGWFR |
| | | QAPGKEREFVAAIYWSNGKTQYTDSVKGRFTISGDNAKNTVYLQMNSLNPEDTAVYYCVADKDETGFRTLPIAYDYWGQGTLVTVSSGGGGSGGG |
| | | GSGGGGSGGGGSGGGGSGGGGSGGGGSEVQLVESGGGVVQPGGSLRLSCVASGYVHKINFYGWYRQAPGKEREKVAHISIGDQTDYADSAKGR |
| | | FTISRDESKNTVYLQMNSLRPEDTALYYCRALSRIWPYDYWGQGTLVTVSSA |
| T017000677 | 290 | DVQLVESGGGLVQAGGPLRLSCAASGRTFSSYVMGWFRQAPGKEREFVAAIYWSNGKTQYTDSVKGRFTISGDNAKNTVYLQMNSLNPEDTAVY |
| | | YCVADKDETGFRTLPIAYDYWGQGTLVTVSSGGGGSGGGGSGGGGSGGGGSGGGGSGGGGSGGGGSEVQLVESGGGVVQPGGSLRLSCVASG |
| | | YVHKINFYGWYRQAPGKEREKVAHISIGDQTDYADSAKGRFTISRDESKNTVYLQMNSLRPEDTALYYCRALSRIWPYDYWGQGTLVTVSSGGGGS |
| | | GGGGGGGGSGGGGSGGGGSGGGGSGGGGSEVQLVESGGGLVQAGGSLRLSCAASGITSKINDMGWYRQTPGNYREWVASITATGTTNYRDS |
| | | VKGRFTISRDNAKSTVYLQMNSLKPEDTTVYYCNTFPPISNFWGQGTLVTVSSA |

TABLE A-4
Serum albumin binding ISVD sequences (″ID″ refers to the SEQ ID NO as used
herein)

| Name | ID | Amino acid sequence |
|---|---|---|
| Alb8 | 361 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVTVSS |
| Alb8-A | 362 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVTVSSA |
| Alb23 | 363 | EVQLLESGGGLVQPGGSLRLSCAASGFTFRSFGMSWVRQAPGKGPEWVSSISG |
| | | SGSDTLYADSVKGRFTISRDNSKNTLYLQMNSLRPEDTAVYYCTIGGSLSRSSQG |
| | | TLVTVSS |
| Alb23-A | 364 | EVQLLESGGGLVQPGGSLRLSCAASGFTFRSFGMSWVRQAPGKGPEWVSSISG |
| | | SGSDTLYADSVKGRFTISRDNSKNTLYLQMNSLRPEDTAVYYCTIGGSLSRSSQG |
| | | TLVTVSSA |
| Alb83 | 365 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTATYYCTIGGSLSRSSQGT |
| | | LVTVSS |
| Alb83-A | 366 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTATYYCTIGGSLSRSSQGT |
| | | LVTVSSA |
| Alb132 | 367 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTATYYCTIGGSLSRSSQ |
| | | GTLVTVSS |
| Alb132-A | 368 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTATYYCTIGGSLSRSSQ |
| | | GTLVTVSSA |
| Alb73 | 369 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVKVSS |
| Alb73-A | 370 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVKVSSA |
| Alb82 | 371 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVTVSS |
| Alb82-A | 372 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVTVSSA |
| Alb199 | 373 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVKVSS |
| Alb199-A | 374 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVKVSSA |
| Alb23002 | 375 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVTVSS |
| Alb223 | 376 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVTVSSA |
| Alb216 | 377 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVKVSS |
| Alb216-A | 378 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVKVSSA |

TABLE A-5
Linker sequences (“ID” refers to the SEQ ID NO as used herein)

| Name | ID | Amino acid sequence |
|---|---|---|
| 3A linker | 322 | AAA |
| 5GS linker | 323 | GGGGS |
| 7GS linker | 324 | SGGSGGS |
| 8GS linker | 325 | GGGGSGGS |
| 9GS linker | 326 | GGGGSGGGS |
| 10GS linker | 327 | GGGGSGGGGS |
| 15GS linker | 328 | GGGGSGGGGSGGGGS |
| 18GS linker | 329 | GGGGSGGGGSGGGGSGGS |
| 20GS linker | 330 | GGGGSGGGGSGGGGSGGGGS |
| 25GS linker | 331 | GGGGSGGGGSGGGGSGGGGSGGGGS |
| 30GS linker | 332 | GGGGSGGGGSGGGGSGGGGSGGGGSGGGGS |
| 35GS linker | 333 | GGGGSGGGGSGGGGSGGGGSGGGGSGGGGSGGGGS |
| 40GS linker | 334 | GGGGSGGGGSGGGGSGGGGGGGGSGGGGSGGGGSGGGGS |
| G1 hinge | 335 | EPKSCDKTHTCPPCP |
| 9GS-G1 hinge | 336 | GGGGSGGGSEPKSCDKTHTCPPCP |
| Llama upper long | 337 | EPKTPKPQPAAA |
| hinge region | | |
| G3 hinge | 338 | ELKTPLGDTTHTCPRCPEPKSCDTPPPCPRCPEPKSCDTPPPCPRCPEPK |
| | | SCDTPPPCPRCP |

TABLE A-6
Amino acid sequences related to CD3, TCR and CD123 (“ID” refers to the given
SEQ ID NO as used herein)

| ID | Name | Amino acid sequence |
|---|---|---|
| 300 | Human CD3 delta | MEHSTFLSGLVLATLLSQVSPFKIPIEELEDRVFVNCNTSITWVEGT |
| | (P04234) | VGTLLSDITRLDLGKRILDPRGIYRCNGTDIYKDKESTVQVHYRMCQ |
| | | SCVELDPATVAGIIVTDVIATLLLALGVFCFAGHETGRLSGAADTQA |
| | | LLRNDQVYQPLRDRDDAQYSHLGGNWARNK |
| 301 | Human CD3 gamma | MEQGKGLAVLILAIILLQGTLAQSIKGNHLVKVYDYQEDGSVLLTCD |
| | (derived from P09693) | AEAKNITWFKDGKMIGFLTEDKKKWNLGSNAKDPRGMYQCKGS |
| | | QNKSKPLQVYYRMCQNCIELNAATISGFLFAEIVSIFVLAVGVYFIA |
| | | GQDGVRQSRASDKQTLLPNDQLYQPLKDREDDQYSHLQGNQLR |
| 302 | Human CD3 epsilon | MQSGTHWRVLGLCLLSVGVWGQDGNEEMGGITQTPYKVSISGT |
| | (derived from P07766) | TVILTCPQYPGSEILWQHNDKNIGGDEDDKNIGSDEDHLSLKEFSE |
| | | LEQSGYYVCYPRGSKPEDANFYLYLRARVCENCMEMDVMSVATIV |
| | | IVDICITGGLLLLVYYWSKNRKAKAKPVTRGAGAGGRQRGQNKER |
| | | PPPVPNPDYEPIRKGQRDLYSGLNQR |
| 303 | Human CD3 zeta | MKWKALFTAAILQAQLPITEAQSFGLLDPKLCYLLDGILFIYGVILTA |
| | (P20963) | LFLRVKFSRSADAPAYQQGQNQLYNELNLGRREEYDVLDKRRGRD |
| | | PEMGGKPQRRKNPQEGLYNELQKDKMAEAYSEIGMKGERRRGK |
| | | GHDGLYQGLSTATKDTYDALHMQALPPR |
| 304 | Human TCR alpha | PNIQNPDPAVYQLRDSKSSDKSVCLFTDFDSQTNVSQSKDSDVYIT |
| | constant domain | DKTVLDMRSMDFKSNSAVAWSNKSDFACANAFNNSIIPEDTFFPS |
| | (derived from P01848) | PESSCDVKLVEKSFETDTNLNFQNLSVIGFRILLLKVAGFNLLMTLRL |
| | | WSS |
| 305 | Human TCR beta | EDLNKVFPPEVAVFEPSEAEISHTQKATLVCLATGFFPDHVELSWW |
| | constant domain | VNGKEVHSGVSTDPQPLKEQPALNDSRYALSSRLRVSATFWQNPR |
| | (derived from P01850) | NHFRCQVQFYGLSENDEWTQDRAKPVTQIVSAEAWGRADCGFT |
| | | SVSYQQGVLSATILYEILLGKATLYAVLVSALVLMAMVKRKDF |
| 306 | Human TCR alpha | IQVEQSPPDLILQEGANSTLRCNFSDSVNNLQWFHQNPWGQLIN |
| | variable domain | LFYIPSGTKQNGRLSATTVATERYSLLYISSSQTTDSGVYFCAALIQG |
| | derived from 2IAN | AQKLVFGQGTRLTIN |
| 307 | Human TCR alpha | QLLEQSPQFLSIQEGENLTVYCNSSSVFSSLQWYRQEPGEGPVLLV |
| | variable domain | TVVTGGEVKKLKRLTFQFGDARKDSSLHITAAQPGDTGLYLCAGA |
| | derived from 2XN9 | GSQGNLIFGKGTKLSVK |
| 308 | Human TCR alpha | GDAKTTQPNSMESNEEEPVHLPCNHSTISGTDYIHWYRQLPSQGP |
| | variable domain | EYVIHGLTSNVNNRMASLAIAEDRKSSTLILHRATLRDAAVYYCTVY |
| | derived from 3TOE | GGATNKLIFGTGTLLAVQ |
| 309 | Human TCR beta | NAGVTQTPKFRILKIGQSMTLQCTQDMNHNYMYWYRQDPGMG |
| | variable domain | LKLIYYSVGAGITDKGEVPNGYNVSRSTTEDFPLRLELAAPSQTSVYF |
| | derived from 2IAN | CASTYHGTGYFGEGSWLTVV |
| 310 | Human TCR beta | DGGITQSPKYLFRKEGQNVTLSCEQNLNHDAMYWYRQDPGQGL |
| | variable domain | RLIYYSQIVNDFQKGDIAEGYSVSREKKESFPLTVTSAQKNPTAFYLC |
| | derived from 2XN9 | ASSSRSSYEQYFGPGTRLTVT |
| 311 | Human TCR beta | VVSQHPSWVIAKSGTSVKIECRSLDFQATTMFWYRQFPKQSLML |
| | variable domain | MATSNEGSKATYEQGVEKDKFLINHASLTLSTLTVTSAHPEDSSFYI |
| | derived from 3TOE | CSARGGSYNSPLHFGNGTRLTVT |
| 312 | Cyno CD3 delta | MEHSTFLSGLVLATLLSQVSPFKIPVEELEDRVFVKCNTSVTWVEGT |
| | (NP_001274617) | VGTLLTNNTRLDLGKRILDPRGIYRCNGTDIYKDKESAVQVHYRMC |
| | | QNCVELDPATLAGIIVTDVIATLLLALGVFCFAGHETGRLSGAADTQ |
| | | ALLRNDQVYQPLRDRDDAQYSRLGGNWARNK |
| 313 | Cyno CD3 gamma | MEQGKGLTGFILAIILLQGSLAQSFEENRKLNVYNQEDGSVLLTCH |
| | (BAJ16168) | VKNTNITWFKEGKMIDILTAHKNKWNLGSNTKDPRGVYQCKGSK |
| | | DKSKTLQVYYRMCQNCIELNAATILGFVFAEIISIFFLAVGVYFIAGQ |
| | | DGVRQSRASDKQTLLPNDQLYQPLKDREDDQYSHLQGNQLRRN |
| 314 | Cyno CD3 epsilon | MQSGTRWRVLGLCLLSIGVWGQDGNEEMGSITQTPYQVSISGTT |
| | (Q95LI5) | VILTCSQHLGSEAQWQHNGKNKEDSGDRLFLPEFSEMEQSGYYV |
| | | CYPRGSNPEDASHHLYLKARVCENCMEMDVMAVATIVIVDICITL |
| | | GLLLLVYYWSKNRKAKAKPVTRGAGAGGRQRGQNKERPPPVPNP |
| | | DYEPIRKGQQDLYSGLNQRRI |
| 315 | Cyno CD3 zeta | MKWKELVTAAILQAQFPITEAQSFGLLDPKLCYLLDGILFLYGVILTA |
| | (XP_005539936) | LFLRAKFSRSADAPAYQQGQNQLYNELNLGRREEYDVLDKRRGRD |
| | | PEMGGKPQRRKNPQEGLYNALQKDKMAEAYSEIGMKGERRRGK |
| | | GHDGLYQGLSTATKDTYDALHMQTLPPR |
| 316 | Rhesus TCR alpha | MLLLLVLVLEVIFTLGGTRAQSVTQLDSQVSVSEGVPVLLRCNYSSS |
| | (AEA41863) | FSPYLFWYVQYPNQGLQLLLKYTSGTTLVKGINGFEAEFKKSETSFH |
| | | LTKASAHVSDAAEYFCALARGALVFGKGTRLSVIPNIQNPDPAVYQ |
| | | LRGSKSNDTSVCLFTDFDSVMNVSQSKDSDVHITDKTVLDMRSM |
| | | DFKSNGAVAWSNKSDFACTSAFKDSVIPADTFFPGTESVCDANLV |
| | | EKSFETDMNLNFQNLSVIGFRILLLKVAGFNLLMTLRLWSS |
| 317 | Rhesus TCR beta | MGFWTLCCVSFCILVAKHTDAGVIQLPRHEVTEMGKEVTLRCEPIS |
| | (AEA41864) | GHSSLFWYRQTMMRGLEFLIYFNNKSPIDDSGMPKDRFSATMPD |
| | | ASFSTLKIQPSEPRDSAVYFCASTPGQGREKLFFGSGTQLSVLEDLK |
| | | KVFPPKVAVFEPSEAEISHTQKATLVCLATGFYPDHVELSWWVNG |
| | | KEVHSGVSTDPQPLKEQPALEDSRYCLSSRLRVSATFWHNPRNHF |
| | | RCQVQFYGLSEDDEWTEDRDKPITQKISAEVWGRADCGFTSVSY |
| | | QQGVLSATILYEILLGKATLYAVLVSALMLMAMVKRKDF |
| 318 | Human TCR-zipper: | MNMRPVTSSVLVLLLMLRRSNGQLLEQSPQFLSIQEGENLTVYCN |
| | alpha chain | SSSVFSSLQWYRQEPGEGPVLLVTVVTGGEVKKLKRLTFQFGDARK |
| | | DSSLHITAAQPGDTGLYLCAGAGSQGNLIFGKGTKLSVKPNIQNPD |
| | | PAVYQLRDSKSSDKSVCLFTDFDSQTNVSQSKDSDVYITDKSVLDM |
| | | RSMDFKSNSAVAWSNKSDFACANAFNNSIIPEDTFFPSPESSCSSA |
| | | DLVPRGSTTAPSAQLKKKLQALKKKNAQLKWKLQALKKKLAQEQK |
| | | LISEEDL |
| 319 | Human TCR-zipper: | MSNTVLADSAWGITLLSWVTVFLLGTSSADGGITQSPKYLFRKEG |
| | beta chain | QNVTLSCEQNLNHDAMYWYRQDPGQGLRLIYYSQIVNDFQKGDI |
| | | AEGYSVSREKKESFPLTVTSAQKNPTAFYLCASSSRSSYEQYFGPGT |
| | | RLTVTEDLKNVFPPEVAVFEPSEAEISHTQKATLVCLATGFYPDHVE |
| | | LSWWVNGKEVHSGVSTDPQPLKEQPALNDSRYSLSSRLRVSATF |
| | | WQNPRNHFRCQVQFYGLSENDEWTQDRAKPVTQIVSAEAWGR |
| | | ADCSSADLVPRGSTTAPSAQLEKELQALEKENAQLEWELQALEKEL |
| | | AQTGHHHHHHHHHH |
| 320 | Cyno TCR-zipper: alpha | MLLITSVLVLWMQLSQVNGQQIMQIPQYQHVQEGEDFTTYCNSS |
| | chain (derived from | TTLSNIQWYKQRPGGHPVFLIMLVKSGEVKKQKRLIFQFGEAKKNS |
| | AEA41865) | SLHITATQTTDVGTYFCATTGVNNLFFGTGTRLTVLPYIQNPDPAV |
| | | YQLRGSKSNDTSVCLFTDFDSVMNVSQSKDSDVHITDKTVLDMRS |
| | | MDFKSNGAVAWSNKSDFACTSAFKDSVIPADTFFPSPESSCSSADL |
| | | VPRGSTTAPSAQLKKKLQALKKKNAQLKWKLQALKKKLAQEQKLIS |
| | | EEDL |
| 321 | Cyno TCR-zipper: beta | MSIGLLCCAALSLLWAGPVNAGVTQTPKFQVLKTGQSMTLQCAQ |
| | chain (derived from | DMNHDYMYWYRQDPGMGLRLIHYSVGEGSTEKGEVPDGYNVT |
| | AEA41866 and | RSNTEDFPLRLESAAPSQTSVYFCASSYWTGRSYEQYFGPGTRLTVI |
| | AEA41868) | EDLKKVFPPKVAVFEPSEAEISHTQKATLVCLATGFYPDHVELSWW |
| | | VNGKEVHSGVSTDPQPLKEQPALEDSRYSLSSRLRVSATFWHNPR |
| | | NHFRCQVQFYGLSEDDEWTEDRDKPITQKISAEAWGRADCSSAD |
| | | LVPRGSTTAPSAQLEKELQALEKENAQLEWELQALEKELAQTGHH |
| | | HHHHHHHH |
| 379 | Human CD123 | MVLLWLTLLLIALPCLLQTKEDPNPPITNLRMKAKAQQLTWDLNR |
| | (NP_002174) | NVTDIECVKDADYSMPAVNNSYCQFGAISLCEVTNYTVRVANPPF |
| | | STWILFPENSGKPWAGAENLTCWIHDVDFLSCSWAVGPGAPADV |
| | | QYDLYLNVANRRQQYECLHYKTDAQGTRIGCRFDDISRLSSGSQSS |
| | | HILVRGRSAAFGIPCTDKFVVFSQIEILTPPNMTAKCNKTHSFMHW |
| | | KMRSHFNRKFRYELQIQKRMQPVITEQVRDRTSFQLLNPGTYTVQ |
| | | IRARERVYEFLSAWSTPQRFECDQEEGANTRAWRTSLLIALGTLLAL |
| | | VCVFVICRRYLVMQRLFPRIPHMKDPIGDSFQNDKLVVWEAGKA |
| | | GLEECLVTEVQVVQKT |
| 291 | Human TCR alpha | PNIQNPDPAVYQLRDSKSSDKSVCLFTDFDSQTNVSQSKDSDVYIT |
| | constant domain | DKTVLDMRSMDFKSNSAVAWSNKSDFACANAFNNSIIPEDTFFPS |
| | (derived from P01848) | PESSC |
| 292 | Human TCR beta | EDLNKVFPPEVAVFEPSEAEISHTQKATLVCLATGFFPDHVELSWW |
| | constant domain | VNGKEVHSGVSTDPQPLKEQPALNDSRYALSSRLRVSATFWQNPR |
| | (derived from P01850) | NHFRCQVQFYGLSENDEWTQDRAKPVTQIVSAEAWGRADC |
| 293 | cynomolgus TCR-alpha | PYIQNPDPAVYQLRGSKSNDTSVCLFTDFDSVMNVSQSKDSDVHI |
| | constant domain | TDKTVLDMRSMDFKSNGAVAWSNKSDFACTSAFKDSVIPADTFF |
| | (derived from | PSPESSC |
| | AEA41865) | |
| 294 | cynomolgus TCR-beta | EDLKKVFPPKVAVFEPSEAEISHTQKATLVCLATGFYPDHVELSWW |
| | constant domain | VNGKEVHSGVSTDPQPLKEQPALEDSRYSLSSRLRVSATFWHNPR |
| | (derived from | NHFRCQVQFYGLSEDDEWTEDRDKPITQKISAEAWGRADC |
| | AEA41868) | |

TABLE A-7
C-termini and C-terminal extensions
(“ID” refers to the given SEQ ID NO as used herein)

| ID | Amino acid sequence |
|---|---|
| 339 | VTVSS |
| 340 | VKVSS |
| 341 | VQVSS |
| 342 | VTVKS |
| 343 | VTVQS |
| 344 | VKVKS |
| 345 | VKVQS |
| 346 | VQVKS |
| 347 | VQVQS |
| 348 | VTVSSA |
| 349 | VKVSSA |
| 350 | VQVSSA |
| 351 | VTVKSA |
| 352 | VTVQSA |
| 353 | VKVKSA |
| 354 | VKVQSA |
| 355 | VQVKSA |
| 356 | VQVQSA |

TABLE A-8
Amino acid sequences of other monovalent ISVDs

| Name | ID | Amino acid sequence |
|---|---|---|
| A0110056A10 | 263 | EVQLVESGGGLVQAGGSLRLSCAASGITSKINDMGWYRQTPGNYREWVAS |
| | | ITATGTTNYRDSVKGRFTISRDNAKSTVYLQMNSLKPEDTTVYYCNTFPPISN |
| | | FWGQGTLVTVSS |
| cAbLys3 | 264 | DVQLQASGGGSVQAGGSLRLSCAASGYTIGPYCMGWFRQAPGKEREGVA |
| | | AINMGGGITYYADSVKGRFTISQDNAKNTVYLLMNSLEPEDTAIYYCAADSTI |
| | | YASYYECGHGLSTGGYGYDSWGQGTQVTVSS |
| A0110055F03 | 265 | EVQLVESGGGLVQAGGPLRLSCAASGRTFSSYVMGWFRQAPGKEREFVAA |
| | | IYWSNGKTQYTDSVKGRFTISGDNAKNTVYLQMNSLNPEDTAVYYCVADK |
| | | DETGFRTLPIAYDYWGQGTLVTVSS |
| RSV007B02 | 266 | EVQLVESGGGLVQAGDSLRLSCAASGRTFSSYAMGWFRQAPGKEREFVAAI |
| (Q108L) | | SWSDGSTYYADSVKGRFTISRDNAKNTVYLQMNSLKPEDTAVYYCAADLTS |
| | | TNPGSYIYIWAYDYWGQGTLVTVSS |
| IRR0028 | 267 | DVQLQASGGGSVQAGGSLRLSCAASGYTIGPYCMGWFRQAPGKEREGVA |
| | | AINMGGGITYYADSVKGRFTISQDNAKNTVYLLMNSLEPEDTAIYYCAADSTI |
| | | YASYYECGHGLSTGGYGYDSWGQGTQVTVSS |

TABLE A-9
Amino acid sequences of selected multivalent polypeptides
(“ID” refers to the given SEQ ID NO)

| SEQ | ID | Sequence |
|---|---|---|
| 434 | A022600096 | DVQLVESGGGLVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSGGGGSGGGGSGGGGSGGGGSGGGGS |
| | | GGGGSEVQLVESGGGLVQPGGSLRLSCVASGFTFSSFAMTWVRRPPGKG |
| | | LEWVATITNGGVTSYRDSVKGRFTISRDNAKNTLYLEMTSLNPEDTAVYIC |
| | | ANARRTGPRAPTDIGSYRGQGTLVTVSSGGGGSGGGGSGGGGSGGGGS |
| | | GGGGSGGGGSGGGGSEVQLVESGGGVVQPGGSLRLSCAASGFTFRSFG |
| | | MSWVRQAPGKGPEWVSSISGSGSDTLYADSVKGRFTISRDNSKNTLYLQ |
| | | MNSLRPEDTALYYCTIGGSLSRSSQGTLVTVSSA |
| 435 | A022600102 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSAAAEVQLVESGGGLVQPGGSLRLSCVASGFTFS |
| | | SFAMTWVRRPPGKGLEWVATITNGGVTSYRDSVKGRFTISRDNAKNTLYL |
| | | EMTSLNPEDTAVYICANARRTGPRAPTDIGSYRGQGTLVTVSSGGGGSGG |
| | | GGSGGGGSGGGGSGGGGSGGGGSGGGGSEVQLVESGGGVVQPGGSLR |
| | | LSCAASGFTFRSFGMSWVRQAPGKGPEWVSSISGSGSDTLYADSVKGRFT |
| | | ISRDNSKNTLYLQMNSLRPEDTALYYCTIGGSLSRSSQGTLVTVSSA |
| 436 | A022600103 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGGSGGGGSGGGGSGGGGSGGGGSGGGGSEVQLVESGGGVVQPGG |
| | | SLRLSCAASGFTFRSFGMSWVRQAPGKGPEWVSSISGSGSDTLYADSVKG |
| | | RFTISRDNSKNTLYLQMNSLRPEDTALYYCTIGGSLSRSSQGTLVTVSSA |
| 437 | A022600104 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSGGGSEVQLVESGGGLVQPGGSLRLSCV |
| | | ASGFTFSSFAMTWVRRPPGKGLEWVATITNGGVTSYRDSVKGRFTISRDN |
| | | AKNTLYLEMTSLNPEDTAVYICANARRTGPRAPTDIGSYRGQGTLVTVSSG |
| | | GGGSGGGGSGGGGSGGGGGGGGSGGGGSGGGGSEVQLVESGGGVV |
| | | QPGGSLRLSCAASGFTFRSFGMSWVRQAPGKGPEWVSSISGSGSDTLYA |
| | | DSVKGRFTISRDNSKNTLYLQMNSLRPEDTALYYCTIGGSLSRSSQGTLVTV |
| | | SSA |
| 438 | A022600105 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSGGGGSGGGGSGGGGSEVQLVESGGGL |
| | | VQPGGSLRLSCVASGFTFSSFAMTWVRRPPGKGLEWVATITNGGVTSYR |
| | | DSVKGRFTISRDNAKNTLYLEMTSLNPEDTAVYICANARRTGPRAPTDIGS |
| | | YRGQGTLVTVSSGGGGSGGGGSGGGGSGGGGGGGGSGGGGSGGGGS |
| | | EVQLVESGGGVVQPGGSLRLSCAASGFTFRSFGMSWVRQAPGKGPEWV |
| | | SSISGSGSDTLYADSVKGRFTISRDNSKNTLYLQMNSLRPEDTALYYCTIGG |
| | | SLSRSSQGTLVTVSSA |
| 439 | A022600122 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGGSGGGGSGGGGSGGGGSGGGGSGGGGSEVQLVESGGGLVQPGG |
| | | SLRLSCVASGSIFRSVFSSSTMEWYRQPPGKKRELVARIAPGDGTNYGALY |
| | | ADSVKGRFTISRDDAKKTVDLQMNSLKPEDTGVYFCASGVAWGQGTLVT |
| | | VSSGGGGSGGGGSGGGGSGGGGSGGGGSGGGGSGGGGSEVQLVESGG |
| | | GVVQPGGSLRLSCAASGFTFRSFGMSWVRQAPGKGPEWVSSISGSGSDT |
| | | LYADSVKGRFTISRDNSKNTLYLQMNSLRPEDTALYYCTIGGSLSRSSQGTL |
| | | VTVSSA |
| 440 | A022600131 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGGSGGGGSGGGGSEVQLVESGGGLVQPGGSLRLSCVASGSIFRSVFSS |
| | | STMEWYRQPPGKKRELVARIAPGDGTNYGALYADSVKGRFTISRDDAKKT |
| | | VDLQMNSLKPEDTGVYFCASGVAWGQGTLVTVSSGGGGSGGGGSGGG |
| | | GSGGGGSGGGGSGGGGSGGGGSEVQLVESGGGVVQPGGSLRLSCAASG |
| | | FTFRSFGMSWVRQAPGKGPEWVSSISGSGSDTLYADSVKGRFTISRDNSK |
| | | NTLYLQMNSLRPEDTALYYCTIGGSLSRSSQGTLVTVSSA |
| 441 | A022600132 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGSEVQLVESGGGLVQPGGSLRLSCVASGSIFRSVFSSSTMEWYRQPPG |
| | | KKRELVARIAPGDGTNYGALYADSVKGRFTISRDDAKKTVDLQMNSLKPE |
| | | DTGVYFCASGVAWGQGTLVTVSSGGGGSGGGGSGGGGSGGGGSGGG |
| | | GSGGGGSGGGGSEVQLVESGGGVVQPGGSLRLSCAASGFTFRSFGMSW |
| | | VRQAPGKGPEWVSSISGSGSDTLYADSVKGRFTISRDNSKNTLYLQMNSL |
| | | RPEDTALYYCTIGGSLSRSSQGTLVTVSSA |
| 442 | A022600133 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGSI |
| | | FRSVFSSSTMEWYRQPPGKKRELVARIAPGDGTNYGALYADSVKGRFTISR |
| | | DDAKKTVDLQMNSLKPEDTGVYFCASGVAWGQGTLVTVSSGGGGSGGG |
| | | GSGGGGSGGGGSGGGGSGGGGSGGGGSEVQLVESGGGLVQPGGSLRL |
| | | SCVASGFTFSSFAMTWVRRPPGKGLEWVATITNGGVTSYRDSVKGRFTIS |
| | | RDNAKNTLYLEMTSLNPEDTAVYICANARRTGPRAPTDIGSYRGQGTLVT |
| | | VSSGGGGSGGGGSGGGGSGGGGSGGGGSGGGGSGGGGSEVQLVESGG |
| | | GVVQPGGSLRLSCAASGFTFRSFGMSWVRQAPGKGPEWVSSISGSGSDT |
| | | LYADSVKGRFTISRDNSKNTLYLQMNSLRPEDTALYYCTIGGSLSRSSQGTL |
| | | VTVSSA |
| 443 | A022600134 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGSI |
| | | FRSVFSSSTMEWYRQPPGKKRELVARIAPGDGTNYGALYADSVKGRFTISR |
| | | DDAKKTVDLQMNSLKPEDTGVYFCASGVAWGQGTLVTVSSGGGGSGGG |
| | | GSGGGGSGGGGSEVQLVESGGGLVQPGGSLRLSCVASGFTFSSFAMTWV |
| | | RRPPGKGLEWVATITNGGVTSYRDSVKGRFTISRDNAKNTLYLEMTSLNPE |
| | | DTAVYICANARRTGPRAPTDIGSYRGQGTLVTVSSGGGGSGGGGSGGGG |
| | | SGGGGSGGGGSGGGGSGGGGSEVQLVESGGGVVQPGGSLRLSCAASGF |
| | | TFRSFGMSWVRQAPGKGPEWVSSISGSGSDTLYADSVKGRFTISRDNSKN |
| | | TLYLQMNSLRPEDTALYYCTIGGSLSRSSQGTLVTVSSA |
| 444 | A022600135 | DVQLVESGGGVVQPGGSLRLSCVASGYVHKINFYGWYRQAPGKEREKVA |
| | | HISIGDQTDYADSAKGRFTISRDESKNTVYLQMNSLRPEDTALYYCRALSRI |
| | | WPYDYWGQGTLVTVSSGGGGSEVQLVESGGGLVQPGGSLRLSCVASGSI |
| | | FRSVFSSSTMEWYRQPPGKKRELVARIAPGDGTNYGALYADSVKGRFTISR |
| | | DDAKKTVDLQMNSLKPEDTGVYFCASGVAWGQGTLVTVSSGGGGSGGG |
| | | SEVQLVESGGGLVQPGGSLRLSCVASGFTFSSFAMTWVRRPPGKGLEWV |
| | | ATITNGGVTSYRDSVKGRFTISRDNAKNTLYLEMTSLNPEDTAVYICANAR |
| | | RTGPRAPTDIGSYRGQGTLVTVSSGGGGSGGGGSGGGGSGGGGSGGGG |
| | | SGGGGSGGGGSEVQLVESGGGVVQPGGSLRLSCAASGFTFRSFGMSWV |
| | | RQAPGKGPEWVSSISGSGSDTLYADSVKGRFTISRDNSKNTLYLQMNSLR |
| | | PEDTALYYCTIGGSLSRSSQGTLVTVSSA |
| 445 | A022600167 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGGSGGGGGGGGSGGGGSGGGGSGGGGSEVQLVESGGGVVQPGG |
| | | SLRLSCAASGFTFRSFGMSWVRQAPGKGPEWVSSISGSGSDTLYADSVKG |
| | | RFTISRDNSKNTLYLQMNSLRPEDTALYYCTIGGSLSRSSQGTLVTVSSA |
| 446 | A022600168 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGGSGGGGSGGGGSGGGGSGGGGGGGGSEVQLVESGGGLVQPGG |
| | | SLRLSCVASGSIFRSVFSSSTMEWYRQPPGKKRELVARIAPGDGTNYGALY |
| | | ADSVKGRFTISRDDAKKTVDLQMNSLKPEDTGVYFCASGVAWGQGTLVT |
| | | VSSGGGGSGGGGSGGGGSGGGGSGGGGSGGGGSGGGGSEVQLVESGG |
| | | GVVQPGGSLRLSCAASGFTFRSFGMSWVRQAPGKGPEWVSSISGSGSDT |
| | | LYADSVKGRFTISRDNSKNTLYLQMNSLRPEDTALYYCTIGGSLSRSSQGTL |
| | | VTVSSA |
| 447 | A022600169 | DVQLVESGGGLVQPGGSLRLSCVASGFTFSSFAMTWVRRPPGKGLEWVA |
| | | TITNGGVTSYRDSVKGRFTISRDNAKNTLYLEMTSLNPEDTAVYICANARRT |
| | | GPRAPTDIGSYRGQGTLVTVSSGGGGSEVQLVESGGGVVQPGGSLRLSCV |
| | | ASGYVHKINFYGWYRQAPGKEREKVAHISIGDQTDYADSAKGRFTISRDES |
| | | KNTVYLQMNSLRPEDTAAYYCRALSRIWPYDYWGQGTLVTVSSGGGGSE |
| | | VQLVESGGGLVQPGGSLRLSCVASGSIFRSVFSSSTMEWYRQPPGKKREL |
| | | VARIAPGDGTNYGALYADSVKGRFTISRDDAKKTVDLQMNSLKPEDTGVY |
| | | FCASGVAWGQGTLVTVSSGGGGSGGGGSGGGGSGGGGSGGGGSGGG |
| | | GSGGGGSEVQLVESGGGVVQPGGSLRLSCAASGFTFRSFGMSWVRQAP |
| | | GKGPEWVSSISGSGSDTLYADSVKGRFTISRDNSKNTLYLQMNSLRPEDTA |
| | | LYYCTIGGSLSRSSQGTLVTVSSA |
| 448 | A022600170 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGGSGGGGSGGGGSGGGGSGGGGSGGGGSEVQLVESGGGLVQPGG |
| | | SLRLSCVASGSIFRSVFSSSTMEWYRQPPGKKRELVARIAPGDGTNYGALY |
| | | ADSVKGRFTISRDDAKKTVDLQMNSLKPEDTGVYFCASGVAWGQGTLVT |
| | | VSSGGGGSGGGSEVQLVESGGGVVQPGGSLRLSCAASGFTFRSFGMSW |
| | | VRQAPGKGPEWVSSISGSGSDTLYADSVKGRFTISRDNSKNTLYLQMNSL |
| | | RPEDTALYYCTIGGSLSRSSQGTLVTVSSA |
| 449 | A022600172 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGSEVQLVESGGGVVQPGGSLRLSCAASGFTFRSFGMSWVRQAPGKG |
| | | PEWVSSISGSGSDTLYADSVKGRFTISRDNSKNTLYLQMNSLRPEDTALYY |
| | | CTIGGSLSRSSQGTLVTVSSGGGGSGGGSEVQLVESGGGLVQPGGSLRLSC |
| | | VASGSIFRSVFSSSTMEWYRQPPGKKRELVARIAPGDGTNYGALYADSVK |
| | | GRFTISRDDAKKTVDLQMNSLKPEDTGVYFCASGVAWGQGTLVTVSSA |
| 450 | A022600174 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGSEVQLVESGGGLVQPGGSLRLSCVASGSIFRSVFSSSTMEWYRQPPG |
| | | KKRELVARIAPGDGTNYGALYADSVKGRFTISRDDAKKTVDLQMNSLKPE |
| | | DTGVYFCASGVAWGQGTLVTVSSGGGGSGGGSEVQLVESGGGVVQPG |
| | | GSLRLSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVSSA |
| 451 | A022600175 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGSEVQLVESGGGLVQPGGSLRLSCVASGSIFRSVFSSSTMEWYRQPPG |
| | | KKRELVARIAPGDGTNYGALYADSVKGRFTISRDDAKKTVDLQMNSLKPE |
| | | DTGVYFCASGVAWGQGTLVTVSSGGGGSGGGGSGGGGSGGGGSGGG |
| | | GSGGGGSGGGGSEVQLVESGGGVVQPGGSLRLSCAASGFTFRSFGMSW |
| | | VRQAPGKGPEWVSSISGSGSDTLYADSVKGRFTISRDNSKNTLYLQMNSL |
| | | RPEDTALYYCTIGGSLSRSSQGTLVTVSSA |
| 452 | A022600178 | DVQLVESGGGLVQPGGSLRLSCVASGFTFSSFAMTWVRRPPGKGLEWVA |
| | | TITNGGVTSYRDSVKGRFTISRDNAKNTLYLEMTSLNPEDTAVYICANARRT |
| | | GPRAPTDIGSYRGQGTLVTVSSGGGGSEVQLVESGGGVVQPGGSLRLSCV |
| | | ASGYVHKINFYGWYRQAPGKEREKVAHISIGDQTDYADSAKGRFTISRDES |
| | | KNTVYLQMNSLRPEDTAAYYCRALSRIWPYDYWGQGTLVTVSSGGGGSG |
| | | GGGSGGGGSGGGGSGGGGSGGGGSGGGGSEVQLVESGGGVVQPGGSL |
| | | RLSCAASGFTFRSFGMSWVRQAPGKGPEWVSSISGSGSDTLYADSVKGRF |
| | | TISRDNSKNTLYLQMNSLRPEDTALYYCTIGGSLSRSSQGTLVTVSSA |
| 453 | A022600179 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGLVQPGGSLRLSCVASGFT |
| | | FSSFAMTWVRRPPGKGLEWVATITNGGVTSYRDSVKGRFTISRDNAKNTL |
| | | YLEMTSLNPEDTAVYICANARRTGPRAPTDIGSYRGQGTLVTVSSGGGGS |
| | | GGGSEVQLVESGGGVVQPGGSLRLSCAASGFTFRSFGMSWVRQAPGKG |
| | | PEWVSSISGSGSDTLYADSVKGRFTISRDNSKNTLYLQMNSLRPEDTALYY |
| | | CTIGGSLSRSSQGTLVTVSSA |
| 454 | T017000698 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSGGGSEVQLVESGGGVVQPGGSLRLSCA |
| | | ASGFTFRSFGMSWVRQAPGKGPEWVSSISGSGSDTLYADSVKGRFTISRD |
| | | NSKNTLYLQMNSLRPEDTALYYCTIGGSLSRSSQGTLVTVSSA |
| 455 | A022600412 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGVVQPGGSLRLSCAASGF |
| | | TFSSFAMTWVRRPPGKGLEWVATITNAGVTSYADSVKGRFTISRDNAKNT |
| | | LYLQMNSLRPEDTALYICANARRTGPRAPTDIGSYRGQGTLVTVSSGGGG |
| | | SGGGGSGGGGSGGGGSEVQLVESGGGVVQPGGSLRLSCAASGFTFRSFG |
| | | MSWVRQAPGKGPEWVSSISGSGSDTLYADSVKGRFTISRDNSKNTLYLQ |
| | | MNSLRPEDTALYYCTIGGSLSRSSQGTLVTVSSA |
| 456 | A022600424 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGVVQPGGSLRLSCAASGF |
| | | TFSSFAMTWVRRPPGKGLEWVATITNKGVTSYADSVKGRFTISRDNAKNT |
| | | LYLQMNSLRPEDTALYICANARRTGPRAPTDIGSYRGQGTLVTVSSGGGG |
| | | SGGGSEVQLVESGGGVVQPGGSLRLSCAASGSIFRSVFSSSTMEWYRQAP |
| | | GKKRELVARIAPGEGTYYGALYADSVKGRFTISRDNAKNTVYLQMNSLRPE |
| | | DTALYYCASGVAWGQGTLVTVSSGGGGSGGGSEVQLVESGGGVVQPGG |
| | | SLRLSCAASGFTFRSFGMSWVRQAPGKGPEWVSSISGSGSDTLYADSVKG |
| | | RFTISRDNSKNTLYLQMNSLRPEDTALYYCTIGGSLSRSSQGTLVTVSSA |
| 457 | A022600425 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGVVQPGGSLRLSCAASGSI |
| | | FRSVFSSSTMEWYRQAPGKKRELVARIAPGEGTYYGALYADSVKGRFTISR |
| | | DNAKNTVYLQMNSLRPEDTALYYCASGVAWGQGTLVTVSSGGGGSGGG |
| | | GSGGGGSGGGGSEVQLVESGGGVVQPGGSLRLSCAASGFTFSSFAMTW |
| | | VRRPPGKGLEWVATITNAGVTSYADSVKGRFTISRDNAKNTLYLQMNSLR |
| | | PEDTALYICANARRTGPRAPTDIGSYRGQGTLVTVSSGGGGSGGGSEVQL |
| | | VESGGGVVQPGGSLRLSCAASGFTFRSFGMSWVRQAPGKGPEWVSSISG |
| | | SGSDTLYADSVKGRFTISRDNSKNTLYLQMNSLRPEDTALYYCTIGGSLSRS |
| | | SQGTLVTVSSA |
| 458 | A022600426 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGVVQPGGSLRLSCAASGSI |
| | | FRSVFSSSTMEWYRQAPGKKRELVARIAPGEGTYYGALYADSVKGRFTISR |
| | | DNAKNTVYLQMNSLRPEDTALYYCASGVAWGQGTLVTVSSGGGGSGGG |
| | | GSGGGGSGGGGSEVQLVESGGGVVQPGGSLRLSCAASGFTFSSFAMTW |
| | | VRRPPGKGLEWVATITNKGVTSYADSVKGRFTISRDNAKNTLYLQMNSLR |
| | | PEDTALYICANARRTGPRAPTDIGSYRGQGTLVTVSSGGGGSGGGSEVQL |
| | | VESGGGVVQPGGSLRLSCAASGFTFRSFGMSWVRQAPGKGPEWVSSISG |
| | | SGSDTLYADSVKGRFTISRDNSKNTLYLQMNSLRPEDTALYYCTIGGSLSRS |
| | | SQGTLVTVSSA |
| 459 | A022600427 | DVQLVESGGGVVQPGGSLRLSCVASGYVHKINFYGWYRQAPGKEREKVA |
| | | HISIGDQTDYADSAKGRFTISRDESKNTVYLQMNSLRPEDTAAYYCRALSRI |
| | | WPYDYWGQGTLVTVSSGGGGSEVQLVESGGGVVQPGGSLRLSCAASGF |
| | | TFSSFAMTWVRRPPGKGLEWVATITNAGVTSYADSVKGRFTISRDNAKNT |
| | | LYLQMNSLRPEDTALYICANARRTGPRAPTDIGSYRGQGTLVTVSSGGGG |
| | | SGGGSEVQLVESGGGVVQPGGSLRLSCAASGSIFRSVFSSSTMEWYRQAP |
| | | GKKRELVARIAPGEGTYYGALYADSVKGRFTISRDNAKNTVYLQMNSLRPE |
| | | DTALYYCASGVAWGQGTLVTVSSGGGGSGGGSEVQLVESGGGVVQPGG |
| | | SLRLSCAASGFTFRSFGMSWVRQAPGKGPEWVSSISGSGSDTLYADSVKG |
| | | RFTISRDNSKNTLYLQMNSLRPEDTALYYCTIGGSLSRSSQGTLVTVSSA |

### 6 EXAMPLES

### 6.1 Example 1: Parental TCR Binding ISVD for Sequence Optimization

Sequence optimisation was undertaken for immunoglobulin single variable domain (ISVD) T0170056G05, which binds to the constant domain of the T cell receptor. The sequence of T0170056G05 is depicted in FIG. 1.

Immunoglobulin single variable domain (ISVD) T0170056G05 was previously developed and described in international application with publication number WO2016180969A1. The aforementioned application describes in its examples the generation of ISVDs capable of binding to the constant domain of the T cell receptor present on a T cell. Upon formatting of such a TCR binding ISVD with a second (and/or third) ISVD that binds to an antigen expressed on a target cell, the ability of those multispecific formats to kill the target cells expressing the antigens was demonstrated. Such a multispecific format is thus able to redirect a T cell to a target cell and subsequently induce T cell activation resulting in lysis of the target cell.

As exemplified in application WO2016180969, ISVD libraries were generated and screened in a binding assay to identify ISVDs capable of binding to the constant domain of the T cell receptor (TCR). Sequence analysis of the identified hits resulted in the identification of 3 distinct clusters of ISVDs. From these ISVDs, representatives belonging to cluster A, such as ISVD T0170056G05, generally demonstrated the best EC50 values for binding to the constant domain of the human TCR. Additionally, some cross-reactivity could be observed for binding to the constant domain of cynomolgus TCR. However, as shown in Example 18.4 in WO2016180969, the affinity of the characterised cluster A representatives for binding to cynomolgus TCR was found to be 10-fold lower compared to binding to human TCR in a biochemical assay.

Given the fact that monkeys are generally considered to be the most suitable animal species for preclinical studies, including efficacy and toxicity studies, an effort was initiated to optimise the sequence of cluster A ISVD T0170056G05 with the aim to improve cross-reactivity for binding to TCR from human and non-human primates (such as cynomolgus or rhesus monkeys) origin.

Additionally, sequences were optimised for purposes of humanisation, knock-out of post-translational modification sites, deimmunisation, improvement of stability and reduction of binding of potential pre-existing antibodies.

### 6.2 Example 2: Tools

### Human TCR(2XN9)-Zipper Protein and Cynomolgus TCR(AEA41865/AEA41866)-Zipper Protein

Soluble human and cynomolgus monkey TCR α/β proteins were generated in house.

The sequences for the extracellular part of the human TCR α/β constant domain were derived from UniProtKB P01848 for TCR α and P01850 for TCR β. The human TCR α/β variable domains were derived from crystal structure sequence with PDB code: 2XN9.

The sequences for the extracellular part of the cynomolgus monkey TCR α/β constant domains were derived from GenBank files AEA41865 and AEA41868 for a and 3 chain, respectively. The sequences for the TCR α/β variable domains were derived from AEA41865 and AEA41866 for α and β chain, respectively. In house sequencing confirmed that aforementioned constant domains sequences originally derived from rhesus monkeys are identical to those from cynomolgus monkeys.

The extracellular domains of human TCR α/β (2XN9) or cynomolgus monkey TCR α/β were fused to a zipper protein coding sequence (O'Shea et al. 1993 Curr. Biol. 3(10): 658-667). A His-tag or Flag-tag was added for purification purposes. The resulting amino acid sequences of the zipper proteins are those with SEQ ID NO's: 318, 319, 320 and 321 for the human a chain, human p chain, cyno a and cyno p chain, respectively.

The zipper proteins were produced by CHOK1SV cells (Lonza) using Lonza's GS Gene Expression System™ and subsequently purified.

### TCR α6/CD3 Transfected Cell Lines

Stable CHO-K1 (ATCC: CCL-61), and HEK293H (Life technologies 11631-017) cell lines with recombinant overexpression of all 6 chains of the full human or cynomolgus T cell Receptor complex were generated (CHO-K1 huTCR(2XN9)/huCD3, CHO-K1 huTCR(3TOE)/huCD3, HEK293H huTCR(2IAN)/huCD3, CHO-K1 cyTCR/cyCD3). For this, the coding sequences of the TCR alpha (α) and TCR beta (β) chain were cloned in a pcDNA3.1-derived vector, downstream of a CMV promotor and a 2A-like viral peptide sequence was inserted between both chains to induce ribosomal skipping during translation of the polyprotein. In the same vector, the coding sequences of the epsilon, delta, gamma and zeta chains of the CD3 complex were cloned downstream of an additional CMV promotor, also using 2A-like viral peptide sequences between the respective chains.

The used sequences for the human CD3 and the human TCR α/p constant domains were derived from UniProtKB (CD3 delta: P04234, CD3 gamma: P09693, CD3 epsilon: P07766, CD3 zeta: P20963, TCR α: P01848 and TCR 3: P01850; SEQ ID NOs: 300 to 305, respectively). The sequences for the human TCR α/β variable domains were derived from crystal structure sequences (PDB codes: 2IAN, 2XN9 and 3TOE) (human TCR α variable domains derived from 2IAN, 2XN9 and 3TOE with SEQ ID NOs: 306 to 308, respectively; human TCR 3 variable domains derived from 2IAN, 2XN9 and 3TOE with SEQ ID NOs: 309 to 311, respectively).

For the cell lines overexpressing the cyno TCR/CD3 complex, following sequences were used.

The sequences for the CD3 domains were derived from cynomolgus monkey, with as UniProtKB for CD3 delta: NP_001274617, CD3 gamma: BAJ16168, CD3 epsilon: Q95L15, CD3 zeta: XP_005539936 (SEQ ID NOs: 312 to 315, respectively). The sequences for the TCR α/β chains (constant and variable domains) were derived from rhesus monkey with as GenBank file numbers AEA41863 (TCR α) and AEA41864 (TCR β) (SEQ ID NOs: 316 and 317, respectively). As mentioned above, it was confirmed in house that the amino acid sequences of the rhesus TCR α/β constant domains are identical to the TCR α/β constant domains from cynomolgus monkeys.

### Human and Cynomolgus Primary T Cells

Human T cells were collected from the Buffy Coat fraction of blood from healthy volunteers (Blood bank Gent) using RosetteSep (StemCell Technologies, 15061) followed by enriching on Ficoll-Paque™ PLUS (GE Healthcare, 17-1440-03) according to manufacturer's instructions. The quality and purity of the purified human T cells was checked with anti-CD3 (eBioscience, 12-0037-73), anti-CD8 (BD Biosciences, 555367), anti-CD4 (BD Biosciences, 345771), anti-CD45RO (BD Biosciences, 555493), anti-CD45RA (BD Biosciences, 550855), anti-CD19 (BD Biosciences, 555413), anti-CD25 (BD Biosciences, 557138) and anti-CD69 (BD Biosciences, 557050) fluorescently labelled antibodies in a flow cytometric assay. Cells were frozen in liquid nitrogen. Cynomolgus T cells were isolated by LPT Laboratory of Pharmacology and Toxicology GmbH & Co. KG, using the Pan T Cell Isolation Kit (MACS, 130-091-993).

CHO Flp-In Cells and CHO FLP-Ln huCD123 Cells

Stable CHO Flp-In (Invitrogen, R758-07) cell lines with recombinant overexpression of CD123 were generated using the Flp-In™ site-directed recombination technology (Flp-In™ System For Generating Stable Mammalian Expression Cell Lines by Flp Recombinase-Mediated Integration (Invitrogen, K601001, K601002)). Hereby, DNA integration occurs at a specific genomic location at an FRT (Flp Recombination Target) site by the Flp recombinase (pOG44) derived from Saccharomyces cerevisiae. The Flp-In™ host cell line and expression plasmid (pcDNA5) both contain this FRT site, thereby allowing a single homologous DNA recombination. The sequence for human CD123 was derived from NCBI RefSeq NP_002174 (SEQ ID NO: 379).

### 6.3 Example 3: Sequence Optimization

Sequence optimization is a process in which a parental ISVD sequence is mutated. This process covers the humanization of the ISVD and knocks out sites for post-translational modifications as well as epitopes for potential pre-existing antibodies.

Humanization is a process in which a parental ISVD sequence is mutated to make it more homologous to the human IGHV3-IGHJ germline consensus sequence. Specific amino acids in the framework regions that differ between the ISVD and the human IGHV3-IGHJ germline consensus are altered to the human counterpart in such a way that the protein structure, activity and stability are kept intact.

In addition, the amino acids present in the CDRs and for which there is experimental evidence that they are sensitive to post-translational modifications (PTM) are altered in such a way that the PTM site is inactivated while the protein structure, activity and stability are kept intact.

A basic sequence optimization variant of the parental ISVD T0170056G05 was generated, which contains three mutations in its framework regions (L11V, K83R and V89L). The His-tagged basic variant (i.e. T0170056G05(L11V, K83R, V89L)-His6) is denominated herein as T0170000351, while the Flag3-His6-tagged basic variant (i.e. T0170056G05(L11V, K83R, V89L)-Flag3-His6) is referred to herein as T017000141. Two additional variants were generated which each contain an additional mutation in their framework regions, either M77T (for variant T017000343) or F91Y (for variant T017000345). All variants were expressed as His6-tagged proteins.

The variants were characterized and compared to the parental ISVD (T0170056G05) and/or the basic sequence optimization variant (T017000351). The characterization was performed in several assays, including assessment of binding to CHO-K1 huTCR(2XN9)/huCD3 and to human primary T cells, off-rate analysis (Proteon) on cynoTCR(AEA41865/AEA41866)-zipper protein and humanTCR(2XN9)-zipper protein and determination of the melting temperature (Tm) in a thermal shift assay (TSA). The assays are described below in more detail.

Dose-dependent binding of the purified monovalent ISVD variants to CHO-K1 huTCR(2XN9)/huCD3 overexpressing cells or human primary T cells was evaluated by flow cytometry. In brief, cells were harvested and transferred to a V-bottom 96-well plate (Greiner Bio-one, 651180; 5×10⁵ cells/well) and incubated with serial dilutions of ISVDs for 30 min at 4° C. in FACS buffer (D-PBS from Gibco, with 2% FBS from Sigma and 0.05% sodium azide from Merck). Next, cells were washed 3 times with FACS buffer and incubated with 1 μg/ml THE™ His Tag Antibody, mAb, Mouse (Genscript, A00186) and washed again. Binding was detected after incubation for 30 min at 4° C. with R-Phycoerythrin-AffiniPure F(ab′)2 Fragment Goat Anti-Mouse IgG, Fc gamma Fragment Specific (Jackson Immunoresearch, 115-116-07). Subsequently, cells were resuspended in FACS buffer supplemented with 5 nM TO-PRO®-3 Iodide to distinguish live from dead cells, which are removed during the gating procedure. Cells were analyzed using a FACS Array flow cytometer (BD Biosciences) and Flowing Software. First a P1 population which represented more than 80% of the total cell population was selected based on FSC-SSC distribution. In this gate, 10000 cells were counted during acquisition. From this population the TO-PRO®-3+ cells (dead cells) were excluded and the mean fluorescence intensity (MFI) PE value was calculated.

Off-rates of the purified tagged monovalent ISVDs for recombinant TCR were determined by SPR on a ProteOn XPR36 instrument. HuTCR-zipper or cyTCR-zipper protein was immobilized on a GLC sensor chip (between 100-200 RU) via amine coupling chemistry, using EDC/NHS for activation of the carboxyl groups on the chip surface (running buffer: HBS-EP+, pH7.4). Purified ISVDs were injected for 2 min (at a flow rate of 45 μL/min) at different concentrations (between 4.1 nM and 1000 nM) and dissociation was followed for 900 s. Data were double referenced by subtracting a reference ligand lane and a blank buffer injection. Processed sensorgrams were fitted with the Langmuir model (1:1 interaction) using ProteOn Manager 3.1.0 (Version 3.1.0.6) software.

The thermal shift assay (TSA) was performed in a 96-well plate on the LightCycler 48011 machine (Roche). Per row, one ISVD was analyzed at the following pH: 3.5/4/4.5/5/5.5/6/6.5/7/7.5/8/8.5/9. Per well, 5 μL of ISVD sample (0.8 mg/mL in PBS) was added to 5 μL of Sypro Orange (40× in MilliQ water; Invitrogen cat. No. S6551) and 10 μL of buffer (100 mM phosphate, 100 mM borate, 100 mM citrate and 115 mM NaCl, pH range 3.5 to 9). The applied temperature gradient (37 to 99° C. at a rate of 0.03° C./s) induces unfolding of the ISVDs, whereby hydrophobic patches become exposed. Sypro Orange binds to those hydrophobic patches, resulting in an increase in fluorescence intensity (Ex/Em=465/580 nm). The inflection point of the first derivative of the fluorescence intensity curve at pH 7 serves as a measure of the melting temperature (Tm).

The introduced mutations had no negative effect on protein structure, activity and stability (see Table 1).

TABLE 1
Characterization data of anti-TCR ISVD T01700056G05 and generated variants. Mutations are
indicated by their amino acid one letter code at their respective position according to
Kabat numbering. A dot reflects that at a certain position the amino acid is identical
to the amino acid at the same position in the CDR as for the parental ISVD T01700056G05.

| | | | | | | binding | | Off-rate analysis | | |
|---|---|---|---|---|---|---|---|---|---|---|
| | | | | | | CHO-K1 | Human | huTCR- | cyTCR- | |
| | | | | | | huTCR(2XN9)/ | primary | zipper | zipper | |
| | L | M | K | V | F | huCD3 | T cells | protein | protein | Tm |
| ISVD ID | 11 | 77 | 83 | 89 | 91 | EC50 (M) | EC50 (M) | k_{d} (1/s) | k_{d} (1/s) | (° C.) |
| T0170056G05 | . | . | . | . | . | 1.2E−08 | 1.1E−07 | — | — | — |
| T017000351 | V | . | R | L | . | 1.0E−08 | 1.1E−07 | 9.4E−04 | 1.2E−02 | 77 |
| T017000343 | V | T | R | L | . | 8.7E−09 | 1.2E−07 | 1.2E−03 | 1.5E−02 | 78 |
| T017000345 | V | . | R | L | Y | 1.9E−08 | 1.6E−07 | 9.9E−04 | 1.2E−02 | 76 |

### 6.4 Example 4: Affinity Maturation

The basic sequence optimization variant of T0170056G05 (i.e. T017000141) containing the three mutations L11V, K83R and V89L, was selected for affinity maturation.

Affinity maturation was performed in two steps. In the first step a pooled single site saturation library of all CDR positions (10 for CDR1, 9 for CDR2 and 9 for CDR3) was constructed for each CDR. Each single site saturation library was constructed using primers that were designed according to the 22c-trick approach (Kille et al. ACS Synth. Biol., 2013, 2 (2), pp 83-92). Individual mutations that resulted in improved binding were screened for via ELISA and off-rate analysis (Proteon) on cyTCR-zipper protein and huTCR-zipper protein.

The ELISA was performed as follows. Maxisorp 96-well ELISA plates were directly coated with huTCR-zipper or cynoTCR-zipper protein in PBS and incubated overnight at 4° C. Plates were washed with PBS+0.05% Tween20 and blocked with PBS+1% Casein for 1 hour. ISVD-containing periplasmic extract, diluted 1/10 in PBS+0.1% Casein+0.05% Tween20, was added to the plates and incubated for 1 hour at room temperature. After washing, binding of ISVDs was detected using Monoclonal ANTI-FLAG® M2-Peroxidase (HRP) antibody (Sigma, A8592), 1/5000 diluted and esTMB (SDT, esTMB) substrate. The reaction was stopped after 25 min incubation using 1 M HCl and OD signals were measured at 450 nm.

Clones with highest binding signals in ELISA were sequenced. Unique ISVD clones, i.e. clones with a unique sequence, were subsequently selected and screened to determine their off-rate. To this purpose, the ISVD containing periplasmic extracts were screened on huTCR-zipper and cyTCR-zipper protein by SPR on a ProteOn XPR36 instrument. The zipper proteins were immobilized on a GLC sensor chip (between 4300 and 5100RU) via amine coupling chemistry, using EDC/sulfo-NHS for activation of the carboxyl groups on the chip surface (running buffer: HBS-EP+, pH7.4). Periplasmic extracts were injected at 1/10 dilution for 2 min (at a flow rate of 45 μL/min) and dissociation was followed for 900 s. A pulse of 100 s of 3 M MgCl2 at 45 μL/min was used for regeneration. Data were double referenced by subtracting a reference ligand lane and a blank buffer injection. Processed sensorgrams were fitted with the Langmuir model (1:1 interaction) using ProteOn Manager 3.1.0 (Version 3.1.0.6) software.

The screening resulted in the identification of 29 unique clones which showed an improved off-rate on humanTCR-zipper protein and/or the cynoTCR-zipper protein. All identified clones, the corresponding mutation in their CDR, the off-rata data and observed effect are depicted in Tables 2, 3 and 4 for the CDR1, CDR2 and CDR3 variants respectively.

TABLE 2
Off-rate analysis of CDR1 variants. Mutations are indicated by their amino acid one letter code at their respective position
in the CDR (according to Kabat numbering). A dot reflects that at a certain position the amino acid is identical to the
amino acid at the same position in the CDR as for the basic sequence optimized variant T017000141. If the mutation results
in an improved off-rate on either human or cyno TCR, this is indicated as a plus in the column listing the effect.

| | | | | | | | | | | | Off-rate analysis | | | |
|---|---|---|---|---|---|---|---|---|---|---|---|---|---|---|
| | | | | | | | | | | | (k_{d} (1/s)) | | | |
| | CDR1 | | | | | | | | | | humanTCR | cynoTCR | effect | |
| ISVD ID | G26 | D27 | V28 | H29 | K30 | I31 | N32 | F33 | L34 | G35 | zipper | zipper | human | cyno |
| T017000141 | . | . | . | . | . | . | . | . | . | . | 1.00E−03 | 1.00E−02 | | |
| T017000164 | . | Y | . | . | . | . | . | . | . | . | 1.08E−03 | 4.12E−03 | − | + |
| T017000166 | . | . | . | . | . | . | . | . | Y | . | 6.41E−04 | 8.25E−03 | + | + |
| T017000161 | . | E | . | . | . | . | . | . | . | . | 6.91E−04 | 6.24E−03 | + | + |
| T017000177 | . | S | . | . | . | . | . | . | . | . | 5.85E−04 | 3.25E−03 | + | + |
| T017000188 | . | A | . | . | . | . | . | . | . | . | 5.77E−04 | 3.89E−03 | + | + |
| T017000174 | W | . | . | . | . | . | . | . | . | . | 3.54E−04 | 7.91E−03 | + | + |
| T017000208 | Y | . | . | . | . | . | . | . | . | . | 4.76E−04 | 1.47E−02 | + | − |
| T017000163 | . | . | . | . | Q | . | . | . | . | . | 1.09E−03 | 6.42E−03 | − | + |
| T017000176 | . | . | . | . | . | L | . | . | . | . | 7.25E−04 | 7.91E−03 | + | + |
| T017000179 | . | . | . | . | . | . | . | . | . | T | 7.66E−04 | 7.75E−03 | + | + |
| T017000180 | Q | . | . | . | . | . | . | . | . | . | 8.52E−04 | 9.98E−03 | + | + |
| T017000196 | . | . | . | . | . | . | F | . | . | . | 1.41E−03 | 4.19E−03 | − | + |
| T017000170 | . | . | . | . | . | . | . | Y | . | . | 1.02E−03 | 8.06E−03 | − | + |
| T017000173 | . | . | . | . | . | . | . | V | . | . | 1.46E−03 | 9.03E−03 | − | + |
| T017000185 | . | . | . | . | . | . | . | . | F | . | 8.82E−04 | 1.14E−02 | + | − |
| T017000200 | . | . | . | . | . | . | . | . | . | R | 6.96E−04 | 5.80E−02 | + | − |
| T017000190 | . | . | . | . | . | . | . | . | . | S | 1.23E−03 | 1.07E−02 | − | − |

TABLE 3
Off-rate analysis of CDR2 variants. Mutations are indicated by their amino acid one letter code
at their respective position in the CDR (according to Kabat numbering). A dot reflects that at
a certain position the amino acid is identical to the amino acid at the same position in the CDR
as for the basic sequence optimized variant T017000141. If the mutation results in an improved
off-rate on either human or cyno TCR, this is indicated as a plus in the column listing the effect.

| | | | | | | | | | | Off-rate analysis | | | |
|---|---|---|---|---|---|---|---|---|---|---|---|---|---|
| | | | | | | | | | | (k_{d} (1/s)) | | | |
| | CDR2 | | | | | | | | | humanTCR | cynoTCR | effect | |
| ISVD ID | H50 | I51 | S52 | I53 | G54 | D55 | Q56 | T57 | D58 | zipper | zipper | human | cyno |
| T017000141 | . | . | . | . | . | . | . | . | . | 1.00E−03 | 1.00E−02 | | |
| T017000231 | V | . | . | . | . | . | . | . | . | 1.04E−03 | 8.67E−03 | − | + |
| T017000245 | . | . | . | . | A | . | . | . | . | 7.84E−04 | 9.36E−03 | + | + |
| T017000238 | . | . | . | . | . | . | E | . | . | 9.32E−04 | 9.57E−03 | + | + |
| T017000249 | . | . | . | . | . | . | V | . | . | 9.59E−04 | 9.39E−03 | + | + |
| T017000232 | . | . | . | . | . | . | . | V | . | 9.91E−04 | 8.95E−03 | + | + |
| T017000233 | . | . | . | . | . | . | . | S | . | 9.93E−04 | 1.02E−02 | + | − |
| T017000234 | M | . | . | . | . | . | . | . | . | 1.39E−03 | 1.18E−02 | − | − |
| T017000258 | . | . | . | . | . | E | . | . | . | 1.19E−03 | 1.07E−02 | − | − |
| T017000235 | . | . | . | . | . | . | H | . | . | 1.11E−03 | 1.10E−02 | − | − |
| T017000250 | . | . | . | . | . | . | K | . | . | 1.11E−03 | 1.11E−02 | − | − |
| T017000254 | . | . | . | . | . | . | R | . | . | 1.26E−03 | 1.18E−02 | − | − |
| T017000223 | . | . | . | . | . | . | . | A | . | 1.02E−03 | 1.09E−02 | − | − |
| T017000241 | . | . | . | . | . | . | . | . | N | 1.35E−03 | 1.67E−02 | − | − |

TABLE 4
Off-rate analysis of CDR3 variants. Mutations are indicated by their amino acid one letter code
at their respective position in the CDR (according to Kabat numbering). A dot reflects that at
a certain position the amino acid is identical to the amino acid at the same position in the CDR
as for the basic sequence optimized variant T017000141. If the mutation results in an improved
off-rate on either human or cyno TCR, this is indicated as a plus in the column listing the effect.

| | | | | | | | | | | Off-rate analysis | | | |
|---|---|---|---|---|---|---|---|---|---|---|---|---|---|
| | | | | | | | | | | (k_{d} (1/s)) | | | |
| | CDR3 | | | | | | | | | humanTCR | cynoTCR | effect | |
| ISVD ID | F95 | S96 | R97 | I98 | Y99 | P100 | Y100a | D101 | Y102 | zipper | zipper | human | cyno |
| T017000141 | . | . | . | . | . | . | . | . | . | 1.00E−03 | 1.00E−02 | | |
| T017000312 | L | . | . | . | . | . | . | . | . | 7.29E−04 | 8.70E−03 | + | + |
| T017000291 | . | . | . | . | W | . | . | . | . | 7.11E−04 | 6.72E−03 | + | + |
| T017000281 | M | . | . | . | . | . | . | . | . | 5.61E−04 | 5.55E−03 | + | + |
| T017000290 | . | . | K | . | . | . | . | . | . | 1.14E−02 | 1.71E−03 | − | + |
| T017000294 | . | . | . | . | . | . | . | . | W | 6.19E−04 | 1.51E−02 | + | − |
| T017000283 | . | . | S | . | . | . | . | . | . | 1.97E−02 | 5.59E−03 | − | + |
| T017000287 | . | . | . | . | . | . | . | Q | . | 9.11E−04 | 1.21E−02 | + | − |
| T017000278 | . | . | . | . | . | . | . | S | . | 1.31E−03 | 1.62E−02 | − | − |
| T017000285 | . | . | . | . | . | . | . | L | . | 1.10E−03 | 2.16E−02 | − | − |
| T017000301 | . | . | . | . | . | . | . | A | . | 1.57E−03 | 1.78E−02 | − | − |
| T017000302 | . | . | . | . | . | . | . | T | . | 1.50E−03 | 1.48E−02 | − | − |

In a second step, 12 single mutations divided over 9 positions in CDR1 and CDR3 were combined in a combinatorial library. This library also included the parental amino acid for all 9 positions, resulting in a library with a theoretical diversity of 1536 variants (design see Table 5). This library contains all possible combinations of these mutations, resulting in variants with no and up to 9 mutations. This library was generated with overlap extension PCR with primers coding for the different desired amino acids per position.

TABLE 5
Combinatorial library design

| | CDR1 | | | | | CDR3 | | | | |
|---|---|---|---|---|---|---|---|---|---|---|
| WT | G26 | D27 | K30 | I31 | L34 | F95 | R97 | Y99 | Y102 | Diversity |
| Mutations | W | S | Q | L | Y | M | K | W | W | |
| | Y | A | | | | | | | | |
| | | Y | | | | | | | | |
| # possibilities | 3 | 4 | 2 | 2 | 2 | 2 | 2 | 2 | 2 | 1536 |

Variants with multiple mutations that resulted in improved binding were screened for via ELISA, as described above. Clones with highest binding signals were subsequently sequenced and clones with a unique sequence were subjected to an off-rate analysis (Proteon) on cyTCR-zipper protein and humanTCR-zipper protein, again as described above. In a later stage, additional variants with a Leucine at position 95 were generated and screened.

The screening effort resulted in the identification of 91 unique clones which showed an improved off-rate on huTCR-zipper protein and/or the cyTCR-zipper protein compared to T017000141. All identified ISVD variants and their corresponding mutations in CDR1 and/or CDR3 and the off-rate data are depicted in Table 6.

For 57 variants, the relative difference in human/cyno cross-reactivity was lower compared to the basic variant T017000141. A total of 34 ISVD variants were identified wherein the gap for human/cyno cross-reactivity was 5-fold or lower. The gap might either be in favor for humanTCR or cynoTCR binding.

TABLE 6
Off-rate analysis for ISVD variants from combinatorial library. Mutations are indicated by their amino acid one
letter code at their respective position in the CDR (according to Kabat numbering). A dot reflects that at a certain
position the amino acid is identical to the amino acid at the same position in the CDR as for the basic sequence
optimized variant T017000141. If the mutation results in an improved off-rate (i.e. slower dissociation, as reflected
by a lower value) on either human or cyno TCR, this is indicated as a plus in the column listing the effect. The
effect column additionally depicts the relative difference for binding to human and cyno TCR.

| | | | | | | | | | | | | Effect | | |
|---|---|---|---|---|---|---|---|---|---|---|---|---|---|---|
| | | | | | | | | | | Off-rate analysis | | Gap in | | |
| | | | | | | | | | | (k_{d} (1/s)) | | off-rate | | |
| | CDR1 | | | | | CDR3 | | | | humanTCR | CynoTCR | human vs | | |
| ISVD ID | G26 | D27 | K30 | I31 | L34 | F95 | R97 | Y99 | Y102 | zipper | zipper | cyno * | human | cyno |
| T017000141 | . | . | . | . | . | . | . | . | . | 1.10E−03 | 8.20E−03 | 7.5 | | |
| T017000613 | W | . | . | . | Y | L | K | W | . | 3.49E−04 | 3.64E−04 | 1.0 | + | + |
| T017000615 | Y | . | . | . | Y | L | K | W | . | 7.93E−04 | 8.70E−04 | 1.1 | + | + |
| T017000505 | . | . | Q | L | . | . | K | . | . | 1.03E−03 | 1.43E−03 | 1.4 | + | + |
| T017000362 | Y | . | . | . | Y | M | K | W | . | 8.04E−04 | 5.77E−04 | 1.4 | + | + |
| T017000440 | W | . | . | L | Y | M | K | W | . | 5.11E−04 | 3.46E−04 | 1.5 | + | + |
| T017000378 | W | . | . | . | Y | M | K | W | . | 3.76E−04 | 2.49E−04 | 1.5 | + | + |
| T017000608 | W | . | . | . | Y | . | K | W | . | 8.01E−04 | 4.72E−04 | 1.7 | + | + |
| T017000374 | Y | . | Q | . | Y | M | K | W | . | 9.21E−04 | 5.23E−04 | 1.8 | + | + |
| T017000607 | W | . | Q | . | Y | . | K | W | . | 6.07E−04 | 3.16E−04 | 1.9 | + | + |
| T017000602 | W | . | Q | . | . | . | K | W | . | 1.11E−03 | 5.50E−04 | 2.0 | − | + |
| T017000359 | W | . | Q | . | Y | . | K | . | . | 8.74E−04 | 4.01E−04 | 2.2 | + | + |
| T017000740 | W | . | Q | L | Y | M | K | . | . | 9.93E−04 | 3.91E−04 | 2.5 | + | + |
| T017000554 | Y | . | Q | . | . | M | K | . | . | 2.06E−03 | 7.67E−04 | 2.7 | − | + |
| T017000413 | W | . | Q | L | Y | M | K | W | . | 6.48E−04 | 2.38E−04 | 2.7 | + | + |
| T017000379 | W | . | Q | . | Y | M | K | W | . | 4.21E−04 | 1.52E−04 | 2.8 | + | + |
| T017000367 | . | Y | . | . | . | M | . | W | . | 3.93E−04 | 1.10E−03 | 2.8 | + | + |
| T017000390 | W | . | Q | . | . | M | K | W | . | 6.73E−04 | 2.21E−04 | 3.0 | + | + |
| T017000443 | W | . | Q | . | Y | M | K | . | . | 6.41E−04 | 2.09E−04 | 3.1 | + | + |
| T017000776 | W | S | Q | . | Y | M | K | W | . | 1.66E−03 | 5.25E−04 | 3.2 | − | + |
| T017000371 | . | Y | . | . | Y | M | . | W | . | 1.88E−04 | 6.12E−04 | 3.3 | + | + |
| T017000759 | . | S | . | . | Y | . | K | W | W | 3.39E−03 | 1.01E−03 | 3.4 | − | + |
| T017000539 | . | Y | . | . | Y | M | . | W | W | 4.29E−04 | 1.51E−03 | 3.5 | + | + |
| T017000499 | . | Y | . | L | . | M | . | W | . | 2.92E−04 | 1.06E−03 | 3.6 | + | + |
| T017000538 | . | Y | . | L | Y | M | . | W | . | 2.09E−04 | 7.66E−04 | 3.7 | + | + |
| T017000410 | . | Y | . | L | Y | . | . | W | W | 3.73E−04 | 1.59E−03 | 4.3 | + | + |
| T017000566 | . | Y | . | L | . | M | . | W | W | 3.83E−04 | 1.64E−03 | 4.3 | + | + |
| T017000589 | . | Y | . | L | Y | M | . | W | W | 2.68E−04 | 1.15E−03 | 4.3 | + | + |
| T017000397 | . | S | Q | L | . | M | . | W | . | 5.69E−04 | 2.45E−03 | 4.3 | + | + |
| T017000757 | . | A | . | L | . | . | K | W | W | 2.65E−03 | 6.03E−04 | 4.4 | − | + |
| T017000372 | . | Y | . | . | Y | M | . | . | . | 2.70E−04 | 1.22E−03 | 4.5 | + | + |
| T017000423 | . | S | Q | . | . | M | . | W | . | 4.05E−04 | 1.84E−03 | 4.5 | + | + |
| T017000521 | . | S | Q | L | Y | M | . | W | . | 5.65E−04 | 2.75E−03 | 4.9 | + | + |
| T017000370 | . | A | Q | . | Y | M | . | W | . | 4.36E−04 | 2.15E−03 | 4.9 | + | + |
| T017000383 | . | . | Q | . | . | M | . | W | . | 4.35E−04 | 2.16E−03 | 5.0 | + | + |
| T017000364 | . | A | Q | . | . | M | . | W | . | 5.06E−04 | 2.60E−03 | 5.1 | + | + |
| T017000442 | . | Y | . | L | Y | M | . | . | . | 2.76E−04 | 1.46E−03 | 5.3 | + | + |
| T017000461 | . | . | Q | . | Y | M | . | W | . | 3.83E−04 | 2.06E−03 | 5.4 | + | + |
| T017000384 | . | A | Q | L | Y | M | . | W | . | 7.11E−04 | 3.83E−03 | 5.4 | + | + |
| T017000593 | . | A | . | L | Y | M | K | W | . | 1.07E−03 | 1.90E−04 | 5.6 | + | + |
| T017000373 | . | S | . | L | . | M | . | W | . | 1.60E−04 | 9.13E−04 | 5.7 | + | + |
| T017000614 | . | Y | . | . | Y | L | . | W | . | 2.03E−04 | 1.16E−03 | 5.7 | + | + |
| T017000509 | . | A | . | . | . | M | K | W | W | 3.51E−03 | 6.09E−04 | 5.8 | − | + |
| T017000361 | . | S | . | . | . | M | . | W | . | 1.67E−04 | 9.82E−04 | 5.9 | + | + |
| T017000498 | . | A | . | . | Y | M | . | W | W | 3.67E−04 | 2.23E−03 | 6.1 | + | + |
| T017000408 | . | S | . | . | Y | M | . | W | . | 1.14E−04 | 7.14E−04 | 6.3 | + | + |
| T017000432 | . | S | . | L | Y | M | K | W | . | 8.95E−04 | 1.42E−04 | 6.3 | + | + |
| T017000381 | . | A | . | . | . | M | . | . | . | 3.30E−04 | 2.16E−03 | 6.5 | + | + |
| T017000754 | . | Y | Q | . | . | M | K | W | . | 1.56E−03 | 2.37E−04 | 6.6 | − | + |
| T017000745 | . | . | Q | . | . | . | . | . | . | 1.03E−03 | 7.01E−03 | 6.8 | + | + |
| T017000515 | . | A | . | . | . | M | . | W | . | 1.85E−04 | 1.26E−03 | 6.8 | + | + |
| T017000360 | W | S | Q | L | Y | M | K | . | W | 3.55E−04 | 2.43E−03 | 6.8 | + | + |
| T017000588 | . | A | . | . | Y | M | K | W | . | 1.09E−03 | 1.58E−04 | 6.9 | + | + |
| T017000401 | . | S | . | . | Y | M | . | W | W | 2.65E−04 | 1.83E−03 | 6.9 | + | + |
| T017000357 | . | Y | Q | L | Y | M | . | W | . | 3.50E−04 | 2.51E−03 | 7.2 | + | + |
| T017000354 | . | A | . | . | Y | M | . | W | . | 1.22E−04 | 8.75E−04 | 7.2 | + | + |
| T017000547 | . | A | . | L | Y | . | . | W | . | 2.14E−04 | 1.56E−03 | 7.3 | + | + |
| T017000531 | . | S | . | . | . | M | . | W | W | 3.18E−04 | 2.32E−03 | 7.3 | + | + |
| T017000391 | . | A | . | . | Y | M | . | . | . | 2.05E−04 | 1.61E−03 | 7.9 | + | + |
| T017000356 | . | S | . | L | Y | M | . | W | . | 1.13E−04 | 8.90E−04 | 7.9 | + | + |
| T017000406 | . | Y | Q | . | Y | M | . | W | . | 1.75E−04 | 1.38E−03 | 7.9 | + | + |
| T017000468 | . | S | . | . | Y | . | . | W | . | 1.76E−04 | 1.41E−03 | 8.0 | + | + |
| T017000358 | . | A | . | L | . | M | . | W | . | 1.43E−04 | 1.15E−03 | 8.0 | + | + |
| T017000369 | . | S | . | L | Y | M | . | W | W | 1.92E−04 | 1.55E−03 | 8.1 | + | + |
| T017000363 | . | S | . | . | Y | M | . | . | . | 1.57E−04 | 1.27E−03 | 8.1 | + | + |
| T017000450 | . | Y | Q | . | . | M | . | W | . | 2.10E−04 | 1.70E−03 | 8.1 | + | + |
| T017000530 | . | A | . | L | . | M | . | . | . | 2.21E−04 | 1.82E−03 | 8.2 | + | + |
| T017000473 | . | Y | Q | . | Y | M | . | . | . | 2.87E−04 | 2.38E−03 | 8.3 | + | + |
| T017000418 | . | A | . | L | Y | M | . | . | . | 2.25E−04 | 1.87E−03 | 8.3 | + | + |
| T017000611 | . | . | Q | . | Y | . | K | . | . | 1.44E−02 | 1.71E−03 | 8.4 | − | + |
| T017000386 | . | S | . | L | . | . | . | W | W | 2.42E−04 | 2.06E−03 | 8.5 | + | + |
| T017000382 | . | S | . | . | Y | M | . | . | W | 3.39E−04 | 2.89E−03 | 8.5 | + | + |
| T017000419 | . | A | . | L | Y | M | . | W | . | 1.28E−04 | 1.10E−03 | 8.6 | + | + |
| T017000774 | . | . | Q | . | Y | M | K | W | . | 3.74E−03 | 4.31E−04 | 8.7 | − | + |
| T017000569 | . | . | . | L | . | M | . | W | . | 3.43E−04 | 3.08E−03 | 9.0 | + | + |
| T017000365 | . | A | . | L | . | M | . | W | W | 2.21E−04 | 2.01E−03 | 9.1 | + | + |
| T017000434 | . | S | . | L | Y | M | . | . | . | 1.68E−04 | 1.53E−03 | 9.1 | + | + |
| T017000612 | . | . | Q | L | . | L | K | . | . | 9.72E−03 | 1.05E−03 | 9.3 | − | + |
| T017000610 | . | . | Q | . | . | L | K | . | . | 8.43E−03 | 8.57E−04 | 9.8 | − | + |
| T017000766 | . | Y | . | . | Y | M | K | . | . | 2.46E−03 | 2.39E−04 | 10.3 | − | + |
| T017000750 | Y | A | Q | . | Y | M | . | W | . | 3.33E−04 | 3.73E−03 | 11.2 | + | + |
| T017000742 | . | Y | Q | . | Y | M | . | W | W | 3.47E−04 | 3.91E−03 | 11.3 | + | + |
| T017000577 | W | . | Q | . | . | M | . | . | . | 1.53E−04 | 1.88E−03 | 12.3 | + | + |
| T017000609 | . | . | Q | . | . | . | K | . | . | 1.63E−02 | 1.14E−03 | 14.3 | − | + |
| T017000355 | W | . | Q | L | Y | M | . | W | . | 9.93E−05 | 1.45E−03 | 14.6 | + | + |
| T017000403 | W | . | Q | . | . | M | . | W | . | 1.01E−04 | 1.52E−03 | 15.0 | + | + |
| T017000578 | Y | . | Q | L | Y | M | . | W | . | 1.99E−04 | 3.49E−03 | 17.5 | + | + |
| T017000415 | W | . | Q. | . | Y | M | . | W | . | 4.90E−05 | 9.18E−04 | 18.7 | + | + |
| T017000368 | W | . | Q | . | Y | M | . | . | . | 6.47E−05 | 1.29E−03 | 19.9 | + | + |
| T017000398 | Y | . | Q | . | Y | M | . | W | . | 1.17E−04 | 2.63E−03 | 22.5 | + | + |
| T017000377 | W | . | . | . | Y | M | . | . | . | 9.22E−05 | 2.14E−03 | 23.2 | + | + |
| T017000411 | W | . | . | . | Y | M | . | W | . | 4.44E−05 | 1.56E−03 | 35.1 | + | + |
| T017000550 | . | S | Q | . | . | M | . | . | W | 1.33E−03 | 9.50E−03 | 7.1 | − | − |
| T017000518 | Y | S | Q | L | . | M | . | W | . | 2.65E−03 | 2.68E−02 | 10.1 | − | − |

* The gap in off-rate for human/cyno cross-reactivity of an ISVD is calculated based on the off-rates measured for the ISVD on humanTCR and cynoTCR zipper protein, by dividing the higher off-rate value with the lower off-rate value.

### 6.5 Example 5: Further Affinity Maturation and Sequence Optimization

In a second wave, a further panel of sequence optimized variants was generated with additional mutations M77T and F91Y in the frameworks, resulting in a set of variants which all contain the following mutations in the framework regions: L11V, M77T, K83R, V89L, and F91Y. On top of these 5 mutations in the framework regions, mutations for affinity maturation in the CDR regions, were some of the following: G26W, G26Y, D27Y, D27E, D27A, D27S, K30Q, L34Y, G35T in CDR1 and F95L, R97K, and Y99W in CDR3. The second wave ISVD variants and their CDR mutations are depicted in Tables 7 and 8 with parental ISVD T0170056G05 and the basic sequence optimized variant T017000141.

The ISVD variants were characterized for binding to CHO-K1 huTCR(2XN9)/huCD3 or to human and cynomolgus primary T cells. In brief, dose-dependent binding of purified monovalent ISVDs (Flag3-His6-tagged) to human or cynomolgus TCR/CD3 expressed on cells was evaluated by flow cytometry. In brief, cells were harvested and transferred to a V-bottom 96-well plate (Greiner Bio-one, 651 180; 5×10⁵ cells/well) and incubated with serial dilutions of ISVDs for 30 min at 4° C. in FACS buffer (D-PBS from Gibco, with 2% FBS from Sigma and 0.05% sodium azide from Merck). Next, cells were washed 3 times with FACS buffer and incubated with 1 μg/ml ANTI-FLAG® M2 Ab (Sigma, F1804) and washed again. The cell binding was detected after incubation for 30 min at 4° C. with R-Phycoerythrin-AffiniPure F(ab′)2 Fragment Goat Anti-Mouse IgG, Fc gamma Fragment Specific (Jackson Immunoresearch, 115-116-07). Subsequently, cells were resuspended in FACS buffer supplemented with 5 nM TO-PRO®-3 Iodide to distinguish live from dead cells, which are removed during the gating procedure. Cells were analyzed using a FACS Array flow cytometer (BD Biosciences) and Flowing Software. First a P1 population which represented more than 80% of the total cell population was selected based on FSC-SSC distribution. In this gate, 10000 cells were counted during acquisition. From this population the TO-PRO®-3+cells (dead cells) were excluded and the mean fluorescence intensity (MFI) PE value was calculated.

The ISVD variants were characterized further by off-rate analysis on humanTCR- and cynoTCR-zipper proteins, and determination of the melting temperature (Tm) as described in Example 4.

The generated variants and obtained results can be found in Tables 7 and 8. Binding data obtained on cynomolgus primary T cells is depicted in FIG. 2.

TABLE 7
Off-rate data for second wave ISVD variants. CDR mutations are indicated by their amino acid one letter
code at their respective position in the CDR (according to Kabat numbering). A dot reflects that
at a certain position the amino acid is identical to the amino acid at the same position in the CDR
as for the basic sequence optimized variant T017000141. If the mutation results in an improved off-
rate on either human or cyno TCR, this is indicated as a plus in the column listing the effect. The
effect column additionally depicts the relative difference for binding to human and cyno TCR.

| | | | | | | | | | | | Effect | | |
|---|---|---|---|---|---|---|---|---|---|---|---|---|---|
| | | | | | | | | | Off-rate analysis | | Gap in | | |
| | | | | | | | | | (k_{d} (1/s)) | | off-rate | | |
| | CDR1 | | | | | CDR3 | | | humanTCR | cynoTCR | human vs | | |
| ISVD ID | G26 | D27 | K30 | L34 | G35 | F95 | R97 | Y99 | zipper | zipper | cyno* | human | cyno |
| T017000141 | . | . | . | . | . | . | . | . | 9.70E−04 | 1.20E−02 | 12.4 | + | + |
| T017000623 | W | . | . | Y | . | L | K | W | 4.50E−04 | 4.70E−04 | 1.0 | + | + |
| T017000624 | . | Y | . | Y | . | L | . | W | 3.30E−04 | 1.50E−03 | 4.5 | + | + |
| T017000625 | Y | . | . | Y | . | L | K | W | 1.10E−03 | 1.10E−03 | 1.0 | − | + |
| T017000635 | W | . | Q | Y | . | L | . | W | <3.4E−05 | 1.90E−03 | >55.9 | + | + |
| T017000638 | W | . | . | Y | T | L | . | W | 7.30E−05 | 6.30E−03 | 86.3 | + | + |
| T017000641 | W | . | . | Y | T | L | K | W | 5.00E−04 | 5.40E−04 | 1.1 | + | + |
| T017000682 | . | A | . | Y | T | L | . | W | 1.70E−04 | 1.69E−03 | 9.9 | + | + |
| T017000685 | . | E | Q | Y | T | L | . | W | 4.95E−04 | 3.99E−03 | 8.1 | + | + |
| T017000686 | . | S | . | Y | T | L | . | W | 1.57E−04 | 1.39E−03 | 8.9 | + | + |
| T017000687 | . | S | Q | Y | T | L | . | W | 5.85E−04 | 4.27E−03 | 7.3 | + | + |
| T017000690 | W | E | . | Y | T | L | . | W | 8.85E−05 | 9.83E−04 | 11.1 | + | + |
| T017000691 | W | E | . | Y | T | L | . | W | <2.5E−05 | 1.54E−03 | >61.1 | + | + |

*The gap in off-rate for human/cyno cross-reactivity of an ISVD is calculated based on the off-rates measured for the ISVD on humanTCR and cynoTCR zipper protein, by dividing the higher off-rate value with the lower off-rate value.

All variants displayed a slower off-rate for both the human TCR- and cyno TCR-zipper proteins. Four second wave variants, i.e. T017000623, T017000624, T017000625 and T017000641, were identified with a gap for human/cyno cross-reactivity of 5-fold or lower. The gap might either be in favor for human TCR or cyno TCR binding. Variants T017000635, T017000638, T017000690 and T017000691 displayed the lowest off-rate on human TCR-zipper protein.

TABLE 8
Flow cytometry binding data and melting temperature for second wave ISVD variants.

| | | | | | | | | | CHO-K1 | | Human | | |
|---|---|---|---|---|---|---|---|---|---|---|---|---|---|
| | CDR1 | | | | | CDR3 | | | huTCR(2XN9)/huCD3 | | primary T cells | | Tm |
| ISVD ID | G26 | D27 | K30 | L34 | G35 | F95 | R97 | Y99 | n | EC50 (M) | n | EC50 (M) | (° C.) |
| T01700056G05 | . | . | . | . | . | . | . | . | 10 | 2.9E−08 | 17 | 1.4E−07 | |
| T017000141 | . | . | . | . | . | . | . | . | 3 | 1.9E−08 | 3 | 1.0E−07 | 77 |
| T017000623 | W | . | . | Y | . | L | K | W | 2 | 4.4E−08 | 3 | 2.8E−07 | 67 |
| T017000624 | . | Y | . | Y | . | L | . | W | 3 | 1.3E−08 | 5 | 4.6E−08 | 79 |
| T017000625 | Y | . | . | Y | . | L | K | W | 2 | 4.3E−08 | 3 | 2.3E−07 | 67 |
| T017000635 | W | . | Q | Y | . | L | . | W | 1 | 2.0E−08 | 2 | 7.0E−08 | 68 |
| T017000638 | W | . | . | Y | T | L | . | W | 1 | 2.0E−08 | 2 | 6.6E−08 | 57 |
| T017000641 | W | . | . | Y | T | L | K | W | 1 | 3.9E−08 | 2 | 2.7E−07 | 58 |
| T017000682 | . | A | . | Y | T | L | . | W | 2 | 1.7E−08 | 4 | 5.8E−08 | 79 |
| T017000685 | . | E | Q | Y | T | L | . | W | 0 | ND | 0 | ND | 78 |
| T017000686 | . | S | . | Y | T | L | . | W | 2 | 2.3E−08 | 4 | 5.5E−08 | 77 |
| T017000687 | . | S | Q | Y | T | L | . | W | 0 | ND | 0 | ND | 78 |
| T017000690 | W | E | . | Y | T | L | . | W | 2 | 2.2E−08 | 4 | 6.6E−08 | 65 |
| T017000691 | W | E | . | Y | T | L | . | W | 2 | 1.9E−08 | 4 | 1.5E−07 | 68 |

ND: not determined

### 6.6 Example 6: Construction and Characterisation of Bivalent CD123/TCR Bispecific ISVD Formats

To explore the impact of affinity maturation and sequence optimization of the TCR building block on the affinity and potency of a T cell engager formats, CD123/TCR bispecific ISVD formats were generated. The different TCR ISVD variants were fused to an anti-CD123 ISVD (A0110056A10) as target binding moiety in both orientations using a 35GS linker. For comparison purposes, similar formats were generated using the parental ISVD T0170056G05.

The generated formats are listed in Table 9.

TABLE 9
Format ID and description of CD123/TCR bispecific ISVD formats and controls

| Format ID | Description |
|---|---|
| T017000128 | T0170056G05(E1D)-35GS-A0110056A10-A |
| T017000131 | A0110056A10(E1D)-35GS-T0170056G05-A |
| T017000627 | T0170056G05(E1D, L11V, G26W, L34Y, M77T, K83R, V89L, F91Y, F95L, R97K, Y99W)- |
| | 35GS-A0110056A10-A |
| T017000628 | T0170056G05(E1D, L11V, G26Y, L34Y, M77T, K83R, V89L, F91Y, F95L, R97K, Y99W)- |
| | 35GS-A0110056A10-A |
| T017000630 | A0110056A10(E1D)-35GS- |
| | T0170056G05(L11V, G26W, L34Y, M77T, K83R, V89L, F91Y, F95L, R97K, Y99W)-A |
| T017000631 | A0110056A10(E1D)-35GS- |
| | T0170056G05(L11V, G26Y, L34Y, M77T, K83R, V89L, F91Y, F95L, R97K, Y99W)-A |
| T017000633 | T0170056G05(L11V, D27Y, L34Y, M77T, K83R, V89L, F91Y, F95L, Y99W)-35GS- |
| | A0110056A10-A |
| T017000634 | A0110056A10(E1D)-35GS- |
| | T0170056G05(L11V, D27Y, L34Y, M77T, K83R, V89L, F91Y, F95L, Y99W)-A |
| T017000636 | T0170056G05(L11V, G26W, K30Q, L34Y, M77T, K83R, V89L, F91Y, M95L, Y99W)- |
| | 35GS-A0110056A10-A |
| T017000637 | A0110056A10(E1D)-35GS- |
| | T0170056G05(L11V, G26W, K30Q, L34Y, M77T, K83R, V89L, F91Y, M95L, Y99W)-A |
| T017000639 | T0170056G05(L11V, G26W, L34Y, G35T, M77T, K83R, V89L, F91Y, F95L, Y99W)- |
| | 35GS-A0110056A10-A |
| T017000640 | A0110056A10(E1D)-35GS- |
| | T0170056G05(L11V, G26W, L34Y, G35T, M77T, K83R, V89L, F91Y, F95L, Y99W)-A |
| T017000642 | T0170056G05(L11V, G26W, L34Y, G35T, M77T, K83R, V89L, F91Y, F95L, R97K, Y99W)- |
| | 35GS-A0110056A10-A |
| T017000643 | A0110056A10(E1D)-35GS- |
| | T0170056G05(L11V, G26W, L34Y, G35T, M77T, K83R, V89L, F91Y, F95L, R97K, |
| | Y99W)-A |

The CD123/TCR bispecific ISVD formats were characterized for redirected T cell mediated killing in a flow cytometry-based cytotoxicity assay using human or cynomolgus primary T cells as effector cells and MOLM-13 (DSMZ ACC 554), i.e. a cell line expressing CD123, as target cells. Target cells were labelled with 4 μM PKH-26 membrane dye using the PKH26 red fluorescent cell linker kit (Sigma, PKH26GL-1KT) according to manufacturer's instructions. Effector cells (2.5×10⁵ cells/well) and PKH-labelled target cells (2.5×10⁴ cells/well) were co-incubated in 96-well V-bottom plates (Greiner Bio-one, #651 180) (effector versus target ratio of 10:1) in assay medium of the target cell line (target growth medium without antibiotics). For analysis of concentration-dependent cell lysis, serial dilutions of ISVD formats in target assay medium were added to the cells and incubated for 18 h in a 5% CO2 atmosphere at 37° C. After incubation, cells were pelleted by centrifugation and washed with FACS buffer (D-PBS from Gibco, with 10% FBS from Sigma and 0.05% sodium azide from Merck). Subsequently, cells were resuspended in FACS buffer supplemented with 5 nM TO-PRO®-3 Iodide (642/661) (ThermoFisher Scientific, T3605) to distinguish live from dead cells. Cells were analyzed using a FACS Array flow cytometer (BD Biosciences). Per sample, a total sample volume of 80 μL was acquired. Gating was set on PKH26 positive cells, and within this population, the TO-PRO®-3 positive cells were determined. Percent specific lysis=((% TO-PRO®-3+no ISVD-% TO-PRO®-3+with ISVD)/(% TO-PRO®-3+no ISVD))×100. EC50 values and % lysis obtained in the MOLM-13 killing assay with human T cells are summarized in Table 10. The data obtained with cyno T cells are depicted in Table 11.

TABLE 10
EC50 (M) and % lysis for CD123/TCR ISVD formats in the flow cytometry based T cell mediated
MOLM-13 cell killing assay using human primary T cells at an E:T ratio of 10:1.

| | Format ID (TCR | | | | Format ID | | | |
|---|---|---|---|---|---|---|---|---|
| ISVD ID (TCR | variant - CD123 | | | | (CD123 ISVD - | | | |
| variant) | ISVD) | n | EC50 (M) | % Lysis | TCR variant) | n | EC50 (M) | % Lysis |
| T0170056G05 | T017000128 | 8 | 2.3E−10 | 32 | T017000131 | 1 | 1.3E−09 | 20 |
| T017000623 | T017000627 | 3 | 2.4E−10 | 36 | T017000630 | 1 | Negative | 0 |
| T017000625 | T017000628 | 1 | 1.6E−10 | 39 | T017000631 | 1 | Negative | 0 |
| T017000624 | T017000633 | 2 | 1.2E−10 | 31 | T017000634 | 2 | 1.4E−10 | 26 |
| T017000635 | T017000636 | 1 | 6.6E−11 | 36 | T017000637 | 1 | Negative | 0 |
| T017000638 | T017000639 | 1 | 3.6E−11 | 42 | T017000640 | 1 | 5.7E−10 | 37 |
| T017000641 | T017000642 | 1 | 3.0E−10 | 32 | T017000643 | 1 | Negative | 0 |

TABLE 11
EC50 (M) and % lysis for CD123/TCR ISVD formats in the flow cytometry based T cell mediated
MOLM-13 cell killing assay using cynomolgus primary T cells at an E:T ratio of 10:1.

| | Format ID (TCR | | | | Format ID | | | |
|---|---|---|---|---|---|---|---|---|
| ISVD ID (TCR | variant - CD123 | | | | (CD123 ISVD - | | | |
| variant) | ISVD) | n | EC50 (M) | % Lysis | TCR variant) | n | EC50 (M) | % Lysis |
| T0170056G05 | T017000128 | 6 | 1.7E−10 | 37 | T017000131 | 1 | 9.0E−10 | 14 |
| T017000623 | T017000627 | 3 | 3.8E−11 | 19 | T017000630 | 1 | 4.5E−10 | 26 |
| T017000625 | T017000628 | 1 | 2.0E−11 | 40 | T017000631 | 1 | 2.0E−10 | 35 |
| T017000624 | T017000633 | 2 | 5.2E−11 | 35 | T017000634 | 2 | 1.2E−10 | 32 |
| T017000635 | T017000636 | 1 | 6.7E−11 | 20 | T017000637 | 1 | No clear fit | 17 |
| T017000638 | T017000639 | 1 | 4.1E−11 | 28 | T017000640 | 1 | Negative | 6 |
| T017000641 | T017000642 | 1 | 4.0E−11 | 37 | T017000643 | 1 | 1.6E−10 | 12 |

All formats containing the different TCR ISVD variants at the N-terminal position were at least as potent as the format containing T0170056G05 as TCR building block in the human T cell mediated MOLM-13 cell killing assay. Compared to format T017000128 with the parental ISVD T01700056G05 at the N-terminus, improved potency could be observed for all the formats with an anti-TCR ISVD variant at N-terminal position in the assay with cynomolgus T cells.

In the set of the formats containing the different anti-TCR ISVD variants at the C-terminal position, the most potent format in the T cell mediated MOLM-13 cell killing assay was T017000634, which comprises the anti-TCR ISVD variant T017000624. The potency was significantly improved compared to T017000131, which comprises the parental ISVD T0170056G05 at the C-terminus, in the assay with human and with cynomolgus primary T cells.

### 6.7 Example 7: Binding of the CD123/TCR Bispecific ISVD Formats to TCR Protein Via SPR

Binding affinities of the CD123/TCR bispecific ISVD formats for recombinant soluble human and cynomolgus TCR were determined by SPR at 25° C. on a ProteOn XPR36 instrument. HuTCR-zipper or cyTCR-zipper protein was immobilized on a GLC sensor chip (between 100-200 RU) via amine coupling chemistry, using EDC/NHS for activation of the carboxyl groups on the chip surface (running buffer: HBS-EP+, pH7.4). Purified ISVD formats were injected for 2 min (at a flow rate of 45 μL/min) at different concentrations (between 4.1 nM and 1000 nM) and dissociation was followed for 900 s. Data were double referenced by subtracting a reference ligand lane and a blank buffer injection. Processed sensorgrams were fitted with the Langmuir model (1:1 interaction) using ProteOn Manager 3.1.0 (Version 3.1.0.6) software.

The data for the formats with anti-TCR ISVD at the N-terminus are shown in Table 12. Those for the formats with anti-TCR ISVD at the C-terminus are depicted in and Table 13.

TABLE 12
Binding affinities of ISVD formats for human TCR-zipper protein
as determined by SPR on a ProteOn XPR36 instrument at 25° C.

| | Format ID | | | | Format ID | | | |
|---|---|---|---|---|---|---|---|---|
| ISVD ID | (TCR variant - | kₐ | k_{d} | KD | (CD123 ISVD - | kₐ | k_{d} | KD |
| (TCR variant) | CD123 ISVD) | (1/Ms) | (1/s) | (M) | TCR variant) | (1/Ms) | (1/s) | (M) |
| T0170056G05 | T017000128 | 3.6E+04 | 8.0E−04 | 2.2E−08 | T017000131 | 1.2E+04 | 9.7E−04 | 7.9E−08 |
| T017000623 | T017000627 | 5.0E+04 | 4.5E−04 | 9.1E−09 | T017000630 | 8.3E+03 | 2.2E−03 | 2.6E−07 |
| T017000625 | T017000628 | 5.1E+04 | 1.1E−03 | 2.2E−08 | T017000631 | 8.5E+03 | 1.8E−03 | 2.1E−07 |
| T017000624 | T017000633 | 6.9E+04 | 3.0E−04 | 4.3E−09 | T017000634 | 2.6E+04 | 6.0E−04 | 2.3E−08 |
| T017000635 | T017000636 | 1.5E+05 | 4.0E−05 | 2.7E−10 | T017000637 | No binding | | |
| T017000638 | T017000639 | 9.8E+04 | 7.8E−05 | 8.0E−10 | T017000640 | 1.7E+04 | 1.0E−03 | 6.1E−08 |
| T017000641 | T017000642 | 5.4E+04 | 4.2E−04 | 7.9E−09 | T017000643 | 6.5E+03 | 4.8E−03 | 7.4E−07 |

TABLE 13
Binding affinities of ISVD formats for cynomolgus TCR-zipper protein
as determined by SPR on a ProteOn XPR36 instrument at 25° C.

| | Format ID | | | | Format ID | | | |
|---|---|---|---|---|---|---|---|---|
| ISVD ID | (TCR variant - | kₐ | k_{d} | KD | (CD123 ISVD - | kₐ | k_{d} | KD |
| (TCR variant) | CD123 ISVD) | (1/Ms) | (1/s) | (M) | TCR variant) | (1/Ms) | (1/s) | (M) |
| T0170056G05 | T017000128 | 5.0E+04 | 1.8E−02 | 3.6E−07 | T017000131 | 1.5E+04 | 2.5E−02 | 1.6E−06 |
| T017000623 | T017000627 | 8.3E+04 | 4.3E−04 | 5.2E−09 | T017000630 | 9.5E+03 | 2.3E−03 | 2.4E−07 |
| T017000625 | T017000628 | 8.0E+04 | 9.1E−04 | 1.1E−08 | T017000631 | 9.0E+03 | 2.6E−03 | 2.9E−07 |
| T017000624 | T017000633 | 1.0E+05 | 1.7E−03 | 1.7E−08 | T017000634 | 2.7E+04 | 5.3E−03 | 2.0E−07 |
| T017000635 | T017000636 | 8.3E+04 | 2.4E−03 | 2.9E−08 | T017000637 | No binding | | |
| T017000638 | T017000639 | 1.1E+05 | 4.9E−03 | 4.4E−08 | T017000640 | 5.8E+03 | 8.6E−02 | 1.5E−05 |
| T017000641 | T017000642 | 6.8E+04 | 3.7E−04 | 5.5E−09 | T017000643 | 6.0E+03 | 5.4E−03 | 9.0E−07 |

All formats containing the different TCR ISVD variants at the N-terminal position have at least similar binding affinity to human TCR as the format containing T0170056G05 as TCR building block. Compared to format T017000128 with the parental ISVD T01700056G05 at the N-terminus, improved binding affinity for cyno TCR could be observed for all the formats with an anti-TCR ISVD variant at N-terminal position.

T017000624 is the only variant for which improved affinity can be observed on both human and cyno recombinant proteins when located at either the C- or the N-terminal position of the bispecific ISVD format, in comparison to the reference formats containing the parental ISVD T0170056G05.

This ISVD variant was therefore further characterized in depth as monovalent and in the context of a CD123/TCR bispecific ISVD format.

### 6.8 Example 8: Analysis of Binding to TCR Protein Via SPR on ProteOn SPR36

The sequence optimized, affinity matured anti-TCR ISVD T017000624 was characterized as purified monovalent ISVD in comparison with parental ISVD T0170056G05 to evaluate affinity and stability.

TABLE 14
List of amino acid differences for parental anti-TCR ISVD (T0170056G05),
the basic sequence optimization variant (T017000141) and the sequence
optimized, affinity matured variant T017000624.

| | ID | | | | | | | | |
|---|---|---|---|---|---|---|---|---|---|
| | FR1 | CDR1 | | FR3 | | | | CDR3 | |
| | VHH2 germline | | | | | | | | |
| | L11 | X27 | X34 | T77 | K83 | V89 | Y91 | X95 | X99 |
| T0170056G05 | L | D | L | M | K | V | F | F | Y |
| T017000141 | V | . | . | . | R | L | . | . | . |
| T017000624 | V | Y | Y | T | R | L | Y | L | W |

Analysis of binding kinetics of T017000624 and T0170056G05 to directly immobilized huTCR-zipper and cyTCR-zipper proteins was performed at 25° C.

Affinities of T0170056G05 for recombinant soluble human and cynomolgus TCR were determined using Bio-Layer Interferometry (BLI) on an Octet RED384 instrument (Pall ForteBio Corp.). HuTCR-zipper or cyTCR-zipper protein was covalently immobilized on amine-reactive sensors via NHS/EDC coupling chemistry. For kinetic analysis, sensors were first dipped into running buffer (10 mM Hepes, 150 mM NaCl, 0.05% p20, pH 7.4 from GE Healthcare Life Sciences) to determine the baseline setting. Subsequently, sensors were dipped into wells containing different concentrations of purified ISVDs (ranging from 1.4 nM to 1 mM) for the association step (180 s) and transferred to wells containing running buffer for the dissociation step (15 min). Affinity constants (KD) were calculated applying a 1:1 interaction model using the ForteBio Data Analysis software.

Affinities of T017000624 for recombinant soluble human and cynomolgus TCR were determined by SPR on a ProteOn XPR36 instrument. HuTCR-zipper or cyTCR-zipper protein was immobilized on a GLC sensor chip (between 100-200 RU) via amine coupling chemistry, using EDC/NHS for activation of the carboxyl groups on the chip surface (running buffer: HBS-EP+, pH7.4). Purified ISVDs were injected for 2 min (at a flow rate of 45 μL/min) at different concentrations (between 4.1 nM and 1000 nM) and dissociation was followed for 900 s. Data were double referenced by subtracting a reference ligand lane and a blank buffer injection. Processed sensorgrams were fitted with the Langmuir model (1:1 interaction) using ProteOn Manager 3.1.0 (Version 3.1.0.6) software.

The binding characteristics are listed in Table 15.

TABLE 15
Binding affinity of ISVDs for huTCR-zipper and cyTCR-zipper protein
as determined by surface plasmon resonance using Proteon XPR36.

| | huTCR-zipper protein | | | cyTCR-zipper protein | | |
|---|---|---|---|---|---|---|
| ISVD ID | kₐ (1/Ms) | k_{d} (1/s) | KD (M) | kₐ (1/Ms) | k_{d} (1/s) | KD (M) |
| T0170056G05 | 4.2E+04 | 1.6E−03 | 3.8E−08 | 7.4E+04 | 1.2E−02 | 1.7E−07 |
| T017000141 | 6.5E+04 | 1.2E−03 | 1.8E−08 | 8.0E+04 | 1.8E−02 | 2.2E−07 |
| T017000624 | 1.4E+05 | 3.3E−04 | 2.4E−09 | 2.4E+05 | 1.5E−03 | 6.3E−09 |

On- and off-rate of T017000624 for huTCR-zipper protein were improved by ˜3 to ˜5-fold, respectively, compared to T0170056G05 resulting in a 15-fold higher affinity (KD). For cyTCR-zipper protein the off-rate of T017000624 was improved by 8-fold compared to T0170056G05, which in combination with a 3-fold improved on-rate resulted in a 26-fold higher affinity (KD). The difference in human-cynomolgus cross-reactivity for T017000624 based on KD is 2.6-fold compared to a 4.5-fold for the parental ISVD.

### 6.9 Example 9: Binding of ISVDs to TCR/CD3 Transfected Cells Via Flow Cytometry

The sequence optimized, affinity matured anti-TCR ISVD T017000624 was characterized for binding to TCR expressed on cells in a flow cytometry assay and compared with the parental ISVD T0170056G05 and an irrelevant anti-egg lysozyme ISVD cAbLys3, the latter taking along as negative control.

Dose-dependent binding of purified monovalent ISVDs (Flag3-His6-tagged) to human or cynomolgus TCR/CD3 expressed on cells was evaluated by flow cytometry. In brief, cells were harvested and transferred to a V-bottom 96-well plate (Greiner Bio-one, 651 180; 5×10¹ cells/well) and incubated with serial dilutions of ISVDs for 30 min at 4° C. in FACS buffer (D-PBS from Gibco, with 2% FBS from Sigma and 0.05% sodium azide from Merck). Next, cells were washed 3 times with FACS buffer and incubated with 1 μg/ml ANTI-FLAG® M2 Ab (Sigma, F1804) and washed again. The cell binding was detected after incubation for 30 min at 4° C. with R-Phycoerythrin-AffiniPure F(ab′)2 Fragment Goat Anti-Mouse IgG, Fc gamma Fragment Specific (Jackson Immunoresearch, 115-116-07). Subsequently, cells were resuspended in FACS buffer supplemented with 5 nM TO-PRO®-3 Iodide to distinguish live from dead cells, which are removed during the gating procedure. Cells were analyzed using a FACS Array flow cytometer (BD Biosciences) and Flowing Software. First a P1 population which represented more than 80% of the total cell population was selected based on FSC-SSC distribution. In this gate, 10000 cells were counted during acquisition. From this population the TO-PRO®-3+ cells (dead cells) were excluded and the mean fluorescence intensity (MFI) PE value was calculated.

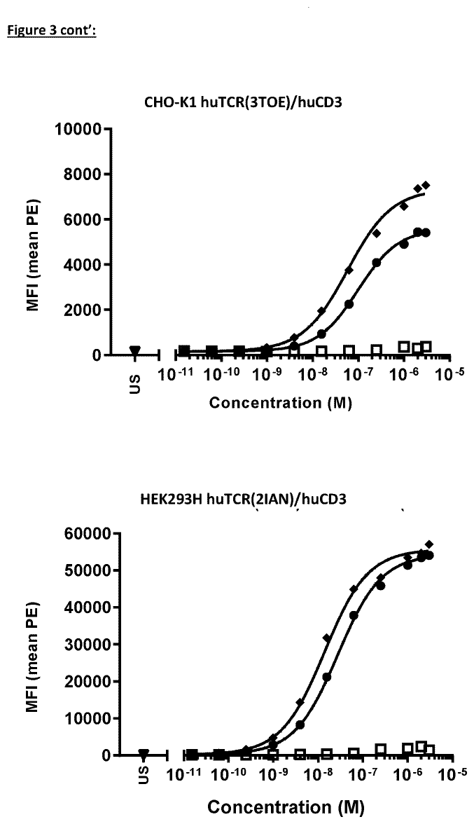

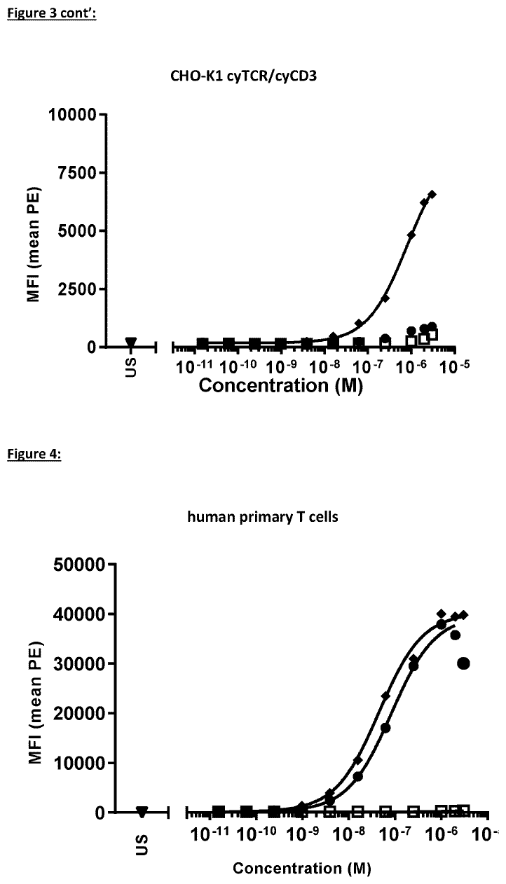

Dose titration curves are shown in FIG. 3. EC50 values obtained from the dose response curves are summarized in Tables 16 and 17.

TABLE 16
EC50 (M) and MFI (@1 μM ISVD concentration) for binding of T0170056G05
and T017000624 to human TCR/CD3 transfected cells determined via flow cytometry.

| | CHO-K1 huTCR(2XN9)/huCD3 | | | | CHO-K1 huTCR(3TOE)/huCD3 | | | |
|---|---|---|---|---|---|---|---|---|
| | | EC50 95% | EC50 95% | MFI at | | EC50 95% | EC50 95% | MFI at |
| ISVD ID | EC50 (M) | LCI (M) | UCI (M) | 1 μM | EC50 (M) | LCI (M) | UCI (M) | 1 μM |
| T0170056G05 | 3.8E−08 | 3.0E−08 | 4.9E−08 | 44278 | 9.8E−08 | 7.9E−08 | 1.2E−07 | 5585 |
| T017000624 | 2.2E−08 | 1.8E−08 | 2.7E−08 | 43178 | 5.8E−08 | 4.7E−08 | 7.2E−08 | 7344 |

LCI: lower confidence interval,
UCI: upper confidence interval

TABLE 17
EC50 (M) and MFI (@1 μM ISVD concentration) for binding of T0170056G05 and T017000624
to human and cynomolgus TCR/CD3 transfected cells determined via flow cytometry.

| | HEK293H huTCR(2IAN)/huCD3 | | | | CHO-K1 cyTCR/cyCD3 | | | |
|---|---|---|---|---|---|---|---|---|
| | | EC50 95% | EC50 95% | MFI at | | EC50 95% | EC50 95% | MFI at |
| ISVD ID | EC50 (M) | LCI (M) | UCI (M) | 1 μM | EC50 (M) | LCI (M) | UCI (M) | 1 μM |
| T0170056G05 | 2.8E−08 | 2.2E−08 | 3.5E−08 | 54285 | No fit | / | / | 888 |
| T017000624 | 1.4E−08 | 1.1E−08 | 1.8E−08 | 55704 | No fit | / | / | 6565 |

LCI: lower confidence interval,
UCI: upper confidence interval

EC50 values indicate that the affinity of the T017000624 was 1.7-fold higher to CHO-K1 huTCR(2XN9)/huCD3, 1.7-fold higher to CHO-K1 huTCR(3TOE)/huCD3 and 2-fold higher to HEK293H huTCR(2IAN)/CD3 cells compared to T01700056G05 with a slightly higher MFI on CHO-K1 huTCR(3TOE)/huCD3. Binding of T017000624 to the cyTCR/cyCD3 transfected cell line was much stronger compared to T01700056G05 but due to incomplete titration curves it was not possible to determine EC50 values.

### 6.10 Example 10: Binding of ISVDs to Primary T Cells Via Flow Cytometry

The sequence optimized, affinity matured anti-TCR ISVD T017000624 was characterized for binding to TCR expressed on primary T cells in a flow cytometry assay and compared with the parental ISVD T0170056G05 and an irrelevant anti-egg lysozyme ISVD cAbLys3, the latter taken along as negative control.

Dose-dependent binding of purified monovalent ISVDs (Flag3-His6-tagged) to human and cynomolgus primary T cells was evaluated by flow cytometry as described in example 5.

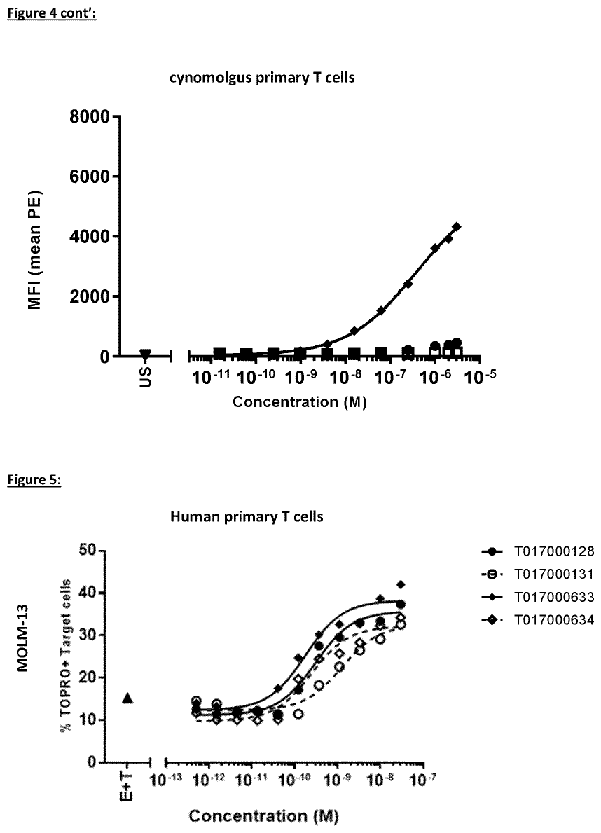

Dose titration curves and shown in FIG. 4. EC50 values obtained from the dose response curves are summarized in Table 18.

TABLE 18
EC50 (M) and MFI (@1 μM ISVD concentration) for
binding of T0170056G05 and T017000624 to human and
cynomolgus primary T cells determined via flow cytometry.

| | human primary T cells | | | | cynomolgus primary T cells | | |
|---|---|---|---|---|---|---|---|
| | | EC50 95% | EC50 95% | MFI @ | | | MFI |
| ISVD ID | EC50 (M) | LCI (M) | UCI (M) | Top | EC50 (M) | Top | @1 μM |
| T0170056G05 | 8.3E−08 | 6.7E−08 | 9.9E−08 | 39682 | No fit | / | 343 |
| T017000624 | 4.5E−08 | 3.8E−08 | 5.3E−08 | 40250 | No fit | / | 3613 |

LCI: lower confidence interval,
UCI: upper confidence interval

The dose titration curves indicate a very similar binding pattern of T017000624 to human and cynomolgus primary T cells compared to the TCR/CD3 transfected cell lines (Example 9). Affinity of T017000624 as indicated by the EC50 value is 1.8-fold higher compared to T0170056G05 for the human primary T cells and markedly improved for the cynomolgus primary T cells, which could not be quantified due to incomplete titration curves.

### 6.11 Example 11: Functional Characterization of CD123/TCR Bispecific ISVD Formats in T Cell Mediated Killing Assays

To further compare the sequence optimized, affinity matured anti-TCR ISVD T017000624 with the parental ISVD T017056G05 in the context of a T cell engager format, the bispecific formats wherein the T017000624 and T0170056G05 anti-TCR ISVDs are fused to the anti-CD123 ISVD A0110056A10, were further characterized in two different T cell mediated killing assays. The formats are listed in Table 19.

TABLE 19
Format ID and description of CD123/TCR bispecific formats

| Format ID | Description |
|---|---|
| T017000128 | T0170056G05(E1D)-35GS-A0110056A10-A |
| T017000131 | A0110056A10(E1D)-35GS-T0170056G05-A |
| T017000633 | T0170056G05(L11V, D27Y, L34Y, M77T, K83R, V89L, F91Y, F95L, Y99W)-35GS- |
| | A0110056A10-A |
| T017000634 | A0110056A10(E1D)-35GS- |
| | T0170056G05(L11V, D27Y, L34Y, M77T, K83R, V89L, F91Y, F95L, Y99W)-A |

The CD123/TCR bispecific ISVD formats were first characterized for redirected T cell mediated killing in a flow cytometry-based cytotoxicity assay using human or cynomolgus primary T cells as effector cells and non-adherent target cells. CD123 positive target cells (MOLM-13, DSMZ ACC 554 or KG-1a, ATCC® CCL246.1™) were labelled with 4 μM PKH-26 membrane dye using the PKH26 red fluorescent cell linker kit (Sigma, PKH26GL-1KT) according to manufacturer's instructions. Effector cells (2.5×10⁵ cells/well) and PKH-labelled target cells (2.5×10⁴ cells/well) were co-incubated in 96-well V-bottom plates (Greiner Bio-one, #651 180) (effector versus target ratio of 10:1) in assay medium of the target cell line (target growth medium without antibiotics). For analysis of concentration-dependent cell lysis, serial dilutions of ISVD formats in target assay medium were added to the cells and incubated for 18 h in a 5% CO2 atmosphere at 37° C. After incubation, cells were pelleted by centrifugation and washed with FACS buffer (D-PBS from Gibco, with 10% FBS from Sigma and 0.05% sodium azide from Merck). Subsequently, cells were resuspended in FACS buffer supplemented with 5 nM TO-PRO®-3 Iodide (642/661) (ThermoFisher Scientific, T3605) to distinguish live from dead cells. Cells were analyzed using a FACS Array flow cytometer (BD Biosciences). Per sample, a total sample volume of 80 μL was acquired. Gating was set on PKH26 positive cells, and within this population, the TO-PRO®-3 positive cells were determined. Percent specific lysis=((% TO-PRO®-3+no ISVD-% TO-PRO®-3+with ISVD)/(% TO-PRO®-3+_{no ISVD}))×100.

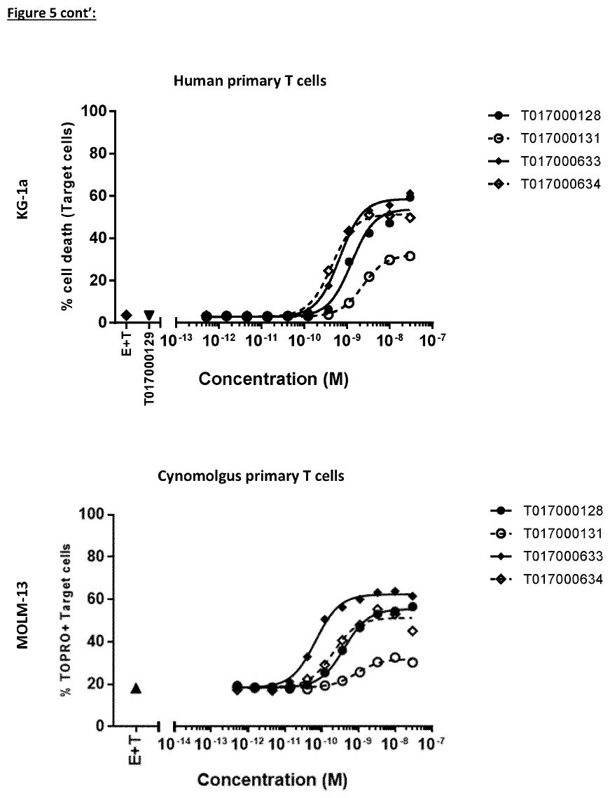

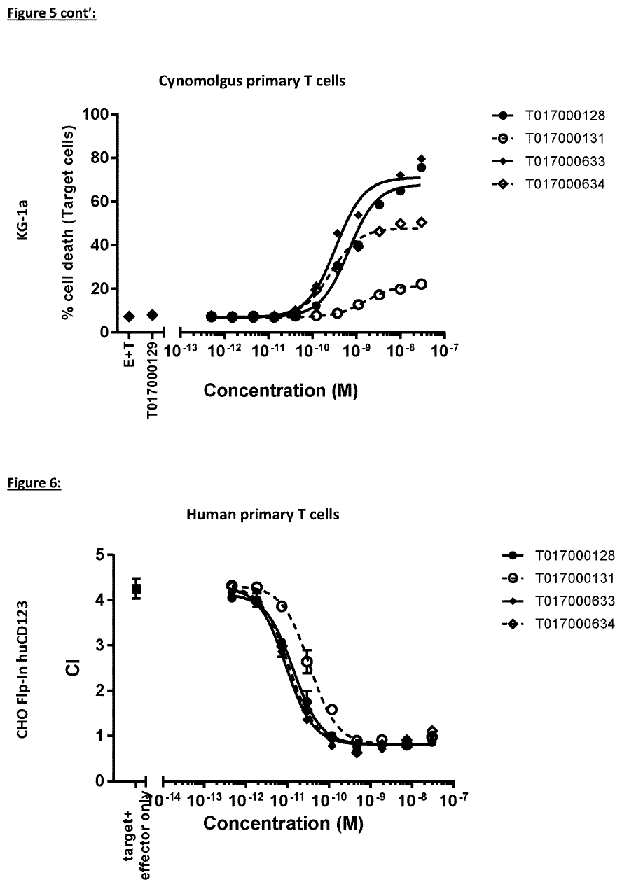

Dose titration curves are shown in FIG. 5 and IC50 values are summarized in Table 20 and Table 21.

TABLE 20
EC50 (M) and % lysis for CD123/TCR ISVD formats in the flow cytometry-based
T cell mediated MOLM-13 cell killing assay using an E:T ratio of 10:1.

| | MOLM-13 | | | | | | | |
|---|---|---|---|---|---|---|---|---|
| | Human T cell mediated killing | | | | Cynomolgus T cell mediated killing | | | |
| | | EC50 95% | EC50 95% | | | EC50 95% | EC50 95% | |
| Format ID | EC50 (M) | LCI (M) | UCI (M) | % lysis | EC50 (M) | LCI (M) | UCI (M) | % lysis |
| T017000128 | 3.0E−10 | 1.7E−10 | 5.6E−10 | 24 | 4.1E−10 | 3.2E−10 | 5.4E−10 | 37 |
| T017000131 | 1.2E−09 | 5.9E−10 | 2.7E−09 | 20 | 8.9E−10 | 4.2E−10 | 1.8E−09 | 14 |
| T017000633 | 1.8E−10 | 7.7E−11 | 4.5E−10 | 26 | 6.9E−11 | 5.6E−11 | 8.5E−11 | 44 |
| T017000634 | 2.5E−10 | 1.1E−10 | 6.0E−10 | 22 | 2.1E−10 | 1.6E−10 | 2.7E−10 | 33 |

LCI: lower confidence interval,
UCI: upper confidence interval

TABLE 21
EC50 (M) and % lysis for CD123/TCR ISVD formats in the flow cytometry-based
T cell mediated KG-1a cell killing assay using an E:T ratio of 10:1.

| | KG-1a | | | | | | | |
|---|---|---|---|---|---|---|---|---|
| | Human T cell mediated killing | | | | Cynomolgus T cell mediated killing | | | |
| | | EC50 95% | EC50 95% | | | EC50 95% | EC50 95% | |
| Format ID | EC50 (M) | LCI (M) | UCI (M) | % lysis | EC50 (M) | LCI (M) | UCI (M) | % lysis |
| T017000128 | 1.3E−09 | 1.0E−09 | 1.6E−09 | 51 | 6.7E−10 | 5.0E−10 | 8.9E−10 | 61 |
| T017000131 | 2.4E−09 | 1.6E−09 | 3.4E−09 | 29 | 1.6E−09 | 9.3E−10 | 2.7E−09 | 14 |
| T017000633 | 6.8E−10 | 5.6E−10 | 8.1E−10 | 56 | 3.4E−10 | 2.6E−10 | 4.5E−10 | 64 |
| T017000634 | 4.4E−10 | 3.6E−10 | 5.4E−10 | 49 | 2.7E−10 | 1.9E−10 | 3.6E−10 | 41 |

LCI: lower confidence interval,
UCI: upper confidence interval

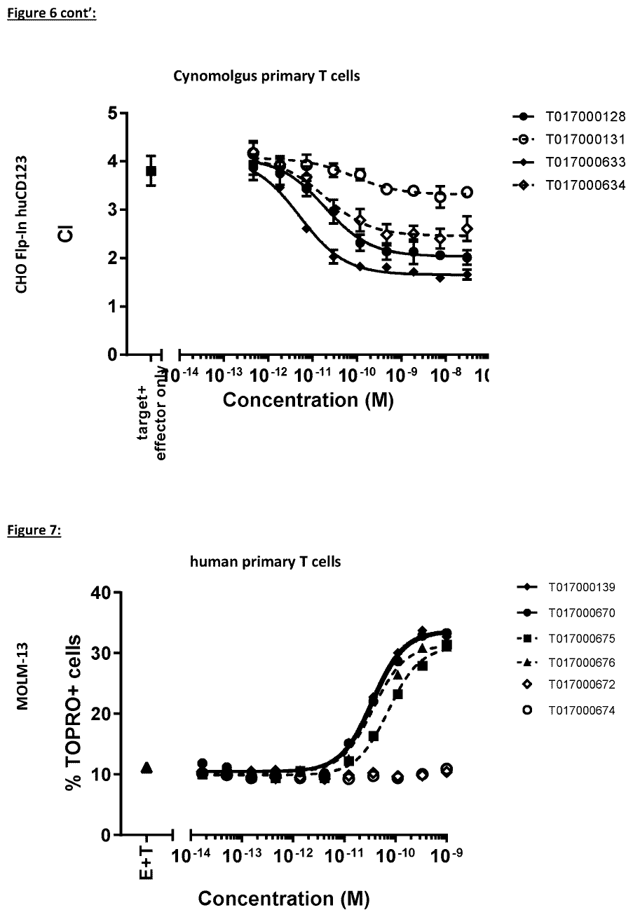

Subsequently, the CD123/TCR bispecific ISVD formats were further characterized for redirected T cell mediated killing in an impedance-based cytotoxicity assay using human or cynomolgus primary effector T cells and adherent CHO Flp-In huCD123 target cells. Changes in impedance induced by the adherence of target cells to the surface of an electrode were measured using the xCELLigence instrument (Roche). T cells are non-adherent and therefore do not impact the impedance measurements. The xCELLigence instrument (Roche) quantifies the changes in electrical impedance, displaying them as a dimensionless parameter termed cell-index (CI), which is directly proportional to the total area of tissue-culture well that is covered by cells. To each well of a 96 E-plate (ACEA Biosciences; 05 232 368 001) 50 μL of target assay medium (target growth medium without antibiotics) was added. The 96 E-plate was placed in the xCELLigence station (in the 37° C. incubator at 5% CO2) and a blank reading on the xCELLigence system was performed to measure background impedance in absence of cells. Subsequently, 50 μL target cells (2×10⁴ cells/well) in target assay medium were seeded onto the 96 E-plate, and 50 μl of serially diluted ISVD solutions in target assay medium was added. After 30 min at RT, 50 μL of primary T cells (3×10⁵ cells/well) in target assay medium were added per well to achieve an effector to target ratio of 15:1. The plate was placed in the xCELLigence station and impedance was measured every 15 min for 3 days. The data were analyzed using a fixed time point indicated in the results. Dose titration curves are shown in FIG. 6 and IC50 values are summarized in Table 22.

TABLE 22
IC50 and % inhibition for CD123/TCR ISVD formats in the
impedance based (xCELLigence) T cell mediated CHO Flp-In
huCD123 cell killing assay using an E:T ratio of 15:1.

| | Human T cell mediated killing | | | Cynomolgus T cell mediated killing | | |
|---|---|---|---|---|---|---|
| | | IC50 95% | IC50 95% | | IC50 95% | IC50 95% |
| Format ID | IC50 (M) | LCI (M) | UCI (M) | IC50 (M) | LCI (M) | UCI (M) |
| T017000128 | 1.4E−11 | 1.2E−11 | 1.6E−11 | 1.9E−11 | 1.3E−11 | 2.8E−11 |
| T017000131 | 3.4E−11 | 2.9E−11 | 3.9E−11 | 8.1E−11 | 2.5E−11 | 2.6E−10 |
| T017000633 | 9.1E−12 | 7.8E−12 | 1.1E−11 | 5.1E−12 | 3.7E−12 | 7.1E−12 |
| T017000634 | 1.1E−11 | 9.0E−12 | 1.2E−11 | 1.9E−11 | 1.1E−11 | 3.2E−11 |

LCI: lower confidence interval,
UCI: upper confidence interval

In general, the replacement of the T0170056G05 by T017000624 resulted in improved potency and efficacy across all T cell mediated cell killing assays. Improvements were more pronounced for the cynomolgus versus the human T cell setup and for formats with C-terminal vs N-terminal positioning of the T017000624 ISVD. N-terminal positioning of the TCR ISVD resulted in higher potency and efficacy. For the CD123/TCR bispecific ISVD formats containing the T0170056G05 (T017000128 and T017000131) a 2-4-fold lower potency and apart from one setup (xCELLigence, hu T cells) a significantly lower efficacy (% cell lysis) was observed when the TCR ISVD was at the C-terminal position (T017000131). These positional differences were slightly less pronounced for potency and efficacy of T017000633 and T017000634, the CD123/TCR bispecific ISVD formats containing the T017000624 ISVD. The most potent and efficacious bispecific compound was T017000633 containing the T017000624 at the N-terminal position. However, also format T017000634 containing the T017000624 at the C-terminal position was more potent than ISVD T017000128 with the T0170056G05 ISVD at the N-terminal position.

### 6.12 Example 12: Construction of Trivalent CD123/TCR Bispecific ISVD Formats

To evaluate the impact of the affinity maturation and sequence optimization of the TCR ISVD in multivalent T cell engager ISVD formats, additional formats were generated containing the TCR ISVD at different positions, and 2 different CD123 effector ISVDs that were linked using a 35GS linker.

To evaluate efficacy and safety of the T017000624, in vitro and in vivo, trivalent CD123/TCR bispecific ISVD formats were generated (Table 23). Irrelevant formats were generated by replacing the tumour antigen binding ISVDs with an irrelevant anti-RSV ISVD. Format T017000139, T0170056G05(E1D)-35GS-A0110055F03-35GS-A0110056A10-A containing the T0170056G05 as TCR building block was taken along for comparison purposes.

TABLE 23
Format ID and description of trivalent CD123/TCR bispecific ISVD formats and controls

| Format ID | Description |
|---|---|
| T017000139 | T0170056G05(E1D)-35GS-A0110055F03-35GS-A0110056A10-A |
| T017000670 | T0170056G05(E1D, L11V, D27Y, L34Y, M77T, K83R, V89L, F91Y, F95L, Y99W)- |
| | 35GS-A0110055F03-35GS-A0110056A10-A |
| T017000671 | A0110056A10(E1D)-35GS- |
| | T0170056G05(L11V, D27Y, L34Y, M77T, K83R, V89L, F91Y, F95L, Y99W)-35GS- |
| | A0110055F03-A |
| T017000672 | T0170056G05(E1D, L11V, D27Y, L34Y, M77T, K83R, V89L, F91Y, F95L, Y99W)- |
| | 35GS-RSV007B02(Q108L)-35GS-RSV007B02(Q108L)-A |
| T017000673 | RSV007B02(E1D, Q108L)-35GS- |
| | T0170056G05(L11V, D27Y, L34Y, M77T, K83R, V89L, F91Y, F95L, Y99W)-35GS- |
| | RSV007B02(Q108L)-A |
| T017000674 | RSV007B02(E1D, Q108L)-35GS-RSV007B02(Q108L)-35GS- |
| | T0170056G05(L11V, D27Y, L34Y, M77T, K83R, V89L, F91Y, F95L, Y99W)-A |
| T017000675 | A0110055F03(E1D)-35GS-A0110056A10-35GS- |
| | T0170056G05(L11V, D27Y, L34Y, M77T, K83R, V89L, F91Y, F95L, Y99W)-A |
| T017000676 | A0110056A10(E1D)-35GS-A0110055F03-35GS- |
| | T0170056G05(L11V, D27Y, L34Y, M77T, K83R, V89L, F91Y, F95L, Y99W)-A |
| T017000677 | A0110055F03(E1D)-35GS- |
| | T0170056G05(L11V, D27Y, L34Y, M77T, K83R, V89L, F91Y, F95L, Y99W)-35GS- |
| | A0110056A10-A |

### 6.13 Example 13: Functional Characterization of Trivalent CD123/TCR Bispecific ISVD Formats in a Flow Cytometry Based T Cell Mediated Killing Assay

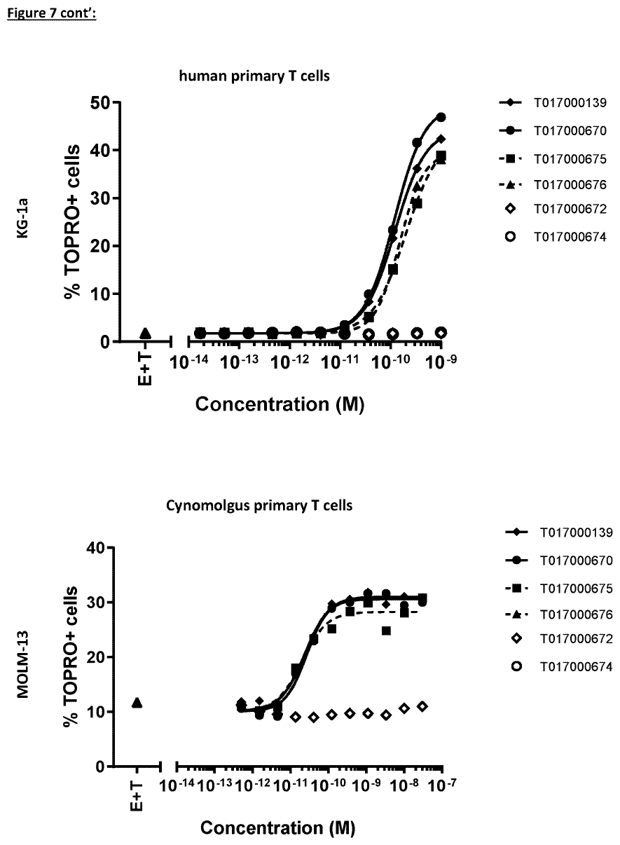

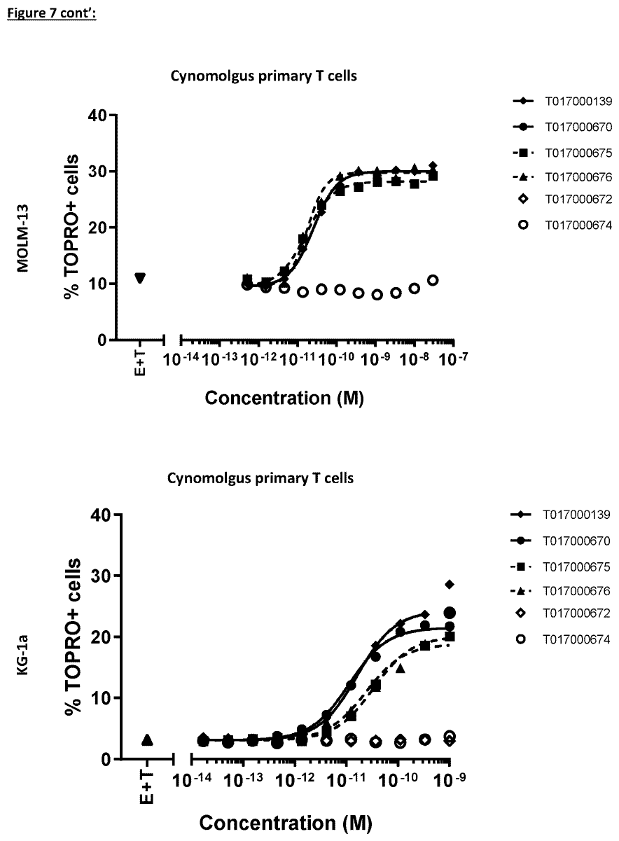

In order to assess whether the position of the T017000624 in a trivalent ISVD format has an impact on functionality, a flow cytometry-based cytotoxicity assay was performed using MOLM-13 or KG-1a as target cells and human or cynomolgus T cells as effector cells as described in example 11. Dose response curves based on the ISVD concentration dependent target cell killing are shown in FIG. 7 and data are summarized in Tables 24 and 25.

TABLE 24
Average EC50 (M) and % lysis for the CD123/TCR trivalent, bispecific ISVD
formats in the T cell mediated MOLM-13 cell killing assay using an effector
to target ratio of 10 to 1 (n = number) of independent experiments).

| | Human T cell mediated killing | | | | | Cynomolgus T cell mediated killing | | | | |
|---|---|---|---|---|---|---|---|---|---|---|
| MOLM-13 | | | EC50 95% | EC50 95% | % | | | EC50 95% | EC50 95% | % |
| Format ID | n | EC50 (M) | LCI (M) | UCI (M) | lysis | n | EC50 (M) | LCI (M) | UCI (M) | lysis |
| T017000139 | 4 | 2.5E−11 | 1.9E−11 | 3.4E−11 | 25 | 3 | 7.2E−11 | 3.0E−11 | 2.2E−10 | 40 |
| T017000670 | 3 | 2.5E−11 | 2.0E−11 | 3.3E−11 | 26 | 2 | 2.0E−11 | 8.5E−12 | 6.4E−11 | 34 |
| T017000671 | 2 | 1.5E−10 | 1.1E−10 | 2.3E−10 | 14 | 2 | 2.1E−11 | 1.8E−11 | 2.6E−11 | 46 |
| T017000675 | 4 | 3.4E−11 | 2.6E−11 | 4.7E−11 | 23 | 3 | 6.9E−11 | 3.7E−11 | 1.4E−10 | 31 |
| T017000676 | 3 | 2.3E−11 | 1.8E−11 | 3.0E−11 | 25 | 1 | 2.0E−11 | 1.7E−11 | 2.3E−11 | 26 |
| T017000677 | 2 | 1.2E−11 | 8.6E−12 | 1.7E−11 | 20 | 2 | 5.1E−12 | 4.3E−12 | 6.1E−12 | 54 |

LCI: lower confidence interval,
UCI: upper confidence interval

TABLE 25
Average EC50 (M) and % lysis for the CD123/TCR trivalent, bispecific ISVD
formats in the T cell mediated KG-1a cell killing assay using an effector
to target ratio of 10 to 1 (n = number of independent experiments).

| | Human T cell mediated killing | | | | | Cynomolgus T cell mediated killing | | | | |
|---|---|---|---|---|---|---|---|---|---|---|
| KG-1a | | | EC50 95% | EC50 95% | % | | | EC50 95% | EC50 95% | % |
| Format ID | n | EC50 (M) | LCI (M) | UCI (M) | lysis | n | EC50 (M) | LCI (M) | UCI (M) | lysis |
| T017000139 | 4 | 7.8E−11 | 6.7E−11 | 9.2E−11 | 51 | 2 | 1.2E−11 | 8.4E−12 | 2.0E−11 | 26 |
| T017000670 | 3 | 7.4E−11 | 6.6E−11 | 8.5E−11 | 52 | 2 | 8.1E−12 | 5.6E−12 | 1.2E−11 | 30 |
| T017000671 | 2 | 1.5E−10 | 1.2E−10 | 2.0E−10 | 21 | 0 | | | | |
| T017000675 | 4 | 9.2E−11 | 7.7E−11 | 1.1E−10 | 46 | 2 | 2.6E−11 | 1.7E−11 | 4.2E−11 | 26 |
| T017000676 | 3 | 8.2E−11 | 7.2E−11 | 9.6E−11 | 41 | 2 | 1.4E−11 | 9.7E−12 | 2.2E−11 | 28 |
| T017000677 | 2 | 1.5E−11 | 1.3E−11 | 1.9E−11 | 65 | 0 | | | | |

LCI: lower confidence interval,
UCI: upper confidence interval

All negative control formats (T017000672, T017000673, T017000674) lacking CD123 specificity were inactive as expected. For all trivalent CD123/TCR bispecific formats (T017000670, T017000675, T017000676) only little difference was observed, with the formats containing the anti-TCR ISVD at the C-terminal position being as potent and efficacious compared to their N-terminal counterpart. At least for the N-terminal TCR format T017000139, replacing T0170056G05 by the T017000624 ISVD had little impact on potency and efficacy (T017000670 vs T017000139).

### 6.14 Example 14: Functional Characterization of Trivalent CD123/TCR Bispecific ISVD Formats in an Impedance-Based Human and Cynomolgus T Cell Mediated CHO-K1 Cell Killing Assay

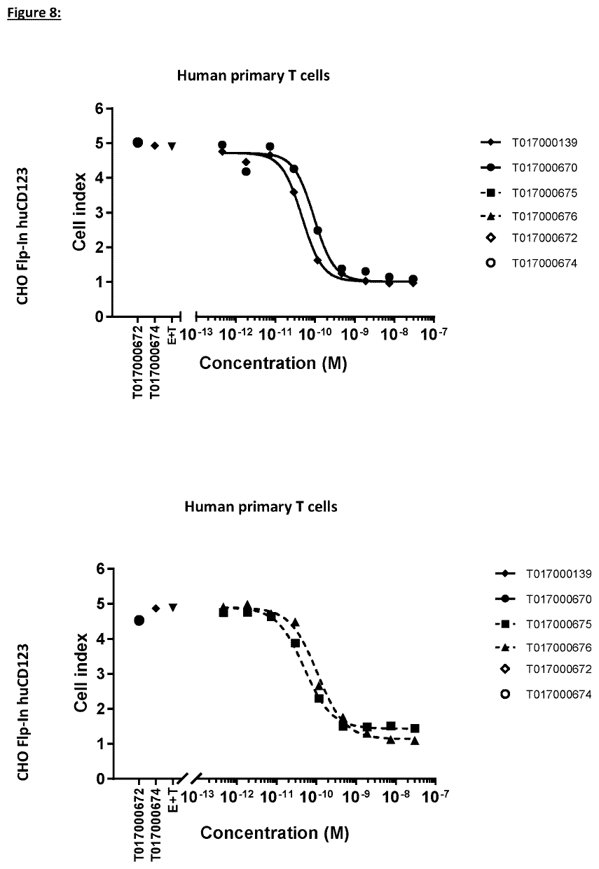

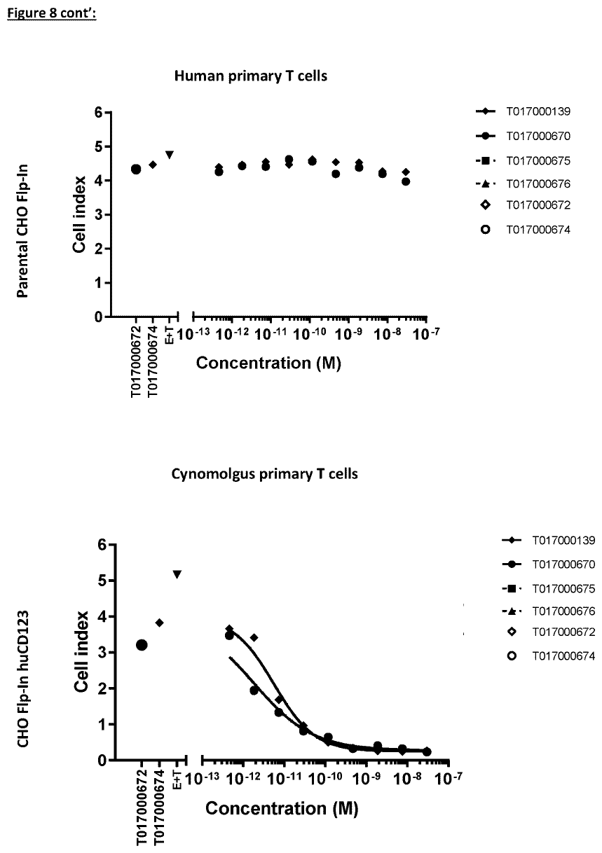

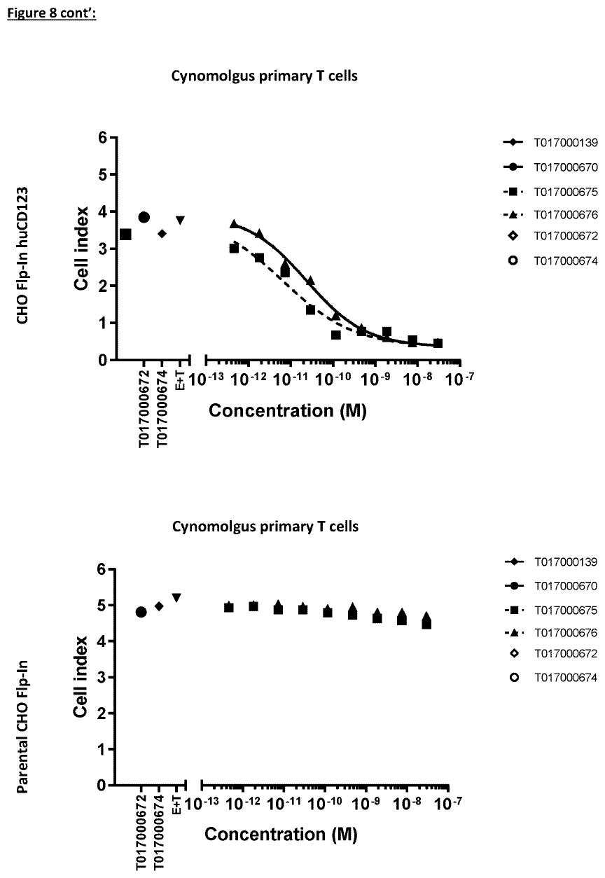

The CD123/TCR bispecific trivalent ISVD formats were further characterized in an impedance-based cytotoxicity assay (xCELLigence) using human or cynomolgus primary effector T cells and adherent CHO Flp-In huCD123 or the CHO Flp-In parental target cells as described in Example 11. Dose response curves are shown in FIG. 8 and results are summarized in Table 26.

TABLE 26
IC50 and % inhibition for the CD123/TCR trivalent, bispecific
ISVD formats in the T cell mediated cell killing assay based
on xCELLigence readout using an E:T ratio of 15:1.

| | Human T cell mediated killing | | |
|---|---|---|---|
| Format ID | IC50 (M) | IC50 95% LCI (M) | I50 95% UCI (M) |
| T017000139 | 4.7E−11 | 3.7E−11 | 6.1E−11 |
| T017000670 | 9.4E−11 | 7.3E−11 | 1.2E−10 |
| T017000675 | 5.0E−11 | 3.9E−11 | 6.2E−11 |
| T017000676 | 1.1E−10 | 8.5E−11 | 1.3E−10 |

LCI: lower confidence interval,
UCI: upper confidence interval

Impedance based cell killing results were in line with the results obtained for the flow cytometry-based cell killing assays. Formats containing the TCR ISVD at the C-terminal position being as potent and efficacious compared to their N-terminal counterpart. Trivalent CD123/TCR bispecific ISVD formats did not show killing of the parental CHO Flp-In cells.

### 6.15 Example 15: Deimmunization

The T017000624 variant was subjected to the process called “Deimmunization”. This process encompasses the identification of potential T cell epitopes and the removal of such epitopes by introducing mutations without impacting the binding properties and the biophysical characteristics of the molecule. One potential epitope was identified, with L89 as P1 anchor and comprising part of CDR3. From in silico analysis, mutation to A or T is a predicted solution to remove the T cell epitope. Therefore, three anti-TCR ISVD variants (T017000679, T017000680, T017000681) (Table 27) with either V89L, V89A, V89T were produced in Pichia pastoris and further characterized.

TABLE 27
ISVD ID and description of TCR variants that were evaluated in the DC-T assay.

| ISVD ID | Description |
|---|---|
| T017000679 | T0170056G05(E1D, L11V, D27Y, L34Y, M77T, K83R, V89L, F91Y, F95L, Y99W) |
| T017000680 | T0170056G05(E1D, L11V, D27Y, L34Y, M77T, K83R, V89A, F91Y, F95L, Y99W) |
| T017000681 | T0170056G05(E1D, L11V, D27Y, L34Y, M77T, K83R, V89T, F91Y, F95L, Y99W) |

### 6.16 Example 16: Binding of T017000680 to TCR Protein Via SPR on ProteOn XPR36

Analysis of binding kinetics of T017000680, to directly immobilized huTCR-zipper and cyTCR-zipper proteins was performed in a SPR based assay on a ProteOn XPR36 at 37° C.

In brief, huTCR-zipper or cyTCR-zipper protein was immobilized on a GLC sensor chip (between 100-200 RU) via amine coupling chemistry, using EDC/NHS for activation of the carboxyl groups on the chip surface (running buffer: HBS-EP+, pH7.4). Purified ISVDs were injected for 2 min (at a flow rate of 45 μL/min) at different concentrations (between 4.1 nM and 1000 nM) and dissociation was followed for 900 s. Data were double referenced by subtracting a reference ligand lane and a blank buffer injection. Processed sensorgrams were fitted with the Langmuir model (1:1 interaction) using ProteOn Manager 3.1.0 (Version 3.1.0.6) software.

The data are shown in Table 28.

TABLE 28
Affinity determination of anti-TCR ISVD to huTCR-zipper and cyTCR-
zipper protein as determined via SPR on ProteOn XPR36 at 37° C.

| | huTCR-zipper protein | | | cyTCR-zipper protein | | |
|---|---|---|---|---|---|---|
| | kₐ (1/Ms) | k_{d} (1/s) | KD (M) | kₐ (1/Ms) | k_{d} (1/s) | KD (M) |
| T017000680 | 3.2E+05 | 2.8E−03 | 8.6E−09 | 1.9E+06 | 9.2E−03 | 4.9E−09 |

The KD of T017000680, is similar as the KD of T017000624 for huTCR-zipper and cyTCR-zipper protein. The difference in on and off-rate is due to the higher temperature used to determine the binding kinetics of the T017000680. The delta for human-cynomolgus cross-reactivity for T017000680, based on KD is 1.8-fold. In conclusion, the V89A mutation (T017000680) had no impact on the binding kinetics compared to T017000624.

### 6.17 Example 17: Binding of the TCR ISVD Variants to T Cells Via Flow Cytometry

Affinity of the tagless TCR ISVD variants to human primary T cells and the HSC-F cyno T cell line (NIBIO JCRB1164) was evaluated using flow cytometry in a competition setup using the Flag3-His6-tagged anti-TCR ISVD as ligand.

In brief, cells were harvested and transferred to a V-bottom 96-well plate (7.5×10⁴ cells per well in 100 μL) and incubated with a serial dilution of ISVD formats and a fixed concentration of ligand for 30 min at 4° C. in FACS buffer. The concentration of ligand used in the assay was below its binding EC50 (data not shown). After an incubation period of 90 min at 4° C., the level of ligand binding was determined via flow cytometry. Thereto, cells were washed 3 times and incubated with 1 μg/ml ANTI-FLAG® M2 Ab (Sigma, F1804) for 30 min at 4° C., washed again, and incubated for 30 min at 4° C. with 5 μg/ml R-Phycoerythrin-AffiniPure F(ab′)2 Fragment Goat Anti-Mouse IgG, Fc gamma Fragment Specific (Jackson Immunoresearch, 115-116-07). Subsequently, cells were resuspended in FACS buffer supplemented with 1 μg/ml Propidium Iodide (PI, Sigma, P4170) to distinguish live from dead cells. Cells were analyzed using a FACS Array flow cytometer (BD Biosciences) using Flowing Software. First a P1 population which represented more than 80% of the total cell population was selected based on FSC-SSC distribution. In this gate, 10000 cells were counted during acquisition. From this population the Propidium iodide or TO-PRO©-3 Iodide positive cells (dead cells) were excluded and the median or Mean PE value was calculated.

Dose titration curves and shown in FIG. 9. IC50 values obtained from the dose response curves are summarized in Table 29.

TABLE 29
IC50 (M) of the TCR ISVD variants in the competition assay using human primary
T cells and HSC-F cynomolgus T cells determined via flow cytometry.

| | human primary T cells | | | HSC-F cells | | |
|---|---|---|---|---|---|---|
| | | IC50 95% | IC50 95% | | IC50 95% | IC50 95% |
| ISVD ID | IC50 (M) | LCI (M) | UCI (M) | IC50 (M) | LCI (M) | UCI (M) |
| T017000679 | 1.0E−07 | 8.3E−08 | 1.3E−07 | 6.20E−07 | 5.00E−07 | 8.30E−07 |
| T017000680 | 1.0E−07 | 8.5E−08 | 1.3E−07 | 7.30E−07 | 5.80E−07 | 9.80E−07 |
| T017000681 | 9.2E−08 | 7.5E−08 | 1.1E−07 | 7.3E−07 | 5.8E−07 | 9.9E−07 |

LCI: lower confidence interval,
UCI: upper confidence interval

Affinity of the TCR ISVD variants were in the same range for the human primary T cells and the cynomolgus HSC-F T cell line.

### 6.18 Example 18: Immunogenicity Profiling in Dendritic Cell—T Cell (DC-T Cell) Assay

The relative immunogenicity of anti-TCR ISVD variants T017000679, T017000680, T017000681 was determined in a Dendritic Cell-T cell proliferation assay.

In essence, the ISVDs were tested against a set of 47 healthy donor cell samples containing the most abundant HLA class II alleles, as such representing the majority of the global population. The immune response was assessed using T cell proliferation as a surrogate marker for anti-drug antibody formation. Keyhole Limpet Hemocyanin (KLH) was used as a positive control. The positive control KLH led to a positive response in all of the 47 donors. The overall immunogenic potential for all 3 ISVDs was low. In none of the donor samples a significant response to ISVDs could be observed (p<0.05). Based on number of responsive donors as well as intensity of response, the immunogenicity of T017000680 was the lowest, followed by the T017000681, the least preferred was the T017000679.

The sequences of the identified sequence optimized, affinity matured variants with confirmed low immunogenicity profile are depicted in Table 30.

In case the anti-TCR ISVD variant is placed at the N-terminal position of an ISVD format, the sequences of T017000679, T017000680 and T017000681 with a D at position 1 are preferred, as to avoid potential pyroglutamate formation. The corresponding variants with an E at position 1, i.e. T01700624, T017000697 and T017000681(D1E), respectively, are preferentially used when not located at the N-terminal position.

TABLE 30
Sequences of optimized anti-TCR variants.

| | SEQ ID | |
|---|---|---|
| ISVD ID | NO | Amino acid sequence |
| T017000624 | 46 | EVQLVESGGGVVQPGGSLRLSCVASGYVHKINFYGWYRQAPGKEREKVAHISIG |
| | | DQTDYADSAKGRFTISRDESKNTVYLQMNSLRPEDTALYYCRALSRIWPYDYWG |
| | | QGTLVTVSS |
| T017000679 | 150 | DVQLVESGGGVVQPGGSLRLSCVASGYVHKINFYGWYRQAPGKEREKVAHISIG |
| | | DQTDYADSAKGRFTISRDESKNTVYLQMNSLRPEDTALYYCRALSRIWPYDYWG |
| | | QGTLVTVSS |
| T017000680 | 151 | DVQLVESGGGVVQPGGSLRLSCVASGYVHKINFYGWYRQAPGKEREKVAHISIG |
| | | DQTDYADSAKGRFTISRDESKNTVYLQMNSLRPEDTAAYYCRALSRIWPYDYWG |
| | | QGTLVTVSS |
| T017000697 | 261 | EVQLVESGGGVVQPGGSLRLSCVASGYVHKINFYGWYRQAPGKEREKVAHISIG |
| | | DQTDYADSAKGRFTISRDESKNTVYLQMNSLRPEDTAAYYCRALSRIWPYDYWG |
| | | QGTLVTVSS |
| T017000681 | 152 | DVQLVESGGGVVQPGGSLRLSCVASGYVHKINFYGWYRQAPGKEREKVAHISIG |
| | | DQTDYADSAKGRFTISRDESKNTVYLQMNSLRPEDTATYYCRALSRIWPYDYWG |
| | | QGTLVTVSS |
| T017000681 | 262 | EVQLVESGGGVVQPGGSLRLSCVASGYVHKINFYGWYRQAPGKEREKVAHISIG |
| (D1E)* | | DQTDYADSAKGRFTISRDESKNTVYLQMNSLRPEDTATYYCRALSRIWPYDYWG |
| | | QGTLVTVSS |

*( ) indicates the amino acid substitutions introduced into the ISVD (e.g.: T017000681(D1E) means that ISVD T017000681 (SEQ ID NO: 152) contains a D1E substitution).

### 6.19 Example 19: In Vivo Efficacy and Safety of CD123/TCR Bispecific ISVD Formats

In vivo efficacy and safety of two multispecific CD123/TCR binding ISVD formats, T017000670 (with N-terminally anti-TCR ISVD T017000697) and T017000675 (with C-terminally anti-TCR ISVD T017000624), were evaluated in a non-human primate model. A Dual-affinity re-targeting antibody (DART) was taken along as a reference in the experiment.

ISVD format T017000139 with the non-sequence optimized and affinity matured anti-TCR building block T01700056G05 at the N-terminal end was used as reference molecule.

Treatment with the irrelevant/TCR binding ISVD formats T017000672 (with N-terminally anti-TCR ISVD T017000697) and T017000674 (with C-terminally anti-TCR ISVD T017000624) was used as specificity control for the CD123-targeting moiety of the multispecific ISVD format.

A description of these formats can be found in Table 23.

All compounds were administered via a 24 h continuous i.v. infusion for 4 weeks after a 7-day NaCl infusion ‘pre-treatment’ of cynomolgus monkeys. Every week, a 4-days on/3-days off cycle was applied with a weekly dose escalation scheme as described in Chichili et al. (Sci Transl Med, 2015, PMID: 26019218). Administered doses are depicted in Table 31.

TABLE 31
Dose escalation scheme for in vivo study

| | Dose levels (ng/kg/day) | | | | |
|---|---|---|---|---|---|
| | Test | Test | Test | Test | Number |
| | week 1 | week 2 | week 3 | week 4 | of male |
| Compound | (d 1-5) | (d 8-12) | (d 15-19) | (d 22-26) | animals |
| DART | 100 | 300 | 600 | 1000 | 2 |
| Reference | | | | | |
| T017000139 | 74.0 | 222.1 | 444.3 | 740.4 | 4 |
| T017000672 | 76.9 | 230.8 | 461.7 | 769.4 | 4 |
| T017000670 | 74.2 | 222.6 | 445.3 | 742.1 | 6 |
| T017000674 | 76.9 | 230.8 | 461.7 | 769.4 | 4 |
| T017000675 | 74.2 | 222.6 | 445.3 | 742.1 | 4 |

The circulating number of CD123′ cells was explored as a pharmacodynamic endpoint to assess in vivo efficacy by quantifying the number of CD123^{hi} and the number of CD123^{int} cells in the PBMC fraction of peripheral blood samples using flow cytometry on test days (TD)-6 or -7, -4, 1 (pre-dose and 4 hrs post-dose start), 4, 8 (pre-dose and 4 hrs post-dose start), 11, 15 (pre-dose and 4 hrs post-dose start), 18, 22 (pre-dose and 4 hrs post-dose start), 25, 29, 36, and 45.

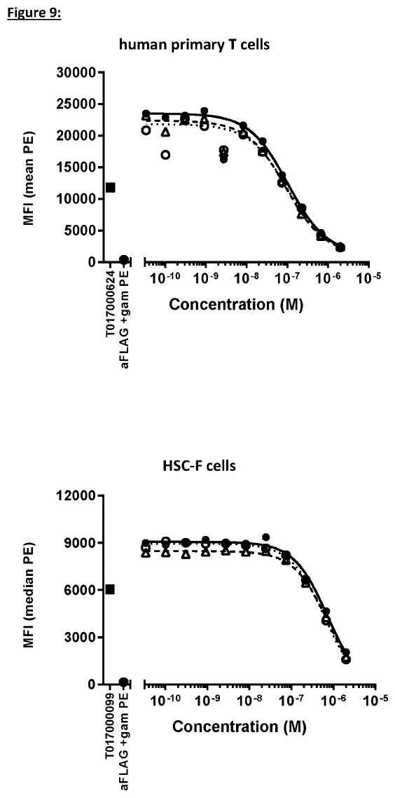

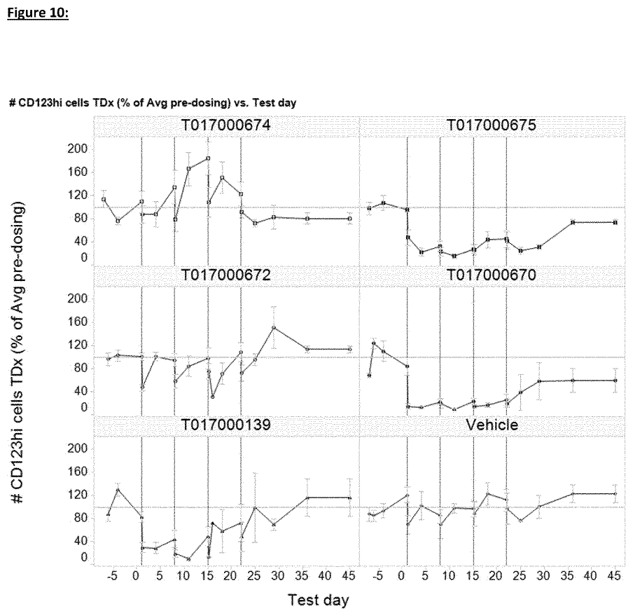

The results are depicted in FIG. 10 and FIG. 11.

CD123^{hi} cells were depleted in the animals treated with compounds T017000670 and T017000139, with a N-terminal TCR binding building block, and compound T017000675 with a C-terminal TCR binding building block. The effect is observed from the first dosing cycle onwards and treatment remains efficacious in all dosing cycles for the sequence-optimized and affinity-matured variants T017000670 and T017000675. T017000139 however, showed some efficacy loss from the 3^{rd} dosing cycle onwards.

CD123^{int} cells were not depleted in the animals treated with compound T017000675 with an C-terminal anti-TCR building block nor in the animals treated with the T017000139 and T017000670.

T cell redistribution from the blood was monitored by quantifying T cell subsets within the PBMC fraction of peripheral blood samples using flow cytometry on test days (TD)-7 or -6, -4, 1 (pre-dose+4 hrs post-dose start), 4, 8 (pre-dose+4 hrs post-dose start), 11, 15 (pre-dose+4 hrs post-dose start), 18, 22 (pre-dose+4 hrs post-dose start), 25, 29, 36, and 45.

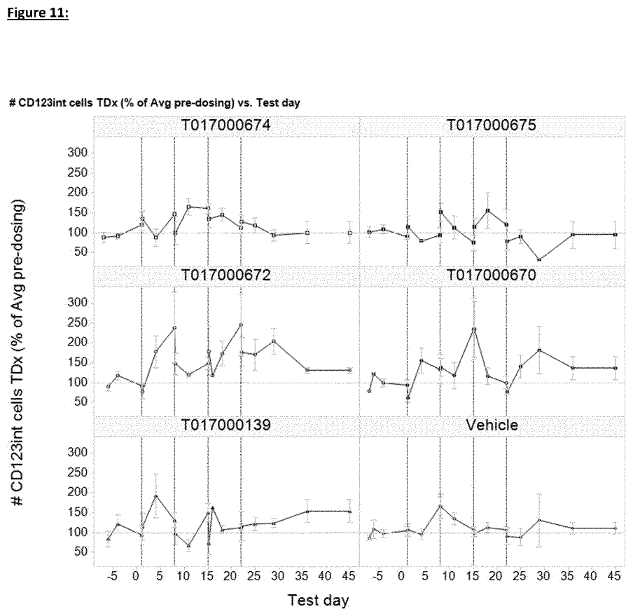

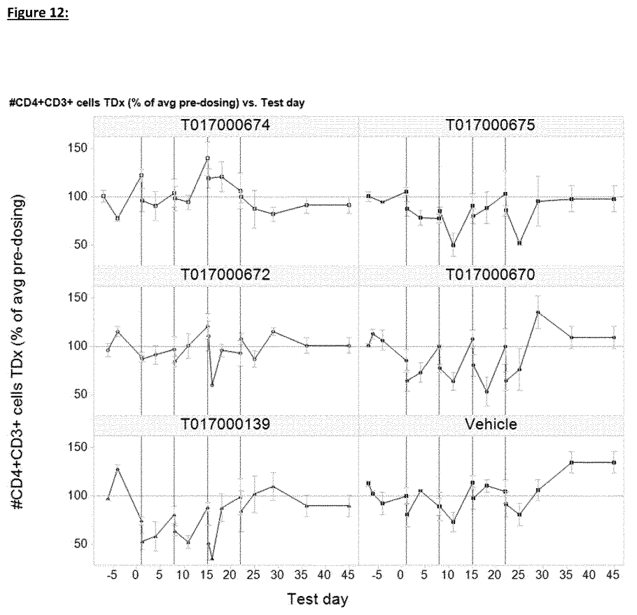

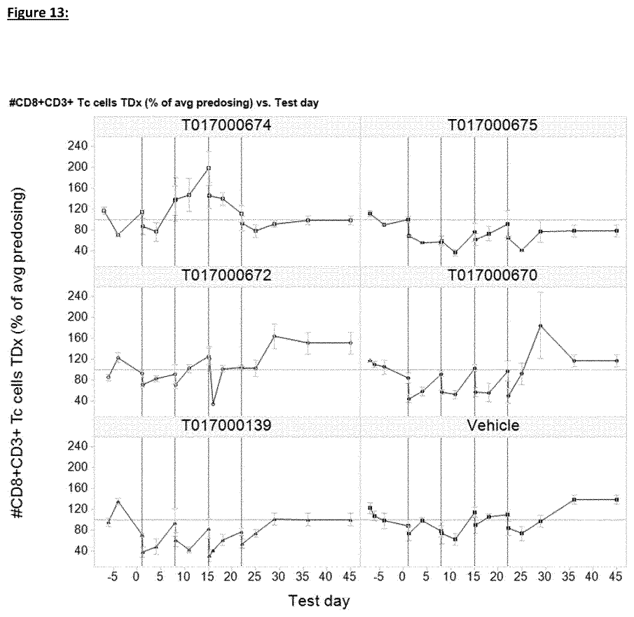

The results are depicted in FIG. 12 and FIG. 13.

T-helper cell (Th, CD4+CD3+) and cytotoxic T cell (Tc, CD8+CD3+) numbers transiently decreased over time during treatment with all CD123/TCR ISVD formats. In the group treated with T0170000670, Th and Tc cell numbers returned to baseline levels during the 3 days off period of each dosing cycle. A similar trend was seen for Tc cells in the animals treated with compound T017000675 and for Th cells in the animals treated with compounds T017000139 and T017000675 but the number of cells only returned to baseline after the 3rd dosing cycle. Th and Tc cell numbers returned to a stable value around the baseline during the recovery period in all treatment groups. The signs of redistribution were far less for the tested DART reference molecule. The transient decrease over time can be suggestive of trafficking of T cells, but does not support a depletion of T cells.

Next, the expression of PD-1 on circulating Th (CD4+CD3+) cells and Tc (CD8+CD3+) cells was explored as a surrogate, phenotypic marker to assess T cell exhaustion in vivo. For this, PD-1 expression was measured in the PBMC fraction of peripheral blood samples of treated animals using flow cytometry on test days (TD)-7 or -6, -4, 1 (pre-dose and 4 hrs post-dose start), 4, 8 (pre-dose and 4 hrs post-dose start), 11, 15 (pre-dose and 4 hrs post-dose start), 18, 22 (pre-dose and 4 hrs post-dose start), 25, 29, 36, and 45.

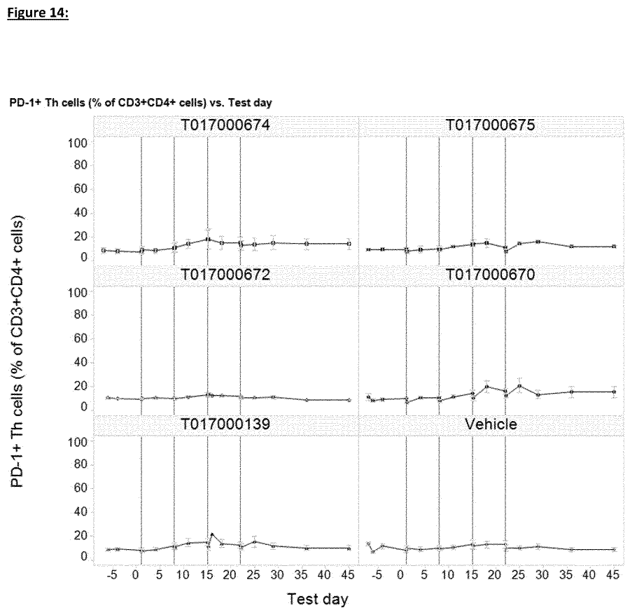

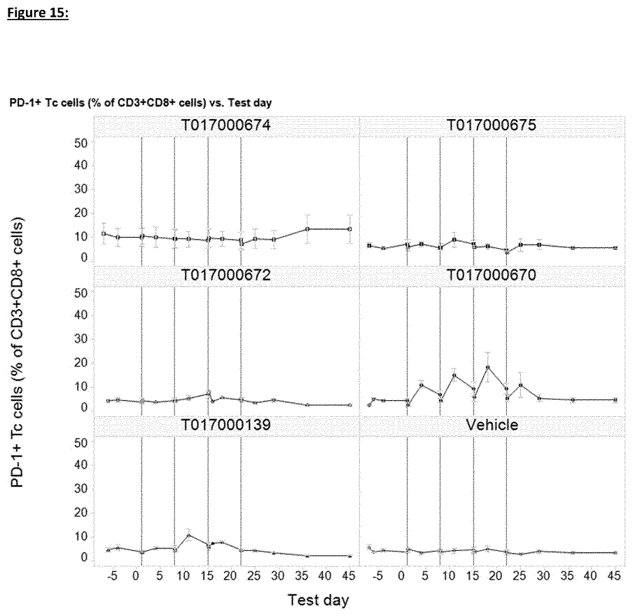

The results are depicted in FIG. 14 and FIG. 15.

The percentage PD-1 positive CD4 T cells or CD8 T cells was similar in animals treated with CD123-targeted compounds T017000670 or T017000675, as in animals treated with the respective non-targeted controls. The small fractions of PD-1 positive cells were far less than those for the reference DART molecule taken along in the examples.

Safety was assessed by evaluation of following cytokines: IL-1p, IL-2, IL-4, IL-5, IL-6, IL-8, IL-10, IL-12(p40), TNF-α, TNF-β and IFN-γ in the serum on test days (TD)-7, -4, 1 (4 hrs post-dose start), 4, 8 (4 hrs post-dose start), 11, 15 (4 hrs post-dose start), 18, 22 (4 hrs post-dose start), 25, 29, 36 and 45.

Levels of IL-1β, IL-2, IL-4, IL-5, IL-10, IL-12(p40), TNF-α, TNF-β and IFN-γ remained below detection limit at all time points in all animals, indicating no sign of a cytokine release syndrome was observed.

A few transient increases above baseline were observed for serum concentrations of IL-6 and IL-8. However, none of these increases was related to the pharmacological action of any of the compounds, as determined based on haematological and coagulation parameters and neurological examination, during the treatment protocol and observations during necropsy.

### 6.20 Example 20: Generation and Characterisation of Trivalent and Tetravalent GPC3/TCR Trispecific ISVD Formats

Additional formats were generated containing the TCR ISVDs T017000679, T017000624, T017000680 and T017000681, one or more further ISVD(s) targeting Glypican-3 (GPC3) (i.e. A0226018C08 and A0226015A08), and an ISVD binding to human serum albumin (i.e. ALB23002). Different linker lengths and orientiations of the ISVDs were used. The generated formats are listed in Table 32. Amino acid sequences are shown in Table A-9.

TABLE 32
Format ID and description of trivalent and tetravalent
GPC3/TCR trispecific ISVD formats and controls

| | SEQ ID | |
|---|---|---|
| Format ID | NO | Description |
| A022600096 | 434 | T017000679-35GS-A0226018C08-35GS-ALB23002-A |
| A022600102 | 435 | T017000679-AAA-A0226018C08-35GS-ALB23002-A |
| A022600103 | 436 | T017000679-5GS-A0226018C08-35GS-ALB23002-A |
| A022600104 | 437 | T017000679-9GS-A0226018C08-35GS-ALB23002-A |
| A022600105 | 438 | T017000679-20GS-A0226018C08-35GS-ALB23002-A |
| A022600122 | 439 | T017000679-5GS-A0226018C08-35GS-A0226015A08-35GS- |
| | | ALB23002-A |
| A022600131 | 440 | T017000679-5GS-A0226018C08-20GS-A0226015A08-35GS- |
| | | ALB23002-A |
| A022600132 | 441 | T017000679-5GS-A0226018C08-9GS-A0226015A08-35GS- |
| | | ALB23002-A |
| A022600133 | 442 | T017000679-5GS-A0226015A08-35GS-A0226018C08-35GS- |
| | | ALB23002-A |
| A022600134 | 443 | T017000679-5GS-A0226015A08-20GS-A0226018C08-35GS- |
| | | ALB23002-A |
| A022600135 | 444 | T017000679-5GS-A0226015A08-9GS-A0226018C08-35GS- |
| | | ALB23002-A |
| A022600167 | 445 | T017000680-5GS-A0226018C08-35GS-ALB23002-A |
| A022600168 | 446 | T017000680-5GS-A0226018C08-35GS-A0226015A08-35GS- |
| | | ALB23002-A |
| A022600169 | 447 | A0226018C08-5GS-T01000697-5GS-A0226015A08-35GS- |
| | | ALB23002-A |
| A022600170 | 448 | T017000680-5GS-A0226018C08-35GS-A0226015A08-9GS- |
| | | ALB23002-A |
| A022600172 | 449 | T017000680-5GS-A0226018C08-9GS-ALB23002-9GS- |
| | | A0226015A08-A |
| A022600174 | 450 | T017000680-5GS-A0226018C08-9GS-A0226015A08-9GS- |
| | | ALB23002-A |
| A022600175 | 451 | T017000680-5GS-A0226018C08-9GS-A0226015A08-35GS- |
| | | ALB23002-A |
| A022600178 | 452 | A0226018C08-5GS-T017000697-35GS-ALB23002-A |
| A022600179 | 453 | T017100680-5GS-A0226018C08-9GS-ALB23002-A |
| T017000698 | 454 | T017000680-9GS-ALB23002-A |

The generated formats were characterised in a T-cell dependent cytotoxicity (TDC) assay. HepG2 (ATCC, clone HB8065), a liver cancer cell line with high expression of GPC3, was labelled with Nuclight Green (Essen Bioscience, cat no. 4624) and used as target for T-cell killing in the presence of the trispecific GPC3/TCR T-cell engager constructs. To this end, plates (96-well F-bottom, Greiner, cat no 655180) were pre-blocked with 200 μL/well assay medium (2 h, 37° C.). Simultaneous addition of each assay component was performed in a total volume of 200 μL/well: (1) 50 μL of diluted/titrated compounds (Nbs; Brefeldin A (Sigma-Aldrich, cat no B7651); (2) 25 μL of diluted HSA (Sigma-Aldrich, cat no A8763-10G) (final concentration: 30 μM); (3) 25 μL of diluted Cytotox Red (Essen Bioscience, cat no 4632) (final concentration: 250 nM) (4) 50 μL of human T-cells (T cells were isolated from buffy coats (Red Cross) using the RosetteSep T cell enrichment cocktail (StemCell, cat no. 15061) and 50 μL of HepG2 Nuclight green (fresh in DNEM, High Glucose, GlutaMAX, Pyruvate, Life Technologies-Gibco, cat no 31966) in a 15:1 ratio. Plates were placed in an IncuCyte ZOOM for readout in all three channels (phase-contrast, green and red) with intervals of 4 or 6 hours, for a total of 72 h.

Analysis of effect of linkers, anti-TCR ISVD, biparatopic targeting of GPC3 and orientiation was performed in 5 steps.

Step 1—Effect of the Linker Length Between the Anti-TCR ISVD and an Anti-GPC3 ISVD (Trispecific Trivalent Format); Step 2—Effect of Biparatopic Targeting of GPC3 ISVD (Trispecific Tetravalent Format) and Optimization of the Linker Length Between the Two GPC3 Binding ISVDs.

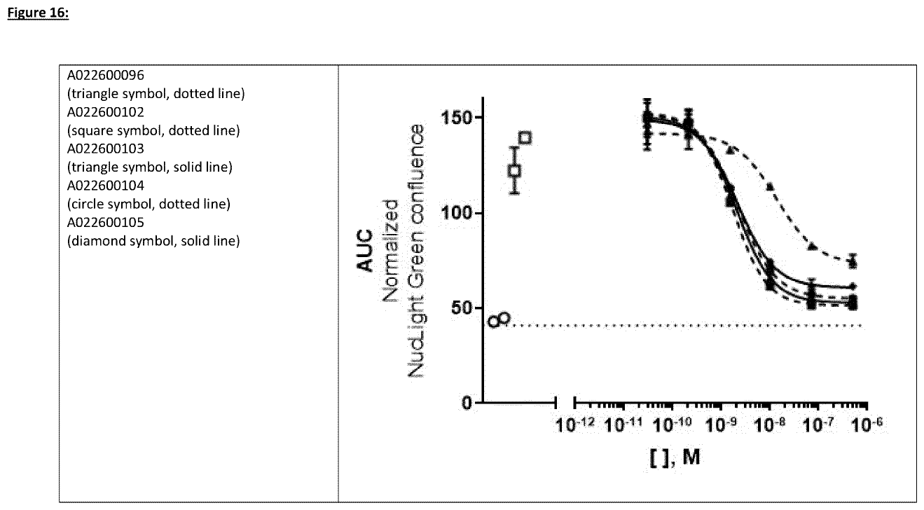

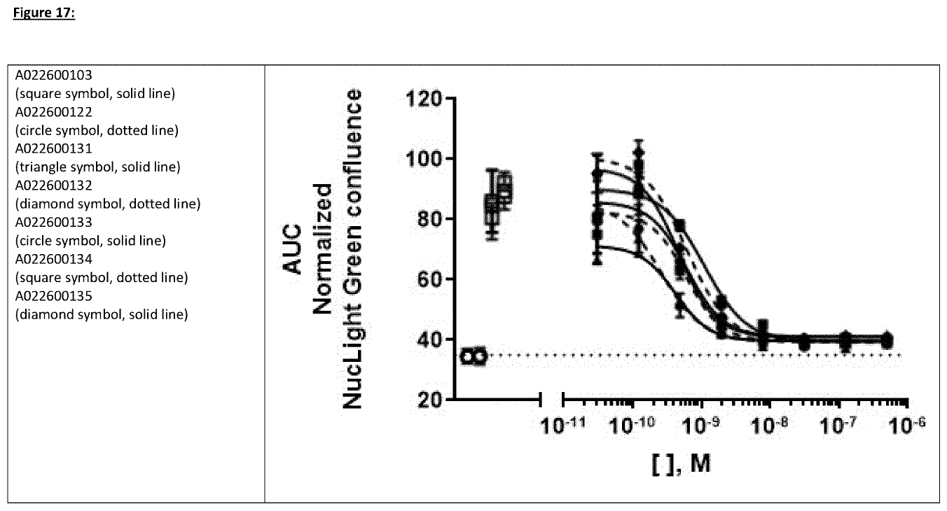

In Step 1, the trispecific GPC3 ISVD T-cell engagers showed cell tumor killing with increased efficacy (increased maximum killing) with decreasing length of the linker between the anti-TCR ISVD and anti-GPC3 ISVD, from 72% (35GS linker) to 94% (AAA linker) (FIG. 16). In Step 2, GPC3 biparatopic T-cell engagers were compared for which anti-GPC3 ISVDs A0226018C08 and A0226015A08 were combined in the same construct and placed in different orientations and with different linker lengths between the two building blocks. Here, no impact of the tested variables on efficacy was observed (FIG. 17).

Tables 33 and 34 show the IC50 values and the maximum % killing for the different formats

TABLE 33
IC50 (M) and maximum percentage of killing (%) of the trispecific GPC3 ISVD
T-cell engagers in the Incucyte based human TDC HepG2-Nuclight green assay
using an effector to target ratio of 15:1, analyzed at 72 h after seeding

| | | SEQ | | IC50 | Max % |
|---|---|---|---|---|---|
| Step | Sample ID | ID NO | Description | (M) | Killing |
| 1 | A022600096 | 434 | T017000679-35GS-A0226018C08-35GS- | 1.4E−09 | 72 |
| | | | ALB23002-A | | |
| | A022600105 | 438 | T017000679-20GS-A0226018C08-35GS- | 2.14E−09 | 85 |
| | | | ALB23002-A | | |
| | A022600104 | 437 | T017000679-9GS-A0226018C08-35GS- | 2.13E−09 | 90 |
| | | | ALB23002-A | | |
| | A022600103 | 436 | T017000679-5GS-A0226018C08-35GS- | 2.04E−09 | 92 |
| | | | ALB23002-A | | |
| | A022600102 | 435 | T017000679-AAA-A0226018C08-35GS- | 1.72E−09 | 94 |
| | | | ALB23002-A | | |

TABLE 34
IC50 (M) and maximum percentage of killing (%) of the trispecific GPC3 ISVD
T-cell engagers in the Incucyte based human TDC HepG2-Nuclight green assay
using an effector to target ratio of 15:1, analyzed at 72 h after seeding

| | | SEQ ID | | | Max % |
|---|---|---|---|---|---|
| Step | Sample ID | NO | Description | IC50 (M) | Killing |
| 2 | A022600103 | 436 | T017000679-5GS-A0226018C08-35GS- | 5.43E−10 | 90 |
| | | | ALB23002-A | | |
| | A022600122 | 439 | T017000679-5GS-A0226018C08-35GS- | 4.45E−10 | 92 |
| | | | A0226015A08-35GS-ALB23002-A | | |
| | A022600131 | 440 | T017000679-5GS-A0226018C08-20GS- | 4.44E−10 | 94 |
| | | | A0226015A08-35GS-ALB23002-A | | |
| | A022600132 | 441 | T017000679-5GS-A0226018C08-9GS- | 7.02E−10 | 91 |
| | | | A0226015A08-35GS-ALB23002-A | | |
| | A022600133 | 442 | T017000679-5GS-A0226015A08-35GS- | 4.08E−10 | 92 |
| | | | A0226018C08-35GS-ALB23002-A | | |
| | A022600134 | 443 | T017000679-5GS-A0226015A08-20GS- | 5.28E−10 | 92 |
| | | | A0226018C08-35GS-ALB23002-A | | |
| | A022600135 | 444 | T017000679-5GS-A0226015A08-9GS- | 6.11E−10 | 91 |
| | | | A0226018C08-35GS-ALB23002-A | | |

To enable evaluation of the ability to kill a liver cancer cell line expressing medium levels of GPC3 compared to the high levels of HepG2, a TDC Huh7 assay was set up. In this assay, the xCELLigence© (Acea) system was used. Firstly, 96-well E-plates (Acea, Cat no 5232368001) containing 50 μL of assay medium with a 4× concentration (120 μM) of Alburex 20 Human serum albumin (CSL Behring, Cat no 2160-979) (final assay concentration 30 μM) were placed inside the xCELLigence® for background measurement. After background measurement, simultaneous addition of each assay component was performed to a total volume of 200 μL/well: (1) 50 μL of diluted/titrated compounds; (2) 50 μL of single cell suspensions of Huh7 (HSRRB, clone JCRB0403); (3) 50 μL of single suspensions of effector cells (human T-cells, obtained as described in Example 3) to match an effector:target ratio of 15:1. Plates were placed in the xCELLigence© with 400 sweeps at 15-minute intervals. At the appropriate timepoint (ca. 60 h), cell indexes (CI) were analyzed, where a Cl of 0 represented 100% killing.

Step 3—Comparison of GPC3 T-Cell Engager Formats with Anti-TCR ISVD T017000679 and Deimmunized Variant T017000680 in Trivalent and Tetravalent Formats.

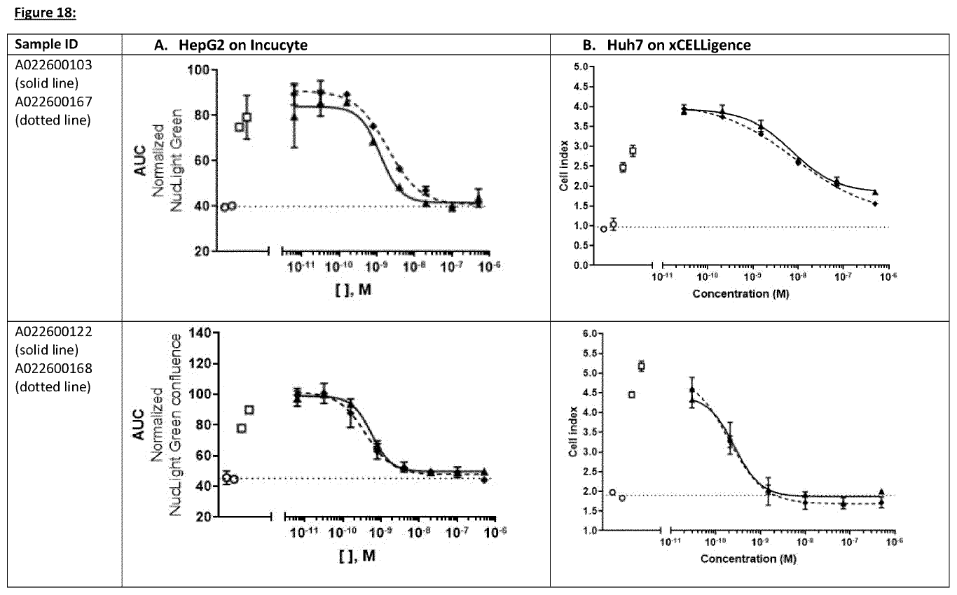

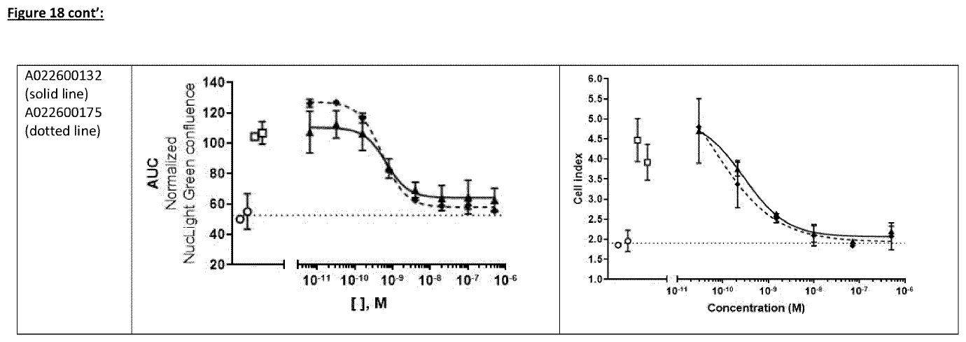

Potency and efficacy of the formats was assessed in the HepG2 and Huh7 TDC assay, as described above. The formats, their description and functionality are summarized in Table 35. Data is depicted in FIG. 18. A small increase in efficacy could be observed for the formats containing the T017000680 ISVD as compared to the formats comprising ISVD T017000679.

TABLE 35
IC50 (M) and maximum percentage of killing (%) of trispecific GPC3 ISVD T-cell engagers in
the Incucyte based human TDC HepG2-Nuclight green assay and in the xCELLigence based human
TDC Huh7 assay, using an effector to target ratio of 15:1, analyzed at 60 h after seeding

| | | | | HepG2 on | | Huh7 on |
|---|---|---|---|---|---|---|
| | | | HepG2 on | Incucyte | Huh7 on | xCELLigence |
| | SEQ | | Incucyte | Max % | xCELLigence | Max % |
| Sample ID | ID NO | Description | IC50 (M) | killing | IC50 (M) | killing |
| A022600103 | 436 | T017000679-5GS- | 1.23E−09 | 96 | 7.11E−09 | 71 |
| | | A0226018C08- | | | | |
| | | 35GS-ALB23002-A | | | | |
| A022600167 | 445 | T017000680-5GS- | 1.92E−09 | 98 | 8.61E−09 | 91 |
| | | A0226018C08- | | | | |
| | | 35GS-ALB23002-A | | | | |
| A022600122 | 439 | T017000679-5GS- | 5.3E−10 | 93 | 2.61E−10 | 101 |
| | | A0226018C08- | | | | |
| | | 35GS-A0226015A08- | | | | |
| | | 35GS-ALB23002-A | | | | |
| A022600168 | 446 | T017000680-5GS- | 3.67E−10 | 103 | 1.98E−10 | 107 |
| | | 5GS-A0226018C08- | | | | |
| | | 35GS-A0226015A08- | | | | |
| | | 35GS-ALB23002-A | | | | |
| A022600132 | 441 | T017000679-5GS- | 6.83E−10 | 83 | 2.9E−10 | 95 |
| | | A0226018C08- | | | | |
| | | 9GS-A0226015A08- | | | | |
| | | 35GS-ALB23002-A | | | | |
| A022600175 | 451 | T017000680-5GS- | 5.27E−10 | 96 | 5.86E−11 | 99 |
| | | A0226018C08- | | | | |
| | | 9GS-A0226015A08- | | | | |
| | | 35GS-ALB23002-A | | | | |

Step 4—Effect of Position of the Anti-TCR ISVD within the GPC3 T-Cell Engager Format.

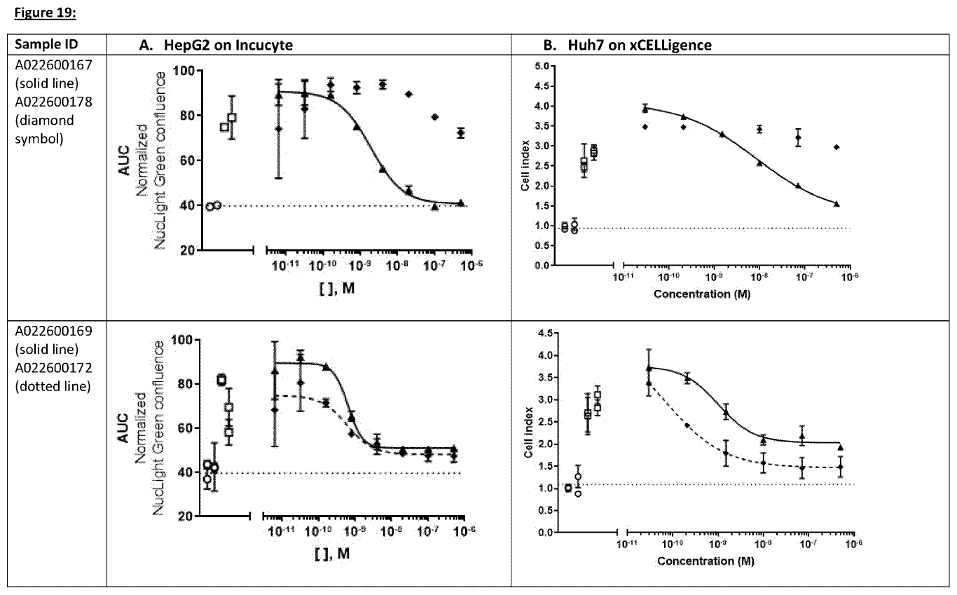

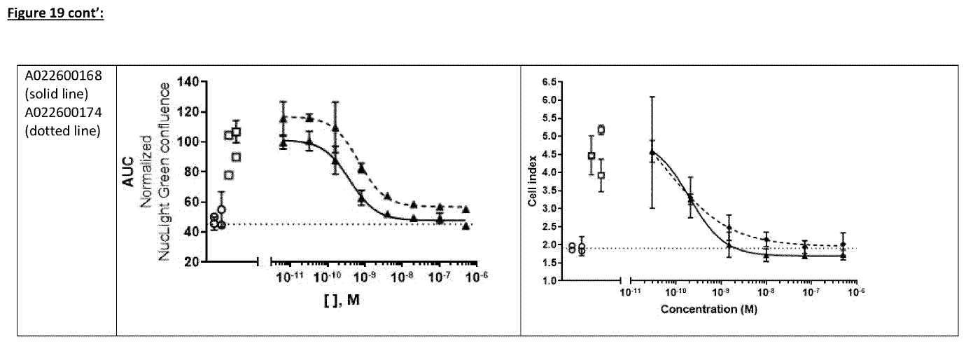

The position of the anti-TCR ISVD in the context of GPC3 T-cell engager formats had an influence on potency on efficacy (Table 36, FIG. 19). For the trivalent format the killing efficacy was lost. For the tetravalent formats functionality could still be observed, be it with a decrease in potency and efficacy; the killing efficacy decreased by 20% on HepG2 Nuclight green and by 40% on Huh7. Moreover, changing orientation of anti-GPC3 ISVD with anti-HSA ISVD also had an influence on efficacy of killing.

TABLE 36
IC50 (M) and maximum percentage of killing (%) of trispecific GPC3 ISVD T-cell engagers in
the Incucyte based human TDC HepG2-Nuclight green assay and in the xCELLigence based human
TDC Huh7 assay, using an effector to target ratio of 15:1, analyzed at 60 h after seeding

| | | | | HepG2 on | | Huh7 on |
|---|---|---|---|---|---|---|
| | | | HepG2 on | Incucyte | Huh7 on | xCELLigence |
| | SEQ | | Incucyte | Max % | xCELLigence | Max % |
| Sample ID | ID NO | Description | IC50 (M) | killing | IC50 (M) | killing |
| A022600167 | 445 | T017000680-5GS- | 1.92E−09 | 98 | 8.61E−09 | 91 |
| | | A0226018C08- | | | | |
| | | 35GS-ALB23002-A | | | | |
| A022600178 | 452 | A0226018C08-5GS- | no effect | | no effect | |
| | | T017000697- | | | | |
| | | 35GS-ALB23002-A | | | | |
| A022600168 | 446 | T017000680-5GS- | 3.67E−10 | 103 | 1.98E−10 | 107 |
| | | A0226018C08- | | | | |
| | | 35GS-A0226015A08- | | | | |
| | | 35GS-ALB23002-A | | | | |
| A022600169 | 447 | A0226018C08-5GS- | 6.41E−10 | 81 | 1.02E−09 | 61 |
| | | T017000697- | | | | |
| | | 5GS-A0226015A08- | | | | |
| | | 35GS-ALB23002-A | | | | |
| A022600174 | 450 | T017000680-5GS- | 6.73E−10 | 96 | 6.26E−11 | 99 |
| | | A0226018C08- | | | | |
| | | 9GS-A0226015A08- | | | | |
| | | 9GS-ALB23002-A | | | | |
| A022600172 | 449 | T017000680-5GS- | 6.01E−10 | 81 | 6.70E−11 | 90 |
| | | A0226018C08- | | | | |
| | | 9GS-ALB23002- | | | | |
| | | 9GS-A0226015A08-A | | | | |

### Step 5—Effect of Linker Lengths Between Anti-GPC3 ISVD and Anti-HSA ISVD.

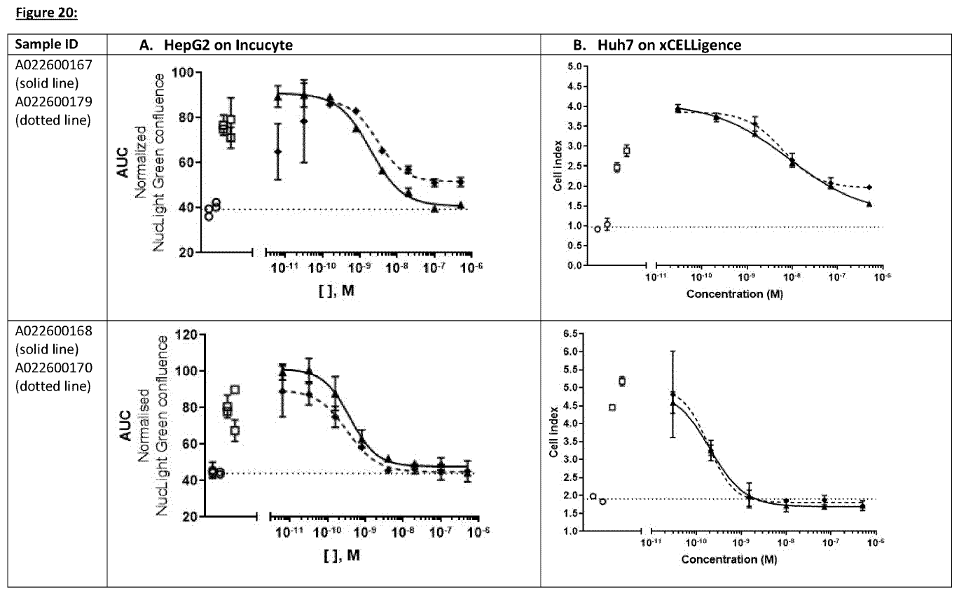

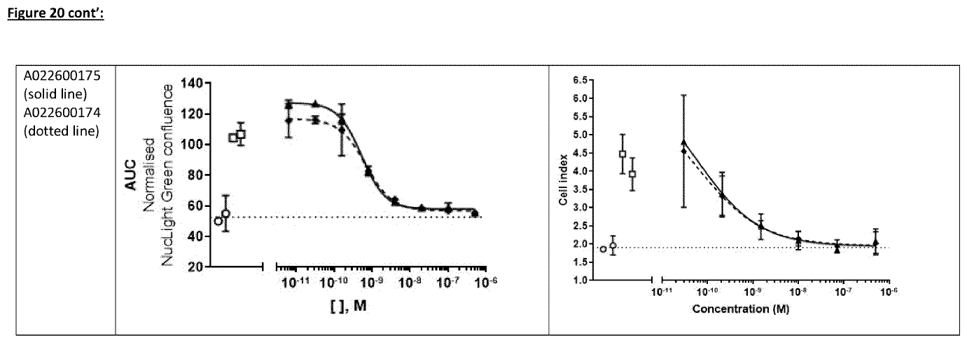

Decreasing the linker length before the anti-HSA ISVD in the trivalent format resulted in decreased efficacy (Table 37, FIG. 20). This is not seen in the tetravalent format, so in this case there is no preference between 9GS and 35GS linker lengths.

From the set of GPC3 T-cell engager formats A022600167 and A022600168 represent the trispecific trivalent and tetravalent formats with the highest potency and efficacy in the TDC assays. While efficacies are comparable, the trivalent format A022600167 and the tetravalent format A022600168 differ in potency. In the HepG2 TDC assay, A022600167 is 5-fold less potent than A022600168 while in the Huh7 TDC assay the difference increases to 50-fold.

TABLE 37
IC50 (M) and maximum percentage of killing (%) of trispecific GPC3 ISVD T-cell engagers in
the Incucyte based human TDC HepG2-Nuclight green assay and in the xCELLigence based human
TDC Huh7 assay, using an effector to target ratio of 15:1, analyzed at 60 h after seeding

| | | | | HepG2 on | | Huh7 on |
|---|---|---|---|---|---|---|
| | | | HepG2 on | Incucyte | Huh7 on | xCELLigence |
| | SEQ | | Incucyte | Max % | xCELLigence | Max % |
| Sample ID | ID NO | Description | IC50 (M) | killing | IC50 (M) | killing |
| A022600167 | 445 | T017100680-5GS- | 1.92E−09 | 98 | 8.61E−09 | 91 |
| | | A0226018C08- | | | | |
| | | 35GS-ALB23002-A | | | | |
| A022600179 | 453 | T017100680-5GS- | 2.92E−09 | 76 | 6.13E−09 | 66 |
| | | A0226018C08- | | | | |
| | | 9GS-ALB23002-A | | | | |
| A022600168 | 446 | T017100680-5GS- | 3.67E−10 | 103 | 1.98E−10 | 107 |
| | | A0226018C08- | | | | |
| | | 35GS-A0226015A08- | | | | |
| | | 35GS-ALB23002-A | | | | |
| A022600170 | 448 | T017100680-5GS- | 3.19E−10 | 97 | 1.86E−10 | 103 |
| | | A0226018C08- | | | | |
| | | 35GS-A0226015A08- | | | | |
| | | 9GS-ALB23002-A | | | | |
| A022600175 | 451 | T017100680-5GS- | 5.27E−10 | 96 | 5.86E−11 | 99 |
| | | A0226018C08- | | | | |
| | | 9GS-A0226015A08- | | | | |
| | | 35GS-ALB23002-A | | | | |
| A022600174 | 450 | T017100680-5GS- | 6.73E−10 | 96 | 6.26E−11 | 99 |
| | | A0226018C08- | | | | |
| | | 9GS-A0226015A08- | | | | |
| | | 9GS-ALB23002-A | | | | |

### 6.21 Example 21: GPC3 Expression Profiling of Cancer Cell Lines and Correlation with Functionality of GPC3 T-Cell Engagers

GPC3 protein expression levels for a panel of cancer cell lines was determined both by immunocytochemistry (ICC) and using the QIFIKIT® (Dako, Cat no K0078) according to the manufacturer's instructions. Additionally, immunohistochemistry (IHC) was performed on hepatocellular carcinoma and normal kidney samples.

ICC and IHC was performed using the Ventana discovery XT robot (Ventana medical system, Roche). The cell lines and the tissue samples were first fixed on 4% formalin and subsequently paraffin embedded. After deparaffinization, cells were conditioned with buffer CC1 standard (Ventana, Cat no 950-124) at a temperature of 95° C. for 48 minutes, followed by a blocking step of 4 minutes with each of Blocker A and B (Ventana, Cat no 760-104). Mouse monoclonal IgG2a anti-GPC3 antibody (Ventana, Cat no 790-4564) was applied for 60 minutes at room temperature, followed by 4 minutes of fixation with 0.05% Glutaraldehyde in 5 M NaCl (Prolabo, Cat no 20879-238). Biotinylated goat anti-mouse IgG2a antibody (Southern Biotech, Cat no 1080-080) at 1/200 dilution in antibody diluent (Ventana; Cat no 760-108) was applied for 32 minutes at room temperature. Detection was performed with the DABMap Kit (Ventana; Cat no 760-124). Sections were counterstained for 4 minutes with Hematoxylin II (Ventana, Cat no 790-2208) and post-counterstained for 4 minutes with bluing agent (Ventana, Cat no 760-2037), followed by deshydratation and mounting with Cytoseal XYL (Richard-Allan Scientific, Cat no 8312-4). Immunohistochemical staining was evaluated by a semi-quantitative assessment of both the intensity of staining of the cells (graded as 0: no staining, 1 (or +): weak, 2 (or ++): moderate; 3 (or +++): strong) and percentage of positive cells in every intensity categories. Histoscore (H-score) was calculated according to following formula: H-score=3×(cell % with grade 3)+2×(cell % with grade 2)+1×(cell % with grade 1).

The range of the possible score was from 0 to 300, as described in literature (Detre et al., J Clin Pathol 1995; 48:876-878 and Lui et al., Journal of Latex Class filed, august 2015, vol 14, No. 8). Determination of H-score in HCC was based on evaluation of membranous expression ofGPC3.

Within the panel of cell lines tested, as determined with the QIFIKIT®, Hep-G2 (ATCC, clone HB-8065; 5.2E5 receptors/cell) showed the highest level of expression of GPC3 followed by NCI-H661 (ATCC, clone HTB-183; 3.4E5 receptors/cell) and Huh-7 (HSRRB, clone JCRB0403; 6.8E4 receptors/cell), the latter considered as medium expressing cell line. These cell lines originated from liver and lung cancers which are relevant GPC3 expressing solid tumors. The low or very low GPC3 expressing cell lines, which did not show any staining in ICC, were MKN-45 (DSMZ, clone ACC409; 1.5E4 receptors/cell), NCI-H23 (ATCC, clone CRL-5800; 2.6E3 receptors/cell), BxPC-3 (ATCC, clone CRL-1687; 1.5E3 receptors/cell) and NCI-H292 (ATCC, clone CRL-1848; 6E2 receptors/cell) (Table 38).

For comparison between the cancer cell lines and patient tumour samples, the H-scores were determined for both. GPC3 positive cancer cell lines in ICC, i.e. Huh-7, NCI-H661 and HepG2, showed H-scores of superior to 80 (Table 38), which corresponds to the average H-score determined for the GPC3 positive hepatocellular carcinoma (HCC) samples in IHC of 80,75 (Table 39). Normal kidney GPC3 positive samples show an average H-score of 0,75 (Table 39) while the cancer cell lines MKN-45, NCI-H23, BxPC-3 and NCI-H292 were negative for GPC3 staining (Table 38). These cell lines were taken as representatives of GPC3 normal-like expression level cells.

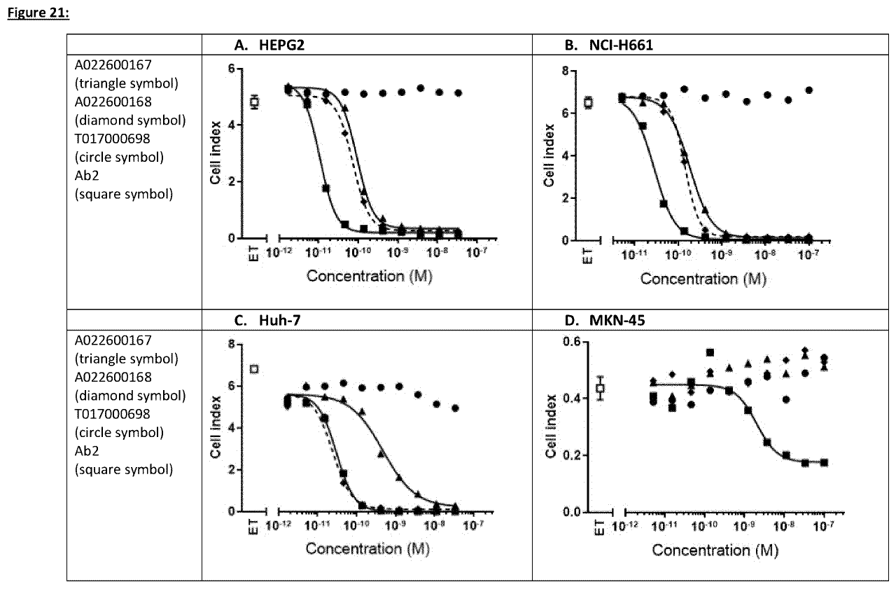

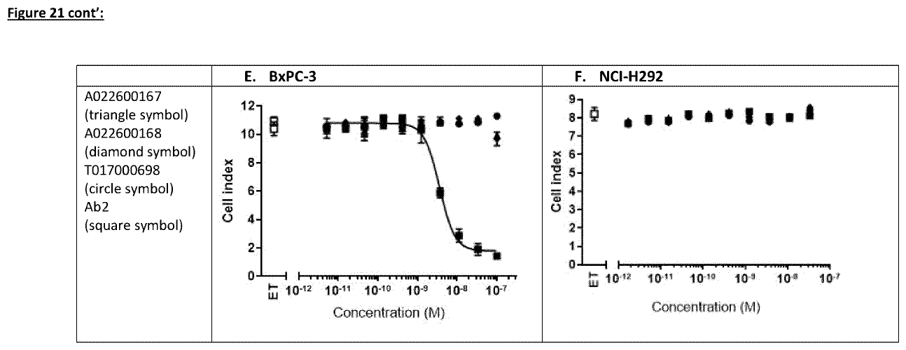

To assess the functionality of the trispecific GPC3 T-cell engagers with the same panel of cancer cell lines, TDC assays were performed using the xCELLigence system, as described in Example 5; results are shown in Table 40 and FIG. 21. For the GPC3 high expressing cell lines, Hep-G2 and NCI-H661, the trispecific GPC3 ISVD T-cell engagers A022600167 and A022600168 show similar potency (NCI-H661) and 10-fold lower potency (Hep-G2), compared to the bispecific T-cell engager benchmark antibody. For the medium expressing cell line, Huh-7, A022600168, the tetravalent format with biparatopic binding to GPC3, shows the same potency as Ab2, while the potency of the trivalent format A022600167 is 10-fold lower. For the GPC3 low expressing cell lines, MKN-45 and BxPC-3, Ab2 shows a potency in the nM range, while the trispecific GPC3 ISVD T-cell engagers show no killing effect. For the very low GPC3 expressing cell line, NCI-H292, none of the compounds show an effect. The T-cell engager lacking a GPC3 binding ISVD, T017000698, does not show any killing effect on any cell line, confirming the GPC3 specific effect of the trispecific GPC3 T-cell engagers.

In conclusion, Ab2 is a potent T-cell engager able to kill cancer cell lines with GPC3 expression levels as low as a thousand receptors per cell and H-score 0. In comparison, the trispecific T-cell engager formats potently kill high and medium GPC3 expressing cancer cell lines with H-scores similar to HCC and large cell lung cancer samples while sparing cell lines expressing GPC3 levels below ten thousand receptors per cell and with H-scores below the

TABLE 38
GPC3 expression levels of different cancer cell lines.

| | | | Expression | Expression |
|---|---|---|---|---|
| | Cancer | Expression RNA | protein | protein |
| Cell line | tissue | FPKM | #GPC3/cell | ICC (H-score) |
| Hep-G2 | Liver | 2253 | 619006 | 70%+++, |
| | | | | 25%++ (260) |
| NCl-H661 | Lung | 237 | 346756 | 40%++ (80) |
| | Liver | 549 | 78027 | 20%++, |
| Huh-7 | | | | 40%+ (80) |
| MKN-45 | Stomach | 20.9 | 7453 | 0 |
| NCl-H23 | Lung | 3.17 | 2255 | 0 |
| BxPC3 | Pancreas | 5.3 | 1332 | 0 |
| NCl-H292 | Lung | 0.04 | 452 | 0 |

TABLE 39
GPC3 H-score determined by immunohistochemistry on Hepatocellular
carcinoma and normal kidney samples (based on evaluation
of membranous expression of GPC3).

| | | | Total | |
|---|---|---|---|---|
| | Total | | GPC3+ | |
| | cases | H-score | cases | H-score |
| HCC | 288 | 52.22 | 187 | 80.75 |
| Normal kidney | 35 | 0.17 | 8 | 0.75 |

TABLE 40
IC50 (M) of the GPC3 T-cell engagers in the xCELLigence based human TDC assay on different
tumor cell lines expressing decreasing expression levels of GPC3 using an effector to target
ratio of 15:1: HepG2 analysed at 60 h, NCI-H661 analysed at 75 h, Huh-7 analysed at 60
h, MKN-45 analysed at 65 h, BxPC-3 analysed at 65 h, NCI-H292 analysed at 60 h.

| | SEQ | | | | | | | |
|---|---|---|---|---|---|---|---|---|
| Sample ID | ID NO | Description | Hep-G2 | NCI-H661 | Huh-7 | MKN-45 | BxPC-3 | NCI-H292 |
| A022600167 | 445 | TCE01-5GS- | 9.2E−11 | 1.8E−10 | 4.7E−10 | n.a. | n.a. | n.a. |
| | | A0226018C08- | | | | | | |
| | | 35GS-ALB23002-A | | | | | | |
| A022600168 | 446 | TCE01-5GS- | 7.3E−11 | 1.4E−10 | 2.4E−11 | n.a. | n.a. | n.a. |
| | | A0226018C08- | | | | | | |
| | | 35GS-A0226015A08- | | | | | | |
| | | 35GS-ALB23002-A | | | | | | |
| T017000698 | 454 | TCE01-9GS- | n.a. | n.a. | n.a. | n.a. | n.a. | n.a. |
| | | ALB23002-A | | | | | | |
| Ab2 | — | Bispecific CD3- | 1.1E−11 | 2.8E−11 | 3.0E−11 | 2.0E−09 | 3.6E−09 | n.a. |
| | | GPC3 | | | | | | |

n.a. = not available

### 6.22 Example 22: Generation of Trispecific Sequence Optimized GPC3 ISVD T-Cell Engagers

The sequence optimized GPC3 ISVDs A022600314 (optimized variant of A0226015A08), A022600345 and A022600351 (optimized variants of A0226018C08) were used for the generation of five trispecific GPC3 T-cell engager formats for assessment of optimal combination of building blocks and linker length as described in Table 41.

TABLE 41
Selection of different trispecific GPC3
ISVD T-cell engager formats evaluated

| | SEQ | |
|---|---|---|
| Sample ID | ID NO | Description |
| A022600427 | 459 | T017000680-5GS-A022600345-9GS- |
| | | A022600314-9GS-ALB23002-A |
| A022600424 | 456 | T017000680-5GS-A022600351-9GS- |
| | | A022600314-9GS-ALB23002-A |
| A022600425 | 457 | T017000680-5GS-A022600314-20GS- |
| | | A022600345-9GS-ALB23002-A |
| A022600426 | 458 | T017000680-5GS-A022600314-20GS- |
| | | A022600351-9GS-ALB23002-A |
| A022600412 | 455 | T017000680-5GS-A022600345-20GS- |
| | | ALB23002-A |

The five selected formats were tested in the xCELLingence based TDC assay with different cancer cell lines for functionality, as described in Example 21. Results are depicted in Table 42. For high and medium GPC3 expressing cell lines HepG2, NCI-H661 and Huh-7, higher potencies were obtained for the tetravalent formats compared to the trivalent format. For high GPC3 expressing cell line NCI-H661, tetravalent formats A022600424 and A022600427 are more potent than A022600425 and A022600426. For the GPC3 low expressing cell lines, NCI-H23 and BxPC-3, the lack of a killing effect was confirmed for all trispecific GPC3 ISVD T-cell engager formats.

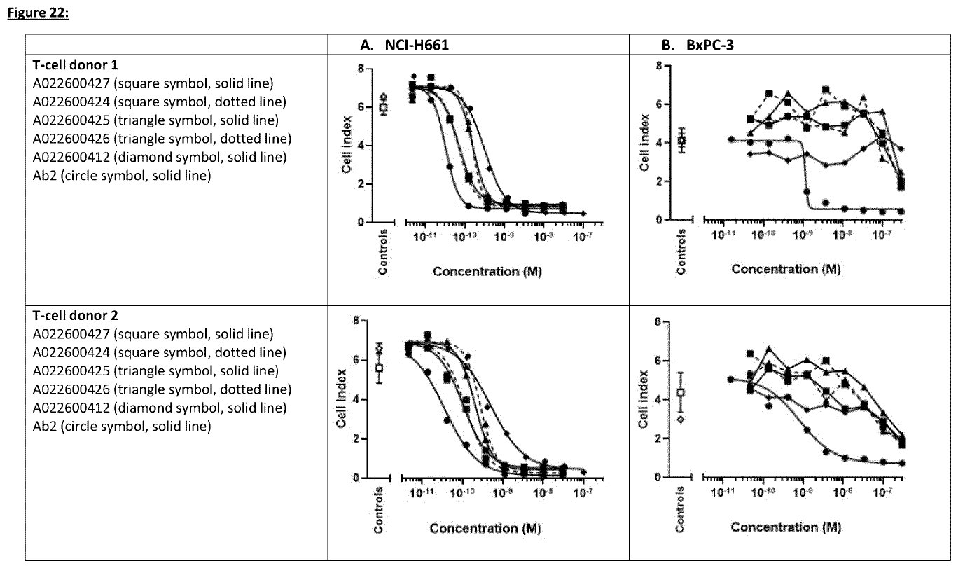

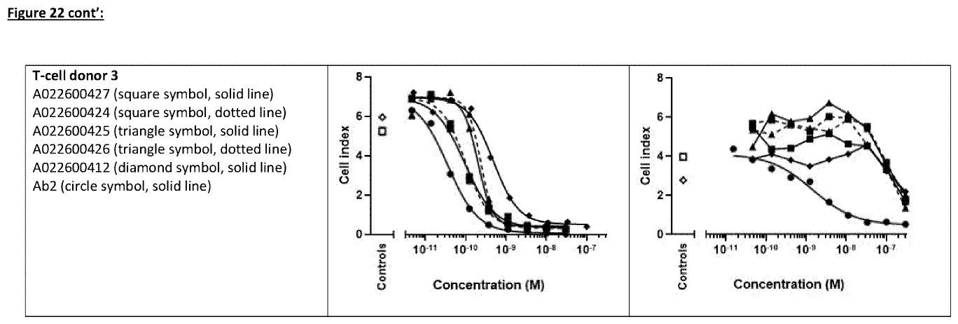

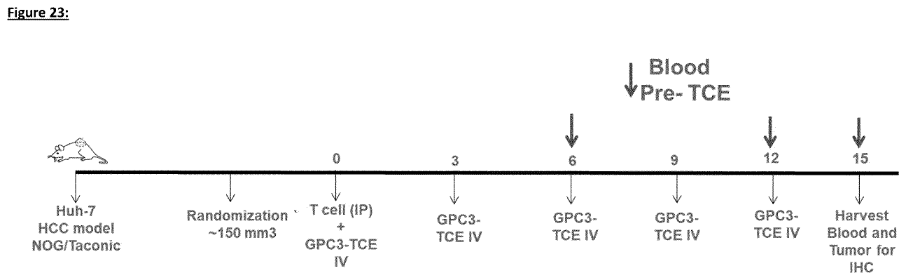

FIG. 22 shows the dose-dependent killing curves for the xCELLingence based TDC assay, exemplified by cell lines NCI-H661 and BxPC-3, using three different T-cell donors for the five selected ISVD formats.

TABLE 42
IC50 (M) of GPC3 T-cell engagers in the xCELLigence based human
TDC assay on different tumor cell lines using an effector to
target ratio of 15:1 and analyzed at 60 h. na = not available;
no fit = no curve fit obtained, IC50 estimated as >1E−7M

| | IC50 (M) | IC50 (M) | IC50 (M) | IC50 (M) | IC50 (M) |
|---|---|---|---|---|---|
| Sample ID | HepG2 | NCl-H661 | Huh-7 | NCl-H23 | BxPC-3 |
| A022600427 | 1.03E−10 | 1.00E−10 | 1.37E−10 | no fit | no fit |
| A022600424 | 1.15E−10 | 1.14E−10 | 1.16E−10 | no fit | no fit |
| A022600425 | 1.11E−10 | 2.59E−10 | 1.58E−10 | no fit | no fit |
| A022600426 | 1.25E−10 | 2.64E−10 | 1.09E−10 | no fit | no fit |
| A022600412 | 3.82E−10 | 6.18E−10 | 1.53E−09 | na | no fit |
| Ab2 | 1.49E−11 | 4.60E−11 | 8.41E−11 | 1.28E−09 | 1.09E−09 |

### 6.23 Example 23: Binding and Affinity of A022600424 to GPC3, the TCR Zipper Proteins and Serum Albumin

The affinity, expressed as the equilibrium dissociation constant (K_{D}), of A022600424 towards human and cyno GPC3 (R&D Systems, cat no 2119-GP and in house produced (accession number P51654, Q3-R358, 5359-H559), respectively), huTCR-zipper and cyTCR-zipper protein and human, cyno and mouse serum albumin (Sigma cat no A8763, in house produced from animal tissue, DivBioScience cat no IMSA, respectively) was quantified by surface plasmon resonance (SPR) using a ProteOn XPR36.

Recombinant GPC3 proteins were captured on a GLC Sensor Chip (Biorad) immobilized with THE anti-His antibody (Genscript, cat no ABIN387699) via amine coupling, using EDC and NHS chemistry (running buffer: HBS-EP+, pH7.4). Purified ISVDs were injected for 2 minutes (flow rate 45 μL/min) at different concentrations (between 0.3 nM and 1000 nM) and dissociation was followed for 900 s. Regeneration was performed by injecting 10 mM Glycine-HCl (pH 1.5) for 1 minute (flow rate 45 μL/min). Data was double referenced by subtracting a reference ligand lane and a blank buffer injection. Processed sensorgrams were analyzed based on the 1:1 interaction model (Langmuir binding model) using ProteOn Manager 3.1.0 (Version 3.1.0.6) software.

Recombinant TCR proteins were immobilized on a GLC Sensor Chip (Biorad) via amine coupling, using EDC and NHS chemistry (running buffer: HBS-EP+, pH 7.4). Purified ISVDs were injected for 2 minutes (flow rate 45 μL/min) at different concentrations (between 0.2 nM and 200 nM) and dissociation was followed for 900 s. Regeneration was performed by injecting 3 M MgCl₂ for 3 minutes (flow rate 90 μL/min). Data was double referenced by subtracting a reference ligand lane and a blank buffer injection. Processed sensorgrams were analyzed based on the 1:1 interaction model (Langmuir binding model) using ProteOn Manager 3.1.0 (Version 3.1.0.6) software.

The results (Table 43) demonstrate that A022600424 binds human and cyno GPC3 with high affinity.

TABLE 43
Binding affinities to human and cyno GPC3,
human and cyno TCR-zipper and human, cyno
and mouse serum albumin. n.a. = not available

| | | KD (M) | KD (M) | KD |
|---|---|---|---|---|
| Antigen | Sample ID | human | cynomolgus | mouse |
| GPC3 | A022600424 | <5.6E−12 | <5.9E−12 | n.a. |
| | Ab2 | 1.8E−09 | 1.7E−09 | n.a. |
| TCRab | A022600424 | 6.3E−09 | 5.4E−09 | n.a. |
| | T017000698 | 8.5E−09 | 1.1E−08 | n.a. |

Binding of A022600424 to cell expressed human and cyno GPC3 was assessed by flow cytometry for CHO-Flp-In cells overexpressing human and cyno GPC3, as well as Huh-7 cells, yielding EC50 values between 1 and 2 nM (Table 44). A022600424 was evaluated for binding to human and cyno T cells in competition with TCRab binding monovalent ISVDs T017000624 and T017000623 (T0170056G05 variants) respectively, at EC30 concentrations. T cells (human T cells obtained as described in Example 3 and cyno T cells purchased from LPT laboratory, Germany) were thawed and counted on the day of the assay and diluted to a concentration of 1E+06 cells/mL, before adding 75 μL to the wells of a V-bottom 96-well plate (Greiner, cat no 651180). Cells were washed once with cold FACS buffer, before adding 25 μL Nb and 25 μL competitor to the wells. T017000624 was diluted to a 2× concentration of 4E-08 M (2E-08 M in well), T017000623 was diluted to a 2× concentration of 1E-07 M (5E-08 M in well) and A022600424 was diluted to final concentrations in the wells ranging between 8 μM and 7.8 nM. Cells were resuspended and plates were incubated at 4° C. for 90 minutes, after which plates were washed twice in cold FACS buffer. Cells were resuspended in 50 μL 1/1000 diluted Monoclonal ANTI-FLAG® M2 (Sigma Aldrich, cat no F1804) in FACS buffer and incubated at 4° C. for 30 minutes. Plates were washed twice in cold FACS buffer. Cells were resuspended in 50 μL 1/100 diluted Allophycocyanin-conjugated AffiniPure Goat Anti-Mouse IgG (subclasses 1+2a+2b+3), Fc Fragment Specific (Jackson Immunoresearch, cat no 115-135-164) in FACS buffer and incubated at 4° C. for 30 minutes. Plates were washed twice in cold FACS buffer. Cells were resuspended in 55 μL 1/1000 diluted Propidium Iodide (Sigma-Aldrich, cat no P4170) in FACS buffer before acquiring data on the MACSQuant X (Miltenyi biotec).

The results are shown in Table 44. A022600424 binds with approx. 200 nM affinity to both, human and cyno T-cells.

TABLE 44
Binding assessment of A022600424 to cell expressed
human and cyno GPC3 and human and cyno TCR.

| | | CHO huGPC3 | CHO cyGPC3 | Huh-7 |
|---|---|---|---|---|
| Antigen | Sample ID | EC50 (M) | EC50 (M) | EC50 (M) |
| GPC3 | A022600424 | 1.82E−09 | 1.11E−09 | 1.25E−09 |
| (binding | Ab2 | 1.69E−08 | 5.4E−09 | 5.2E−09 |
| FACS) | | | | |

| | | Primary | Primary |
|---|---|---|---|
| | | human | cynomolgus |
| | | T cells | T cells |
| | | IC50 (M) | IC50 (M) |
| TCR | A022600424 | 1.7E−07 | 2.5E−07 |
| (competition | T017000698 | 1.9E−07 | 2.5E−07 |
| FACS) | | | |

To assess A022600424 functionality with cyno T-cells, a xCELLigence based TDC assay on Huh-7 was performed using cyno T-cells (LPT laboratory, Germany), as described in Example 21. IC50 values for cyno and human T cells were found to be comparable (Table 45).

TABLE 45
Functionality of A022600424 using cynomolgus T-cells
in the xCELLigence based TDC assay on Huh-7, with an
effector to target ratio of 15:1 and analyzed at 60 h.

| | Primary hu T | Primary cy T |
|---|---|---|
| Sample ID | cells IC50 (M) | cells IC50 (M) |
| A022600424 | 7.59E−11 | 2.29E−10 |
| Ab2 | 7.88E−11 | 3.57E−11 |

### 6.24 Example 24: In Vivo Proof-of-Concept for A022600424 in Huh-7 Tumor Bearing NOG Mice Engrafted with In Vitro Expanded T Cells

In an in vivo efficacy study in tumor bearing NOG mouse, Hepatocellular Carcinoma Huh-7 tumor cells were injected subcutaneously, and tumors were allowed to grow until a mean tumor volume of ˜150 mm³ was reached. At this point, in vitro expanded T cells were injected intraperitoneally into the mice. Tumor cell killing by ISVD-mediated recruitment of T cells was evaluated by measuring the tumor volume and analyzing the tumor growth kinetics. The in vivo efficacy of A022600424 on tumor cell killing was evaluated and compared with the control T-cell engager T017000698 (SEQ ID NO: 454, Table A-9) lacking the GPC3 specificity.

In detail, 2×10⁶ Huh-7 tumor cells resuspended in 100 μL of HBSS were subcutaneously injected in NOG mice. The tumors grew until the mean tumor volume of approximately 150 mm³ was reached. At this point, 10⁷ in vitro expanded T cells resuspended in 200 μL of PBS were injected into each mouse intraperitoneally (DO). This injection of T cells took place 24 hours after mice were randomized into different groups. The treatment with A022600424 injected intravenously started on DO, 3 h after T cell injection and continued D3, D6, D9 and D12 (q3d; FIG. 23). Four dose levels of the TCR/GPC3 binding polypeptides were tested (0.1 mg/kg, 0.2 mg/kg, 0.7 mg/kg and 2 mg/kg). T017000698 was injected in a control group at 2 mg/kg on DO, D3, D6, D9 and D12 (q3d). Survival blood sampling in heparin containing tubes was done on D6 and D12 to measure antibody exposure. Mice were sacrificed on D15, and blood and tumor tissue were collected. Blood was used for the antibody exposure measurements and tumor tissue was used for analysis of target expression (GPC3) and T cell infiltration analysis.

Results for tumor growth kinetics are shown in FIG. 24. Mice treated with T017000698 were used as control group for analyses on D24, when all control mice were alive as they did not reach end point criteria (2000 mm³ tumor volume). A dose response pattern was seen in tumor growth profile for A022600424 versus the control T017000698 for inducing tumor stasis. The 0.7 mg/kg (**, p=0.0016) and 2 mg/kg (*, p=0.0415) dose levels for the A022600424 were significantly different from the control T017000698. The 0.1 mg/kg, 0.2 mg/kg doses had lower effects on controlling tumor growth and were not significantly different to the control group. Statistical analysis has been performed with one-way ANOVA, using the Dunnett's multiple comparisons test for analysis.

In conclusion, the results demonstrate that A022600424 can dose-dependently induce statistically significant tumor stasis in this model. This confirms the concept of polypeptide-induced T cell-mediated killing via a GPC3 ISVD T-cell engager by cross-linking T cells to GPC3 on Huh-7 tumor cells.

### 6.25 Example 25: Generation of a Multispecific TCRαβ-CD33-CD123 ISVD Construct and Binding Affinity to TCRαβ, CD33, CD123 and Serum Albumin

The TCRαβ-CD33-CD123 multispecific ISVD construct as set forth in SEQ ID NO: 460 was generated.

### 6.25.1 Affinity Determination Towards Human and Cynomolgus CD33 and CD123 Proteins

The affinities, expressed as the association rate constant (kₐ), the dissociation rate constant (k_{d}) and the equilibrium dissociation constant (K_{D}), of the TCRαβ-CD33-CD123 multispecific ISVD construct (SEQ ID NO: 460) towards recombinant huCD33L-Fc, cyCD33L-Fc (via capturing setup) or huCD123 (via capturing setup) proteins were measured at 37° C. by means of a surface plasmon resonance (SPR)-based assay on a ProteOn XPR36 instrument (BioRad Laboratories, Inc.).

### Setup for Measuring Binding Affinity Towards CD33

Anti-hulgG (Fc) was immobilized on a GLH (long matrix, high capacity) sensor chip via amine coupling, using EDC (1-Ethyl-3-(3-dimethylaminopropyl)carbodiimide) and NHS (N-hydroxysuccinimide esters) chemistry. Next, huCD33L-Fc and cyCD33L-Fc were captured (association: 120 s, 25 μL/min). Purified TCRαβ-CD33-CD123 multispecific ISVD construct as set forth in SEQ ID NO: 460 was injected at different concentrations (0.4 to 625 nM) for 120 s and dissociation was followed for 900 s.

### Setup for Measuring Binding Affinity Towards CD123

Anti-hulgG (Fc) was immobilized on a GLH (long matrix, high capacity) sensor chip via amine coupling, using EDC (1-Ethyl-3-(3-dimethylaminopropyl)carbodiimide) and NHS (N-hydroxysuccinimide esters) chemistry. Next, huCD123-Fc and cyCD123-Fc were captured (association: 120 s, 25 μL/min). Purified TCRαβ-CD33-CD123 multispecific ISVD construct as set forth in SEQ ID NO: 460 was injected at different concentrations (1.2 to 300 nM) for 120 s and dissociation was followed for 900 s, at 45μl/min.

Data was double referenced by subtracting a reference analyte lane and a blank buffer injection. Affinity constants (kₐ, k_{d}, K_{D}) were calculated applying the Langmuir 1:1 interaction model using ProteOn Manager 3.1.0 (Version 3.1.0.6) software.

The results of the affinity measurement of TCRαβ-CD33-CD123 multispecific ISVD construct as set forth in SEQ ID NO: 460 to human and cynomolgus CD33 and human and cynomolgus CD123 are summarized in Table 46 and Table 47 below.

TABLE 46
SPR based affinity determination of TCRαβ-CD33-CD123 multispecific
ISVD construct for human and cynomolgus CD33 protein

| | Human CD33L-Fc | | | Cynomolgus CD33L-Fc | | |
|---|---|---|---|---|---|---|
| ISVD | kₐ (1/Ms) | k_{d} (1/s) | K_{D} (M) | kₐ (1/Ms) | k_{d} (1/s) | K_{D} (M) |
| TCRαβ-CD33-CD123 | 3.5E+05 | 2.7E−03 | 7.6E−09 | 2.8E+05 | 1.9E−03 | 6.9E−09 |
| (SEQ ID NO: 460) | | | | | | |

Reactivity of TCRαβ-CD33-CD123 multispecific ISVD construct towards cynomolgus CD33 (K_{D} = 6.9 nM) was comparable to human CD33 (K_{D} = 7.6 nM).

TABLE 47
SPR based kinetic determination of TCRαβ-CD33-CD123 multispecific
ISVD construct for human and cynomolgus CD123 protein

| | Human CD123-Fc | | | Cynomolgus CD123-Fc | | |
|---|---|---|---|---|---|---|
| ISVD | kₐ (1/Ms) | k_{d} (1/s) | K_{D} (M) | kₐ (1/Ms) | k_{d} (1/s) | K_{D} (M) |
| TCRαβ-CD33-CD123 | 1.3E+06 | 7.4E−04 | 5.9E−10 | 1.2E+06 | 2.4E−03 | 2.0E−09 |
| (SEQ ID NO: 460) | | | | | | |

Reactivity of TCRαβ-CD33-CD123 multispecific ISVD construct towards cynomolgus CD123 (K_{D} = 2 nM) was 3.3-fold lower than to the human CD123 (K_{D} = 0.59 nM).

The results (Table 46 and Table 47) demonstrate that the multispecific ISVD construct binds human/cynomolgus CD33 and human/cynomolgus CD123 with high affinity.

### 6.25.2 Affinity Determination Towards Human and Cynomolgus TCRαβ Protein

The affinities, expressed as the association rate constant (kₐ), the dissociation rate constant (k_{d}) and the equilibrium dissociation constant (K_{D}), of the TCRap-CD33-CD123 multispecific ISVD construct (SEQ ID NO: 460) towards recombinant huTCR(2XN9)-zipper, cyTCR(AEA41865)-zipper (via coated setup) were evaluated at 37° C. by means of an SPR based assay on a ProteOn XPR36 instrument (BioRad Laboratories, Inc.).

### Setup for Measuring Binding Affinity Towards TCRαβ

huTCR(2XN9)-zipper, cyTCR(AEA41865)-zipper proteins were coated a GLC (short matrix, normal capacity) sensor chip. Purified TCRαβ-CD33-CD123 multispecific ISVD construct as set forth in SEQ ID NO: 460 was injected at different concentrations (0.4 to 625 nM) for 120 s and dissociation was followed for 900 s.

Data was double referenced by subtracting a reference analyte lane and a blank buffer injection. Affinity constants (kₐ, k_{d}, K_{D}) were calculated applying the Langmuir 1:1 interaction model using ProteOn Manager 3.1.0 (Version 3.1.0.6) software.

The results of the affinity measurement of TCRαβ-CD33-CD123 multispecific ISVD construct as set forth in SEQ ID NO: 460 to human and cynomolgus TCRαβ are summarized in Table 48 below. As a reference an ISVD consisting only of the TCRαβ building block (SEQ ID NO: 461) linked to the ALB23002 (SEQ ID NO: 464) was taken along.

TABLE 48
SPR based kinetic determination of TCRαβ-CD33-CD123 multispecific
ISVD construct and reference TCR-ISVD construct for human and cynomolgus TCRαβ

| | Human TCRαβ-zipper | | | | Cynomolgus TCRαβ-zipper | | | |
|---|---|---|---|---|---|---|---|---|
| ISVD | n | kₐ (1/Ms) | k_{d} (1/s) | K_{D} (M) | n | Kₐ (1/Ms) | k_{d} (1/s) | K_{D} (M) |
| TCRαβ-CD33-CD123 | 1 | 8.0E+04 | 1.7E−03 | 2.1E−08 | 1 | 4.9E+05 | 3.3E−03 | 6.7E−09 |
| (SEQ ID NO: 460) | | | | | | | | |
| TCR | Experiment1 | 1.4E+05 | 1.7E−03 | 1.2E−08 | Experiment1 | 4.2E+05 | 4.4E−03 | 1.1E−08 |
| (reference) | Experiment2 | 2.6E+05 | 1.8E−03 | 7.0E−09 | Experiment2 | 8.2E+05 | 3.5E−03 | 4.3E−09 |

Reactivity of TCRαβ-CD33-CD123 multispecific ISVD construct towards cyno TCRαβ (K_{D} = 6.7 nM) was 3-fold higher than to the human TCRαβ (K_{D} = 21 nM.)

The results (Table 48) demonstrate that the multispecific ISVD construct binds human/cynomolgus TCRαβ with high affinity.

### 6.25.3 Affinity Determination Towards Human, Mouse and Cynomolgus Serum Albumin

Binding affinity, expressed as the association rate constant (kₐ), the dissociation rate constant (k_{d}) and the equilibrium dissociation constant (K_{D}), of the TCRαβ-CD33-CD123 multispecific ISVD construct (SEQ ID NO: 460) towards recombinant to human, cynomolgus and mouse serum albumin via the C-terminal ALB23002 half-life extension (via coated setup) were evaluated at 37° C. by means of an SPR based assay on a ProteOn XPR36 instrument (BioRad Laboratories, Inc.).

### Setup for Measuring Binding Affinity Serum Albumin

Human, cynomolgus and mouse serum albumin were immobilized on a ProteOn GLC sensor chip using amine coupling using amine coupling, using EDC and NHS chemistry (running buffer used: HBS-EP+, pH 7.4). The albumins were immobilized at two concentrations of 2.5 μg/mL (HSA & MSA) and 5 μg/mL (CSA) in pH4.5 Acetate buffer, giving rise to immobilization levels of up to 220 RUs for CSA, 150 RUs for MSA and 110 RUs for HSA. Purified multivalent V_{HH}S were injected for 2 minutes (flow rate 45 μL/min) at different concentrations (between 4.3 nM and 416 nM) and dissociation was followed for 900 s. Regeneration between cycles consisted of an injection of 10 mM glycine-HCL, pH1.5 for 47 s at 100 μL/min.

Data was double referenced by subtracting a reference ligand lane and a buffer injection. Processed curves were evaluated via fitting with the Langmuir 1:1 interaction model using ProteOn Manager 3.1.0 (Version 3.1.0.6) software model and affinity constants (kₐ, k_{d}, K_{D}) were calculated.

The results of the affinity measurement of TCRαβ-CD33-CD123 multispecific ISVD construct as set forth in SEQ ID NO: 460 to human, mouse and cynomolgus serum albumin are summarized in Table 49 below.

TABLE 49
SPR based affinity determination of TCRαβ-CD33-CD123 multispecific ISVD construct for serum albumin

| | MSA | | | CSA | | | HSA | | |
|---|---|---|---|---|---|---|---|---|---|
| | kₐ | k_{d} | K_{D} | kₐ | k_{d} | KD | kₐ | k_{d} | K_{D} |
| ISVD | (1/Ms) | (1/s) | (M) | (1/Ms) | (1/s) | (M) | (1/Ms) | (1/s) | (M) |

| | | | | | | | |
|---|---|---|---|---|---|---|---|
| TCRαβ-CD33- | KD < 5E−07 | 2.5E+05 | 2.0E−03 | 7.9E−09 | 2.2E+05 | 2.1E−03 | 9.7E−09 |
| CD123 | | | | | | | |
| (SEQ ID NO: 460) | | | | | | | |

Cross-reactivity towards CSA was confirmed. In addition, although no kinetic parameters were reported, affinity for MSA was good enough to obtain half-life extension and adopt the half-life of the serum albumin.

The results (Table 49) demonstrate that the multispecific ISVD construct binds human/mouse/cynomolgus TCRαβ with high affinity.

### 6.26 Example 26: Multispecific ISVD Construct Binding Affinity to Membrane Bound CD33 and/or CD123

### Setup for Measuring Binding Affinity to CD33/CD123 on Target Cell Lines

The binding affinity of TCRαβ-CD33-CD123 multispecific ISVD construct towards target cell lines expressing CD33 and/or CD123 was evaluated using flow cytometry. The target cell lines expressing CD123 are described in detail in WO2018/091606A1.

Transfected CD33 cells were generated as follows. Stable CHO Flp-In (Invitrogen, R758-07) cell lines with recombinant overexpression of CD33, were generated using the Flp-In™ site-directed recombination technology (Flp-In™ System For Generating Stable Mammalian Expression Cell Lines by Flp Recombinase-Mediated Integration (Invitrogen, K601001, K601002)). Hereby, DNA integration occurs at a specific genomic location at an FRT (Flp Recombination Target) site by the Flp recombinase (pOG44) derived from Saccharomyces cerevisiae. The Flp-In™ host cell line and expression plasmid (pcDNA5) both contain this FRT site, thereby allowing a single homologous DNA recombination. The sequence for human CD33 was derived from NCBI RefSeq NP_001763, the sequence of cynomolgus CD123 was derived from NCBI genbank no. XP_005590138.

In brief, cells were harvested and transferred to a V-bottom 96-well plate (5×10⁴ cells per well) and incubated with a serial dilution of TCRαβ-CD123-CD33 multispecific ISVD construct for 30 min at 4° C. in FACS buffer (D-PBS (Gibco, 14190) with 10% FBS (Sigma, F7524) and 0.05% sodium azide (Acros organics, 19038)) in the presence of 30 μM clinical grade HSA (CSL Behring, 2160-679) in a final volume of 100 μL. Next, cells were washed three times with FACS buffer and incubated with 1 μg/mL mouse monoclonal ANTI-FLAG® M2 antibody (Sigma-Aldrich, F1804) for 30 min at 4° C. for detection of FLAG3His₆-tagged CD123-CD33-TCR multispecific ISVD construct or with 3 μg/mL mAb anti-V_{HH} antibody (ABH0077) (APS+In House, A-0006-00_ABH0077_SF_AB1891) for detection of the NANOBODY® ISVD with an ALB BB. Next, cells were washed 3 times with FACS buffer and incubated for 30 min at 4° C. with 5 μg/mL Allophycocyanin (APC) AffiniPure Goat Anti-Mouse IgG (subclasses 1+2a+2b+3), Fcγ Fragment Specific (Jackson Immunoresearch, 115-136-071), in a final volume of 100 μL. Subsequently, cells were resuspended in 50 μL cold FACS buffer supplemented with 1 μg/mL the Propidium iodide (PI, Sigma P4170) to distinguish live from dead cells. After staining, cells were acquired using the MACSQuant X flow cytometer (Miltenyi Biotec) and analyzed using FlowLogic (Miltenyi Biotec). First a P1 population which represented more than 80% of the total events was selected based on FSC-SSC distribution, to distinguish cells from debris. From this population (P1) the PI positive (dead) cells were excluded and the median APC fluorescence intensity of the PI negative cells was evaluated.

### Setup for Measuring Binding Affinity to Primary T Cells

The binding affinity of TCRαβ-CD33-CD123 multispecific ISVD construct towards primary T cells was evaluated using flow cytometry in a competition setup using the FLAG3His₆ tagged monovalent TCR ISVD as ligand.

In brief, human or cynomolgus primary T cells were thawed and transferred to a V-bottom 96-well plate (7.5×10⁴ cells per well in 100 μL) and incubated with a serial dilution of TCRαβ-CD33-CD123 multispecific ISVD construct and a fixed concentration of ligand for 30 min at 4° C. in FACS buffer (D-PBS (Gibco, 14190) with 10% FBS (Sigma, F7524) and 0.05% sodium azide (Acros organics, 19038)) in the presence of 30 μM clinical grade HSA CSL Behring, 2160-679) in a final volume of 100 μL. The concentration of ligand used in the assay was below its binding EC₅₀. After an incubation period of 90 min at 4° C., the level of ligand binding was determined via flow cytometry. Thereto, cells were washed 3 times and incubated with 1 μg/ml mouse monoclonal ANTI-FLAG® M2 antibody (Sigma-Aldrich, F1804) for 30 min at 4° C., washed again, and incubated for 30 min at 4° C. with 5 μg/ml Allophycocyanin (APC) AffiniPure Goat Anti-Mouse IgG (subclasses 1+2a+2b+3), Fcγ fragment specific (Jackson Immunoresearch, 115-136-071), in a final volume of 100 μL. Subsequently, cells were resuspended in FACS buffer supplemented with 1 μg/ml Propidium Iodide (PI, Sigma, P4170) to distinguish live from dead cells. After staining, cells were acquired using the MACSQuant X flow cytometer (Miltenyi Biotec) and analyzed using FlowLogic (Miltenyi Biotec). First a P1 population which represented more than 80% of the total events was selected based on FSC-SSC distribution, to distinguish cells from debris. From this population (P1) the PI positive (dead) cells were excluded and the median APC fluorescence intensity of the PI negative cells was evaluated.

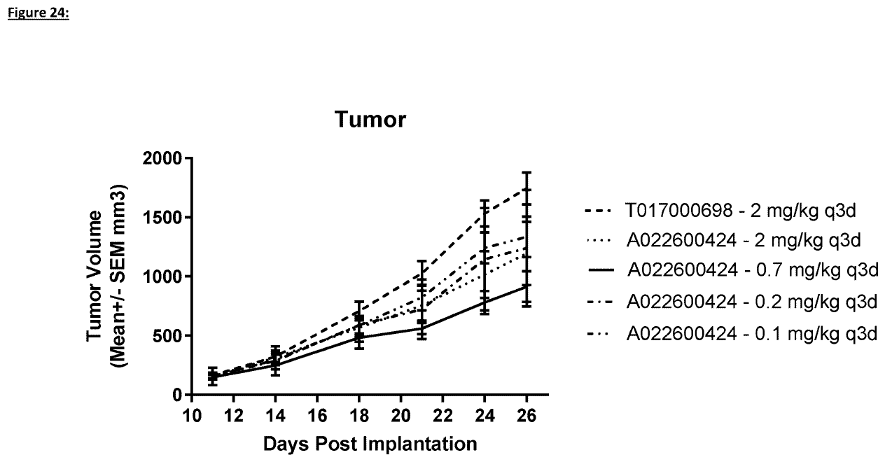

The human-cynomolgus cross-reactivity was evaluated by testing the binding of the monovalent CD33 building block (SEQ ID NO: 463) and the monovalent CD123 building block (SEQ ID NO: 462) to human or cynomolgus CD33 transfected cell lines or to human or cynomolgus CD123 transfected cell lines using flow cytometry as described above. The results are graphically represented in FIG. 26. The EC₅₀ values of the different experiments are summarized in Table 50 (CD33 transfected target cells) and Table 51 (CD123 transfected target cells).

TABLE 50
EC₅₀ values for binding of the CD33 building block (SEQ
ID NO: 463) to cells expressing huCD33 or cyCD33.

| | | EC50 | 95% LCI | 95% UCI |
|---|---|---|---|---|
| Sample ID | Cell line | (M) | (M) | (M) |
| CD33 binding ISVD | CHO cy CD33 | 5.7E−09 | 3.2E−09 | 5.1E−08 |
| CD33 binding ISVD | CHO cy CD33 | 1.2E−08 | 8.2E−09 | 2.2E−08 |
| CD33 binding ISVD | CHO hu CD33 | 6.2E−09 | 4.6E−09 | 8.5E−09 |
| CD33 binding ISVD | CHO hu CD33 | 5.5E−09 | 4.1E−09 | 7.7E−09 |
| CD33 binding ISVD | CHO hu CD33 | 7.7E−09 | 6.8E−09 | 8.8E−09 |

TABLE 51
EC₅₀ values for binding of the CD123 building block (SEQ
ID NO: 462) to cells expressing huCD123 or cyCD123.

| | | EC50 | 95% LCI | 95% UCI |
|---|---|---|---|---|
| Sample ID | Cell line | (M) | (M) | (M) |
| CD123 binding ISVD | CHO human CD123 | 3.1E−10 | 2.4E−10 | 4.1E−10 |
| CD123 binding ISVD | CHO human CD123 | 2.0E−10 | 2.9E−11 | undetermined |
| CD123 binding ISVD | HEK293 cyno CD123 | 8.0E−10 | 7.2E−10 | 9.1E−10 |

Binding of the monovalent CD33 and CD123 building blocks to human and cyno membrane target was confirmed. The difference in EC₅₀ between human and cynomolgus monkey was less than 2-fold decrease for binding to CD33 and 3-fold decrease for binding to CD123 cells) (Table 51).

In conclusion, in addition to the binding affinity towards recombinant human and cyno CD33 and CD123 protein dose-dependent binding of TCRαβ-CD33-CD123 multispecific ISVD construct to human and cyno cell-expressed CD33 and CD123 was also confirmed (n=1).

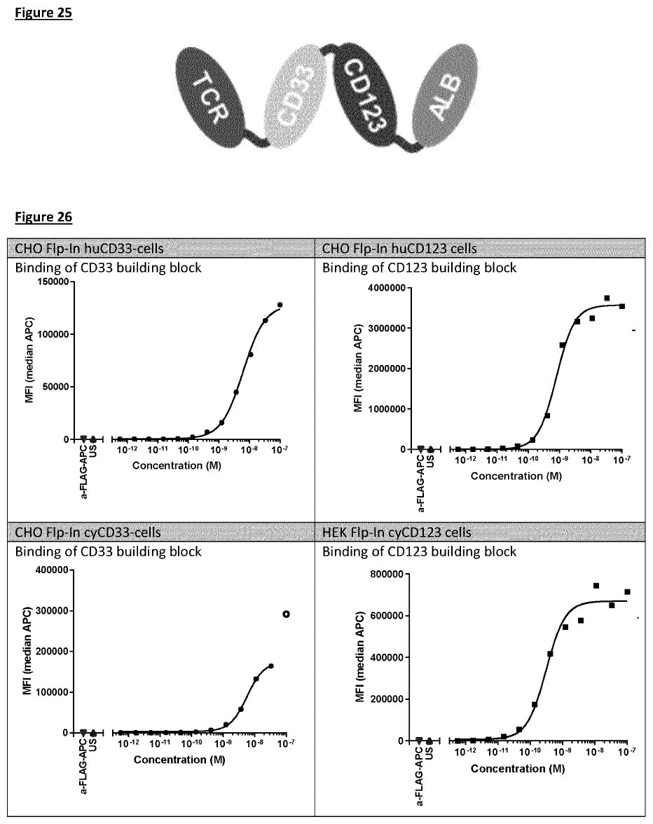

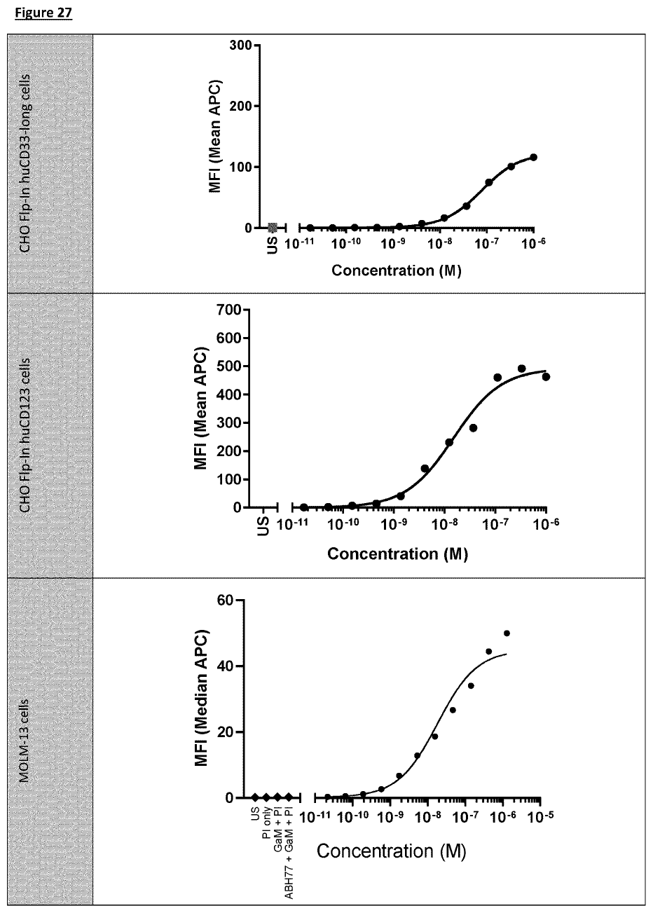

The binding of the CD123-CD33-TCR multispecific ISVD construct and the reference TCR-ISVD construct (consisting only of the TCRαβ building block (SEQ ID NO: 461) linked to the ALB23002 (SEQ ID NO: 464)) to human and cynomolgus T cells was evaluated in a competition setup using flow cytometry as described above (FIG. 27).

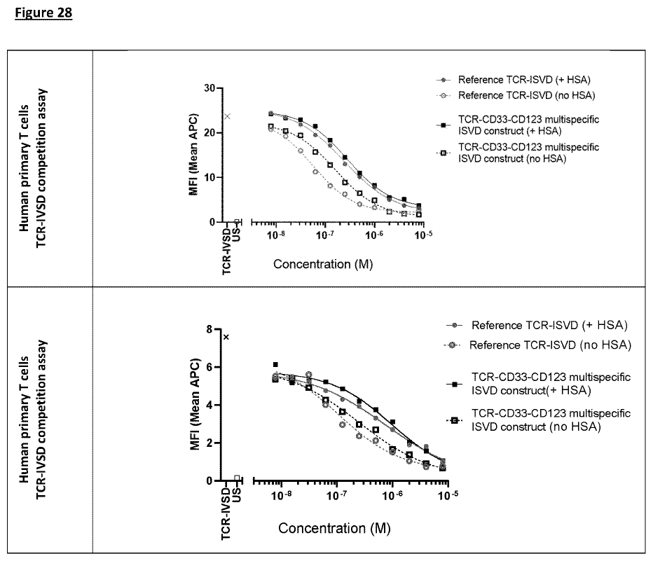

An exemplary example of the DRC is depicted in FIG. 28. The ISVD constructs were tested on multiple healthy donor T cells. Global IC₅₀ is shown in Table 52.

TABLE 52
Global IC₅₀ (M) of TCRαβ-CD33-CD123 multispecific ISVD
construct and reference TCR-ISVD construct in the human and
cynomolgus T cell competition assay as determined in flow cytometry

| | Primary human T | Primary cynomolgus T | Fold difference cyno |
|---|---|---|---|
| ISVD | cells Global IC50* | cells Global IC50* | versus human |
| TCRαβ-CD33-CD123 | 218 nM | 729 nM | 3.3 |
| (SEQ ID NO: 460) | [190 nM-249 nM] | [545 nM-976 nM] | |
| | n = 4 | n = 7 | |
| reference | 187 nM | 493 nM | 2.6 |
| TCR-ISVD | [134 nM-261 nM] | [314 nM-772 nM] | |
| | n = 4 | n = 7 | |

*global IC₅₀ (weighted)

In summary, the cross-reactivity of TCRαβ-CD33-CD123 multispecific ISVD construct towards cynomolgus primary T cells was confirmed. The EC₅₀ (=729 nM) on primary cynomolgus T cells was ˜3-fold higher than on the human primary T-cells (EC₅₀ (=218 M)

### 6.27 Example 27: Multispecific ISVD Construct Induced T Cell Mediated Target Cell Killing

### 6.27.1 Impedance-Based Cytotoxicity Assay

ISVD constructs were characterized for redirected T cell mediated killing in an impedance-based cytotoxicity assay (e.g. as described in WO2018091606A1) using human or cynomolgus primary effector T cells and adherent target cells. Changes in impedance induced by the adherence of target cells to the surface of an electrode were measured using the xCELLigence instrument (Roche). T cells are non-adherent and therefore do not impact the impedance measurements. The xCELLigence® RTCA MP instrument quantifies the changes in electrical impedance, displaying them as a dimensionless parameter termed cell index, which is directly proportional to the total area of tissue-culture well that is covered by cells. To each well of a 96 E-plate (ACEA Biosciences; 05 232 368 001) 50 μL of a 4× concentration HSA solution (in some assays 200 μM was used to have a final concentration of 50 μM, in other assays 120 μM was used to have a final concentration of 30 μM) in assay medium (target cell growth medium (without selection antibiotics)+1% penicillin/streptomycin (Life technologies Cat #15140) was added. Outer wells were not used and were filled with 200 μL medium or D-PBS. The 96 E-plate was placed in the xCELLigence® station (in the 37° C. incubator at 5% CO2) and a single measurement was performed to measure background impedance of the assay medium, in absence of cells. Subsequently, 50 μL target cells (2×10⁴ cells/well) in assay medium were seeded onto the 96 E-plate, and 50 μL of serially diluted ISVD construct solutions (4× concentration) in assay medium was added. (Final volume=200 μL). After 30 min at room temperature, 50 μL of primary T cells (3×10⁵ cells/well) in assay medium were added per well to achieve an effector to target ratio of 15:1. The plate was placed in the xCELLigence© station and impedance was measured every 15 min for 4 days. The data was analyzed at a fixed time point indicated in the results.

### 6.27.2 Flow Cytometry-Based Cytotoxicity Assay

ISVD constructs were characterized for redirected T cell mediated killing in a flow cytometry-based cytotoxicity assay using human or cynomolgus primary T cells as effector cells and non-adherent target cells. Target cells were labelled with 4 μM PKH26 membrane dye using the PKH26 red fluorescent cell linker kit (Sigma, PKH26GL-1KT) according to manufacturer's instruction. Effector cells (2.5×10¹ cells/well) and PKH-labelled target cells (2.5×10⁴ cells/well) were co-incubated in 96-well V-bottom plates (Greiner Bio-one, #651 180) (effector versus target ratio of 10:1) in assay medium of the target cell line (target growth medium with 1% Penicillin/streptomycin (Life Technologies, 15140) and 50 μM Alburex HSA (CSL Behring, 2160-679)). For analysis of concentration-dependent cell lysis, serial dilutions of ISVD constructs in target assay medium were added to the cells and incubated for 18 h in a 5% CO2 atmosphere at 37° C. After incubation, cells were pelleted by centrifugation and washed with FACS buffer (D-PBS (Gibco, 14190) with 10% FBS (Sigma, F7524) and 0.05% sodium azide (Acros organics, 19038)). Subsequently, cells were resuspended in 100 μL FACS buffer supplemented with 5 nM TO-PRO®-3 Iodide (642/661) (ThermoFisher Scientific, T3605) to distinguish live from dead cells. Cells were analyzed using a MACSQuant X flow cytometer (Miltenyi Biotec). Per sample, a total sample volume of 70 μL was acquired. Gating was set on PKH26 positive cells, and within this population, the TO-PRO®-3 positive cells were determined. The percent specific lysis=((% TO-PRO-3+no construct-% TO-PRO-3+with construct)/% TO-PRO-3+no construct))×100.

Functional cross-reactivity analysis of TCRαβ-CD33-CD123 multispecific ISVD construct towards CD33, CD123 and TCR was determined in the impedance-based cytotoxicity assay (xCELLigence) using human or cynomolgus primary T cells and adherent human or cynomolgus transfected CD33 or CD123 cells.

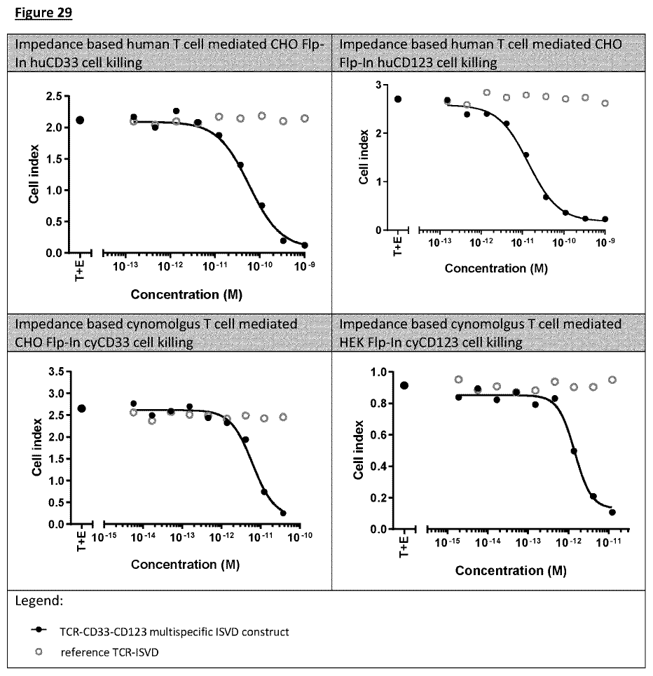

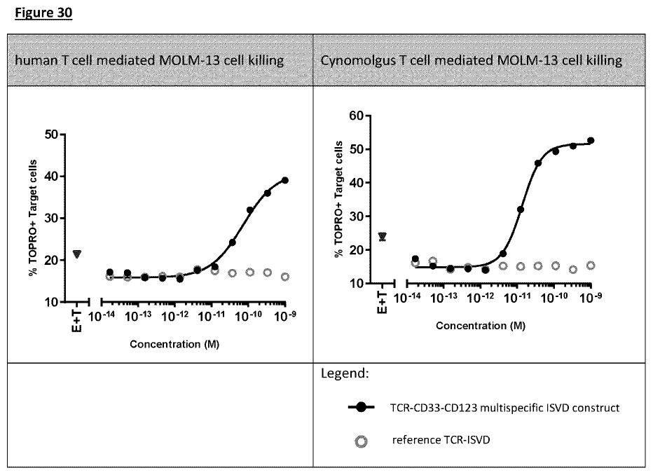

All assays, as described above in 6.27.1 and 6.27.2, were run in the presence of an excess amount of HSA, for the NANOBODY® ISVD to be fully saturated with HSA as described above. The reference TCR-ISVD was used as negative control. The results are shown in FIGS. 29 and 30 and Table 53.

### 6.27.3 Results

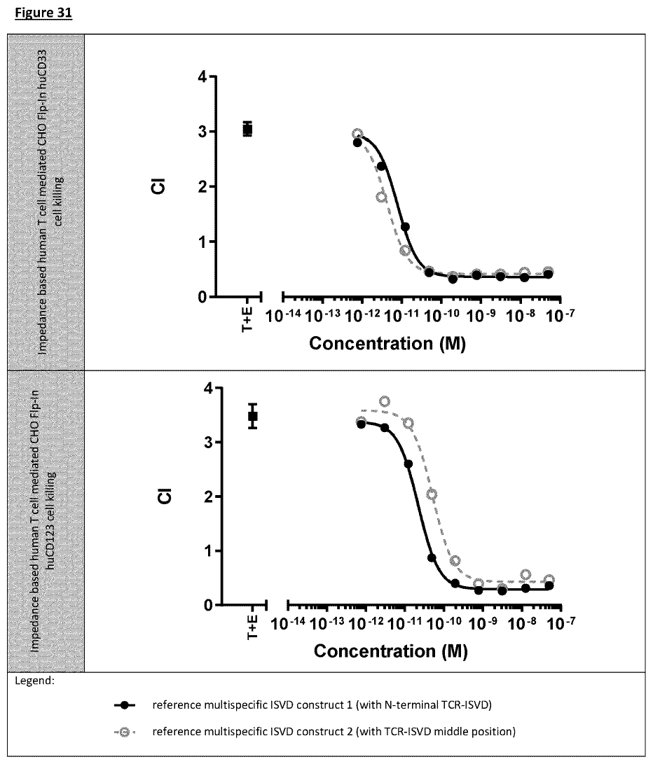

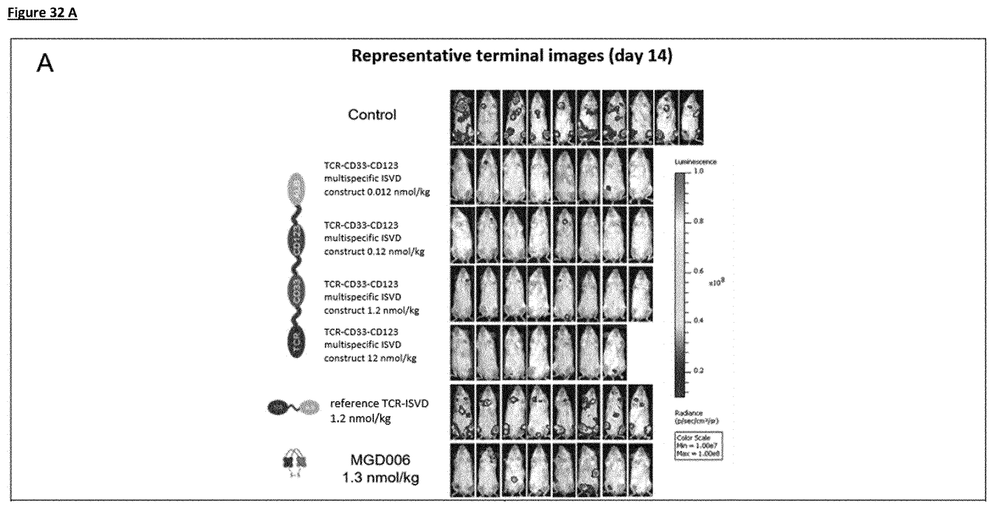

The assay results comparing human versus cynomolgus primary T cells are graphically illustrated in FIGS. 29 and 31. The evaluation of human T cell mediated cell killing of human CD33 and human CD123 transfected cells was performed using T cells from different human donors, which allowed to calculate the global IC₅₀ values (Table 53). The evaluation of cynomolgus T cell mediated killing of human CD33 and human CD123 transfected cells was performed using T cells from 1 cynomolgus monkey. The IC₅₀ values are summarized in Table 54.

TABLE 53
Global IC₅₀ (M) of TCRαβ-CD33-CD123 in the impedance-based (xCELLigence)
human T cell mediated CD33+ or CD123+ cell killing assay
using an effector to target ratio of 15 to 1 in the presence of 50 μM HSA.

| | Impedance based human T cell mediated | | | | Impedance based human T cell mediated | | | |
|---|---|---|---|---|---|---|---|---|
| | CHO hu CD33 cell killing | | | | CHO hu CD123 cell killing | | | |
| | | Global | IC50 (M) | IC50 (M) | | Global | IC50 (M) | IC50 (M) |
| ISVD | n | IC50 (M) | 95% LCI | 95% UCI | n | IC50 (M) | 95% LCI | 95% UCI |
| TCRαβ-CD33- | 4 | 1.1E−10 | 5.4E−11 | 2.4E−10 | 7 | 4.4E−11 | 2.5E−11 | 8.0E−11 |
| CD123 | | | | | | | | |
| (SEQ ID NO: 460) | | | | | | | | |

Global IC₅₀'s towards human target expressing cells were 5,4.10⁻¹¹ M and 2,5.10⁻¹¹ M respectively for CD33 and CD123.

TABLE 54
IC₅₀ (M) of TCRαβ-CD33-CD123 in the impedance-based (xCELLigence) cynomolgus T cell mediated CD33+
or CD123+ cell killing assay using an effector to target ratio of 15 to 1 in the presence of 50 μM HSA.

| | Impedance based cynomolgus T cell mediated | | | Impedance based cynomolgus T cell mediated | | |
|---|---|---|---|---|---|---|
| | CHO Flp-In cynoCD33 cell killing | | | CHO Flp-In cynoCD123 cell killing | | |
| | | IC50 (M) | IC50 (M) | | IC50 (M) | IC50 (M) |
| ISVD | IC50 (M) | 95% LCI | 95% UCI | IC50 (M) | 95% LCI | 95% UCI |
| TCRαβ-CD33- | 6.2E−12 | 5.1E−12 | 7.4E−12 | 1.4E−12 | 1.2E−12 | 1.7E−12 |
| CD123 | | | | | | |
| (SEQ ID NO: 460) | | | | | | |

To confirm the human cynomolgus cross-reactivity of TCRαβP-CD33-CD123 ISVD construct towards TCR, the ISVD construct was evaluated in a flow cytometry-based T cell mediated MOLM-13 cell killing using either human or primary T cells in combination with the CD33, CD123 double expressing human MOLM-13 target cell line in the presence of 50 μM HSA as described above. Graphical illustration of these results is shown in FIG. 30. Additional data of the TCRαβ-CD33-CD123 ISVD construct tested in the human T cell mediated killing assay using different donors were available, which allowed to calculate global EC₅₀ values (Table 55). The EC₅₀ values for the cynomolgus T cell mediated killing assays is shown in Table 56.

TABLE 55
Global EC₅₀ (M) of A025001562 in the flow cytometry human
T cell mediated MOLM-13 cell killing assay using an effector
to target ratio of 10 to 1 in the presence of 50 μM HSA.

| | Impedance based human T cell | | | |
|---|---|---|---|---|
| | mediated MOLM-13 cell killing | | | |
| | | Global | EC₅₀ (M) | EC₅₀ (M) |
| ISVD | n | EC₅₀ (M) | 95% LCI | 95% UCI |
| TCRαβ-CD33-CD123 | 13 | 1.80E−11 | 1.16E−11 | 2.78E−11 |
| (SEQ ID NO: 460) | | | | |

TABLE 56
EC₅₀ (M) for TCRαβ-CD33-CD123 ISVD construct
in the cynomolgus T cell mediated MOLM-13 cell killing
assay using an effector to target ratio of 10 to 1.

| | Cynomolgus T cell mediated | | |
|---|---|---|---|
| | MOLM-13 cell killing | | |
| ISVD | EC₅₀ (M) | 95% LCI | 95% UCI |
| TCRαβ-CD33-CD123 | 1.36E−11 | 1.15E−11 | 1.62E−11 |
| (SEQ ID NO: 460) | | | |

In conclusion, TCRαβ-CD33-CD123 ISVD construct was functional both in the human and cynomolgus human target cell mediated killing assay. The global killing potency of the described ISVD towards the CD33/CD123 double positive AML cell line was 1,8.10⁻¹¹ M.

### 6.28 Example 28: Preclinical In Vivo-Efficacy of TCRαβ-CD33-CD123 Multispecific ISVD Construct in CD123′ Molm-13-Luc Disseminated AML Model Implanted in T Cell Humanized NSG Mice

### 6.28.1 Materials and Methods

### Cell Line and Human Material

The human AML-derived cell lines expressing CD123 Molm-13 was obtained from Deutsche Sammlung von Mikroorganismen und Zellkulturen (Braunschweig, Germany). Molm-13 cells were grown in culture (37° C., 5% C02, 95% humidity) in RPM11640 Glutamax medium (completed with foetal cow serum 20%). Cells were infected with a luciferase vector (SV40-PGL4-Puro) carried by a non-replicative lentivirus; polyclonal Molm-13-luc were selected using 2 μg/ml puromycin.

### Human T-Cell Purification and Amplification for In Vivo Administration

Fresh human peripheral blood from healthy donors was supplied by EFS (Etablissement Français du Sang, Île-de-France, France.

Fresh human peripheral blood mononuclear cells (PBMCs) were isolated by Ficoll gradient centrifugation at 200 g for 40 min at room temperature without brake. The pellet was washed and resuspended in a final volume of 50 ml completed by phosphate-buffered saline (PBS). The total viable PBMCs number was defined by Vi-CELL counter (Beckman Coulter Life Sciences, Brea, CA, USA). The pellet was recovered in autoMACS Running Buffer (Miltenyi Biotec). T cells were isolated from PBMCs using the Pan T Cell Isolation Kit (Miltenyi Biotec) and autoMACS according to manufacturer instructions. Purified T cells were activated and expanded in vitro over 14 days using the T Cell TRANSACT matrix Activation/Expansion Kit (Miltenyi Biotec) based on CD3 and CD28 co stimulation. According with Miltenyi procedure, the activation protocol involved culturing T cells for 2 weeks in presence of TRANSACT matrix in TexMACS medium (Miltenyi Biotec), supplemented with 20 000 IU of soluble IL-2 and 1% penicillin-streptomycin (Gibco). At day 14 of expansion, T cells were harvested and resuspended in PBS at a final concentration of 5×10⁷ cells/ml, with 10⁷ cells administered through intraperitoneal (IP) injection to each animal. T cell viability prior to animal injection being tested to be higher than 85%.

### In Vivo Characterization

All in vivo experiments were approved by the Sanofi Ethical Committee and conducted in accordance with local and institutional Laws, Ethics and guidance in AAALAC accredited facilities.

TCRαβ-CD33-CD123 multispecific ISVD construct (SEQ ID NO:460) anti-tumor activity was evaluated after Molm13-luc AML engraftment in non-irradiated NOD.Cg-Prkdc^{scid} Il2rg^{tm1Wjl}/SzJ (NSG) mice (Charles River Laboratories, Saint-Germain-Nuelles, France). Female animals aged 6-8 weeks were implanted intravenously (IV) with 10⁶/0.2 ml Molm-13-luc cells per mouse on day 0. Same animals were intraperitoneally implanted with 107/0.2 ml human T cells per mouse on day 1.

Animals were distributed among groups based on all-body bioluminescence imaging (BLI) signal homogeneity and tumor myeloid engraftment evaluated by long bones signal segmentation on day 3. Mice were treated IV with TCRαβ-CD33-CD123 multispecific ISVD construct (SEQ ID NO:460) at 12, 1.2, 0.12, and 0.012 nmol/kg Q2D from day 4 to day 12 or with a reference, not having a CD33 or CD123 binding ISVD, (TCRαβ-ISVD (SEQ ID NO: 461) linked to Alb-ISVD (SEQ ID NO:464)) at 1.2 nmol/kg QD (days 4-13) or MGD006 (flotetuzumab, humanized DART recognizing CD3 and CD123) 1.3 nmol/kg QD (days 4-13), see Table 57. Longitudinal in vivo bioluminescence imaging (BLI) was performed to monitor disseminated tumor growth. Mice were sacrificed on day 14 and autopsied under BLI for impact evaluation in deep soft tissues such as liver, spleen, ovaries, and abdominal fat.

TABLE 57
Compound evaluation study design

| Treatment | Dose | Volume/inj | | Animal |
|---|---|---|---|---|
| Group | (nmol/Kg) | Route | Schedule | number |
| Control | — | — | — | 10 |
| TCR-CD33- | 12 | 0.2 ml IV | Q2dx5 | 7^{a} |
| CD123 (SEQ | | | (4, 6, 8, 10, 12) | |
| ID NO: 460) | 1.2 | 0.2 ml IV | Q2dx5 | 8 |
| | | | (4, 6, 8, 10, 12) | |
| | 0.12 | 0.2 ml IV | Q2dx5 | 8 |
| | | | (4, 6, 8, 10, 12) | |
| | 0.012 | 0.2 ml IV | Q2dx5 | 8 |
| | | | (4, 6, 8, 10, 12) | |
| TCR ISVD | 1.2 | 0.2 ml IV | Qdx10 (4-13) | 8 |
| construct | | | | |
| MGD006 | 1.3 | 0.2 ml IV | Qdx10 (4-13) | 8 |
| (CD3-CD123) | | | | |

^{a}one non-responding animal due to lack of T cell engraftment was not include in analysis

### Data Collection and Efficacy Criteria

Animal body weight was monitored from day 3 to the end of assay to follow impact of therapy. A dosage producing a 20% weight loss or 15% weight loss for 3 consecutive day or 10% or more drug deaths, was considered an excessively toxic dosage. Animal body weights included the tumor weights.

Tumor growth was assessed by in vivo BLI using the IVIS Lumina XRMS imager (PerkinElmer, Waltham, MA, USA) with the Living Image 4.5.2 acquisition software (PerkinElmer) through luciferase activity measurement in vivo on days 3, 7, 10, and 14 post-tumor injections, using beetle luciferin potassium salt 160 mg/kg lp injection 15 minutes before image processing of animals anesthetized with Ketamine®/Xylazine® (120 mg/kg; 6 mg/kg IM, 5 ml/kg) 5 minutes before image. Tumor growth was based on bioluminescence signal curves (expressed in photons/second).

Tumor growth was followed in all body and long bones in posteriors legs by BLI signal measurements at days 7, 10 and 14 after tumor implantation. The primary efficacy end points are ratio of change in tumor signal changes from baseline between treated and control groups (T/C), partial regression (PR) and complete regression (CR).

Tumor growth based on bioluminescence signal curves (expressed in Phot/sec) in time was drawn for each animal of each treatment group and represented as median curve±MAD, both for all body (linear scale) and bone segmented signals (Log scale). Changes in tumor bioluminescence signal for each treated (T) and control (C) are calculated for each animal and each day by subtracting the tumor signal on the day of first treatment (staging day) from the tumor signal on the specified observation day. The median T is calculated for the treated group and the median C is calculated for the control group. Then the ratio T/C is calculated and expressed as a percentage:

dT/dC=[(medianTend−medianTday3)/(medianCend−medianCday3)]×100

The dose is considered as therapeutically active when dT/dC is lower than 42% and very active when dT/dC is lower than 10%. If dT/dC is lower than 0, the dose is considered as highly active and the percentage of regression is dated: Percent tumor regression is defined as the % of tumor signal decrease in the treated group at a specified observation day compared to its signal on the first day of treatment.

For each animal, % regression is calculated at specific time-points. Considering the risk of signal variability due to luciferin kinetics variability with ip miss injection, true regressions are considered when they are observed at least at two consecutive time points for each animal.

$$\% regression ( at t ) = \frac{{Signal}_{t 0} - S i g n a {l}_{t}}{S i g n a {l}_{t 0}} \times 100$$

Partial regression (PR): Regressions are defined as partial if the tumor Signal decreases bellow the tumor Signal at the start of treatment at two consecutive time points, one being below 50% of start signal. Complete regression (CR): Regressions are defined as Complete if the tumor Signal decreases below 80% of start signal.

### Biostatistical Analysis

Tumor growth based on bioluminescence signal curves over time was measured for each animal of each treatment group. For longitudinal invivo BLI data, a two non-parametric two-way analysis of variance-Type (ANOVA-Type), followed by two contrast analyses with Bonferroni-Holm adjustment for multiplicity were performed with repeated measures by day: p>0.05: NS, 0.05<p>0.01: *, p<0.01: **. For terminal ex vivo BLI data, a one-way ANOVA with factor group on rank-transformed bioluminescence signal was performed. Descriptive statistics with medians±median absolute deviation was provided by group and day of measurement: p>0.05: NS, 0.05<p>0.01: *, p<0.01: **.

### 6.28.2 Results

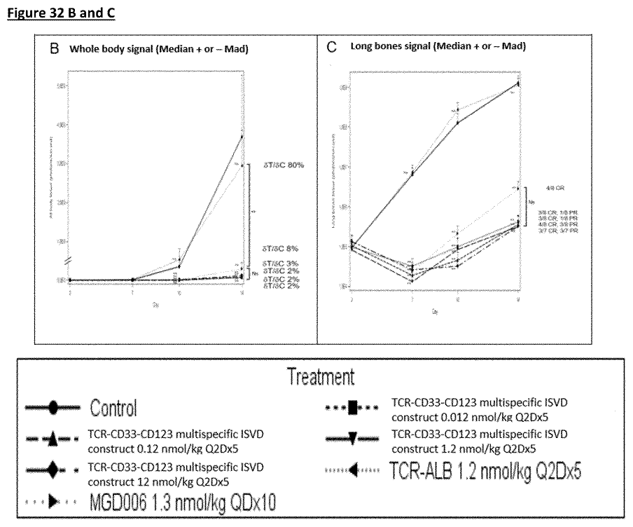

TCRαβ-CD33-CD123 multispecific ISVD construct induces anti-leukemic effects in Molm-13-luc AML xenograft model in vivo (FIG. 32A).

TCRαβ-CD33-CD123 multispecific ISVD construct administered intravenously every 2 days in the Molm-13-luc xenograft model in the presence of human effector T cells (T cell/Tumor ratio R=10) was well tolerated at all doses. No evidence of adverse events or alterations of body weight were observed under treatment. TCRαβ-CD33-CD123 multispecific ISVD construct inhibited whole body tumor growth at all tested doses with same activity (dT/dC of 2% (p<0.0001) Vs 2% (p<0.0001), 2% (p<0.0001) and 3% (p<0.0001) at 0.012; 0.12; 12 and 12 nmol/kg respectively (FIG. 32A, 32B).

A sum of the longest diameter (LD) for all target lesions was the baseline sum LD. The baseline sum LD was used as reference by which to characterize the objective tumor response.

In long bones, a 3/8 Complete Response (CR; disappearance of all lesions) and a 1/8 Partial Response (PR; at least a 30% decrease in the sum of the LD of target lesions, taking as reference the baseline sum LD) were observed at 0.012 nmol/kg.

A 3/8CR and 1/8PR were observed at 0.12 nmol/kg, a 4/8CR and 3/8PR were observed at 1.2 nmol/kg, and a 3/7CR and 3/7PR were observed at 12 nmol/kg (FIG. 32C). Reference TCR-ISVD construct was overall inactive on Molm13-luc tumor growth at 1.2 nmol/kg (dT/dC of 80%). MGD006 1.3 nmol/kg inhibited tumor growth with dT/dC 8% (NS compared to A025001562 treatments) associated with 4/8 CR in long bones (FIG. 32A, 32B).

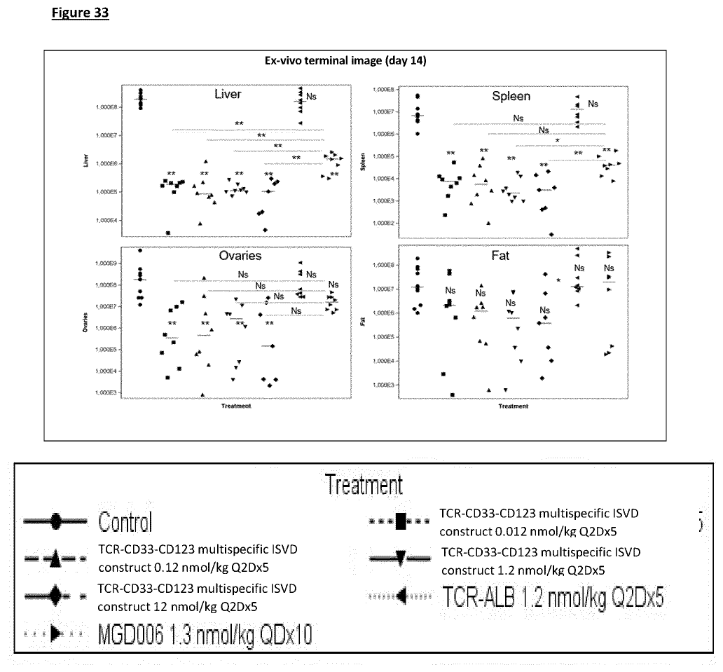

Based on terminal ex vivo bioluminescence imaging, TCRαβP-CD33-CD123 multispecific ISVD construct significantly inhibited tumor growth in liver (p<0.0001), spleen (p<0.0001) and ovaries (p<0.0001) but not in abdominal fat at all tested doses, when reference ISVD TCR-HLE was inactive in all tissues, and MGD006 inhibited significantly the tumor load in liver (p<0.0001) and spleen (p<0.0001) but not in ovaries (NS) or abdominal fat tissues. (FIG. 33)

### 6.29 Example 29: KG-1a or U-937 Cell Killing

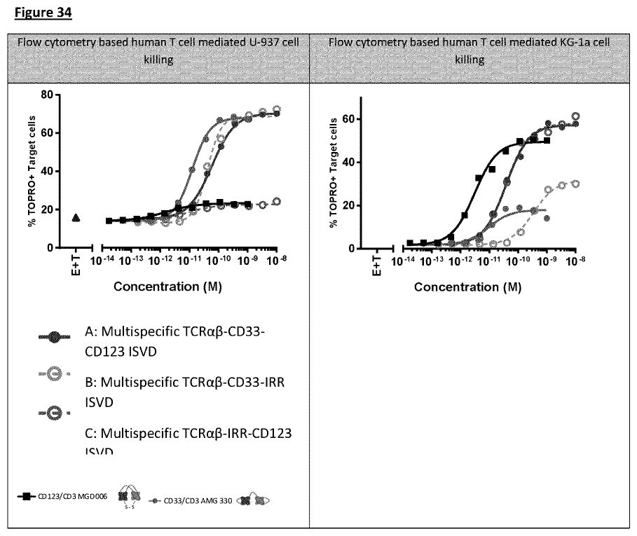

Multispecific TCRαβP-CD33-CD123 ISVD construct and the corresponding constructs, where either the CD33 binding ISVD or the CD123 binding ISVD was replaced by an irrelevant ISVD IRR (not binding to CD33 and not binding to CD123; Table 58), were also characterized using the single target expressing cell lines KG-1a (CD33-CD123+) and U-937(CD33+CD123−) (FIG. 34). Killing potencies and percentages of lysis are depicted in 59.

TABLE 58
Sample ID and the composition of the NANOBODY ® ISVDs
and linkers

| | Target | | Target | | Target |
|---|---|---|---|---|---|
| Sample ID | ISVD1 | Linker | ISVD2 | Linker | ISVD3 |
| A | TCR | 9GS | CD33 | 9GS | CD123 |
| B | TCR | 9GS | CD33 | 9GS | IRR |
| C | TCR | 9GS | IRR | 9GS | CD123 |

TABLE 59
EC₅₀ (M) and % lysis for the trivalent NANOBODY ® ISVDs in the human T cell
mediated U937 or KG1a cells cell killing assay using an effector to target ratio of 10 to 1.

| | Flow cytometry based human T cell mediated | | | | Flow cytometry based human T cell mediated | | | |
|---|---|---|---|---|---|---|---|---|
| | U-937 cell killing | | | | KG-1a cell killing | | | |
| | | EC₅₀ (M) | EC₅₀ (M) | % | | EC₅₀ (M) | EC₅₀ (M) | % |
| sample ID | EC₅₀ (M) | 95% LCI | 95% UCI | Lysis | EC₅₀ (M) | 95% LCI | 95% UCI | Lysis |
| CD33/CD3 BiTE | 1.1E−11 | 9.8E−12 | 1.3E−11 | 55 | 8.7E−12 | 6.9E−12 | 1.1E−11 | 15 |
| AMG 330 | | | | | | | | |
| CD123/CD3 | 8.9E−13 | 7.0E−13 | 1.1E−12 | 13 | 3.0E−12 | 2.4E−12 | 4.0E−12 | 58 |
| DART MGD006 | | | | | | | | |
| A | 5.9E−11 | 4.6E−11 | 7.9E−11 | 57 | 3.6E−11 | 2.8E−11 | 4.9E−11 | 54 |
| B | 4.7E−11 | 4.2E−11 | 5.2E−11 | 55 | 3.0E−10 | 2.3E−10 | 4.2E−10 | 30 |
| C | ~4.4E−12 | / | / | 17 | 3.8E−11 | 2.9E−11 | 5.1E−11 | 64 |

The TCR-CD123 single targeting ISVD hardly induced killing of CD123-U-937 cells, as was also observed for the CD123/CD3 DART MGD006 benchmark. Similarly, the CD33 single targeting ISVD induced only low level killing of CD33−/+KG1a cells, as was also observed for the CD33/CD3 BiTE AMG 330 benchmark. The dual-targeting TCRαβ-CD33-CD123 ISVD (construct A) on the other hand, demonstrated potent tumor cell killing on both CD33- and CD123-cell lines, thus exemplifying the advantage of the dual-targeting approach.

### 7 INDUSTRIAL APPLICABILITY

The polypeptides, nucleic acid molecules encoding the same, vectors comprising the nucleic acids and compositions described herein may be used for example in the treatment of subjects suffering from proliferative, inflammatory, infectious and autoimmune diseases.

1. Polypeptide comprising a first and a second immunoglobulin single variable domain (ISVD), wherein
said first ISVD specifically binds to a constant domain of a human and of a non-human primate T cell receptor (TCR) present on a T cell;
said second ISVD specifically binds to a first antigen on a target cell;
wherein said first antigen is different from said TCR;
wherein said target cell is different from said T cell; and
wherein said first ISVD essentially consists of 4 framework regions (FR1 to FR4, respectively) and 3 complementarity determining regions (CDR1 to CDR3, respectively), wherein
(i) the amino acid sequence of CDR1 is Xaa Xaa Val His Xaa Xaa Xaa Xaa Xaa Xaa (SEQ ID NO: 295), wherein
Xaa at position 1 is Gly, Trp, Gln, or Tyr,
Xaa at position 2 is Asp, Glu, Tyr, Ser, or Ala,
Xaa at position 5 is Lys, or Gln,
Xaa at position 6 is lie, or Leu,
Xaa at position 7 is Asn, or Phe,
Xaa at position 8 is Phe, Val, or Tyr,
Xaa at position 9 is Leu, Tyr, or Phe,
Xaa at position 10 is Gly, Thr, or Arg;
(ii) the amino acid sequence of CDR2 is Xaa lie Ser lie Xaa Asp Xaa Xaa Asp (SEQ ID NO: 296), wherein
Xaa at position 1 is His, or Val,
Xaa at position 5 is Gly, or Ala,
Xaa at position 7 is Gln, Val, or Glu,
Xaa at position 8 is Thr, Ser, or Val; and
(iii) the amino acid sequence of CDR3 is Xaa Ser Xaa lie Xaa Pro Tyr Xaa Xaa (SEQ ID NO: 297), wherein
Xaa at position 1 is Phe, Leu or Met,
Xaa at position 3 is Arg, Lys, or Ser,
Xaa at position 5 is Tyr, or Trp,
Xaa at position 8 is Asp, or Gln,
Xaa at position 9 is Tyr, or Trp, and
wherein CDR1, CDR2, CDR3 of said first ISVD are not simultaneously the amino acid sequences of SEQ ID NO's: 153, 209 and 223, respectively.

2. The polypeptide according claim 1, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 154 to 169.

3. The polypeptide according to claim 1 or 2, wherein said first ISVD essentially consists of 4 framework regions (FR1 to FR4, respectively) and 3 complementarity determining regions (CDR1 to CDR3, respectively), in which CDR2 is chosen from the group of amino acid sequences of SEQ ID NO's: 210 to 215.

4. The polypeptide according to any of claims 1 to 3, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 224 to 230.

5. The polypeptide according to claim 1, wherein said first ISVD essentially consists of 4 framework regions (FR1 to FR4, respectively) and 3 complementarity determining regions (CDR1 to CDR3, respectively), in which:
(i) the amino acid sequence of CDR1 is Xaa Xaa Val His Xaa Xaa Asn Phe Xaa Xaa (SEQ ID NO: 298), wherein
Xaa at position 1 is Gly, Trp, or Tyr,
Xaa at position 2 is Asp, Glu, Tyr, Ser, or Ala,
Xaa at position 5 is Lys, or Gln,
Xaa at position 6 is lie, or Leu,
Xaa at position 9 is Leu, or Tyr,
Xaa at position 10 is Gly, or Thr,
(ii) the amino acid sequence of CDR2 is His lie Ser lie Gly Asp Gln Thr Asp (SEQ ID NO: 209), and
(iii) the amino acid sequence of CDR3 is Xaa Ser Xaa lie Xaa Pro Tyr Asp Xaa (SEQ ID NO: 299), wherein
Xaa at position 1 is Phe, Leu or Met,
Xaa at position 3 is Arg, or Lys,
Xaa at position 5 is Tyr, or Trp, and
Xaa at position 9 is Tyr, or Trp.

6. The polypeptide according to claim 5, wherein said first ISVD essentially consists of 4 framework regions (FR1 to FR4, respectively) and 3 complementarity determining regions (CDR1 to CDR3, respectively), in which CDR1 is chosen from the group of amino acid sequences of SEQ ID NO's: 171 to 207.

7. The polypeptide according to claim 5 or 6, wherein said first ISVD essentially consists of 4 framework regions (FR1 to FR4, respectively) and 3 complementarity determining regions (CDR1 to CDR3, respectively), in which CDR3 is chosen from the group of amino acid sequences of SEQ ID NO's: 235 to 247.

8. The polypeptide according to any of claim 1, 5, 6 or 7, wherein said first ISVD essentially consists of 4 framework regions (FR1 to FR4, respectively) and 3 complementarity determining regions (CDR1 to CDR3, respectively), in which:
a)
CDR1 consists of the amino acid sequence of
(SEQ ID NO: 171)
GYVHKINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
b)
CDR1 consists of the amino acid sequence of
(SEQ ID NO: 172)
WDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
c)
CDR1 consists of the amino acid sequence of
(SEQ ID NO: 173)
YDVHKINFYG,
CDR2 consists of the amino acid sequence of
(SEQ ID NO: 209)
HISIGDQTD,
and
CDR3 consists of the amino acid sequence of
(SEQ ID NO: 236)
LSKIWPYDY;
or
d)
CDR1 consists of the amino acid sequence of
(SEQ ID NO: 174)
WDVHQINFYG,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
e)
CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 235)
LSRIWPYDY;
or
f)
CDR1 consists of the amino acid sequence of
(SEQ ID NO: 175)
WDVHKINFYT,
CDR2 consists of the amino acid sequence of
(SEQ ID NO: 209)
HISIGDQTD,
and
CDR3 consists of the amino acid sequence of
(SEQ ID NO: 236)
LSKIWPYDY.

9. The polypeptide according to any of claims 1 to 8, wherein the amino acid sequence of the first ISVD has at least 80% sequence identity with at least one of the amino acid sequences of SEQ ID NO's: 1 to 152, 261 or 262 in which for the purposes of determining the degree of sequence identity, the amino acid residues that form the CDR sequences are disregarded.

10. The polypeptide according to any of claims 1 to 9, wherein said first ISVD is chosen from the group of amino acid sequences of SEQ ID NO's: 46, 150 to 152, 261 and 262.

11. The polypeptide according to any of claims 1 to 10, further comprising a third ISVD, which specifically binds to a second antigen on a target cell.

12. The polypeptide according to any of claims 1 to 11, wherein said polypeptide further comprises one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units, in which said one or more other groups, residues, moieties or binding units is an ISVD that can bind to human serum albumin.

13. A nucleic acid encoding the polypeptide as defined in any one of claims 1 to 12, or a vector comprising a nucleic acid encoding the polypeptide as defined in any one of claims 1 to 12.

14. A host cell transformed or transfected with the nucleic acid or the vector as defined in claim 13.

15. A process for the production of the polypeptide according to any one of claims 1 to 12, said process comprising culturing a host cell as defined in claim 14 under conditions allowing the expression of the polypeptide as defined in any one of claims 1 to 12 and recovering the produced polypeptide from the culture.

16. A composition comprising the polypeptide according to any of claims 1 to 12, or the polypeptide produced according to the process of claim 15.

17. The polypeptide according to any one of claims 1 to 12, or the composition according to claim 16, for use as a medicament.

18. The polypeptide according to any one of claims 1 to 12, or the composition according to claim 16, for use in the prevention, treatment or amelioration of a disease selected from the group consisting of a proliferative disease, an inflammatory disease, an infectious disease and an autoimmune disease.

19. The polypeptide or the composition for use according to claim 18, wherein said proliferative disease is a cancer chosen from the group consisting of carcinomas, gliomas, mesotheliomas, melanomas, lymphomas, leukemias (including acute myeloid leukemia, chronic myeloid leukemia, acute B lymphoblastic leukemia, chronic lymphocytic leukemia and hairy cell leukemia), myelodysplastic syndrome, blastic plasmacytoid dendritic cell neoplasm, systemic mastocytosis, adenocarcinomas: breast cancer, ovarian cancer, cervical cancer, glioblastoma, multiple myeloma (including monoclonal gammopathy of undetermined significance, asymptomatic and symptomatic myeloma), prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease or AIDS-associated primary effusion lymphoma, neuroectodermal tumors, rhabdomyosarcoma; as well as any metastasis of any of the above cancers.

20. The polypeptide or the composition for use according to claim 18, wherein said inflammatory disease is chosen from the group consisting of Autoimmune Lupus (SLE), allergy, asthma and rheumatoid arthritis.


SEQUENCE LISTING (464 sequences)

SEQ ID NO: 1 - protein, 117 aa, Artificial Sequence
  monovalent ISVD
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSS

SEQ ID NO: 2 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 3 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNTVYLQMNSLRPEDTALYFCRAFSRIYPYDYWGQGTLVTVSS

SEQ ID NO: 4 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYYCRAFSRIYPYDYWGQGTLVTVSS

SEQ ID NO: 5 - protein, 117 aa, Artificial Sequence
  monovalent ISVD
EVQLVESGGGVVQPGGSLRLSCVASGYVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 6 - protein, 117 aa, Artificial Sequence
  monovalent ISVD
EVQLVESGGGVVQPGGSLRLSCVASGDVHKINFYGWYRQAPGKEREKVAHISIGDQTDYADSAKGRFTISRDESKNMVYLQMNSLRPEDTALYFCRAFSRIYPYDYWGQGTLVTVSS

SEQ ID NO: 7 - protein, 117 aa, Artificial Sequence
  monovalent ISVD
EVQLVESGGGVVQPGGSLRLSCVASGEVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 8 - protein, 117 aa, Artificial Sequence
  monovalent ISVD
EVQLVESGGGVVQPGGSLRLSCVASGSVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 9 - protein, 117 aa, Artificial Sequence
  monovalent ISVD
EVQLVESGGGVVQPGGSLRLSCVASGAVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 10 - protein, 117 aa, Artificial Sequence
  monovalent ISVD
EVQLVESGGGVVQPGGSLRLSCVASWDVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 11 - protein, 117 aa, Artificial Sequence
  monovalent ISVD
EVQLVESGGGVVQPGGSLRLSCVASYDVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 12 - protein, 117 aa, Artificial Sequence
  monovalent ISVD
EVQLVESGGGVVQPGGSLRLSCVASGDVHQINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 13 - protein, 117 aa, Artificial Sequence
  monovalent ISVD
EVQLVESGGGVVQPGGSLRLSCVASGDVHKLNFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 14 - protein, 117 aa, Artificial Sequence
  monovalent ISVD
EVQLVESGGGVVQPGGSLRLSCVASGDVHKINFLTWYRQAPGKEREKVAHISIGDQTDYADSAKGRFTISRDESKNMVYLQMNSLRPEDTALYFCRAFSRIYPYDYWGQGTLVTVSS

SEQ ID NO: 15 - protein, 117 aa, Artificial Sequence
  monovalent ISVD
EVQLVESGGGVVQPGGSLRLSCVASQDVHKINFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 16 - protein, 117 aa, Artificial Sequence
  monovalent ISVD
EVQLVESGGGVVQPGGSLRLSCVASGDVHKIFFLGWYRQAPGKEREKVAHISIGDQTDYADSAKGRFTISRDESKNMVYLQMNSLRPEDTALYFCRAFSRIYPYDYWGQGTLVTVSS

SEQ ID NO: 17 - protein, 117 aa, Artificial Sequence
  monovalent ISVD
EVQLVESGGGVVQPGGSLRLSCVASGDVHKINYLGWYRQAPGKEREKVAHISIGDQTDYADSAKGRFTISRDESKNMVYLQMNSLRPEDTALYFCRAFSRIYPYDYWGQGTLVTVSS

SEQ ID NO: 18 - protein, 117 aa, Artificial Sequence
  monovalent ISVD
EVQLVESGGGVVQPGGSLRLSCVASGDVHKINVLGWYRQAPGKEREKVAHISIGDQTDYADSAKGRFTISRDESKNMVYLQMNSLRPEDTALYFCRAFSRIYPYDYWGQGTLVTVSS

SEQ ID NO: 19 - protein, 117 aa, Artificial Sequence
  monovalent ISVD
EVQLVESGGGVVQPGGSLRLSCVASGDVHKINFFGWYRQAPGKEREKVAHISIGDQTDYADSAKGRFTISRDESKNMVYLQMNSLRPEDTALYFCRAFSRIYPYDYWGQGTLVTVSS

SEQ ID NO: 20 - protein, 117 aa, Artificial Sequence
  monovalent ISVD
EVQLVESGGGVVQPGGSLRLSCVASGDVHKINFLRWYRQAPGKEREKVAHISIGDQTDYADSAKGRFTISRDESKNMVYLQMNSLRPEDTALYFCRAFSRIYPYDYWGQGTLVTVSS

SEQ ID NO: 21 - protein, 117 aa, Artificial Sequence
  monovalent ISVD
EVQLVESGGGVVQPGGSLRLSCVASGDVHKINFLSWYRQAPGKEREKVAHISIGDQTDYADSAKGRFTISRDESKNMVYLQMNSLRPEDTALYFCRAFSRIYPYDYWGQGTLVTVSS

SEQ ID NO: 22 - protein, 117 aa, Artificial Sequence
  monovalent ISVD
EVQLVESGGGVVQPGGSLRLSCVASGDVHKINFLGWYRQAPGKEREKVAVISIGDQTDYADSAKGRFTISRDESKNMVYLQMNSLRPEDTALYFCRAFSRIYPYDYWGQGTLVTVSS

SEQ ID NO: 23 - protein, 117 aa, Artificial Sequence
  monovalent ISVD
EVQLVESGGGVVQPGGSLRLSCVASGDVHKINFLGWYRQAPGKEREKVAHISIADQTDYADSAKGRFTISRDESKNMVYLQMNSLRPEDTALYFCRAFSRIYPYDYWGQGTLVTVSS

SEQ ID NO: 24 - protein, 117 aa, Artificial Sequence
  monovalent ISVD
EVQLVESGGGVVQPGGSLRLSCVASGDVHKINFLGWYRQAPGKEREKVAHISIGDETDYADSAKGRFTISRDESKNMVYLQMNSLRPEDTALYFCRAFSRIYPYDYWGQGTLVTVSS

SEQ ID NO: 25 - protein, 117 aa, Artificial Sequence
  monovalent ISVD
EVQLVESGGGVVQPGGSLRLSCVASGDVHKINFLGWYRQAPGKEREKVAHISIGDVTDYADSAKGRFTISRDESKNMVYLQMNSLRPEDTALYFCRAFSRIYPYDYWGQGTLVTVSS

SEQ ID NO: 26 - protein, 117 aa, Artificial Sequence
  monovalent ISVD
EVQLVESGGGVVQPGGSLRLSCVASGDVHKINFLGWYRQAPGKEREKVAHISIGDQVDYADSAKGRFTISRDESKNMVYLQMNSLRPEDTALYFCRAFSRIYPYDYWGQGTLVTVSS

SEQ ID NO: 27 - protein, 117 aa, Artificial Sequence
  monovalent ISVD
EVQLVESGGGVVQPGGSLRLSCVASGDVHKINFLGWYRQAPGKEREKVAHISIGDQSDYADSAKGRFTISRDESKNMVYLQMNSLRPEDTALYFCRAFSRIYPYDYWGQGTLVTVSS

SEQ ID NO: 28 - protein, 117 aa, Artificial Sequence
  monovalent ISVD
EVQLVESGGGVVQPGGSLRLSCVASGDVHKINFLGWYRQAPGKEREKVAMISIGDQTDYADSAKGRFTISRDESKNMVYLQMNSLRPEDTALYFCRAFSRIYPYDYWGQGTLVTVSS

SEQ ID NO: 29 - protein, 117 aa, Artificial Sequence
  monovalent ISVD
EVQLVESGGGVVQPGGSLRLSCVASGDVHKINFLGWYRQAPGKEREKVAHISIGEQTDYADSAKGRFTISRDESKNMVYLQMNSLRPEDTALYFCRAFSRIYPYDYWGQGTLVTVSS

SEQ ID NO: 30 - protein, 117 aa, Artificial Sequence
  monovalent ISVD
EVQLVESGGGVVQPGGSLRLSCVASGDVHKINFLGWYRQAPGKEREKVAHISIGDHTDYADSAKGRFTISRDESKNMVYLQMNSLRPEDTALYFCRAFSRIYPYDYWGQGTLVTVSS

SEQ ID NO: 31 - protein, 117 aa, Artificial Sequence
  monovalent ISVD
EVQLVESGGGVVQPGGSLRLSCVASGDVHKINFLGWYRQAPGKEREKVAHISIGDKTDYADSAKGRFTISRDESKNMVYLQMNSLRPEDTALYFCRAFSRIYPYDYWGQGTLVTVSS

SEQ ID NO: 32 - protein, 117 aa, Artificial Sequence
  monovalent ISVD
EVQLVESGGGVVQPGGSLRLSCVASGDVHKINFLGWYRQAPGKEREKVAHISIGDRTDYADSAKGRFTISRDESKNMVYLQMNSLRPEDTALYFCRAFSRIYPYDYWGQGTLVTVSS

SEQ ID NO: 33 - protein, 117 aa, Artificial Sequence
  monovalent ISVD
EVQLVESGGGVVQPGGSLRLSCVASGDVHKINFLGWYRQAPGKEREKVAHISIGDQADYADSAKGRFTISRDESKNMVYLQMNSLRPEDTALYFCRAFSRIYPYDYWGQGTLVTVSS

SEQ ID NO: 34 - protein, 117 aa, Artificial Sequence
  monovalent ISVD
EVQLVESGGGVVQPGGSLRLSCVASGDVHKINFLGWYRQAPGKEREKVAHISIGDQTNYADSAKGRFTISRDESKNMVYLQMNSLRPEDTALYFCRAFSRIYPYDYWGQGTLVTVSS

SEQ ID NO: 35 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRALSRIYPYDYWGQGTLVTVSS

SEQ ID NO: 36 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIWPYDYWGQGTLVTVSS

SEQ ID NO: 37 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAMSRIYPYDYWGQGTLVTVSS

SEQ ID NO: 38 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSKIYPYDYWGQGTLVTVSS

SEQ ID NO: 39 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYDWWGQGTLVTVSS

SEQ ID NO: 40 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSSIYPYDYWGQGTLVTVSS

SEQ ID NO: 41 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYQYWGQGTLVTVSS

SEQ ID NO: 42 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYSYWGQGTLVTVSS

SEQ ID NO: 43 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYLYWGQGTLVTVSS

SEQ ID NO: 44 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYAYWGQGTLVTVSS

SEQ ID NO: 45 - protein, 117 aa, Artificial Sequence
  monovalent ISVD
EVQLVESGGGVVQPGGSLRLSCVASGDVHKINFLGWYRQAPGKEREKVAHISIGDQTDYADSAKGRFTISRDESKNMVYLQMNSLRPEDTALYFCRAFSRIYPYTYWGQGTLVTVSS

SEQ ID NO: 46 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 47 - protein, 117 aa, Artificial Sequence
  monovalent ISVD
EVQLVESGGGVVQPGGSLRLSCVASWDVHKINFYGWYRQAPGKEREKVAHISIGDQTDYADSAKGRFTISRDESKNTVYLQMNSLRPEDTALYYCRALSKIWPYDYWGQGTLVTVSS

SEQ ID NO: 48 - protein, 117 aa, Artificial Sequence
  monovalent ISVD
EVQLVESGGGVVQPGGSLRLSCVASYDVHKINFYGWYRQAPGKEREKVAHISIGDQTDYADSAKGRFTISRDESKNTVYLQMNSLRPEDTALYYCRALSKIWPYDYWGQGTLVTVSS

SEQ ID NO: 49 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 50 - protein, 117 aa, Artificial Sequence
  monovalent ISVD
EVQLVESGGGVVQPGGSLRLSCVASWDVHKINFYTWYRQAPGKEREKVAHISIGDQTDYADSAKGRFTISRDESKNTVYLQMNSLRPEDTALYYCRALSKIWPYDYWGQGTLVTVSS

SEQ ID NO: 51 - protein, 117 aa, Artificial Sequence
  monovalent ISVD
EVQLVESGGGVVQPGGSLRLSCVASGSVHQINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 52 - protein, 117 aa, Artificial Sequence
  monovalent ISVD
EVQLVESGGGVVQPGGSLRLSCVASWDVHKINFYGWYRQAPGKEREKVAHISIGDQTDYADSAKGRFTISRDESKNMVYLQMNSLRPEDTALYFCRALSKIWPYDYWGQGTLVTVSS

SEQ ID NO: 53 - protein, 117 aa, Artificial Sequence
  monovalent ISVD
EVQLVESGGGVVQPGGSLRLSCVASYDVHKINFYGWYRQAPGKEREKVAHISIGDQTDYADSAKGRFTISRDESKNMVYLQMNSLRPEDTALYFCRALSKIWPYDYWGQGTLVTVSS

SEQ ID NO: 54 - protein, 117 aa, Artificial Sequence
  monovalent ISVD
EVQLVESGGGVVQPGGSLRLSCVASGDVHQLNFLGWYRQAPGKEREKVAHISIGDQTDYADSAKGRFTISRDESKNMVYLQMNSLRPEDTALYFCRAFSKIYPYDYWGQGTLVTVSS

SEQ ID NO: 55 - protein, 117 aa, Artificial Sequence
  monovalent ISVD
EVQLVESGGGVVQPGGSLRLSCVASYDVHKINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 56 - protein, 117 aa, Artificial Sequence
  monovalent ISVD
EVQLVESGGGVVQPGGSLRLSCVASWDVHKLNFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 57 - protein, 117 aa, Artificial Sequence
  monovalent ISVD
EVQLVESGGGVVQPGGSLRLSCVASWDVHKINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 58 - protein, 117 aa, Artificial Sequence
  monovalent ISVD
EVQLVESGGGVVQPGGSLRLSCVASWDVHKINFYGWYRQAPGKEREKVAHISIGDQTDYADSAKGRFTISRDESKNMVYLQMNSLRPEDTALYFCRAFSKIWPYDYWGQGTLVTVSS

SEQ ID NO: 59 - protein, 117 aa, Artificial Sequence
  monovalent ISVD
EVQLVESGGGVVQPGGSLRLSCVASYDVHQINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 60 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNMVYLQMNSLRPEDTALYFCRAFSKIWPYDYWGQGTLVTVSS

SEQ ID NO: 61 - protein, 117 aa, Artificial Sequence
  monovalent ISVD
EVQLVESGGGVVQPGGSLRLSCVASWDVHQINFLGWYRQAPGKEREKVAHISIGDQTDYADSAKGRFTISRDESKNMVYLQMNSLRPEDTALYFCRAFSKIWPYDYWGQGTLVTVSS

SEQ ID NO: 62 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNMVYLQMNSLRPEDTALYFCRAFSKIYPYDYWGQGTLVTVSS

SEQ ID NO: 63 - protein, 117 aa, Artificial Sequence
  monovalent ISVD
EVQLVESGGGVVQPGGSLRLSCVASWDVHQLNFYGWYRQAPGKEREKVAHISIGDQTDYADSAKGRFTISRDESKNMVYLQMNSLRPEDTALYFCRAMSKIYPYDYWGQGTLVTVSS

SEQ ID NO: 64 - protein, 117 aa, Artificial Sequence
  monovalent ISVD
EVQLVESGGGVVQPGGSLRLSCVASYDVHQINFLGWYRQAPGKEREKVAHISIGDQTDYADSAKGRFTISRDESKNMVYLQMNSLRPEDTALYFCRAMSKIYPYDYWGQGTLVTVSS

SEQ ID NO: 65 - protein, 117 aa, Artificial Sequence
  monovalent ISVD
EVQLVESGGGVVQPGGSLRLSCVASWDVHQLNFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 66 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 67 - protein, 117 aa, Artificial Sequence
  monovalent ISVD
EVQLVESGGGVVQPGGSLRLSCVASGYVHKINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 68 - protein, 117 aa, Artificial Sequence
  monovalent ISVD
EVQLVESGGGVVQPGGSLRLSCVASWDVHQINFLGWYRQAPGKEREKVAHISIGDQTDYADSAKGRFTISRDESKNMVYLQMNSLRPEDTALYFCRAMSKIWPYDYWGQGTLVTVSS

SEQ ID NO: 69 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNMVYLQMNSLRPEDTALYFCRAMSKIYPYDYWGQGTLVTVSS

SEQ ID NO: 70 - protein, 117 aa, Artificial Sequence
  monovalent ISVD
EVQLVESGGGVVQPGGSLRLSCVASWSVHQINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 71 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 72 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNMVYLQMNSLRPEDTALYFCRAFSKIWPYDWWGQGTLVTVSS

SEQ ID NO: 73 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNMVYLQMNSLRPEDTALYFCRAMSRIWPYDWWGQGTLVTVSS

SEQ ID NO: 74 - protein, 117 aa, Artificial Sequence
  monovalent ISVD
EVQLVESGGGVVQPGGSLRLSCVASGYVHKLNFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 75 - protein, 117 aa, Artificial Sequence
  monovalent ISVD
EVQLVESGGGVVQPGGSLRLSCVASGYVHKLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 76 - protein, 117 aa, Artificial Sequence
  monovalent ISVD
EVQLVESGGGVVQPGGSLRLSCVASGYVHKLNFYGWYRQAPGKEREKVAHISIGDQTDYADSAKGRFTISRDESKNMVYLQMNSLRPEDTALYFCRAFSRIWPYDWWGQGTLVTVSS

SEQ ID NO: 77 - protein, 117 aa, Artificial Sequence
  monovalent ISVD
EVQLVESGGGVVQPGGSLRLSCVASGYVHKLNFLGWYRQAPGKEREKVAHISIGDQTDYADSAKGRFTISRDESKNMVYLQMNSLRPEDTALYFCRAMSRIWPYDWWGQGTLVTVSS

SEQ ID NO: 78 - protein, 117 aa, Artificial Sequence
  monovalent ISVD
EVQLVESGGGVVQPGGSLRLSCVASGYVHKLNFYGWYRQAPGKEREKVAHISIGDQTDYADSAKGRFTISRDESKNMVYLQMNSLRPEDTALYFCRAMSRIWPYDWWGQGTLVTVSS

SEQ ID NO: 79 - protein, 117 aa, Artificial Sequence
  monovalent ISVD
EVQLVESGGGVVQPGGSLRLSCVASGSVHQLNFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 80 - protein, 117 aa, Artificial Sequence
  monovalent ISVD
EVQLVESGGGVVQPGGSLRLSCVASGAVHKLNFLGWYRQAPGKEREKVAHISIGDQTDYADSAKGRFTISRDESKNMVYLQMNSLRPEDTALYFCRAFSKIWPYDWWGQGTLVTVSS

SEQ ID NO: 81 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 82 - protein, 117 aa, Artificial Sequence
  monovalent ISVD
EVQLVESGGGVVQPGGSLRLSCVASGSVHQINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 83 - protein, 117 aa, Artificial Sequence
  monovalent ISVD
EVQLVESGGGVVQPGGSLRLSCVASGSVHQLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 84 - protein, 117 aa, Artificial Sequence
  monovalent ISVD
EVQLVESGGGVVQPGGSLRLSCVASGAVHQINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 85 - protein, 117 aa, Artificial Sequence
  monovalent ISVD
EVQLVESGGGVVQPGGSLRLSCVASGDVHQINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 86 - protein, 117 aa, Artificial Sequence
  monovalent ISVD
EVQLVESGGGVVQPGGSLRLSCVASGAVHQINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 87 - protein, 117 aa, Artificial Sequence
  monovalent ISVD
EVQLVESGGGVVQPGGSLRLSCVASGYVHKLNFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 88 - protein, 117 aa, Artificial Sequence
  monovalent ISVD
EVQLVESGGGVVQPGGSLRLSCVASGDVHQINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 89 - protein, 117 aa, Artificial Sequence
  monovalent ISVD
EVQLVESGGGVVQPGGSLRLSCVASGAVHQLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 90 - protein, 117 aa, Artificial Sequence
  monovalent ISVD
EVQLVESGGGVVQPGGSLRLSCVASGAVHKLNFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 91 - protein, 117 aa, Artificial Sequence
  monovalent ISVD
EVQLVESGGGVVQPGGSLRLSCVASGSVHKLNFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 92 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNMVYLQMNSLRPEDTALYFCRALSRIWPYDYWGQGTLVTVSS

SEQ ID NO: 93 - protein, 117 aa, Artificial Sequence
  monovalent ISVD
EVQLVESGGGVVQPGGSLRLSCVASGAVHKINFLGWYRQAPGKEREKVAHISIGDQTDYADSAKGRFTISRDESKNMVYLQMNSLRPEDTALYFCRAMSKIWPYDWWGQGTLVTVSS

SEQ ID NO: 94 - protein, 117 aa, Artificial Sequence
  monovalent ISVD
EVQLVESGGGVVQPGGSLRLSCVASGSVHKINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 95 - protein, 117 aa, Artificial Sequence
  monovalent ISVD
EVQLVESGGGVVQPGGSLRLSCVASGAVHKINFYGWYRQAPGKEREKVAHISIGDQTDYADSAKGRFTISRDESKNMVYLQMNSLRPEDTALYFCRAMSRIWPYDWWGQGTLVTVSS

SEQ ID NO: 96 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 97 - protein, 117 aa, Artificial Sequence
  monovalent ISVD
EVQLVESGGGVVQPGGSLRLSCVASGSVHKLNFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 98 - protein, 117 aa, Artificial Sequence
  monovalent ISVD
EVQLVESGGGVVQPGGSLRLSCVASGAVHKINFLGWYRQAPGKEREKVAHISIGDQTDYADSAKGRFTISRDESKNMVYLQMNSLRPEDTALYFCRAMSRIYPYDYWGQGTLVTVSS

SEQ ID NO: 99 - protein, 117 aa, Artificial Sequence
  monovalent ISVD
EVQLVESGGGVVQPGGSLRLSCVASGYVHQINFLGWYRQAPGKEREKVAHISIGDQTDYADSAKGRFTISRDESKNMVYLQMNSLRPEDTALYFCRAMSKIWPYDYWGQGTLVTVSS

SEQ ID NO: 100 - protein, 117 aa, Artificial Sequence
  monovalent ISVD
EVQLVESGGGVVQPGGSLRLSCVASGAVHKINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 101 - protein, 117 aa, Artificial Sequence
  monovalent ISVD
EVQLVESGGGVVQPGGSLRLSCVASWSVHQLNFYGWYRQAPGKEREKVAHISIGDQTDYADSAKGRFTISRDESKNMVYLQMNSLRPEDTALYFCRAMSKIYPYDWWGQGTLVTVSS

SEQ ID NO: 102 - protein, 117 aa, Artificial Sequence
  monovalent ISVD
EVQLVESGGGVVQPGGSLRLSCVASGAVHKINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 103 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNMVYLQMNSLRPEDTALYFCRAMSRIWPYDWWGQGTLVTVSS

SEQ ID NO: 104 - protein, 117 aa, Artificial Sequence
  monovalent ISVD
EVQLVESGGGVVQPGGSLRLSCVASGYVHQLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 105 - protein, 117 aa, Artificial Sequence
  monovalent ISVD
EVQLVESGGGVVQPGGSLRLSCVASGAVHKINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 106 - protein, 117 aa, Artificial Sequence
  monovalent ISVD
EVQLVESGGGVVQPGGSLRLSCVASGAVHKLNFYGWYRQAPGKEREKVAHISIGDQTDYADSAKGRFTISRDESKNMVYLQMNSLRPEDTALYFCRAFSRIWPYDYWGQGTLVTVSS

SEQ ID NO: 107 - protein, 117 aa, Artificial Sequence
  monovalent ISVD
EVQLVESGGGVVQPGGSLRLSCVASGSVHKINFLGWYRQAPGKEREKVAHISIGDQTDYADSAKGRFTISRDESKNMVYLQMNSLRPEDTALYFCRAMSRIWPYDWWGQGTLVTVSS

SEQ ID NO: 108 - protein, 117 aa, Artificial Sequence
  monovalent ISVD
EVQLVESGGGVVQPGGSLRLSCVASGAVHKINFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 109 - protein, 117 aa, Artificial Sequence
  monovalent ISVD
EVQLVESGGGVVQPGGSLRLSCVASGSVHKLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 110 - protein, 117 aa, Artificial Sequence
  monovalent ISVD
EVQLVESGGGVVQPGGSLRLSCVASGYVHQINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 111 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNMVYLQMNSLRPEDTALYFCRAFSRIWPYDYWGQGTLVTVSS

SEQ ID NO: 112 - protein, 117 aa, Artificial Sequence
  monovalent ISVD
EVQLVESGGGVVQPGGSLRLSCVASGAVHKLNFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 113 - protein, 117 aa, Artificial Sequence
  monovalent ISVD
EVQLVESGGGVVQPGGSLRLSCVASGEVHQINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 114 - protein, 117 aa, Artificial Sequence
  monovalent ISVD
EVQLVESGGGVVQPGGSLRLSCVASGSVHKLNFYGWYRQAPGKEREKVAHISIGDQTDYADSAKGRFTISRDESKNMVYLQMNSLRPEDTALYFCRAMSRIWPYDWWGQGTLVTVSS

SEQ ID NO: 115 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 116 - protein, 117 aa, Artificial Sequence
  monovalent ISVD
EVQLVESGGGVVQPGGSLRLSCVASGYVHQINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 117 - protein, 117 aa, Artificial Sequence
  monovalent ISVD
EVQLVESGGGVVQPGGSLRLSCVASGAVHKLNFLGWYRQAPGKEREKVAHISIGDQTDYADSAKDRFTISRDESKNMVYLQMNSLRPEDTALYFCRAMSRIYPYDYWGQGTLVTVSS

SEQ ID NO: 118 - protein, 117 aa, Artificial Sequence
  monovalent ISVD
EVQLVESGGGVVQPGGSLRLSCVASGYVHQINFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 119 - protein, 117 aa, Artificial Sequence
  monovalent ISVD
EVQLVESGGGVVQPGGSLRLSCVASGAVHKLNFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 120 - protein, 117 aa, Artificial Sequence
  monovalent ISVD
EVQLVESGGGVVQPGGSLRLSCVASGDVHQINFYGWYRQAPGKEREKVAHISIGDQTDYADSAKGRFTISRDESKNMVYLQMNSLRPEDTALYFCRAFSKIYPYDYWGQGTLVTVSS

SEQ ID NO: 121 - protein, 117 aa, Artificial Sequence
  monovalent ISVD
EVQLVESGGGVVQPGGSLRLSCVASGSVHKLNFLGWYRQAPGKEREKVAHISIGDQTDYADSAKGRFTISRDESKNMVYLQMNSLRPEDTALYFCRAFSRIWPYDWWGQGTLVTVSS

SEQ ID NO: 122 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNMVYLQMNSLRPEDTALYFCRAMSRIYPYDWWGQGTLVTVSS

SEQ ID NO: 123 - protein, 117 aa, Artificial Sequence
  monovalent ISVD
EVQLVESGGGVVQPGGSLRLSCVASGAVHKLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 124 - protein, 117 aa, Artificial Sequence
  monovalent ISVD
EVQLVESGGGVVQPGGSLRLSCVASGDVHQINFYGWYRQAPGKEREKVAHISIGDQTDYADSAKGRFTISRDESKNMVYLQMNSLRPEDTALYFCRAMSKIWPYDYWGQGTLVTVSS

SEQ ID NO: 125 - protein, 117 aa, Artificial Sequence
  monovalent ISVD
EVQLVESGGGVVQPGGSLRLSCVASGSVHKINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 126 - protein, 117 aa, Artificial Sequence
  monovalent ISVD
EVQLVESGGGVVQPGGSLRLSCVASGDVHKLNFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 127 - protein, 117 aa, Artificial Sequence
  monovalent ISVD
EVQLVESGGGVVQPGGSLRLSCVASGAVHKLNFLGWYRQAPGKEREKVAHISIGDQTDYADSAKGRFTISRDESKNMVYLQMNSLRPEDTALYFCRAMSRIWPYDWWGQGTLVTVSS

SEQ ID NO: 128 - protein, 117 aa, Artificial Sequence
  monovalent ISVD
EVQLVESGGGVVQPGGSLRLSCVASGSVHKLNFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 129 - protein, 117 aa, Artificial Sequence
  monovalent ISVD
EVQLVESGGGVVQPGGSLRLSCVASGDVHQLNFLGWYRQAPGKEREKVAHISIGDQTDYADSAKGRFTISRDESKNMVYLQMNSLRPEDTALYFCRALSKIYPYDYWGQGTLVTVSS

SEQ ID NO: 130 - protein, 117 aa, Artificial Sequence
  monovalent ISVD
EVQLVESGGGVVQPGGSLRLSCVASGDVHQINFLGWYRQAPGKEREKVAHISIGDQTDYADSAKGRFTISRDESKNMVYLQMNSLRPEDTALYFCRALSKIYPYDYWGQGTLVTVSS

SEQ ID NO: 131 - protein, 117 aa, Artificial Sequence
  monovalent ISVD
EVQLVESGGGVVQPGGSLRLSCVASGAVHKINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 132 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNMVYLQMNSLRPEDTALYFCRAMSKIYPYDYWGQGTLVTVSS

SEQ ID NO: 133 - protein, 117 aa, Artificial Sequence
  monovalent ISVD
EVQLVESGGGVVQPGGSLRLSCVASWEVHKINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 134 - protein, 117 aa, Artificial Sequence
  monovalent ISVD
EVQLVESGGGVVQPGGSLRLSCVASYAVHQINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 135 - protein, 117 aa, Artificial Sequence
  monovalent ISVD
EVQLVESGGGVVQPGGSLRLSCVASGYVHQINFYGWYRQAPGKEREKVAHISIGDQTDYADSAKGRFTISRDESKNMVYLQMNSLRPEDTALYFCRAMSRIWPYDWWGQGTLVTVSS

SEQ ID NO: 136 - protein, 117 aa, Artificial Sequence
  monovalent ISVD
EVQLVESGGGVVQPGGSLRLSCVASWDVHQINFLGWYRQAPGKEREKVAHISIGDQTDYADSAKGRFTISRDESKNMVYLQMNSLRPEDTALYFCRAMSRIYPYDYWGQGTLVTVSS

SEQ ID NO: 137 - protein, 117 aa, Artificial Sequence
  monovalent ISVD
EVQLVESGGGVVQPGGSLRLSCVASGDVHQINFLGWYRQAPGKEREKVAHISIGDQTDYADSAKGRFTISRDESKNMVYLQMNSLRPEDTALYFCRAFSKIYPYDYWGQGTLVTVSS

SEQ ID NO: 138 - protein, 117 aa, Artificial Sequence
  monovalent ISVD
EVQLVESGGGVVQPGGSLRLSCVASWDVHQLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 139 - protein, 117 aa, Artificial Sequence
  monovalent ISVD
EVQLVESGGGVVQPGGSLRLSCVASWDVHQINFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 140 - protein, 117 aa, Artificial Sequence
  monovalent ISVD
EVQLVESGGGVVQPGGSLRLSCVASYDVHQLNFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 141 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 142 - protein, 117 aa, Artificial Sequence
  monovalent ISVD
EVQLVESGGGVVQPGGSLRLSCVASWDVHQINFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 143 - protein, 117 aa, Artificial Sequence
  monovalent ISVD
EVQLVESGGGVVQPGGSLRLSCVASYDVHQINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 144 - protein, 117 aa, Artificial Sequence
  monovalent ISVD
EVQLVESGGGVVQPGGSLRLSCVASWDVHKINFYGWYRQAPGKEREKVAHISIGDQTDYADSAKGRFTISRDESKNMVYLQMNSLRPEDTALYFCRAMSRIYPYDYWGQGTLVTVSS

SEQ ID NO: 145 - protein, 117 aa, Artificial Sequence
  monovalent ISVD
EVQLVESGGGVVQPGGSLRLSCVASWDVHKINFYGWYRQAPGKEREKVAHISIGDQTDYADSAKGRFTISRDESKNMVYLQMNSLRPEDTALYFCRAMSRIWPYDYWGQGTLVTVSS

SEQ ID NO: 146 - protein, 117 aa, Artificial Sequence
  monovalent ISVD
EVQLVESGGGVVQPGGSLRLSCVASWEVHQINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 147 - protein, 117 aa, Artificial Sequence
  monovalent ISVD
EVQLVESGGGVVQPGGSLRLSCVASWDVHKINFYTWYRQAPGKEREKVAHISIGDQTDYADSAKGRFTISRDESKNTVYLQMNSLRPEDTALYYCRALSRIWPYDYWGQGTLVTVSS

SEQ ID NO: 148 - protein, 117 aa, Artificial Sequence
  monovalent ISVD
EVQLVESGGGVVQPGGSLRLSCVASGSVHQINFLGWYRQAPGKEREKVAHISIGDQTDYADSAKGRFTISRDESKNMVYLQMNSLRPEDTALYFCRAMSRIYPYDWWGQGTLVTVSS

SEQ ID NO: 149 - protein, 117 aa, Artificial Sequence
  monovalent ISVD
EVQLVESGGGVVQPGGSLRLSCVASYSVHQLNFLGWYRQAPGKEREKVAHISIGDQTDYADSAKGRFTISRDESKNMVYLQMNSLRPEDTALYFCRAMSRIWPYDYWGQGTLVTVSS

SEQ ID NO: 150 - protein, 117 aa, Artificial Sequence
  monovalent ISVD
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSS

SEQ ID NO: 151 - protein, 117 aa, Artificial Sequence
  monovalent ISVD
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 152 - protein, 117 aa, Artificial Sequence
  monovalent ISVD
DVQLVESGGGVVQPGGSLRLSCVASGYVHKINFYGWYRQAPGKEREKVAHISIGDQTDYADSAKGRFTISRDESKNTVYLQMNSLRPEDTATYYCRALSRIWPYDYWGQGTLVTVSS

SEQ ID NO: 153 - protein, 10 aa, Artificial Sequence
  CDR1
GDVHKINFLG

SEQ ID NO: 154 - protein, 10 aa, Artificial Sequence
  CDR1
GYVHKINFLG

SEQ ID NO: 155 - protein, 10 aa, Artificial Sequence
  CDR1
GDVHKINFYG

SEQ ID NO: 156 - protein, 10 aa, Artificial Sequence
  CDR1
GEVHKINFLG

SEQ ID NO: 157 - protein, 10 aa, Artificial Sequence
  CDR1
GSVHKINFLG

SEQ ID NO: 158 - protein, 10 aa, Artificial Sequence
  CDR1
GAVHKINFLG

SEQ ID NO: 159 - protein, 10 aa, Artificial Sequence
  CDR1
WDVHKINFLG

SEQ ID NO: 160 - protein, 10 aa, Artificial Sequence
  CDR1
YDVHKINFLG

SEQ ID NO: 161 - protein, 10 aa, Artificial Sequence
  CDR1
GDVHQINFLG

SEQ ID NO: 162 - protein, 10 aa, Artificial Sequence
  CDR1
GDVHKLNFLG

SEQ ID NO: 163 - protein, 10 aa, Artificial Sequence
  CDR1
GDVHKINFLT

SEQ ID NO: 164 - protein, 10 aa, Artificial Sequence
  CDR1
QDVHKINFLG

SEQ ID NO: 165 - protein, 10 aa, Artificial Sequence
  CDR1
GDVHKIFFLG

SEQ ID NO: 166 - protein, 10 aa, Artificial Sequence
  CDR1
GDVHKINYLG

SEQ ID NO: 167 - protein, 10 aa, Artificial Sequence
  CDR1
GDVHKINVLG

SEQ ID NO: 168 - protein, 10 aa, Artificial Sequence
  CDR1
GDVHKINFFG

SEQ ID NO: 169 - protein, 10 aa, Artificial Sequence
  CDR1
GDVHKINFLR

SEQ ID NO: 170 - protein, 10 aa, Artificial Sequence
  CDR1
GDVHKINFLS

SEQ ID NO: 171 - protein, 10 aa, Artificial Sequence
  CDR1
GYVHKINFYG

SEQ ID NO: 172 - protein, 10 aa, Artificial Sequence
  CDR1
WDVHKINFYG

SEQ ID NO: 173 - protein, 10 aa, Artificial Sequence
  CDR1
YDVHKINFYG

SEQ ID NO: 174 - protein, 10 aa, Artificial Sequence
  CDR1
WDVHQINFYG

SEQ ID NO: 175 - protein, 10 aa, Artificial Sequence
  CDR1
WDVHKINFYT

SEQ ID NO: 176 - protein, 10 aa, Artificial Sequence
  CDR1
GSVHQINFYG

SEQ ID NO: 177 - protein, 10 aa, Artificial Sequence
  CDR1
GDVHQLNFLG

SEQ ID NO: 178 - protein, 10 aa, Artificial Sequence
  CDR1
WDVHKLNFYG

SEQ ID NO: 179 - protein, 10 aa, Artificial Sequence
  CDR1
YDVHQINFYG

SEQ ID NO: 180 - protein, 10 aa, Artificial Sequence
  CDR1
WDVHQINFLG

SEQ ID NO: 181 - protein, 10 aa, Artificial Sequence
  CDR1
WDVHQLNFYG

SEQ ID NO: 182 - protein, 10 aa, Artificial Sequence
  CDR1
YDVHQINFLG

SEQ ID NO: 183 - protein, 10 aa, Artificial Sequence
  CDR1
WSVHQINFYG

SEQ ID NO: 184 - protein, 10 aa, Artificial Sequence
  CDR1
GSVHKINFYG

SEQ ID NO: 185 - protein, 10 aa, Artificial Sequence
  CDR1
GYVHKLNFLG

SEQ ID NO: 186 - protein, 10 aa, Artificial Sequence
  CDR1
GYVHKLNFYG

SEQ ID NO: 187 - protein, 10 aa, Artificial Sequence
  CDR1
GSVHQLNFLG

SEQ ID NO: 188 - protein, 10 aa, Artificial Sequence
  CDR1
GAVHKLNFLG

SEQ ID NO: 189 - protein, 10 aa, Artificial Sequence
  CDR1
GSVHQINFLG

SEQ ID NO: 190 - protein, 10 aa, Artificial Sequence
  CDR1
GSVHQLNFYG

SEQ ID NO: 191 - protein, 10 aa, Artificial Sequence
  CDR1
GAVHQINFYG

SEQ ID NO: 192 - protein, 10 aa, Artificial Sequence
  CDR1
GAVHQINFLG

SEQ ID NO: 193 - protein, 10 aa, Artificial Sequence
  CDR1
GDVHQINFYG

SEQ ID NO: 194 - protein, 10 aa, Artificial Sequence
  CDR1
GAVHQLNFYG

SEQ ID NO: 195 - protein, 10 aa, Artificial Sequence
  CDR1
GAVHKLNFYG

SEQ ID NO: 196 - protein, 10 aa, Artificial Sequence
  CDR1
GSVHKLNFLG

SEQ ID NO: 197 - protein, 10 aa, Artificial Sequence
  CDR1
GAVHKINFYG

SEQ ID NO: 198 - protein, 10 aa, Artificial Sequence
  CDR1
GSVHKLNFYG

SEQ ID NO: 199 - protein, 10 aa, Artificial Sequence
  CDR1
GYVHQINFLG

SEQ ID NO: 200 - protein, 10 aa, Artificial Sequence
  CDR1
WSVHQLNFYG

SEQ ID NO: 201 - protein, 10 aa, Artificial Sequence
  CDR1
GYVHQLNFYG

SEQ ID NO: 202 - protein, 10 aa, Artificial Sequence
  CDR1
GYVHQINFYG

SEQ ID NO: 203 - protein, 10 aa, Artificial Sequence
  CDR1
GEVHQINFYG

SEQ ID NO: 204 - protein, 10 aa, Artificial Sequence
  CDR1
WEVHKINFYG

SEQ ID NO: 205 - protein, 10 aa, Artificial Sequence
  CDR1
YAVHQINFYG

SEQ ID NO: 206 - protein, 10 aa, Artificial Sequence
  CDR1
YDVHQLNFYG

SEQ ID NO: 207 - protein, 10 aa, Artificial Sequence
  CDR1
WEVHQINFYG

SEQ ID NO: 208 - protein, 10 aa, Artificial Sequence
  CDR1
YSVHQLNFLG

SEQ ID NO: 209 - protein, 9 aa, Artificial Sequence
  CDR2
HISIGDQTD

SEQ ID NO: 210 - protein, 9 aa, Artificial Sequence
  CDR2
VISIGDQTD

SEQ ID NO: 211 - protein, 9 aa, Artificial Sequence
  CDR2
HISIADQTD

SEQ ID NO: 212 - protein, 9 aa, Artificial Sequence
  CDR2
HISIGDETD

SEQ ID NO: 213 - protein, 9 aa, Artificial Sequence
  CDR2
HISIGDVTD

SEQ ID NO: 214 - protein, 9 aa, Artificial Sequence
  CDR2
HISIGDQVD

SEQ ID NO: 215 - protein, 9 aa, Artificial Sequence
  CDR2
HISIGDQSD

SEQ ID NO: 216 - protein, 9 aa, Artificial Sequence
  CDR2
MISIGDQTD

SEQ ID NO: 217 - protein, 9 aa, Artificial Sequence
  CDR2
HISIGEQTD

SEQ ID NO: 218 - protein, 9 aa, Artificial Sequence
  CDR2
HISIGDHTD

SEQ ID NO: 219 - protein, 9 aa, Artificial Sequence
  CDR2
HISIGDKTD

SEQ ID NO: 220 - protein, 9 aa, Artificial Sequence
  CDR2
HISIGDRTD

SEQ ID NO: 221 - protein, 9 aa, Artificial Sequence
  CDR2
HISIGDQAD

SEQ ID NO: 222 - protein, 9 aa, Artificial Sequence
  CDR2
HISIGDQTN

SEQ ID NO: 223 - protein, 9 aa, Artificial Sequence
  CDR3
FSRIYPYDY

SEQ ID NO: 224 - protein, 9 aa, Artificial Sequence
  CDR3
LSRIYPYDY

SEQ ID NO: 225 - protein, 9 aa, Artificial Sequence
  CDR3
FSRIWPYDY

SEQ ID NO: 226 - protein, 9 aa, Artificial Sequence
  CDR3
MSRIYPYDY

SEQ ID NO: 227 - protein, 9 aa, Artificial Sequence
  CDR3
FSKIYPYDY

SEQ ID NO: 228 - protein, 9 aa, Artificial Sequence
  CDR3
FSRIYPYDW

SEQ ID NO: 229 - protein, 9 aa, Artificial Sequence
  CDR3
FSSIYPYDY

SEQ ID NO: 230 - protein, 9 aa, Artificial Sequence
  CDR3
FSRIYPYQY

SEQ ID NO: 231 - protein, 9 aa, Artificial Sequence
  CDR3
FSRIYPYSY

SEQ ID NO: 232 - protein, 9 aa, Artificial Sequence
  CDR3
FSRIYPYLY

SEQ ID NO: 233 - protein, 9 aa, Artificial Sequence
  CDR3
FSRIYPYAY

SEQ ID NO: 234 - protein, 9 aa, Artificial Sequence
  CDR3
FSRIYPYTY

SEQ ID NO: 235 - protein, 9 aa, Artificial Sequence
  CDR3
LSRIWPYDY

SEQ ID NO: 236 - protein, 9 aa, Artificial Sequence
  CDR3
LSKIWPYDY

SEQ ID NO: 237 - protein, 9 aa, Artificial Sequence
  CDR3
MSKIWPYDY

SEQ ID NO: 238 - protein, 9 aa, Artificial Sequence
  CDR3
FSKIWPYDY

SEQ ID NO: 239 - protein, 9 aa, Artificial Sequence
  CDR3
MSKIYPYDY

SEQ ID NO: 240 - protein, 9 aa, Artificial Sequence
  CDR3
MSRIWPYDY

SEQ ID NO: 241 - protein, 9 aa, Artificial Sequence
  CDR3
FSKIWPYDW

SEQ ID NO: 242 - protein, 9 aa, Artificial Sequence
  CDR3
MSRIWPYDW

SEQ ID NO: 243 - protein, 9 aa, Artificial Sequence
  CDR3
FSRIWPYDW

SEQ ID NO: 244 - protein, 9 aa, Artificial Sequence
  CDR3
MSKIWPYDW

SEQ ID NO: 245 - protein, 9 aa, Artificial Sequence
  CDR3
MSKIYPYDW

SEQ ID NO: 246 - protein, 9 aa, Artificial Sequence
  CDR3
MSRIYPYDW

SEQ ID NO: 247 - protein, 9 aa, Artificial Sequence
  CDR3
LSKIYPYDY

SEQ ID NO: 248 - protein, 25 aa, Artificial Sequence
  FR1
EVQLVESGGGLVQPGGSLRLSCVAS

SEQ ID NO: 249 - protein, 25 aa, Artificial Sequence
  FR1
EVQLVESGGGVVQPGGSLRLSCVAS

SEQ ID NO: 250 - protein, 25 aa, Artificial Sequence
  FR1
DVQLVESGGGVVQPGGSLRLSCVAS

SEQ ID NO: 251 - protein, 14 aa, Artificial Sequence
  FR2
WYRQAPGKEREKVA

SEQ ID NO: 252 - protein, 39 aa, Artificial Sequence
  FR3
YADSAKGRFTISRDESKNMVYLQMNSLKPEDTAVYFCRA

SEQ ID NO: 253 - protein, 39 aa, Artificial Sequence
  FR3
YADSAKGRFTISRDESKNMVYLQMNSLRPEDTALYFCRA

SEQ ID NO: 254 - protein, 39 aa, Artificial Sequence
  FR3
YADSAKGRFTISRDESKNTVYLQMNSLRPEDTALYFCRA

SEQ ID NO: 255 - protein, 39 aa, Artificial Sequence
  FR3
YADSAKGRFTISRDESKNMVYLQMNSLRPEDTALYYCRA

SEQ ID NO: 256 - protein, 39 aa, Artificial Sequence
  FR3
YADSAKGRFTISRDESKNTVYLQMNSLRPEDTALYYCRA

SEQ ID NO: 257 - protein, 39 aa, Artificial Sequence
  FR3
YADSAKDRFTISRDESKNMVYLQMNSLRPEDTALYFCRA

SEQ ID NO: 258 - protein, 39 aa, Artificial Sequence
  FR3
YADSAKGRFTISRDESKNTVYLQMNSLRPEDTAAYYCRA

SEQ ID NO: 259 - protein, 39 aa, Artificial Sequence
  FR3
YADSAKGRFTISRDESKNTVYLQMNSLRPEDTATYYCRA

SEQ ID NO: 260 - protein, 11 aa, Artificial Sequence
  FR4
WGQGTLVTVSS

SEQ ID NO: 261 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 262 - protein, 117 aa, Artificial Sequence
  monovalent ISVD
EVQLVESGGGVVQPGGSLRLSCVASGYVHKINFYGWYRQAPGKEREKVAHISIGDQTDYADSAKGRFTISRDESKNTVYLQMNSLRPEDTATYYCRALSRIWPYDYWGQGTLVTVSS

SEQ ID NO: 263 - protein, 115 aa, Artificial Sequence
  monovalent ISVD
EVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSS

SEQ ID NO: 264 - protein, 133 aa, Artificial Sequence
  monovalent ISVD
DVQLQASGGGSVQAGGSLRLSCAASGYTIGPYCMGWFRQAPGKEREGVAAINMGGGITYYADSVKGRFTISQDNAKNTVYLLMNSLEPEDTAIYYCAADSTIYASYYECGHGLSTGGYGYDSWGQGTQVTVSS

SEQ ID NO: 265 - protein, 125 aa, Artificial Sequence
  monovalent ISVD
EVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSS

SEQ ID NO: 266 - protein, 127 aa, Artificial Sequence
  monovalent ISVD
EVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSS

SEQ ID NO: 267 - protein, 133 aa, Artificial Sequence; identical to SEQ ID NO: 264
  monovalent ISVD

SEQ ID NO: 268 - protein, 268 aa, Artificial Sequence
  ISVD format
DVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 269 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSA

SEQ ID NO: 270 - protein, 428 aa, Artificial Sequence
  ISVD format
DVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSGGGGSGGGGSGGGGSGGGGSGGGGSGGGGSEVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 271 - protein, 268 aa, Artificial Sequence
  ISVD format
DVQLVESGGGVVQPGGSLRLSCVASWDVHKINFYGWYRQAPGKEREKVAHISIGDQTDYADSAKGRFTISRDESKNTVYLQMNSLRPEDTALYYCRALSKIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 272 - protein, 268 aa, Artificial Sequence
  ISVD format
DVQLVESGGGVVQPGGSLRLSCVASYDVHKINFYGWYRQAPGKEREKVAHISIGDQTDYADSAKGRFTISRDESKNTVYLQMNSLRPEDTALYYCRALSKIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 273 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASWDVHKINFYGWYRQAPGKEREKVAHISIGDQTDYADSAKGRFTISRDESKNTVYLQMNSLRPEDTALYYCRALSKIWPYDYWGQGTLVTVSSA

SEQ ID NO: 274 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASYDVHKINFYGWYRQAPGKEREKVAHISIGDQTDYADSAKGRFTISRDESKNTVYLQMNSLRPEDTALYYCRALSKIWPYDYWGQGTLVTVSSA

SEQ ID NO: 275 - protein, 268 aa, Artificial Sequence
  ISVD format
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 276 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSA

SEQ ID NO: 277 - protein, 268 aa, Artificial Sequence
  ISVD format
DVQLVESGGGVVQPGGSLRLSCVASWDVHQINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 278 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASWDVHQINFYGWYRQAPGKEREKVAHISIGDQTDYADSAKGRFTISRDESKNTVYLQMNSLRPEDTALYYCRALSRIWPYDYWGQGTLVTVSSA

SEQ ID NO: 279 - protein, 268 aa, Artificial Sequence
  ISVD format
DVQLVESGGGVVQPGGSLRLSCVASWDVHKINFYTWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 280 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASWDVHKINFYTWYRQAPGKEREKVAHISIGDQTDYADSAKGRFTISRDESKNTVYLQMNSLRPEDTALYYCRALSRIWPYDYWGQGTLVTVSSA

SEQ ID NO: 281 - protein, 268 aa, Artificial Sequence
  ISVD format
DVQLVESGGGVVQPGGSLRLSCVASWDVHKINFYTWYRQAPGKEREKVAHISIGDQTDYADSAKGRFTISRDESKNTVYLQMNSLRPEDTALYYCRALSKIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 282 - protein, 268 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASWDVHKINFYTWYRQAPGKEREKVAHISIGDQTDYADSAKGRFTISRDESKNTVYLQMNSLRPEDTALYYCRALSKIWPYDYWGQGTLVTVSSA

SEQ ID NO: 283 - protein, 428 aa, Artificial Sequence
  ISVD format
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 284 - protein, 428 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSSA

SEQ ID NO: 285 - protein, 442 aa, Artificial Sequence
  ISVD format
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSSGGGGSGGGGSGGGGSGGGGSGGGGSGGGGSGGGGSEVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSSA

SEQ ID NO: 286 - protein, 442 aa, Artificial Sequence
  ISVD format
DVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSSA

SEQ ID NO: 287 - protein, 442 aa, Artificial Sequence
  ISVD format
DVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSSGGGGSGGGGSGGGGSGGGGSGGGGSGGGGSGGGGSEVQLVESGGGLVQAGDSLRLSCAASGRTFSSYAMGWFRQAPGKEREFVAAISWSDGSTYYADSVKGRFTISRDNAKNTVYLQMNSLKPEDTAVYYCAADLTSTNPGSYIYIWAYDYWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSA

SEQ ID NO: 288 - protein, 428 aa, Artificial Sequence
  ISVD format
DVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSA

SEQ ID NO: 289 - protein, 428 aa, Artificial Sequence
  ISVD format
DVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSGGGGSGGGGSGGGGSGGGGSGGGGSGGGGSGGGGSEVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSA

SEQ ID NO: 290 - protein, 428 aa, Artificial Sequence
  ISVD format
DVQLVESGGGLVQAGGPLRLSCAASGRTFSSYVMGWFRQAPGKEREFVAAIYWSNGKTQYTDSVKGRFTISGDNAKNTVYLQMNSLNPEDTAVYYCVADKDETGFRTLPIAYDYWGQGTLVTVSSGGGGSGGGGSGGGGSGGGGSGGGGSGGGGSGGGGSEVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQAGGSLRLSCAASGITSKINDMGWYRQTPGNYREWVASITATGTTNYRDSVKGRFTISRDNAKSTVYLQMNSLKPEDTTVYYCNTFPPISNFWGQGTLVTVSSA

SEQ ID NO: 291 - protein, 96 aa, Artificial Sequence
  Human TCR alpha constant domain (derived from P01848)
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSC

SEQ ID NO: 292 - protein, 131 aa, Artificial Sequence
  Human TCR beta constant domain (derived from P01850)
EDLNKVFPPEVAVFEPSEAEISHTQKATLVCLATGFFPDHVELSWWVNGKEVHSGVSTDPQPLKEQPALNDSRYALSSRLRVSATFWQNPRNHFRCQVQFYGLSENDEWTQDRAKPVTQIVSAEAWGRADC

SEQ ID NO: 293 - protein, 96 aa, Artificial Sequence
  cynomolgus TCR-alpha constant domain (derived from AEA41865)
PYIQNPDPAVYQLRGSKSNDTSVCLFTDFDSVMNVSQSKDSDVHITDKTVLDMRSMDFKSNGAVAWSNKSDFACTSAFKDSVIPADTFFPSPESSC

SEQ ID NO: 294 - protein, 131 aa, Artificial Sequence
  cynomolgus TCR-beta constant domain (derived from AEA41868)
EDLKKVFPPKVAVFEPSEAEISHTQKATLVCLATGFYPDHVELSWWVNGKEVHSGVSTDPQPLKEQPALEDSRYSLSSRLRVSATFWHNPRNHFRCQVQFYGLSEDDEWTEDRDKPITQKISAEAWGRADC

SEQ ID NO: 295 - protein, 10 aa, Artificial Sequence
  CDR1
  VARIANT (1)..(1): Xaa at position 1 is Gly, Trp, Gln, or Tyr
  VARIANT (2)..(2): Xaa at position 2 is Asp, Glu, Tyr, Ser, or Ala
  VARIANT (5)..(5): Xaa at position 5 is Lys, or Gln
  VARIANT (6)..(6): Xaa at position 6 is Ile, or Leu
  VARIANT (7)..(7): Xaa at position 7 is Asn, or Phe
  VARIANT (8)..(8): Xaa at position 8 is Phe, Val, or Tyr
  VARIANT (9)..(9): Xaa at position 9 is Leu, Tyr, or Phe
  VARIANT (10)..(10): Xaa at position 10 is Gly, Thr, or Arg
XXVHXXXXXX

SEQ ID NO: 296 - protein, 9 aa, Artificial Sequence
  CDR2
  VARIANT (1)..(1): Xaa at position 1 is His, or Val
  VARIANT (5)..(5): Xaa at position 5 is Gly, or Ala
  VARIANT (7)..(7): Xaa at position 7 is Gln, Val, or Glu
  VARIANT (8)..(8): Xaa at position 8 is Thr, Ser, or Val
XISIXDXXD

SEQ ID NO: 297 - protein, 9 aa, Artificial Sequence
  CDR3
  VARIANT (1)..(1): Xaa at position 1 is Phe, Leu or Met
  VARIANT (3)..(3): Xaa at position 3 is Arg, Lys, or Ser
  VARIANT (5)..(5): Xaa at position 5 is Tyr, or Trp
  VARIANT (8)..(8): Xaa at position 8 is Asp, or Gln
  VARIANT (9)..(9): Xaa at position 9 is Tyr, or Trp
XSXIXPYXX

SEQ ID NO: 298 - protein, 10 aa, Artificial Sequence
  CDR1
  VARIANT (1)..(1): Xaa at position 1 is Gly, Trp, or Tyr
  VARIANT (2)..(2): Xaa at position 2 is Asp, Glu, Tyr, Ser, or Ala
  VARIANT (5)..(5): Xaa at position 5 is Lys, or Gln
  VARIANT (6)..(6): Xaa at position 6 is Ile, or Leu
  VARIANT (9)..(9): Xaa at position 9 is Leu, or Tyr
  VARIANT (10)..(10): Xaa at position 10 is Gly, or Thr
XXVHXXNFXX

SEQ ID NO: 299 - protein, 9 aa, Artificial Sequence
  CDR3
  VARIANT (1)..(1): Xaa at position 1 is Phe, Leu or Met
  VARIANT (3)..(3): Xaa at position 3 is Arg, or Lys
  VARIANT (5)..(5): Xaa at position 5 is Tyr, or Trp
  VARIANT (9)..(9): Xaa at position 9 is Tyr, or Trp
XSXIXPYDX

SEQ ID NO: 300 - protein, 171 aa, Artificial Sequence
  Human CD3 delta (P04234)
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 301 - protein, 180 aa, Artificial Sequence
  Human CD3 gamma (derived from P09693)
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLR

SEQ ID NO: 302 - protein, 205 aa, Artificial Sequence
  Human CD3 epsilon (derived from P07766)
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQR

SEQ ID NO: 303 - protein, 164 aa, Artificial Sequence
  Human CD3 zeta (P20963)
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 304 - protein, 142 aa, Artificial Sequence
  Human TCR alpha constant domain (derived from P01848)
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 305 - protein, 177 aa, Artificial Sequence
  Human TCR beta constant domain (derived from P01850)
EDLNKVFPPEVAVFEPSEAEISHTQKATLVCLATGFFPDHVELSWWVNGKEVHSGVSTDPQPLKEQPALNDSRYALSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 306 - protein, 107 aa, Artificial Sequence
  Human TCR alpha variable domain derived from 2IAN
IQVEQSPPDLILQEGANSTLRCNFSDSVNNLQWFHQNPWGQLINLFYIPSGTKQNGRLSATTVATERYSLLYISSSQTTDSGVYFCAALIQGAQKLVFGQGTRLTIN

SEQ ID NO: 307 - protein, 108 aa, Artificial Sequence
  Human TCR alpha variable domain derived from 2XN9
QLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVK

SEQ ID NO: 308 - protein, 110 aa, Artificial Sequence
  Human TCR alpha variable domain derived from 3TOE
GDAKTTQPNSMESNEEEPVHLPCNHSTISGTDYIHWYRQLPSQGPEYVIHGLTSNVNNRMASLAIAEDRKSSTLILHRATLRDAAVYYCTVYGGATNKLIFGTGTLLAVQ

SEQ ID NO: 309 - protein, 110 aa, Artificial Sequence
  Human TCR beta variable domain derived from 2IAN
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASTYHGTGYFGEGSWLTVV

SEQ ID NO: 310 - protein, 112 aa, Artificial Sequence
  Human TCR beta variable domain derived from 2XN9
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVT

SEQ ID NO: 311 - protein, 114 aa, Artificial Sequence
  Human TCR beta variable domain derived from 3TOE
VVSQHPSWVIAKSGTSVKIECRSLDFQATTMFWYRQFPKQSLMLMATSNEGSKATYEQGVEKDKFLINHASLTLSTLTVTSAHPEDSSFYICSARGGSYNSPLHFGNGTRLTVT

SEQ ID NO: 312 - protein, 171 aa, Artificial Sequence
  Cyno CD3 delta (NP_001274617)
MEHSTFLSGLVLATLLSQVSPFKIPVEELEDRVFVKCNTSVTWVEGTVGTLLTNNTRLDLGKRILDPRGIYRCNGTDIYKDKESAVQVHYRMCQNCVELDPATLAGIIVTDVIATLLLALGVFCFAGHETGRLSGAADTQALLRNDQVYQPLRDRDDAQYSRLGGNWARNK

SEQ ID NO: 313 - protein, 181 aa, Artificial Sequence
  Cyno CD3 gamma (BAJ16168)
MEQGKGLTGFILAIILLQGSLAQSFEENRKLNVYNQEDGSVLLTCHVKNTNITWFKEGKMIDILTAHKNKWNLGSNTKDPRGVYQCKGSKDKSKTLQVYYRMCQNCIELNAATILGFVFAEIISIFFLAVGVYFIAGQDGVRQSRASDKQTLLPNDQLYQPLKDREDDQYSHLQGNQLRRN

SEQ ID NO: 314 - protein, 198 aa, Artificial Sequence
  Cyno CD3 epsilon (Q95LI5)
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 315 - protein, 164 aa, Artificial Sequence
  Cyno CD3 zeta (XP_005539936)
MKWKELVTAAILQAQFPITEAQSFGLLDPKLCYLLDGILFLYGVILTALFLRAKFSRSADAPAYQQGQNQLYNELNLGRREEYDVLDKRRGRDPEMGGKPQRRKNPQEGLYNALQKDKMAEAYSEIGMKGERRRGKGHDGLYQGLSTATKDTYDALHMQTLPPR

SEQ ID NO: 316 - protein, 270 aa, Artificial Sequence
  Rhesus TCR alpha (AEA41863)
MLLLLVLVLEVIFTLGGTRAQSVTQLDSQVSVSEGVPVLLRCNYSSSFSPYLFWYVQYPNQGLQLLLKYTSGTTLVKGINGFEAEFKKSETSFHLTKASAHVSDAAEYFCALARGALVFGKGTRLSVIPNIQNPDPAVYQLRGSKSNDTSVCLFTDFDSVMNVSQSKDSDVHITDKTVLDMRSMDFKSNGAVAWSNKSDFACTSAFKDSVIPADTFFPGTESVCDANLVEKSFETDMNLNFQNLSVIGFRILLLKVAGFNLLMTLRLWSS

SEQ ID NO: 317 - protein, 310 aa, Artificial Sequence
  Rhesus TCR beta (AEA41864)
MGFWTLCCVSFCILVAKHTDAGVIQLPRHEVTEMGKEVTLRCEPISGHSSLFWYRQTMMRGLEFLIYFNNKSPIDDSGMPKDRFSATMPDASFSTLKIQPSEPRDSAVYFCASTPGQGREKLFFGSGTQLSVLEDLKKVFPPKVAVFEPSEAEISHTQKATLVCLATGFYPDHVELSWWVNGKEVHSGVSTDPQPLKEQPALEDSRYCLSSRLRVSATFWHNPRNHFRCQVQFYGLSEDDEWTEDRDKPITQKISAEVWGRADCGFTSVSYQQGVLSATILYEILLGKATLYAVLVSALMLMAMVKRKDF

SEQ ID NO: 318 - protein, 281 aa, Artificial Sequence
  Human TCR-zipper alpha chain
MNMRPVTSSVLVLLLMLRRSNGQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKSVLDMRSMDFKSNSAVAWSNKSDFACANAFNNSIIPEDTFFPSPESSCSSADLVPRGSTTAPSAQLKKKLQALKKKNAQLKWKLQALKKKLAQEQKLISEEDL

SEQ ID NO: 319 - protein, 329 aa, Artificial Sequence
  Human TCR-zipper beta chain
MSNTVLADSAWGITLLSWVTVFLLGTSSADGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCSSADLVPRGSTTAPSAQLEKELQALEKENAQLEWELQALEKELAQTGHHHHHHHHHH

SEQ ID NO: 320 - protein, 277 aa, Artificial Sequence
  Cyno TCR-zipper alpha chain (derived from AEA41865)
MLLITSVLVLWMQLSQVNGQQIMQIPQYQHVQEGEDFTTYCNSSTTLSNIQWYKQRPGGHPVFLIMLVKSGEVKKQKRLIFQFGEAKKNSSLHITATQTTDVGTYFCATTGVNNLFFGTGTRLTVLPYIQNPDPAVYQLRGSKSNDTSVCLFTDFDSVMNVSQSKDSDVHITDKTVLDMRSMDFKSNGAVAWSNKSDFACTSAFKDSVIPADTFFPSPESSCSSADLVPRGSTTAPSAQLKKKLQALKKKNAQLKWKLQALKKKLAQEQKLISEEDL

SEQ ID NO: 321 - protein, 321 aa, Artificial Sequence
  Cyno TCR-zipper beta chain (derived from AEA41866 and AEA41868)
MSIGLLCCAALSLLWAGPVNAGVTQTPKFQVLKTGQSMTLQCAQDMNHDYMYWYRQDPGMGLRLIHYSVGEGSTEKGEVPDGYNVTRSNTEDFPLRLESAAPSQTSVYFCASSYWTGRSYEQYFGPGTRLTVIEDLKKVFPPKVAVFEPSEAEISHTQKATLVCLATGFYPDHVELSWWVNGKEVHSGVSTDPQPLKEQPALEDSRYSLSSRLRVSATFWHNPRNHFRCQVQFYGLSEDDEWTEDRDKPITQKISAEAWGRADCSSADLVPRGSTTAPSAQLEKELQALEKENAQLEWELQALEKELAQTGHHHHHHHHHH

SEQ ID NO: 322 - protein, 3 aa, Artificial Sequence
  Linker
AAA

SEQ ID NO: 323 - protein, 5 aa, Artificial Sequence
  Linker
GGGGS

SEQ ID NO: 324 - protein, 7 aa, Artificial Sequence
  Linker
SGGSGGS

SEQ ID NO: 325 - protein, 8 aa, Artificial Sequence
  Linker
GGGGSGGS

SEQ ID NO: 326 - protein, 9 aa, Artificial Sequence
  Linker
GGGGSGGGS

SEQ ID NO: 327 - protein, 10 aa, Artificial Sequence
  Linker
GGGGSGGGGS

SEQ ID NO: 328 - protein, 15 aa, Artificial Sequence
  Linker
GGGGSGGGGSGGGGS

SEQ ID NO: 329 - protein, 18 aa, Artificial Sequence
  Linker
GGGGSGGGGSGGGGSGGS

SEQ ID NO: 330 - protein, 20 aa, Artificial Sequence
  Linker
GGGGSGGGGSGGGGSGGGGS

SEQ ID NO: 331 - protein, 25 aa, Artificial Sequence
  Linker
GGGGSGGGGSGGGGSGGGGSGGGGS

SEQ ID NO: 332 - protein, 30 aa, Artificial Sequence
  Linker
GGGGSGGGGSGGGGSGGGGSGGGGSGGGGS

SEQ ID NO: 333 - protein, 35 aa, Artificial Sequence
  Linker
GGGGSGGGGSGGGGSGGGGSGGGGSGGGGSGGGGS

SEQ ID NO: 334 - protein, 40 aa, Artificial Sequence
  Linker
GGGGSGGGGSGGGGSGGGGSGGGGSGGGGSGGGGSGGGGS

SEQ ID NO: 335 - protein, 15 aa, Artificial Sequence
  Linker
EPKSCDKTHTCPPCP

SEQ ID NO: 336 - protein, 24 aa, Artificial Sequence
  Linker
GGGGSGGGSEPKSCDKTHTCPPCP

SEQ ID NO: 337 - protein, 12 aa, Artificial Sequence
  Linker
EPKTPKPQPAAA

SEQ ID NO: 338 - protein, 62 aa, Artificial Sequence
  Linker
ELKTPLGDTTHTCPRCPEPKSCDTPPPCPRCPEPKSCDTPPPCPRCPEPKSCDTPPPCPRCP

SEQ ID NO: 339 - protein, 5 aa, Artificial Sequence
  C-terminus
VTVSS

SEQ ID NO: 340 - protein, 5 aa, Artificial Sequence
  C-terminus
VKVSS

SEQ ID NO: 341 - protein, 5 aa, Artificial Sequence
  C-terminus
VQVSS

SEQ ID NO: 342 - protein, 5 aa, Artificial Sequence
  C-terminus
VTVKS

SEQ ID NO: 343 - protein, 5 aa, Artificial Sequence
  C-terminus
VTVQS

SEQ ID NO: 344 - protein, 5 aa, Artificial Sequence
  C-terminus
VKVKS

SEQ ID NO: 345 - protein, 5 aa, Artificial Sequence
  C-terminus
VKVQS

SEQ ID NO: 346 - protein, 5 aa, Artificial Sequence
  C-terminus
VQVKS

SEQ ID NO: 347 - protein, 5 aa, Artificial Sequence
  C-terminus
VQVQS

SEQ ID NO: 348 - protein, 6 aa, Artificial Sequence
  C-terminus
VTVSSA

SEQ ID NO: 349 - protein, 6 aa, Artificial Sequence
  C-terminus
VKVSSA

SEQ ID NO: 350 - protein, 6 aa, Artificial Sequence
  C-terminus
VQVSSA

SEQ ID NO: 351 - protein, 6 aa, Artificial Sequence
  C-terminus
VTVKSA

SEQ ID NO: 352 - protein, 6 aa, Artificial Sequence
  C-terminus
VTVQSA

SEQ ID NO: 353 - protein, 6 aa, Artificial Sequence
  C-terminus
VKVKSA

SEQ ID NO: 354 - protein, 6 aa, Artificial Sequence
  C-terminus
VKVQSA

SEQ ID NO: 355 - protein, 6 aa, Artificial Sequence
  C-terminus
VQVKSA

SEQ ID NO: 356 - protein, 6 aa, Artificial Sequence
  C-terminus
VQVQSA

SEQ ID NO: 357 - protein, 10 aa, Artificial Sequence
  CDR1
GFTFRSFGMS

SEQ ID NO: 358 - protein, 10 aa, Artificial Sequence
  CDR1
GFTFSSFGMS

SEQ ID NO: 359 - protein, 10 aa, Artificial Sequence
  CDR2
SISGSGSDTL

SEQ ID NO: 360 - protein, 6 aa, Artificial Sequence
  CDR3
GGSLSR

SEQ ID NO: 361 - protein, 115 aa, Artificial Sequence [sequence omitted]

SEQ ID NO: 362 - protein, 116 aa, Artificial Sequence
  Serum albumin binding ISVD
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSA

SEQ ID NO: 363 - protein, 115 aa, Artificial Sequence [sequence omitted]

SEQ ID NO: 364 - protein, 116 aa, Artificial Sequence
  Serum albumin binding ISVD
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSA

SEQ ID NO: 365 - protein, 115 aa, Artificial Sequence
  Serum albumin binding ISVD
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSS

SEQ ID NO: 366 - protein, 116 aa, Artificial Sequence
  Serum albumin binding ISVD
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 367 - protein, 115 aa, Artificial Sequence
  Serum albumin binding ISVD
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSS

SEQ ID NO: 368 - protein, 116 aa, Artificial Sequence
  Serum albumin binding ISVD
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 369 - protein, 115 aa, Artificial Sequence
  Serum albumin binding ISVD
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSS

SEQ ID NO: 370 - protein, 116 aa, Artificial Sequence
  Serum albumin binding ISVD
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 371 - protein, 115 aa, Artificial Sequence
  Serum albumin binding ISVD
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 372 - protein, 116 aa, Artificial Sequence
  Serum albumin binding ISVD
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 373 - protein, 115 aa, Artificial Sequence
  Serum albumin binding ISVD
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVKVSS

SEQ ID NO: 374 - protein, 116 aa, Artificial Sequence
  Serum albumin binding ISVD
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVKVSSA

SEQ ID NO: 375 - protein, 115 aa, Artificial Sequence
  Serum albumin binding ISVD
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 376 - protein, 116 aa, Artificial Sequence
  Serum albumin binding ISVD
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 377 - protein, 115 aa, Artificial Sequence
  Serum albumin binding ISVD
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVKVSS

SEQ ID NO: 378 - protein, 116 aa, Artificial Sequence
  Serum albumin binding ISVD
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVKVSSA

SEQ ID NO: 379 - protein, 378 aa, Artificial Sequence
  Human CD123 (NP_002174)
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 380 - protein, 4 aa, Artificial Sequence
  Motif from Table B-2
KERE

SEQ ID NO: 381 - protein, 4 aa, Artificial Sequence
  Motif from Table B-2
KQRE

SEQ ID NO: 382 - protein, 4 aa, Artificial Sequence
  Motif from Table B-2
GLEW

SEQ ID NO: 383 - protein, 5 aa, Artificial Sequence
  Motif from Table B-2
KEREL

SEQ ID NO: 384 - protein, 5 aa, Artificial Sequence
  Motif from Table B-2
KEREF

SEQ ID NO: 385 - protein, 5 aa, Artificial Sequence
  Motif from Table B-2
KQREL

SEQ ID NO: 386 - protein, 5 aa, Artificial Sequence
  Motif from Table B-2
KQREF

SEQ ID NO: 387 - protein, 5 aa, Artificial Sequence
  Motif from Table B-2
KEREG

SEQ ID NO: 388 - protein, 5 aa, Artificial Sequence
  Motif from Table B-2
KQREW

SEQ ID NO: 389 - protein, 5 aa, Artificial Sequence
  Motif from Table B-2
KQREG

SEQ ID NO: 390 - protein, 4 aa, Artificial Sequence
  Motif from Table B-2
TERE

SEQ ID NO: 391 - protein, 5 aa, Artificial Sequence
  Motif from Table B-2
TEREL

SEQ ID NO: 392 - protein, 4 aa, Artificial Sequence
  Motif from Table B-2
TQRE

SEQ ID NO: 393 - protein, 5 aa, Artificial Sequence
  Motif from Table B-2
TQREL

SEQ ID NO: 394 - protein, 4 aa, Artificial Sequence
  Motif from Table B-2
KECE

SEQ ID NO: 395 - protein, 5 aa, Artificial Sequence
  Motif from Table B-2
KECEL

SEQ ID NO: 396 - protein, 5 aa, Artificial Sequence
  Motif from Table B-2
KECER

SEQ ID NO: 397 - protein, 4 aa, Artificial Sequence
  Motif from Table B-2
KQCE

SEQ ID NO: 398 - protein, 5 aa, Artificial Sequence
  Motif from Table B-2
KQCEL

SEQ ID NO: 399 - protein, 4 aa, Artificial Sequence
  Motif from Table B-2
RERE

SEQ ID NO: 400 - protein, 5 aa, Artificial Sequence
  Motif from Table B-2
REREG

SEQ ID NO: 401 - protein, 4 aa, Artificial Sequence
  Motif from Table B-2
RQRE

SEQ ID NO: 402 - protein, 5 aa, Artificial Sequence
  Motif from Table B-2
RQREL

SEQ ID NO: 403 - protein, 5 aa, Artificial Sequence
  Motif from Table B-2
RQREF

SEQ ID NO: 404 - protein, 5 aa, Artificial Sequence
  Motif from Table B-2
RQREW

SEQ ID NO: 405 - protein, 4 aa, Artificial Sequence
  Motif from Table B-2
QERE

SEQ ID NO: 406 - protein, 5 aa, Artificial Sequence
  Motif from Table B-2
QEREG

SEQ ID NO: 407 - protein, 4 aa, Artificial Sequence
  Motif from Table B-2
QQRE

SEQ ID NO: 408 - protein, 5 aa, Artificial Sequence
  Motif from Table B-2
QQREW

SEQ ID NO: 409 - protein, 5 aa, Artificial Sequence
  Motif from Table B-2
QQREL

SEQ ID NO: 410 - protein, 5 aa, Artificial Sequence
  Motif from Table B-2
QQREF

SEQ ID NO: 411 - protein, 4 aa, Artificial Sequence
  Motif from Table B-2
KGRE

SEQ ID NO: 412 - protein, 5 aa, Artificial Sequence
  Motif from Table B-2
KGREG

SEQ ID NO: 413 - protein, 4 aa, Artificial Sequence
  Motif from Table B-2
KDRE

SEQ ID NO: 414 - protein, 5 aa, Artificial Sequence
  Motif from Table B-2
KDREV

SEQ ID NO: 415 - protein, 5 aa, Artificial Sequence
  Motif from Table B-2
DECKL

SEQ ID NO: 416 - protein, 5 aa, Artificial Sequence
  Motif from Table B-2
NVCEL

SEQ ID NO: 417 - protein, 4 aa, Artificial Sequence
  Motif from Table B-2
GVEW

SEQ ID NO: 418 - protein, 4 aa, Artificial Sequence
  Motif from Table B-2
EPEW

SEQ ID NO: 419 - protein, 4 aa, Artificial Sequence
  Motif from Table B-2
GLER

SEQ ID NO: 420 - protein, 4 aa, Artificial Sequence
  Motif from Table B-2
DQEW

SEQ ID NO: 421 - protein, 4 aa, Artificial Sequence
  Motif from Table B-2
DLEW

SEQ ID NO: 422 - protein, 4 aa, Artificial Sequence
  Motif from Table B-2
GIEW

SEQ ID NO: 423 - protein, 4 aa, Artificial Sequence
  Motif from Table B-2
ELEW

SEQ ID NO: 424 - protein, 4 aa, Artificial Sequence
  Motif from Table B-2
GPEW

SEQ ID NO: 425 - protein, 4 aa, Artificial Sequence
  Motif from Table B-2
EWLP

SEQ ID NO: 426 - protein, 4 aa, Artificial Sequence
  Motif from Table B-2
GPER

SEQ ID NO: 427 - protein, 4 aa, Artificial Sequence
  Motif from Table B-2
GLER

SEQ ID NO: 428 - protein, 4 aa, Artificial Sequence
  Motif from Table B-2
ELEW

SEQ ID NO: 429 - protein, 5 aa, Artificial Sequence
  CDR
  VARIANT (2)..(2): Xaa at position 2 is Asp or Glu
  VARIANT (5)..(5): Xaa at position 5 is Lys or Gln
GXVHX

SEQ ID NO: 430 - protein, 5 aa, Artificial Sequence
  CDR
GDVHK

SEQ ID NO: 431 - protein, 5 aa, Artificial Sequence
  CDR
GEVHK

SEQ ID NO: 432 - protein, 5 aa, Artificial Sequence
  CDR
GDVHQ

SEQ ID NO: 433 - protein, 5 aa, Artificial Sequence
  CDR
GEVHQ

SEQ ID NO: 434 - protein, 426 aa, Artificial Sequence
  Multivalent polypeptide
DVQLVESGGGLVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSGGGGSGGGGSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 435 - protein, 394 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSAAAEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 436 - protein, 396 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 437 - protein, 400 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 438 - protein, 411 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSGGGGSGGGGSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 439 - protein, 550 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 440 - protein, 535 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 441 - protein, 524 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 442 - protein, 550 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 443 - protein, 535 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 444 - protein, 524 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTALYYCRALSRIWPYDYWGQGTLVTVSSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 445 - protein, 396 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 446 - protein, 550 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 447 - protein, 520 aa, Artificial Sequence
  Multivalent polypeptide
DVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSEVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 448 - protein, 524 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGGSGGGGSGGGGSGGGGSGGGGSGGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 449 - protein, 498 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSGGGGSGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSA

SEQ ID NO: 450 - protein, 498 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 451 - protein, 524 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGSEVQLVESGGGLVQPGGSLRLSCVASGSIFRSVFSSSTMEWYRQPPGKKRELVARIAPGDGTNYGALYADSVKGRFTISRDDAKKTVDLQMNSLKPEDTGVYFCASGVAWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 452 - protein, 396 aa, Artificial Sequence
  Multivalent polypeptide
DVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSEVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGGSGGGGSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 453 - protein, 370 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGLVQPGGSLRLSCVASGFTFSSFAMTWVRRPPGKGLEWVATITNGGVTSYRDSVKGRFTISRDNAKNTLYLEMTSLNPEDTAVYICANARRTGPRAPTDIGSYRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 454 - protein, 242 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 455 - protein, 381 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGFTFSSFAMTWVRRPPGKGLEWVATITNAGVTSYADSVKGRFTISRDNAKNTLYLQMNSLRPEDTALYICANARRTGPRAPTDIGSYRGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 456 - protein, 498 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGFTFSSFAMTWVRRPPGKGLEWVATITNKGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 457 - protein, 509 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGGSGGGGSGGGGSEVQLVESGGGVVQPGGSLRLSCAASGFTFSSFAMTWVRRPPGKGLEWVATITNAGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 458 - protein, 509 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGGSGGGGSGGGGSEVQLVESGGGVVQPGGSLRLSCAASGFTFSSFAMTWVRRPPGKGLEWVATITNKGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 459 - protein, 498 aa, Artificial Sequence
  Multivalent polypeptide
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGFTFSSFAMTWVRRPPGKGLEWVATITNAGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 460 - protein, 508 aa, Artificial Sequence
  TCR-CD33-CD123 multispecific ISVD construct
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGGVFRLNSMGWFRQAPGKQRELVAIITSDGDTNYADSVKGRFTISRDQAKNTVYLQMNSLRPEDTALYYCQITYSTSSYSFPINTWGQGTLVTVSSGGGGSGGGSEVQLVESGGGVVQPGGSLRLSCAASGVIFSGNVMGWYRRQAPGKEREWVAAIAEGGSILYRDSVKGRFTISRDNAKNTVYLQMNSLRPEDTALYYCNSHPPVLPYWGQGTLVTVSSGGGGSGGGGSGGGGSGGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 461 - protein, 117 aa, Artificial Sequence; identical to SEQ ID NO: 151
  TCR building block

SEQ ID NO: 462 - protein, 116 aa, Artificial Sequence
  monovalent CD123 building block
EVQLVESGGGVVQPGGSLRLSCAASGVIFSGNVMGWYRRQAPGKEREWVAAIAEGGSILYRDSVKGRFTISRDNAKNTVYLQMNSLRPEDTALYYCNSHPPVLPYWGQGTLVTVSS

SEQ ID NO: 463 - protein, 121 aa, Artificial Sequence
  monovalent CD33 building block
EVQLVESGGGVVQPGGSLRLSCAASGGVFRLNSMGWFRQAPGKQRELVAIITSDGDTNYADSVKGRFTISRDQAKNTVYLQMNSLRPEDTALYYCQITYSTSSYSFPINTWGQGTLVTVSS

SEQ ID NO: 464 - protein, 115 aa, Artificial Sequence; identical to SEQ ID NO: 375
  ALB23002